(19) United States
(12) Patent Application Publication
(10) Pub. No.: US 2018/0263985 A1
(43) Pub. Date: Sep. 20, 2018

(54) MODULATORS OF TOLL-LIKE RECEPTORS FOR THE TREATMENT OF HIV

(71) Applicant: Gilead Sciences, Inc., Foster City, CA (US)

(72) Inventors: ROMAS GELEZIUNAS, Belmont, CA (US); JOSEPH E. HESSELGESSER, San Mateo, CA (US); JASMINE KAUR, Fremont, CA (US); JEFFREY PATRICK MURRY, Castro Valley, CA (US); DEREK DEAN SLOAN, Belmont, CA (US)

(21) Appl. No.: 15/759,177

(22) PCT Filed: Sep. 13, 2016
(86) PCT No.: PCT/US2016/051545

§ 371 Date: Mar. 9, 2018

Related U.S. Application Data
(60) Provisional application No. 62/218,858, filed on Sep. 15, 2015.
(60) Provisional application No. 62/382,550, filed on Sep. 1, 2016.

(51) Int. Cl. A61K 31/519; A61P 31/18

(52) U.S. Cl. CPC A61K 31/519; A61K 39/3955; A61K 2300/00; A61P 31/18

(57) ABSTRACT

Provided are methods, uses, pharmaceutical regimens, pharmaceutical compositions, and kits comprising modulators of TLR8 and pharmaceutically acceptable salts thereof, useful in treating HIV infections.

### CROSS REFERENCES TO RELATED APPLICATIONS

This application claims priority to U.S. Provisional Application Nos. 62/382,550, filed Sep. 1, 2016, and 62/218,858, filed Sep. 15, 2015, each of which is incorporated herein in its entirety for all purposes.

### FIELD

This application relates generally to compounds and pharmaceutical compositions which selectively modulate toll-like receptors (such as TLR8) and methods of using such compounds in the treatment of Human Immunodeficiency Virus (HIV) infections.

### BACKGROUND

The innate immune system provides the body with a first line defense against invading pathogens. In an innate immune response, an invading pathogen is recognized by a germline-encoded receptor, the activation of which initiates a signaling cascade that, among other functions, leads to the induction of cytokine expression and stimulation of multiple immune cell subsets. Innate immune system receptors have broad specificity, recognizing molecular structures that are highly conserved among different pathogens. One family of these receptors is known as Toll-like receptors (TLRs), due to their homology with receptors that were first identified and named in Drosophila, and are present in cells such as macrophages, dendritic cells, and epithelial cells.

The toll-like receptor (TLR) family plays a fundamental role in pathogen recognition and activation of innate immunity. Toll-like receptor 8 (TLR8) is predominantly expressed by myeloid immune cells and activation of this receptor stimulates a broad immunological response. Agonists of TLR8 activate myeloid dendritic cells, monocytes, natural killer (NK) cells, leading to the production of proinflammatory cytokines and chemokines, such as interleukin-18 (IL-18), interleukin-12 (IL-12), tumor necrosis factor-alpha (TNF-α), and interferon-gamma (IFN-γ). Such agonists also promote the increased expression of co-stimulatory molecules on CD8⁺ cells, major histocompatibility complex molecules (MATT, NK cells), and chemokine receptors. Collectively, activation of these innate and adaptive immune responses induces an immune response and regulation of these processes may provide a therapeutic benefit in various conditions involving autoimmunity, inflammation, allergy, asthma, graft rejection, graft versus host disease (GvHD), infection, cancer, and immunodeficiency

Around the world more than thirty million people are infected by the HIV virus. Numerous drugs and combination therapies have been developed for the treatment of HIV infections in humans. While combination antiretroviral therapies (cART) and highly active antiretroviral therapies (HAART) have been able to reduce HIV viral activation, often below 50 copies of HIV RNA/ml of plasma, no therapy has provided elimination of HIV infected cells which are not actively replicating HIV, commonly referred to as a patient's latent reservoir of HIV. “Kick and kill” strategies have been proposed for reservoir reduction and/or elimination. Compounds with “kick” activity have the potential to reverse latency and increase HIV protein expression in infected cells, making them more susceptible to immune-mediated killing. Compounds with “kill” activity have the potential to enhance killing of HIV-infected cells, e.g. by enhancing immune effector cell function. Prospects for Treatment of Latent HIV, Barton et al., Clin. Pharm. & Therap., Vol. 93, Issue 1, pp. 46-56; Neutralizing the HIV Reservoir, Marsden et al., Cell, 158, Aug. 28, 2014, pp.971-972; HIV-1 Latency: An Update of Molecular Mechanisms and Therapeutic Strategies, Battistini et al., Viruses 2014, 6, 1715-1758; and Quantification of HIV-1 latency reversal in resting CD4+ T cells from patients on suppressive antiretroviral therapy, Cillo et al., PNAS, May 13 2014, Vol. 111, No. 19, pp. 7078-7083.

There remains a need for new agents and therapies capable of assisting in the activation of the latent HIV-infected cells to enhance the activity of antiretroviral therapies and immune responses.

### SUMMARY

Provided herein are methods of treatment, regimens, pharmaceutical formulations, and kits which may be useful in treating HIV infections in a human, wherein each of the methods of treatment, regimens, pharmaceutical formulations, and kits comprise the use of a TLR8 modulating compound, including for example a compound of Formula (J) or (I), or a pharmaceutically acceptable salt thereof:

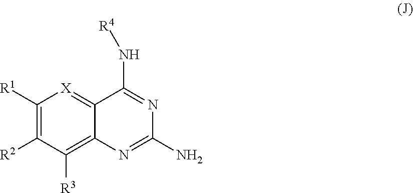

wherein:

X is N or CR¹⁰;

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;

R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

wherein each C₃₋₆cycloalkyl, 3 to 6 membered heterocyclyl, C₆₋₁₀ aryl, and 5 to 10 membered heteroaryl is optionally substituted with 1 to 5 R²¹ groups;

R¹⁰ is selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups

each R²⁰ is independently selected from the group consisting of halogen, C₁₋₆haloalkyl, CN, —NR^{a}R^{b}, S(O)₁₋₂R^{a}, and OR^{a};

each R²¹ is independently selected from the group consisting of halogen, C₁₋₆alkyl, C₁₋₆haloalkyl, CN, —NR^{a}R^{b}, S(O)₁₋₂R^{a}, and OR^{a};

each R^{a} and R^{b} are independently selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl;

provided that when X is N, R¹ is Cl, R² is H and R³ is H then R⁴ is not CH₂CH₂OMe or CH₂CH₂SO₂Me.

In certain embodiments, the TLR8 modulating compound is a compound of Formula (I)

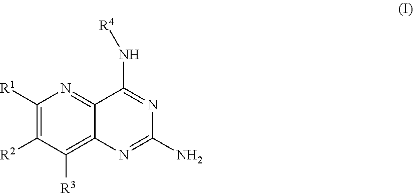

wherein:

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;

R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

wherein each C₃₋₆cycloalkyl, 3 to 6 membered heterocyclyl, C₆₋₁₀ aryl, and 5 to 10 membered heteroaryl is optionally substituted with 1 to 5 R²¹ groups;

each R²⁰ is independently selected from the group consisting of halogen, C₁₋₆haloalkyl, CN, —NR^{a}R^{b}, S(O)₁₋₂R^{a}, and OR^{a};

each R²¹ is independently selected from the group consisting of halogen, C₁₋₆alkyl, C₁₋₆haloalkyl, CN, —NR^{a}R^{b}, S(O)₁₋₂R^{a}, and OR^{a};

each R^{a} and R^{b} are independently selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl;

provided that when R¹ is C1, R² is H and R³ is H then R⁴ is not CH₂CH₂OMe or CH₂CH₂SO₂Me.

In certain embodiments, the TLR8 modulating compound is a compound of Formula (IV):

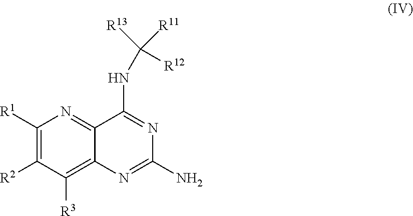

wherein:

R¹ is selected from the group consisting of hydrogen, halogen, C₁₋₆ alkyl, CN, and OR^{a}, wherein C₁₋₆ alkyl is optionally substituted with 1 to 5 R²⁰ groups;

R² is selected from the group consisting of hydrogen, halogen, C₁₋₆ alkyl, CN, and OR^{a}, wherein C₁₋₆ alkyl optionally substituted with 1 to 5 R²⁰ groups;

R³ is selected from the group consisting of hydrogen, halogen, C₁₋₆ alkyl, CN, and OR^{a}, wherein C₁₋₆ alkyl is optionally substituted with 1 to 5 R²⁰ groups;

R¹¹ is selected from the group consisting of C₁₋₂ alkyl, C₃₋₆ cycloalkyl, and C₁₋₃ haloalkyl;

R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 or 2 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 2 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

each R²⁰ is independently selected from the group consisting of halogen, CN, —NR^{a}R^{b}, and OR^{a}; and

each R^{a} and R^{b} is independently selected from the group consisting of hydrogen and C₁₋₃ alkyl, wherein each C₁₋₃ alkyl is optionally substituted with 1 to 3 substituents independently selected from halogen, —OH, and NH₂.

Another aspect of the present disclosure includes a method for treating an HIV infection in a human, the method comprising administering to a human in need thereof a therapeutically effective amount of a TLR8 modulating compound.

The present disclsore includes combinations of aspects and embodiments, as well as preferences, as herein described throughout the present specification.

### BRIEF DESCRIPTION OF THE DRAWINGS

FIG. 1 depicts the induced cytokines and chemokines by TLR agonists as determined according to Example 121.

FIG. 2A and FIG. 2B depicts the induction of HIV-specific polyfunctional CD8+ T cells in PBMC cultures from HIV-1 Infected Subjects on cART treated with the TLR8 Agonist of Example 15.

FIG. 3 depicts the percent reduction of HIV-infected (i.e. p24+) CD4+ T cells after administration of the compound of Example 15 and PGT121 as described in Example 125

FIG. 4 depicts the percent reduction of p24+ CD4+ T cells after administration of the compound of Example 15 and PGT121 as described in Example 125.

FIGS. 5A, 5B, 5C and 5D depict induction of cytokines and chemokines by the compound of Example 65 in Rhesus Macaques as described in Example 126.

### DETAILED DESCRIPTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

All documents referenced herein are each incorporated by reference in their entirety for all purposes.

TLR8 modulating compounds (TLR8 modulating agents) which may be used in the uses, methods, combinations, pharmaceutical formulations/compositions, kits, and regimens described herein include compounds that modulate TLR8 as described herein, including without limitation, motolimod, 3M-051, 3M-052, MCT-465, IMO-4200, VTX-763, VTX-1463, and the TLR8 modulating compounds found in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), the contents of each of which is incorporated herein in their entirety by reference.

Provided for each of the uses, methods, combinations, pharmaceutical formulations/compositions, kits, and regimens described herein there are separate embodiments comprising the use of a TLR8 modulating compound Formula (J), (I), (II), (IIa), (IIb), (III), (IIIa), (IIIb), (IV), (IVa), (IVb), (IVc), or (IVd).

In certain embodiments, the TLR8 modulating compound is a compound of Formula (J):

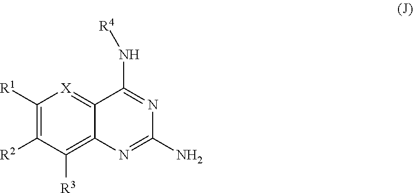

or a pharmaceutically acceptable salt thereof, wherein:

X is N or CR¹⁰;

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;

R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

wherein each C₃₋₆cycloalkyl, 3 to 6 membered heterocyclyl, C₆₋₁₀ aryl, and 5 to 10 membered heteroaryl is optionally substituted with 1 to 5 R²¹ groups;

R¹⁰ is selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups

each R²⁰ is independently selected from the group consisting of halogen, C₁₋₆haloalkyl, CN, —NR^{a}R^{b}, S(O)₁₋₂R^{a}, and OR^{a};

each R²¹ is independently selected from the group consisting of halogen, C₁₋₆alkyl, C₁₋₆haloalkyl, CN, —NR^{a}R^{b}, S(O)₁₋₂R^{a}, and OR^{a};

each R^{a} and R^{b} are independently selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl;

provided that when X is N, R¹ is Cl, R² is H and R³ is H then R⁴ is not CH₂CH₂OMe or CH₂CH₂SO₂Me.

In certain embodiments of Formula (J), X is CR¹⁰. In certain embodiments of Formula (J), X is N.

In certain embodiments, the TLR8 modulating compound is a compound of Formula (I):

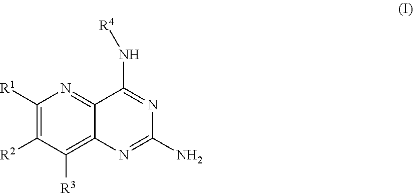

or a pharmaceutically acceptable salt thereof, wherein:

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;

R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

wherein each C₃₋₆cycloalkyl, 3 to 6 membered heterocyclyl, C₆₋₁₀ aryl, and 5 to 10 membered heteroaryl is optionally substituted with 1 to 5 R²¹ groups;

each R²⁰ is independently selected from the group consisting of halogen, C₁₋₆haloalkyl, CN, —NR^{a}R^{b}, S(O)₁₋₂R^{a}, and OR^{a};

each R²¹ is independently selected from the group consisting of halogen, C₁₋₆alkyl, C₁₋₆haloalkyl, CN, —NR^{a}R^{b}, S(O)₁₋₂R^{a}, and OR^{a};

each R^{a} and R^{b} are independently selected from the group consisting of H and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl;

provided that when R¹ is Cl, R² is H and R³ is H then R⁴ is not CH₂CH₂OMe or CH₂CH₂SO₂Me.

In certain embodiments of a compound of Formula (J) or (I), R⁴ is C₁₋₈ alkyl which is optionally substituted with 1 to 5 substituents independently selected from the group consisting of halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

and wherein each C₃₋₆cycloalkyl, 3 to 6 membered heterocyclyl, C₆₋₁₀ aryl, and 5 to 10 membered heteroaryl is optionally substituted with 1 to 5 R²¹ groups.

In certain embodiments of a compound of Formula (J) or (I), R⁴ is C₁₋₆ alkyl optionally substituted with 1 to 5 substituents independently selected from the group consisting of halogen, —OR^{a}, —C(O)OR^{a}, —C(O)NR^{a}R^{b},—SR^{a}, C₁₋₆haloalkyl, C₃₋₆cycloalkyl, 3 to 6 membered heterocyclyl, and C₆₋₁₀ aryl; wherein each C₃₋₆cycloalkyl, 3 to 6 membered heterocyclyl, and C₆₋₁₀ aryl is optionally substituted with 1 to 5 R²¹ groups. In certain embodiments of a compound of Formula (J) or (I), R⁴ is C₃₋₈ alkyl optionally substituted with 1 to 5 substituents independently selected from the group consisting of halogen, —OR^{a}, —C(O)OR^{a}, —NR^{a}C(O)R^{b},—SR^{a}, C₁₋₆haloalkyl, C₃₋₆cycloalkyl, 3 to 6 membered heterocyclyl, and C₆₋₁₀ aryl; wherein each C₃₋₆cycloalkyl, 3 to 6 membered heterocyclyl, and C₆₋₁₀ aryl is optionally substituted with 1 to 5 R²¹ groups.

In certain embodiments of a compound of Formula (J) or (I), R⁴ is C₁₋₆ alkyl optionally substituted with 1 to 3 substituents independently selected from the group consisting of halogen, —OR^{a}, —C(O)OR^{a}, —C(O)NR^{a}R^{b}, —SR^{a}, —C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups. In certain embodiments of a compound of Formula (J) or (I), R⁴ is C₃₋₈ alkyl optionally substituted with 1 to 3 substituents independently selected from the group consisting of halogen, —OR^{a}, —C(O)OR^{a}, —NR^{a}C(O)R^{b}, —SR^{a}, —C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups.

In certain embodiments of a compound of Formula (J) or (I), R⁴ is C₁₋₆ alkyl optionally substituted with 1 or 2 substituents independently selected halogen, —OR^{a}, —C(O)OR^{a}, —C(O)NR^{a}R^{b}, —SR^{a}, C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups and wherein R^{a} and R^{b} are each independently hydrogen or C₁₋₄alkyl, wherein the C₁₋₄ alkyl is optionally substituted with —NH₂, OH, or pyridyl. In certain embodiments of a compound of Formula (J) or (I), R⁴ is C₃₋₈ alkyl which is optionally substituted with 1 or 2 substituents independently selected from the group consisting of halogen, —OR^{a}, —C(O)OR^{a}, —NR^{a}C(O)R^{b}, —SR^{a}, C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²⁰ groups and wherein R^{a} and R^{b} are each independently hydrogen or C₁₋₄alkyl, wherein each C₁₋₄ alkyl is optionally substituted with —NH₂, OH, or pyridyl.

In certain embodiments of a compound of Formula (J) or (I), R⁴ is C₁₋₆ alkyl optionally substituted with 1 or 2 substituents independently selected from the group consisting of OH, CF₃ —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl. In certain embodiments of a compound of Formula (J) or (I), R⁴ is C₃₋₈ alkyl which is optionally substituted with 1 or 2 substituents independently selected from OH, CF_{3,}—C(O)OH, —C(O)OCH₃, SCH₃, —NHC(O)CH₃, —NHC(O)CH₂CH₂NH₂, —NHC(O)CH₂CH₂OH, —NHC(O)CH₂-pyridyl, phenyl, tetrahydrofuranyl, and cyclopropyl.

In certain embodiments of a compound of Formula (J) or (I), R⁴ is C₃₋₆ alkyl optionally substituted with 1 or 2 substituents independently selected from the group consisting of OH, CF_{3,}—C(O)OH, —C(O)OCH₃, —C(O)NH₂, SCH₃, —C(O)NHCH₃, —C(O)NHCH₂CH₂NH₂, —C(O)NHCH₂CH₂OH, and —C(O)NHCH₂-pyridyl. In certain embodiments of a compound of Formula (J) or (I), R⁴ is C₃₋₆ alkyl which is optionally substituted with 1 or 2 substituents independently selected from OH, CF_{3,}—C(O)OH, —C(O)OCH₃, SCH₃, —NHC(O)CH₃, —NHC(O)CH₂CH₂NH₂, —NHC(O)CH₂CH₂OH, —NHC(O)CH₂-pyridyl, phenyl, tetrahydrofuranyl, and cyclopropyl.

In certain embodiments of a compound of Formula (J) or (I), R⁴ is C₁₋₆ alkyl which is optionally substituted with OH. In certain embodiments of a compound of Formula (J) or (I), R⁴ is C₃₋₈ alkyl which is optionally substituted with OH. In certain embodiments of a compound of Formula (J) or (I), R⁴ is C₃₋₈ alkyl which is substituted with —NHC(O)CH₃.

In certain embodiments of a compound of Formula (J) or (I), R⁴ is C₃₋₆ alkyl which is optionally substituted with OH. In certain embodiments of a compound of Formula (J) or (I), R⁴ is C₃₋₆ alkyl which is substituted with —NHC(O)CH₃.

In certain embodiments of a compound of Formula (J) or (I), R⁴ has at least one chiral center. In certain embodiments, the at least one chiral center is in the S configuration. In certain embodiments, the at least one chiral center is in the R configuration.

In certain embodiments of a compound of Formula (J) or (I), R⁴ is selected from the group consisting of:

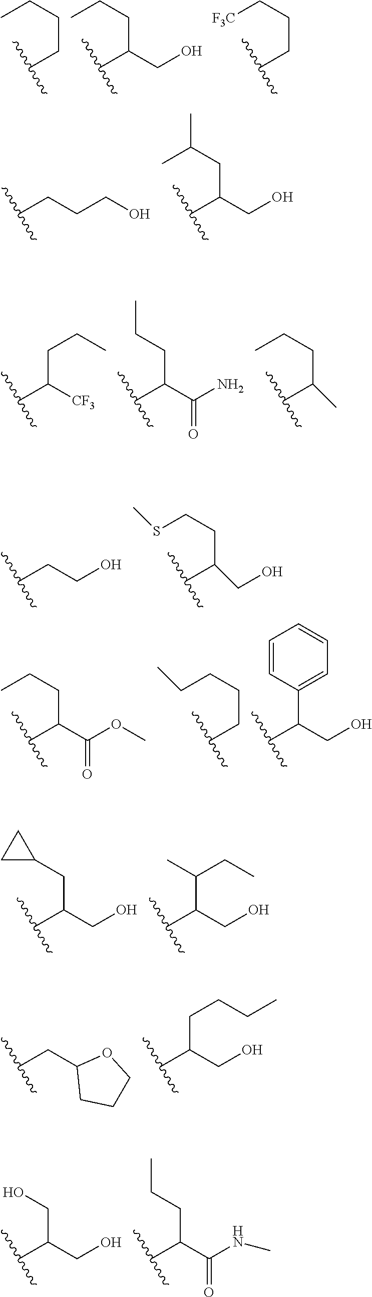

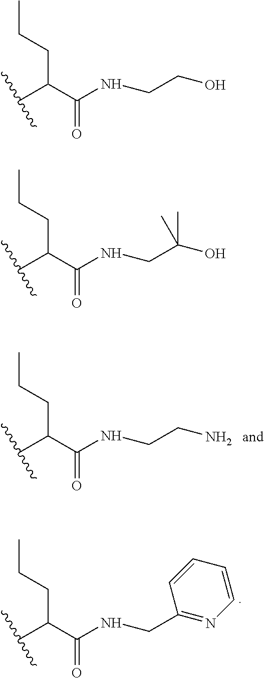 [SMILES: *C(CCC)C(=O)NCCO ; *C(CCC)C(=O)NCC(C)(C)O ; *C(CCC)C(=O)NCCN ; "and" ; *C(CCC)C(=O)NCc1ccccn1]

In certain embodiments of a compound of Formula (J) or (I), R⁴ is selected from the group consisting of:

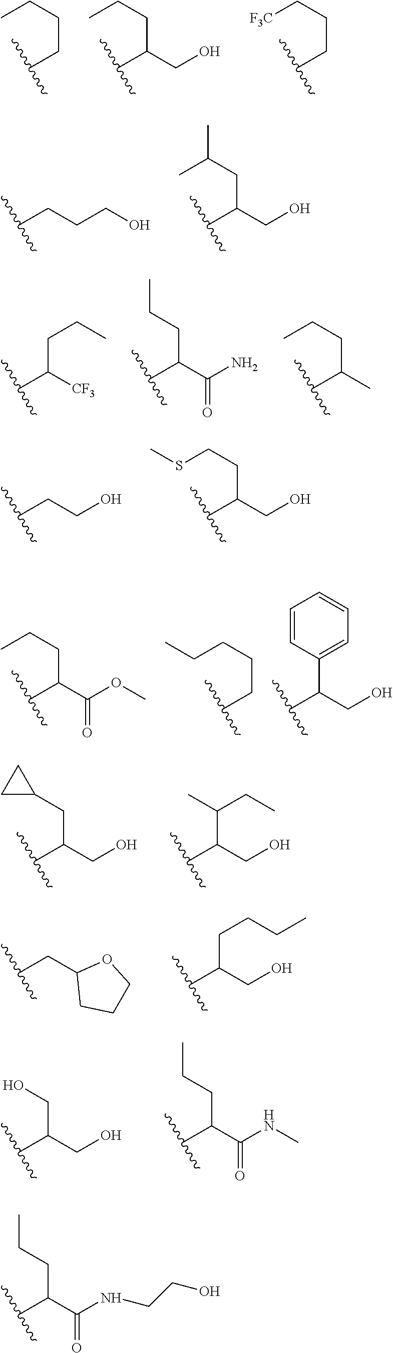

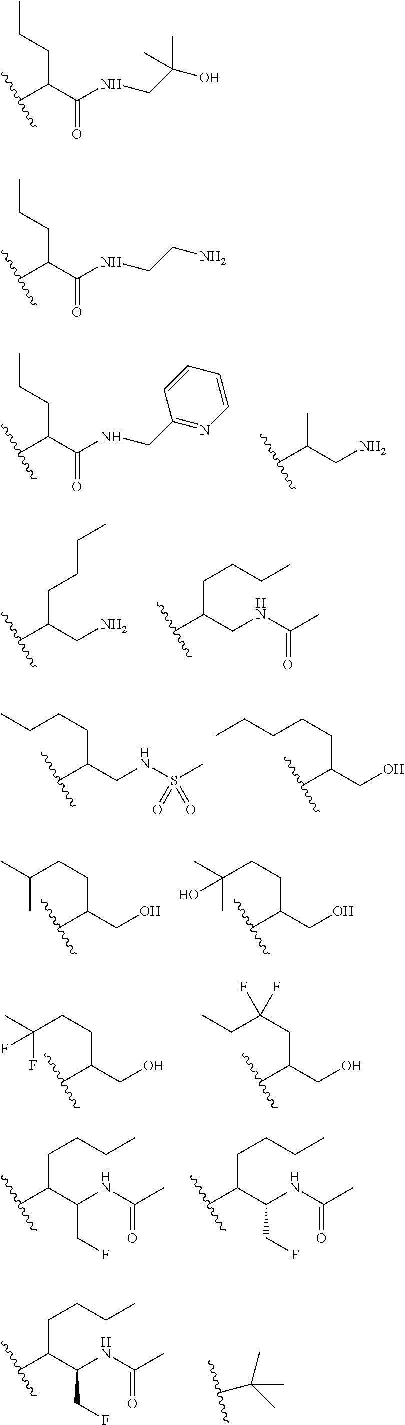

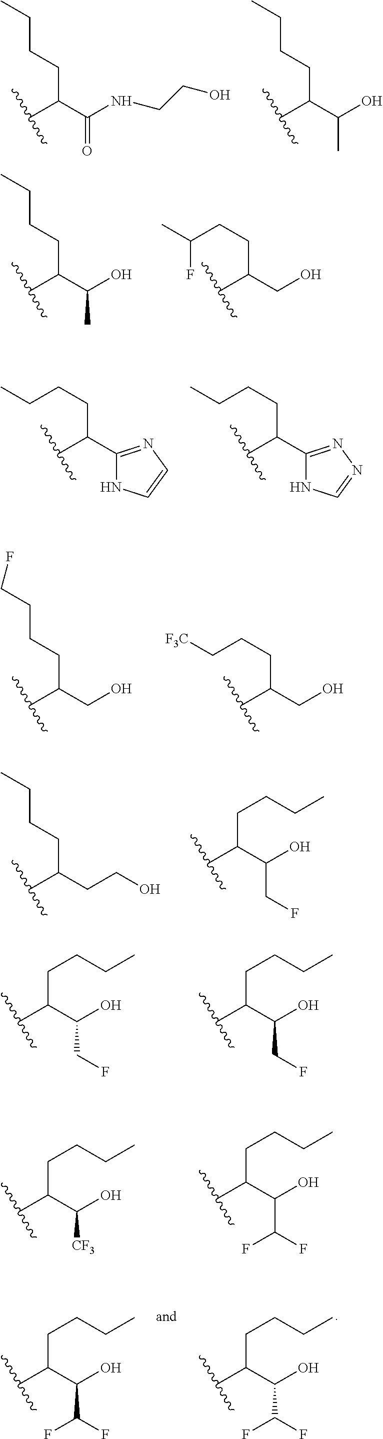

In certain embodiments of a compound of Formula (J) or (I), R⁴ is selected from the group consisting of:

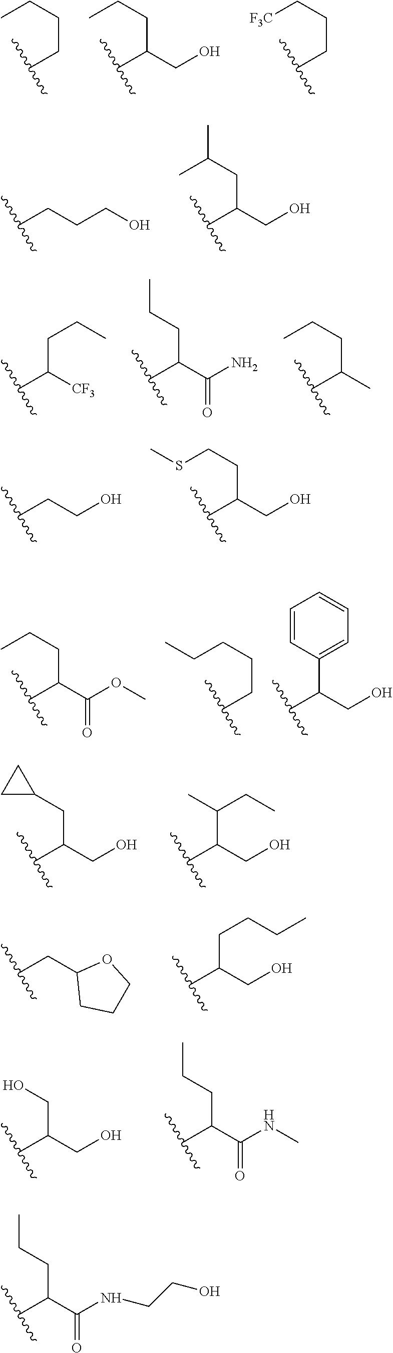

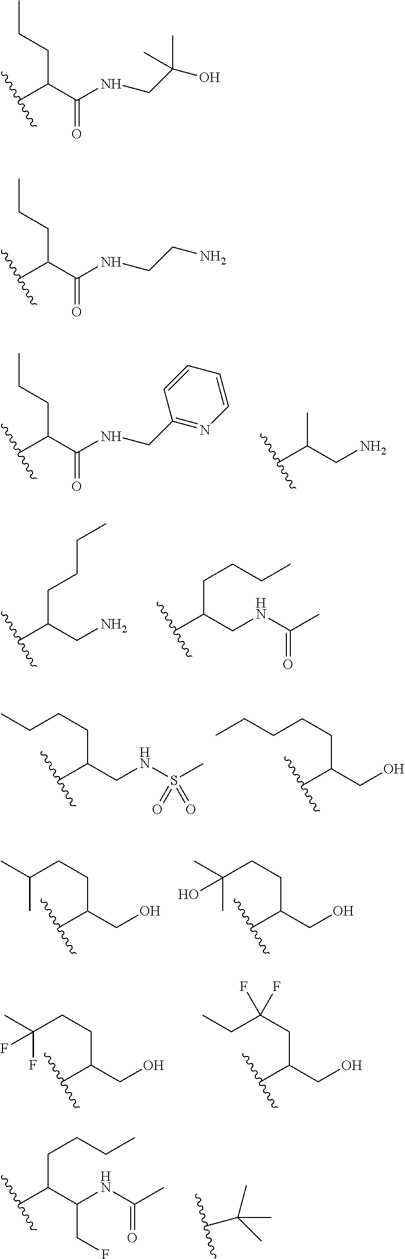

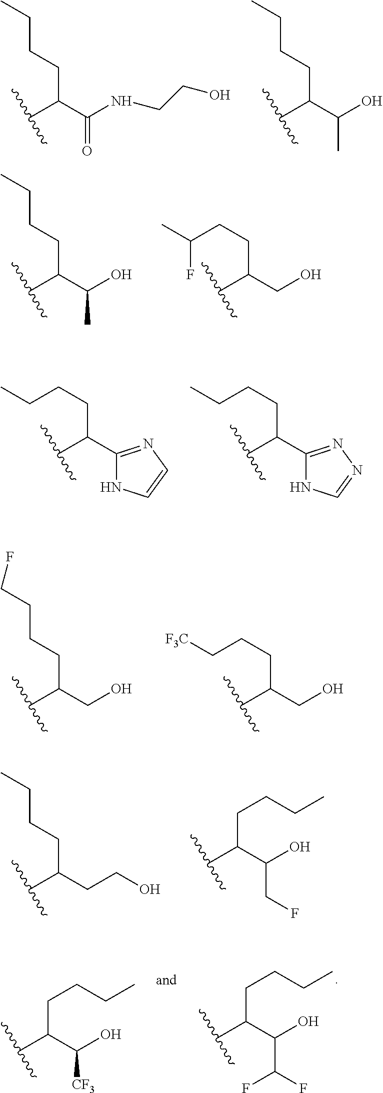

In certain embodiments of a compound of Formula (J) or (I), R⁴ is selected from the group consisting of:

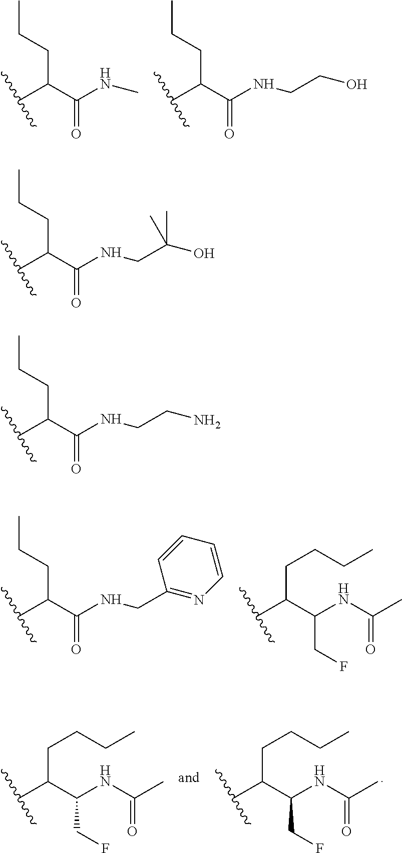

In certain embodiments of a compound of Formula (J) or (I), R⁴ is selected from the group consisting of:

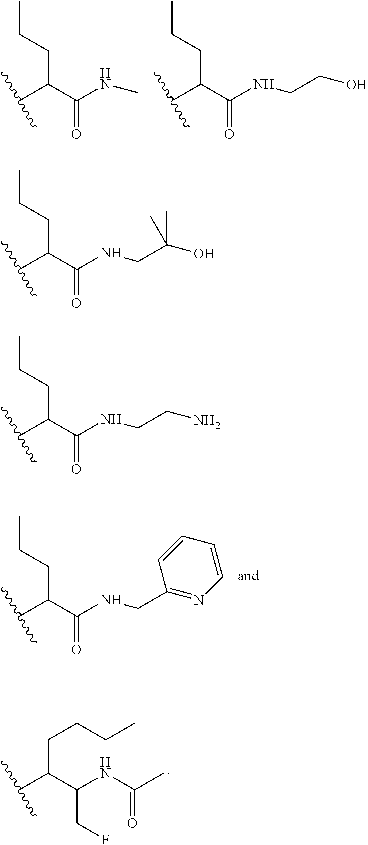

In certain embodiments of a compound of Formula (J) or (I), R⁴ is selected from the group consisting of:

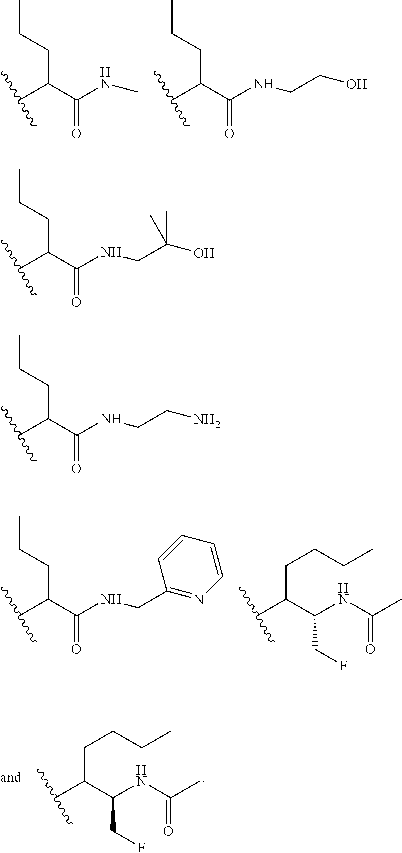

In certain embodiments of a compound of Formula (J) or (I), R⁴ is selected from the group consisting of:

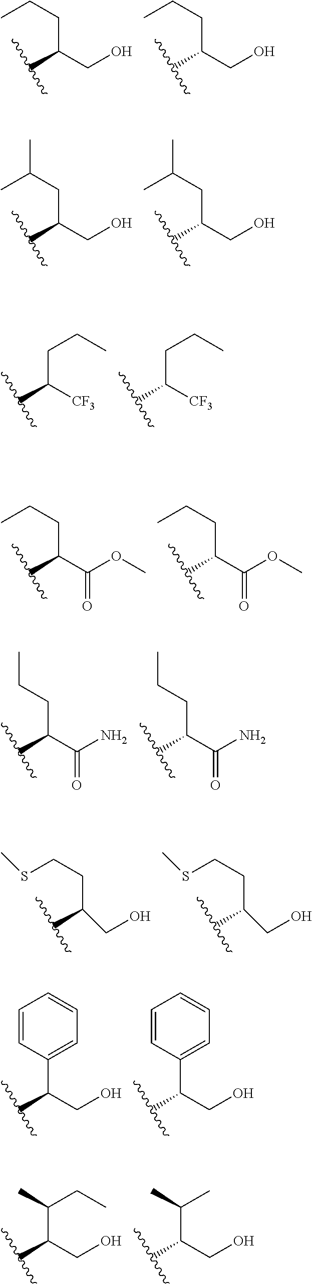

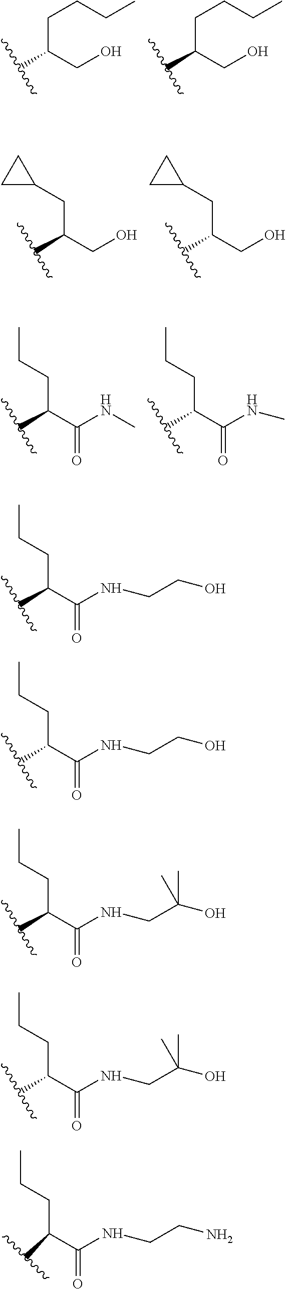

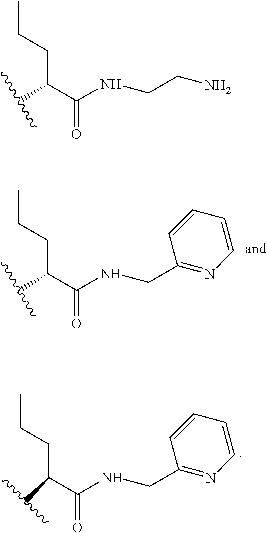 [SMILES: *[C@H](CCC)C(=O)NCCN ; *[C@H](CCC)C(=O)NCc1ccccn1 ; *[C@@H](CCC)C(=O)NCc1ccccn1]

In certain embodiments of a compound of Formula (J) or (I), R⁴ is selected from the group consisting of:

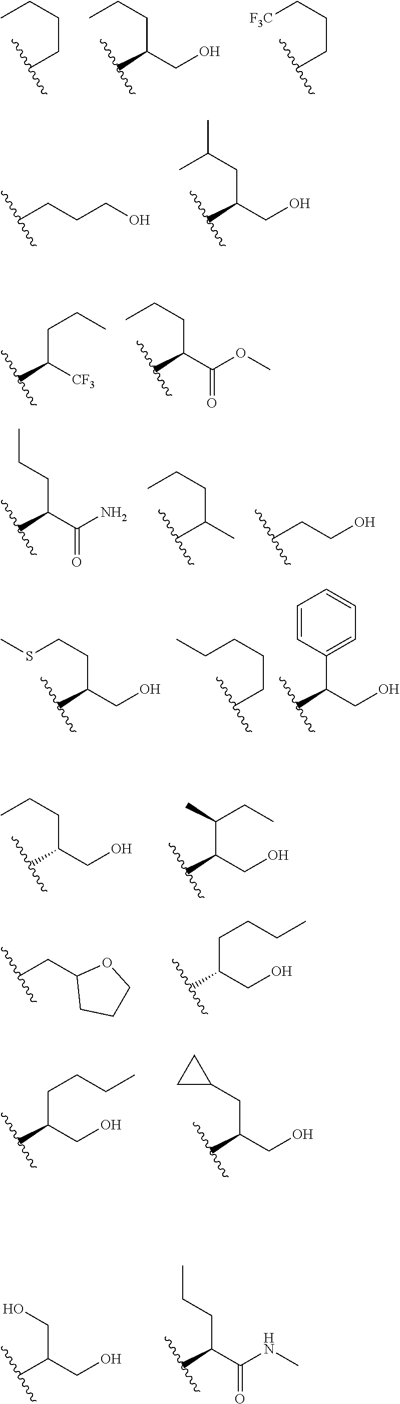

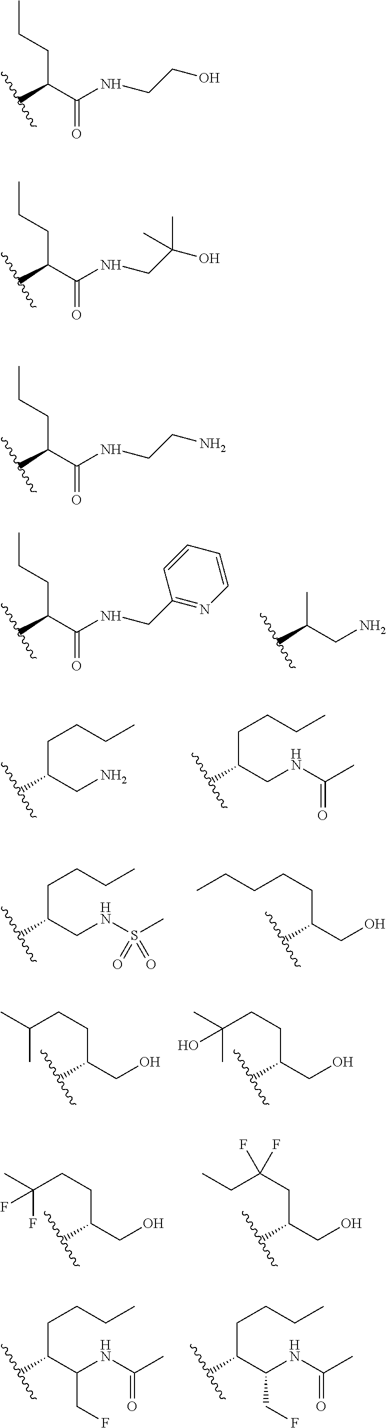

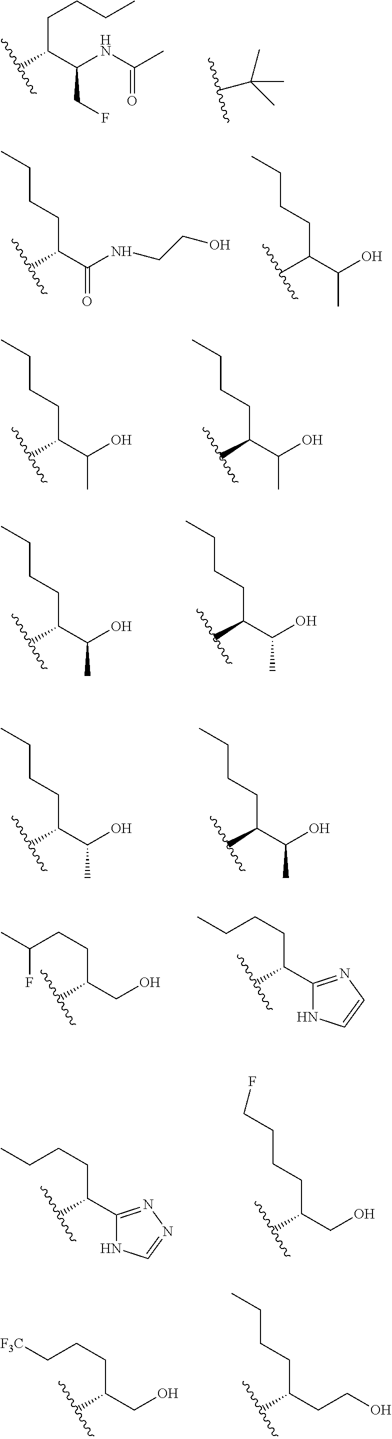

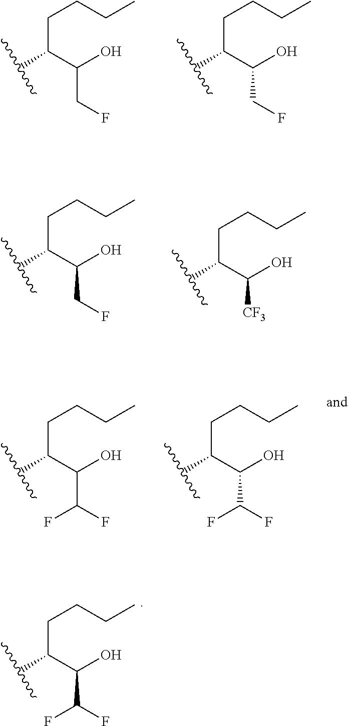

In certain embodiments of a compound of Formula (J) or (I), R⁴ is selected from the group consisting of:

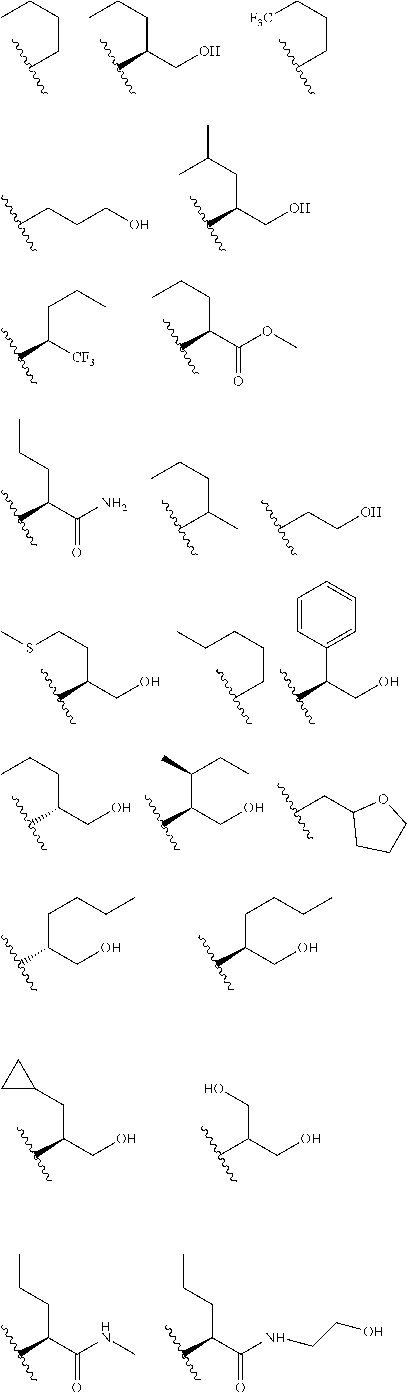

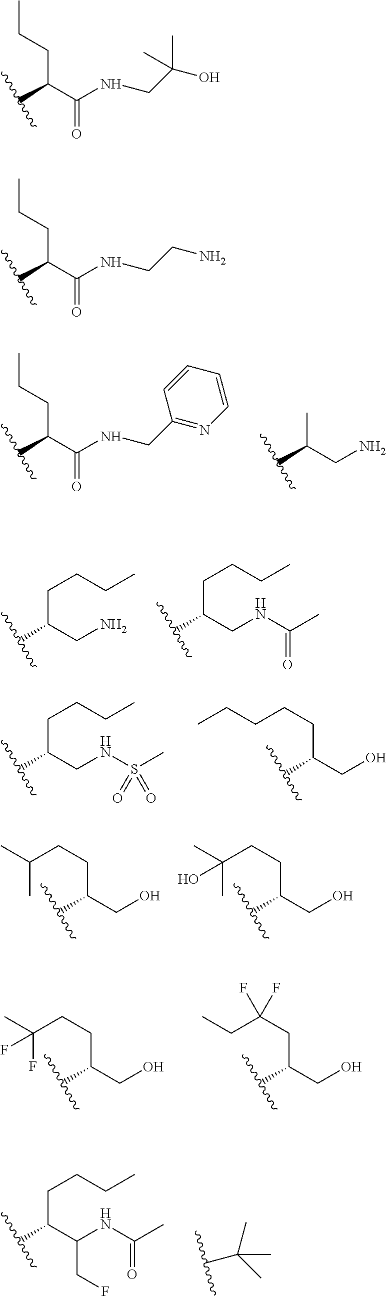

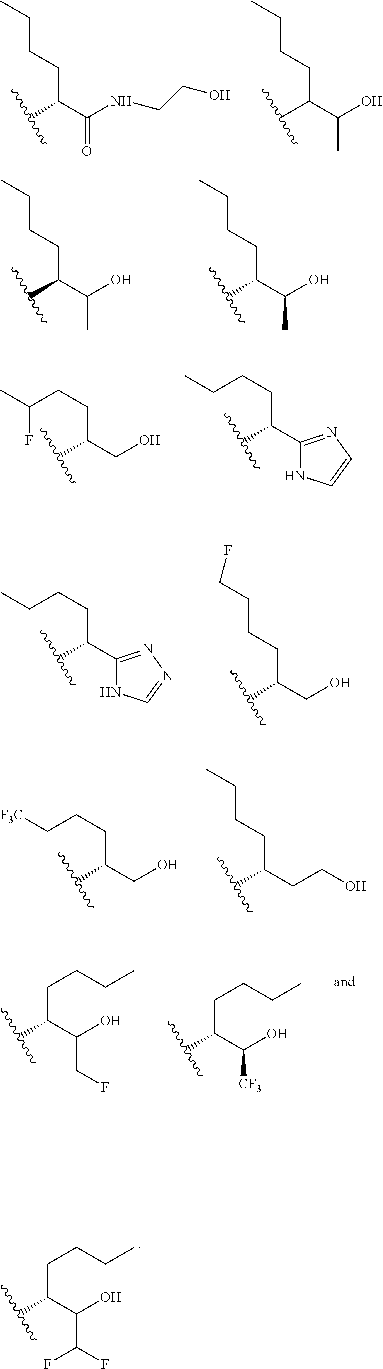

In certain embodiments of a compound of Formula (J) or (I), R⁴ is selected from the group consisting of:

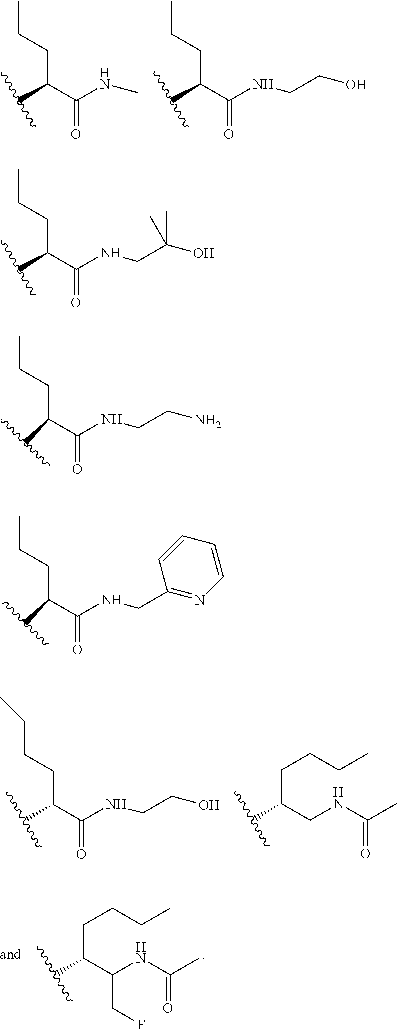

In certain embodiments of a compound of Formula (J) or (I), R⁴ is selected from the group consisting of:

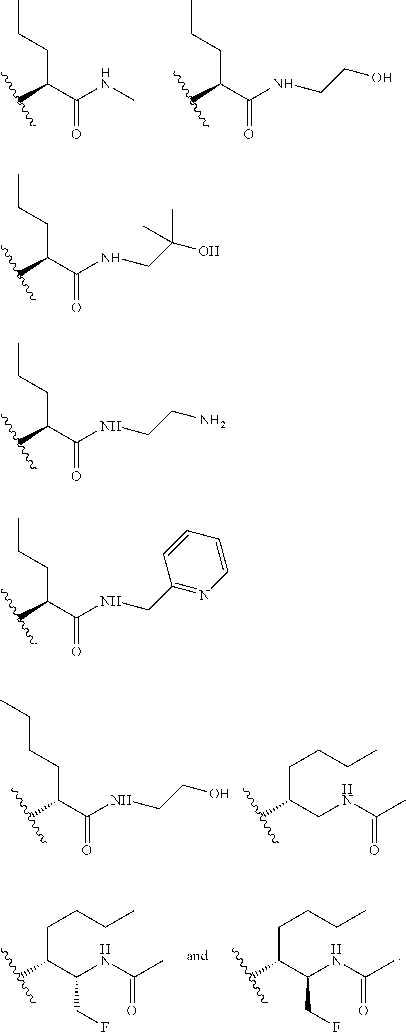

In certain embodiments of a compound of Formula (J) or (I), R⁴ is selected from the group consisting of:

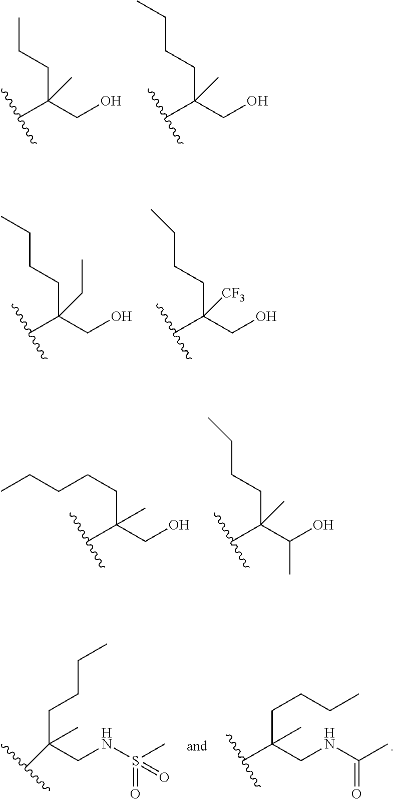

In certain embodiments of a compound of Formula (J) or (I), R⁴ is selected from the group consisting of:

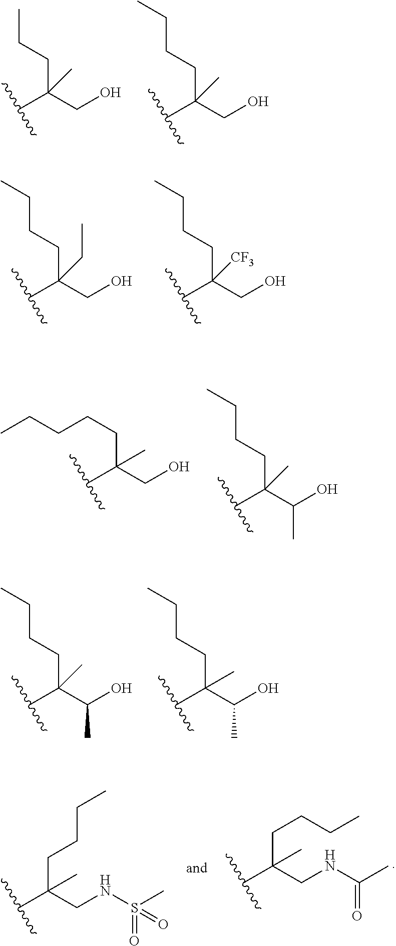

In certain embodiments of a compound of Formula (J) or (I), R⁴ is selected from the group consisting of:

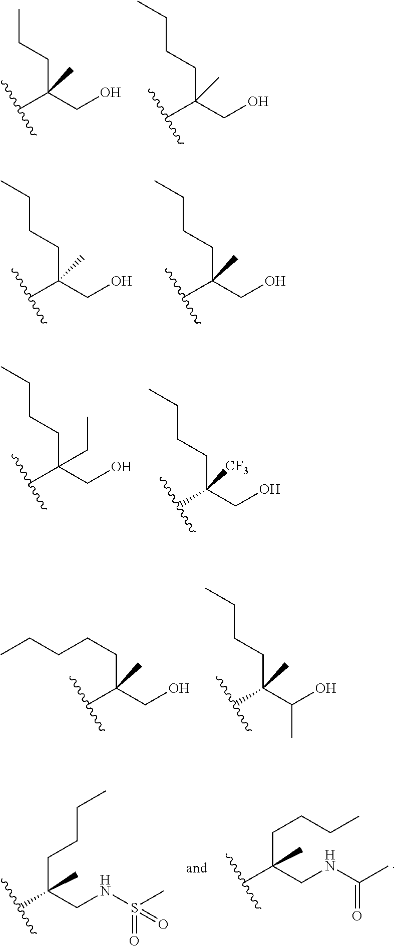

In certain embodiments of a compound of Formula (J) or (I), R⁴ is selected from the group consisting of:

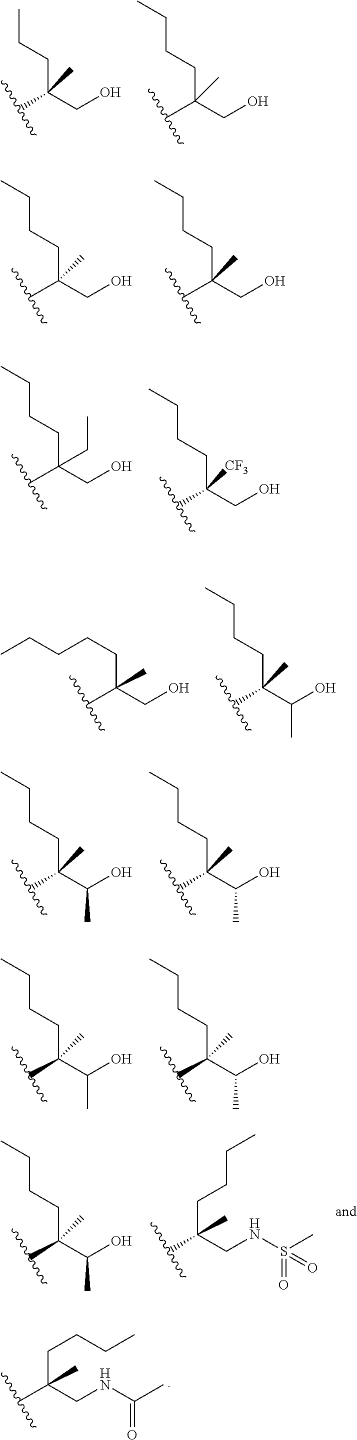

In certain embodiments of a compound of Formula (J) or (I), R⁴ is selected from the group consisting of:

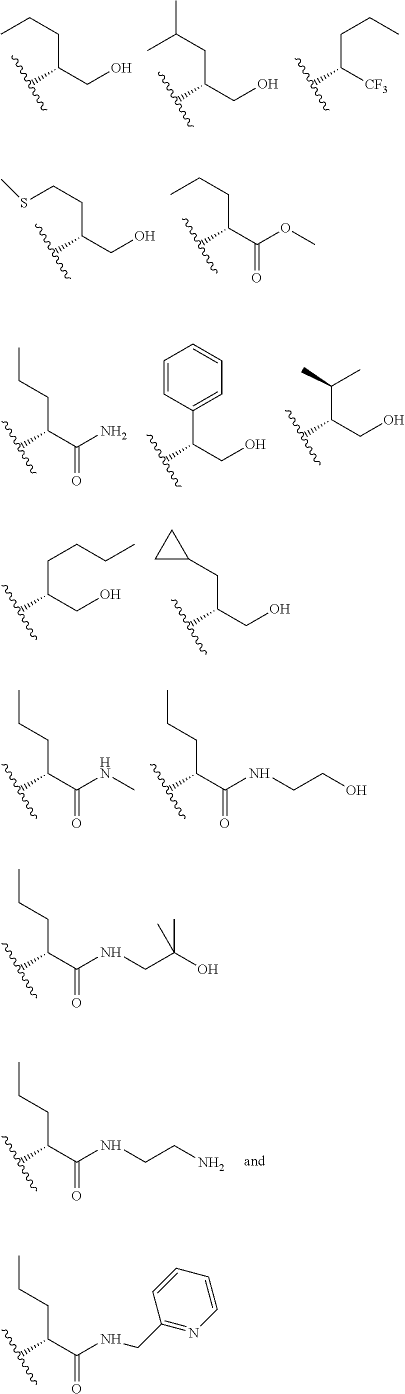

In certain embodiments of a compound of Formula (J) or (I), R⁴ is selected from the group consisting of:

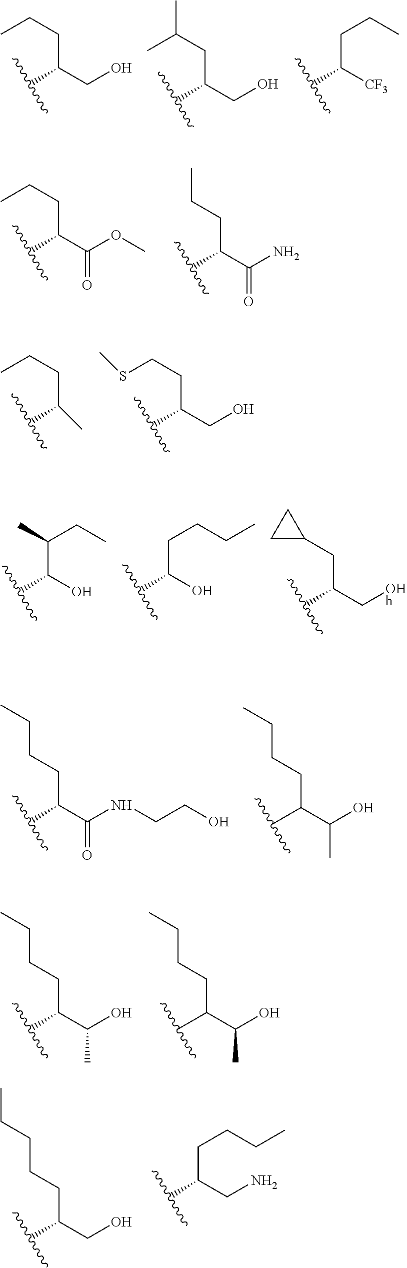

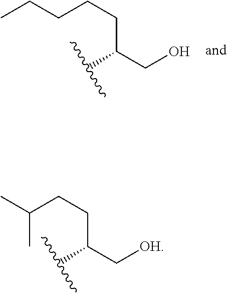

In certain embodiments of a compound of Formula (J) or (I), R⁴ is selected from the group consisting of:

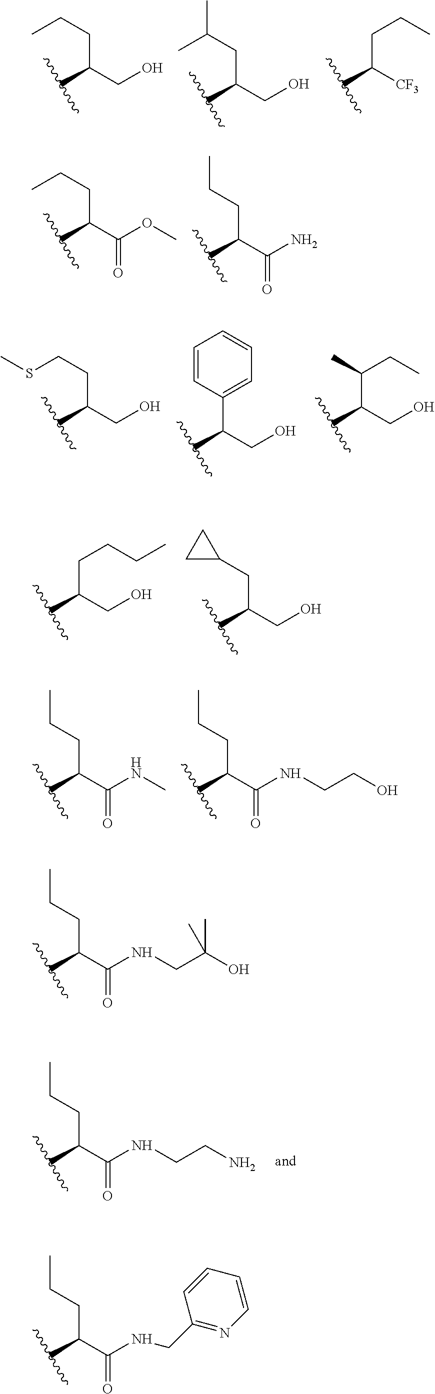

In certain embodiments of a compound of Formula (J) or (I), R⁴ is selected from the group consisting of

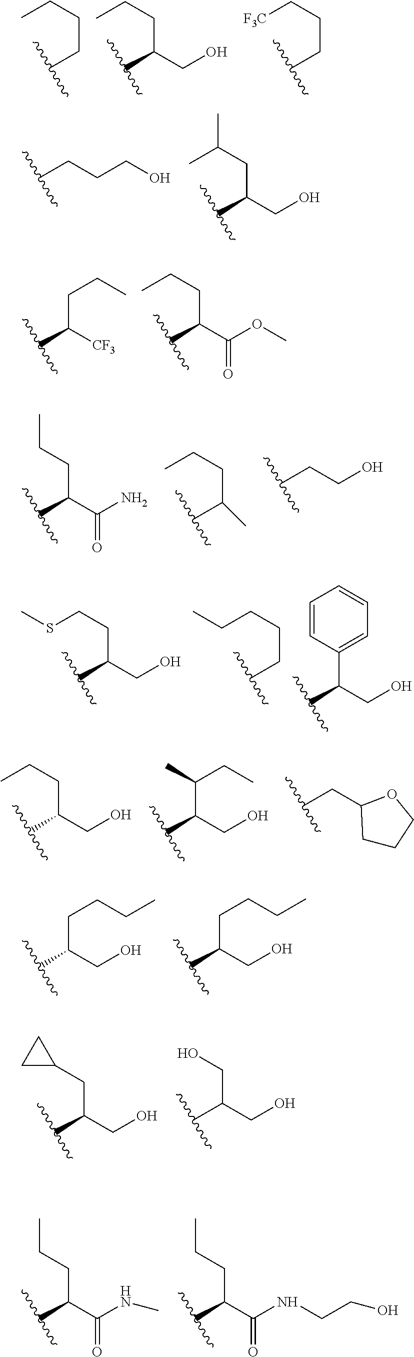

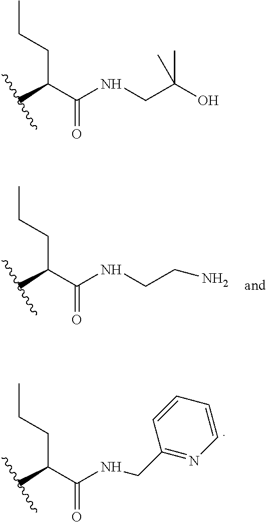 [SMILES: *[C@@H](CCC)C(=O)NCC(C)(C)O ; *[C@@H](CCC)C(=O)NCCN ; *[C@@H](CCC)C(=O)NCc1ccccn1]

In certain embodiments of a compound of Formula (J) or (I), R⁴ is

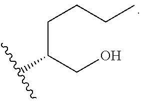 [SMILES: *[C@@H](CO)CCCC]

In certain embodiments of a compound of Formula (J) or (I), R⁴ is

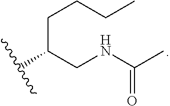 [SMILES: *[C@H](CCCC)CNC(C)=O]

In certain embodiments of a compound of Formula (J) or (I), R⁴ is

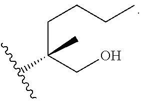

In certain embodiments of a compound of Formula (J) or (I), R⁴ is

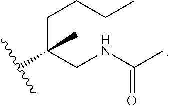

In certain embodiments of a compound of Formula (J) or (I), R⁴ is

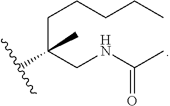

In certain embodiments, the compound of Formula (J) or (I) is a compound of Formula (II)

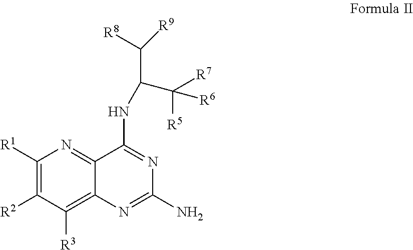

or a pharmaceutically acceptable salt thereof, wherein:

R⁵ is selected from the group consisting of hydrogen, halogen, and methyl;

R⁶ is selected from the group consisting of hydrogen, halogen, and methyl; or R⁵ and R⁶ together form an oxo group;

R⁷ is selected from the group consisting of hydrogen, halogen, OR^{a} and NR^{a}R^{b};

R⁸ is selected from the group consisting of hydrogen and methyl;

R⁹ is is selected from the group consisting of C₁₋₄ alkyl, C₃₋₆cycloalkyl, and —S—C₁₋₄alkyl;

R^{a} and R^{b} are independently selected from the group consisting of hydrogen and C₁₋₆alkyl; wherein each C₁₋₆alkyl is optionally substituted with 1 to 3 substituents independently selected from the group consisting of halogen, hydroxyl, and pyridyl; and R¹, R², and R³ are as otherwise defined herein.

For example, in Formula (II), (IIa), and (IIb), R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups; R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups; and R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁹ groups;

In certain embodiments, the compound of Formula (II) is a compound of Formula (IIa)

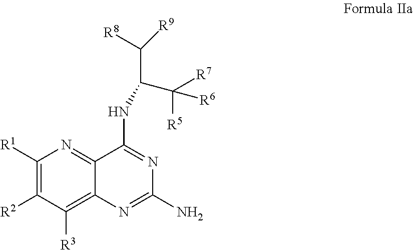

In certain embodiments, the compound of Formula (II) is a compound of Formula (IIb)

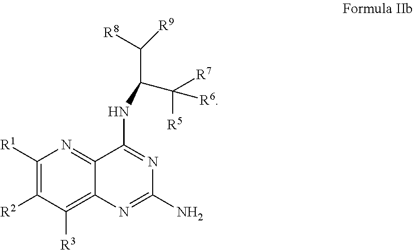

In certain embodiments of the compound of Formula (II), (IIa), or (IIb), R⁵ is hydrogen; R⁶ is hydrogen; or R⁵ and R⁶ together form an oxo group; R⁷ is OR^{a} or NR^{a}R^{b}; R⁸ is hydrogen; R⁹ is C₁₋₄alkyl, cyclopropyl or —SCH₃; R^{a} and R^{b} are independently selected from the group consisting of hydrogen and C₁₋₄alkyl; wherein each C₁₋₄alkyl is optionally substituted with 1 to 3 substituents independently selected from halogen, hydroxyl, pyrid-2-yl, and CF₃, and R¹, R², and R³ are as otherwise defined herein. In certain embodiments, R^{a} and R^{b} are hydrogen. In certain embodiments, R⁷ is OH or NH₂. In certain embodiments, R¹ and R² are hydrogen.

In certain embodiments of a compound of Formula (IIa),

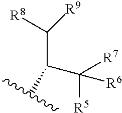

is selected from

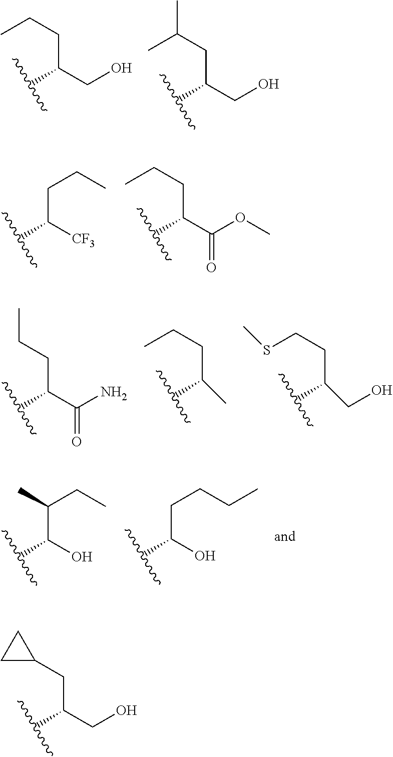

In certain embodiments of a compound of Formula (IIa),

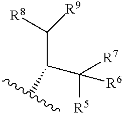

is selected from

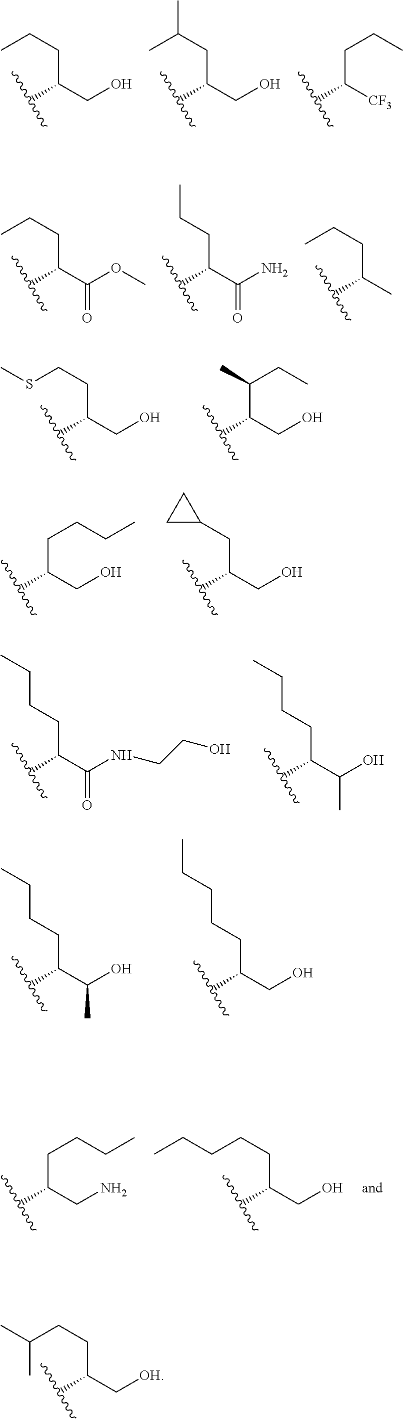

In certain embodiments of a compound of formula (IIb),

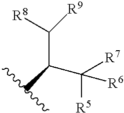

is selected from

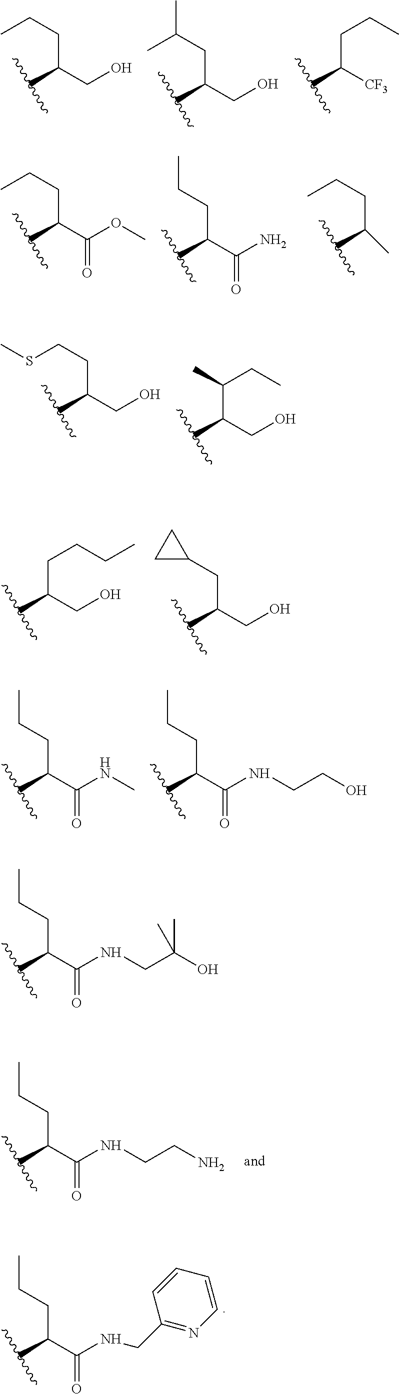

In certain embodiments of a compound of formula (IIb),

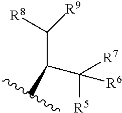

is selected from

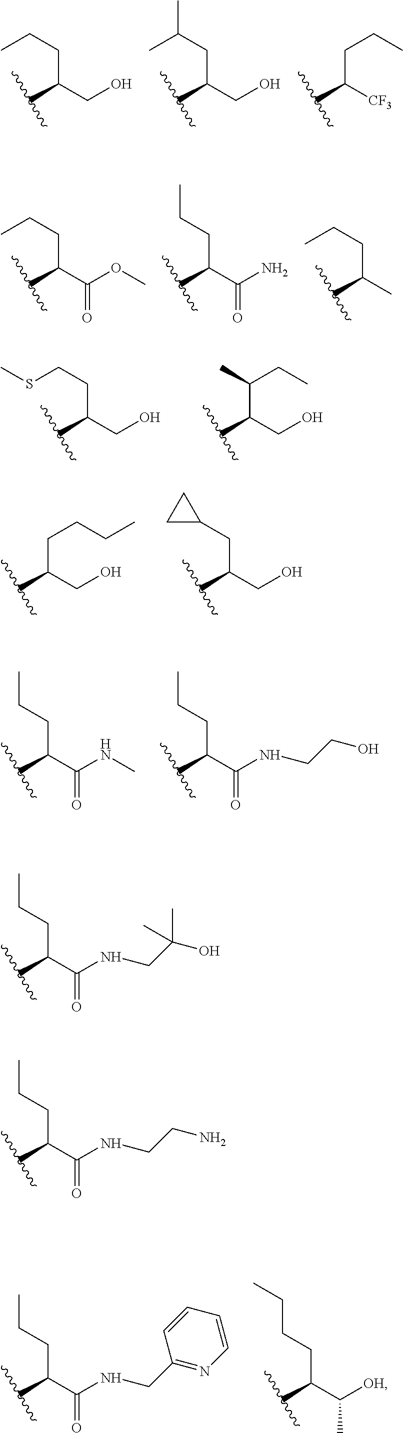

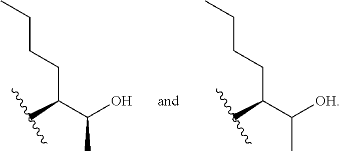

In certain embodiments of the compound of Formula (II), (IIa), or (IIb), R⁵ is hydrogen, R⁶ is hydrogen, or R⁵ and R⁶ together form an oxo group, R⁷ is OR^{a} or NR^{a}R^{b}, R⁸ is hydrogen, R⁹ is C₁₋₄alkyl, cyclopropyl or —SCH3, and R^{a} and R^{b} are independently selected from the group consisting of hydrogen and C₁₋₄alkyl; wherein each C₁₋₄alkyl is optionally substituted with 1 to 3 substituents independently selected from halogen, hydroxyl, pyrid-2-yl, and CF₃. In certain embodiments of the compound of Formula (II), (IIa), or (IIb), R⁷ is OH or NH₂.

In certain embodiments of a compound of Formula (J), Formula (I), or Formula (II), the compound is a compound of Formula (III)

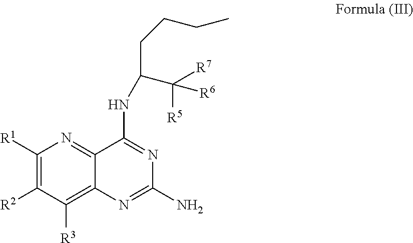

wherein R⁵ is hydrogen;

R⁶ is hydrogen; or R⁵ and R⁶ together form an oxo group;

R⁷ is selected from the group consisting of OR^{a} and NR^{a}R^{b};

R^{a} and R^{b} are independently selected from the group consisting of hydrogen and C₁₋₃alkyl; wherein each C₁₋₃alkyl is optionally substituted with 1 to 3 substituents independently selected from the group consisting of halogen and hydroxyland R¹, R², and R³ are as otherwise defined herein.

In certain embodiments the compound of Formula (III) is a compound of Formula (IIIa)

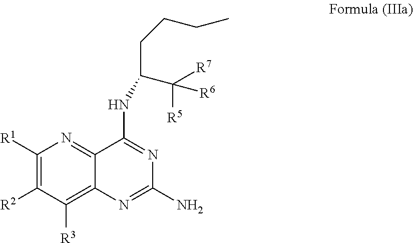

In certain embodiments the compound of Formula (III) is a compound of Formula (IIIb)

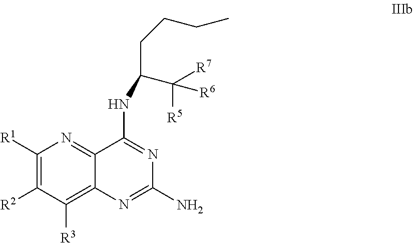

In certain embodiments of the compound of Formula (III), (IIIa), or (IIIb), R⁵ and R⁶ are both hydrogen and R⁷ is OR^{a}, wherein R^{a} is hydrogen or C₁₋₃alkyl. In certain embodiments of the compound of Formula (III), (IIIa), or (IIIb), R⁵ and R⁶ are both hydrogen and R⁷ is OH. In certain embodiments of the compound of Formula (III), (IIIa), or (IIIb), R¹, R², R⁵, and R⁶ are each hydrogen, and R⁷ is OH.

In certain embodiments of the compound of Formula (III), (IIIa), or (IIIb), R⁵ and R⁶ together form an oxo group and R⁷ is selected from the group consisting of OR^{a} and NR^{a}R^{b}, wherein R^{a} and R^{b} are independently selected from the group consisting of hydrogen and C₁₋₃alkyl. In certain embodiments of the compound of Formula (III), (IIIa), or (IIIb), R⁵ and R⁶ together form an oxo group and R⁷ is selected from the group consisting of OR^{a} and NR^{a}R^{b}, wherein R^{a} and R^{b} are independently selected from the group consisting of hydrogen and methyl.

In certain embodiments of a compound of Formula (J), or Formula (I), the compound is a compound of Formula (IV):

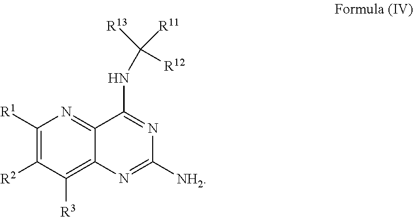

The R¹, R², and R³ groups of Formula (IV) are as defined above for Formula (J) or (I). The R¹¹, R¹² and R¹³ groups are as defined above for R4 in Formula (J) or Formula (I).

In certain embodiments, the compound of Formula (IV), or a pharmaceutically acceptable salt thereof, is a compound of Formula (IVa):

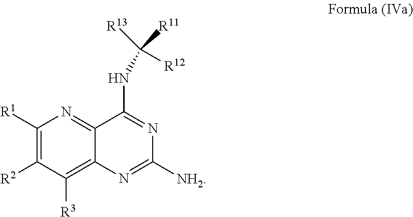

In certain embodiments, the compound of Formula (IV), or a pharmaceutically acceptable salt thereof, is a compound of Formula (IVb):

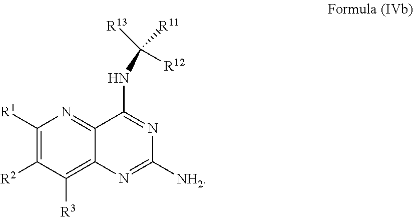

The groups R¹, R², R³, R¹¹, R¹² and R¹³ of Formula (IVa) and (IVb) are as defined for Formula (J), (I) or (IV) above, or as defined below, or any combination thereof.

R¹ of Formula (IV), (IVa) and (IVb) can be any suitable group selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups. In certain embodiments, R¹ is selected from hydrogen, halogen, C₁₋₆ alkyl, CN, and OR^{a}, wherein C₁₋₆ alkyl is optionally substituted with 1 to 5 R²⁰ groups. In certain embodiments, R¹ can be hydrogen, halogen, and C₁₋₃ alkyl, wherein C₁₋₃ alkyl is optionally substituted with 1 to 5 halogen groups. In certain embodiments, R¹ can be hydrogen, fluoro, chloro, bromo, methyl or ethyl, wherein each methyl or ethyl group is optionally substituted with 1 to 5 halogen groups. In certain embodiments, R¹ can be hydrogen, fluoro, chloro, bromo, methyl or ethyl, wherein each methyl or ethyl group is optionally substituted with 1 to 5 fluoro groups. In certain embodiments, R¹ can be hydrogen, methyl, fluoro, chloro, and CF₃. In certain embodiments, R¹ can be hydrogen. In certain embodiments, R¹ is selected from hydrogen, halogen, NH₂, C₁₋₆ alkyl, CN, and OR^{a}, wherein C₁₋₆ alkyl is optionally substituted with 1 to 5 R²⁰ groups.

R² of Formula (IV), (IVa) and (IVb) can be any suitable group selected from hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups. In certain embodiments, R² is selected from hydrogen, halogen, C₁₋₆ alkyl, CN, and OR^{a}, wherein C₁₋₆ alkyl optionally substituted with 1 to 5 R²⁰ groups. In certain embodiments, R² is selected from hydrogen, halogen, C₁₋₃ alkyl, CN and OR^{a}, wherein C₁₋₃ alkyl is optionally substituted with 1 to 5 halogen groups. In certain embodiments, R² is selected from hydrogen, methyl, ethyl, fluoro, chloro, bromo, CF₃, CN, OH, OMe, and OEt. In certain embodiments, R² is selected from hydrogen, methyl, fluoro, and chloro. In certain embodiments, R² is selected from hydrogen and fluoro. In certain embodiments, R² is selected from hydrogen, halogen, NH₂, C₁₋₆ alkyl, CN, and OR^{a}, wherein C₁₋₆ alkyl is optionally substituted with 1 to 5 R²⁰ groups. In certain embodiments, R² is selected from hydrogen, methyl, ethyl, NH₂, fluoro, chloro, bromo, CF₃, CN, OH, OMe, and OEt.

R³ of Formula (IV), (IVa) and (IVb) can be any suitable group selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups. In certain embodiments, R³ is selected from hydrogen, halogen, C₁₋₆ alkyl, CN, and OR^{a}, wherein C₁₋₆ alkyl is optionally substituted with 1 to 5 R²⁰ groups. In certain embodiments, R³ can be selected from hydrogen, halogen, and C₁₋₃ alkyl. In certain embodiments, R³ can be selected from hydrogen, methyl, fluoro, and chloro. In certain embodiments, R³ can be selected from hydrogen and methyl. In certain embodiments, R³ is selected from hydrogen, halogen, NH₂, C₁₋₆ alkyl, CN, and OR^{a}, wherein C₁₋₆ alkyl is optionally substituted with 1 to 5 R²⁰ groups.

In certain embodiments, the compound of Formula (IV), (IVa) or (IVb), or a pharmaceutically acceptable salt thereof, is the compound wherein R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups, R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups, and R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups.

In certain embodiments, the compound of Formula (IV), (IVa) or (IVb), or a pharmaceutically acceptable salt thereof, is the compound wherein R¹ is selected from the group consisting of hydrogen, halogen, and C₁₋₃ alkyl, wherein C₁₋₃ alkyl is optionally substituted with 1 to 5 halogen groups, R² is selected from the group consisting of hydrogen, halogen, C₁₋₃ alkyl, CN and OR^{a}, wherein C₁₋₃ alkyl is optionally substituted with 1 to 5 halogen groups, and R³ is selected from the group consisting of hydrogen, halogen, and C₁₋₃ alkyl.

In certain embodiments, the compound of Formula (IV), (IVa) or (IVb), or a pharmaceutically acceptable salt thereof, is the compound wherein R¹ is selected from the group consisting of hydrogen, methyl, fluoro, chloro, and CF₃, R² is selected from the group consisting of hydrogen, methyl, ethyl, fluoro, chloro, bromo, CF₃, CN, OH, OMe, and OEt, and R³ is selected from the group consisting of hydrogen, methyl, fluoro, and chloro.

In certain embodiments, the compound of Formula (IV), (IVa) or (IVb), or a pharmaceutically acceptable salt thereof, is the compound wherein R¹ is selected from the group consisting of hydrogen, methyl, fluoro, chloro, and CF₃, R² is selected from the group consisting of hydrogen, methyl, ethyl, NH₂, fluoro, chloro, bromo, CF₃, CN, OH, OMe, and OEt, and R³ is selected from the group consisting of hydrogen, methyl, fluoro, and chloro.

In certain embodiments, the compound of Formula (IV), (IVa) or (IVb), or a pharmaceutically acceptable salt thereof, is the compound wherein R¹ is hydrogen, R² is selected from the group consisting of hydrogen, methyl, ethyl, fluoro, chloro, and bromo, and R³ is selected from the group consisting of hydrogen and methyl.

In certain embodiments, the compound of Formula (IV), (IVa) or (IVb), or a pharmaceutically acceptable salt thereof, is the compound wherein R¹ is hydrogen. R² is selected from the group consisting of hydrogen and fluoro, and R³ is selected from the group consisting of hydrogen and methyl.

In certain embodiments, R¹¹ of Formula (IV), (IVa) and (IVb) can be any suitable group selected from hydrogen, C₁₋₂ alkyl, C₃₋₆ cycloalkyl, and C₁₋₃ haloalkyl. In certain embodiments, the compound of Formula (IV), (IVa) or (IVb), or a pharmaceutically acceptable salt thereof, is the compound wherein R¹¹ is selected from the group consisting of hydrogen, C₁₋₂ alkyl and C₁₋₂ haloalkyl. In certain embodiments, the compound of Formula (IV), (IVa) or (IVb), or a pharmaceutically acceptable salt thereof, is the compound wherein R¹¹ is selected from the group consisting of C₁₋₂ alkyl and C₁₋₂ haloalkyl. In certain embodiments, the compound of Formula (IV), (IVa) or (IVb), or a pharmaceutically acceptable salt thereof, is the compound wherein R¹¹ can be selected from hydrogen, methyl, ethyl or CF₃. In certain embodiments, the compound of Formula (IV), (IVa) or (IVb), or a pharmaceutically acceptable salt thereof, is the compound wherein R¹¹ can be selected from methyl, ethyl or CF₃. In certain embodiments, the compound of Formula (IV), (IVa) or (IVb), or a pharmaceutically acceptable salt thereof, is the compound wherein R¹¹ can be selected from hydrogen, methyl, or CF₃. In certain embodiments, the compound of Formula (IV), (IVa) or (IVb), or a pharmaceutically acceptable salt thereof, is the compound wherein R¹¹ can be selected from methyl, or CF₃. In certain embodiments, the compound of Formula (IV), (IVa) or (IVb), or a pharmaceutically acceptable salt thereof, is the compound wherein R¹¹ can be selected from hydrogen or methyl. In certain embodiments, the compound of Formula (IV), (IVa) or (IVb), or a pharmaceutically acceptable salt thereof, wherein R¹¹ is selected from the group consisting of methyl and CF₃. In certain embodiments, the compound of Formula (IV), (IVa) or (IVb), or a pharmaceutically acceptable salt thereof, is the compound wherein R¹¹ is methyl. In certain embodiments, the compound of Formula (IV), (IVa) or (IVb), or a pharmaceutically acceptable salt thereof, is the compound wherein R¹¹ is hydrogen.

R¹² of Formula (IV), (IVa) and (IVb) can be any suitable group selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b},—NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur.

In certain embodiments, the compound of Formula (IV), (IVa) or (IVb), or a pharmaceutically acceptable salt thereof, wherein R¹² can be selected from C₁₋₂ alkyl, —C(O)NR^{a}R^{b}, and 5 membered heteroaryl having 1 to 3 nitrogen heteroatoms, wherein C₁₋₂ alkyl is optionally substituted with 1 to 5 substituents independently selected from halogen, —OH, —NR^{a}R^{b}, —NR^{a}C(O)R^{b}, —NR^{a}S(O)₂R^{b}, and C₁₋₃ haloalkyl, and each R^{a} and R^{b} is independently selected from the group consisting of hydrogen and C₁₋₃ alkyl, wherein each C₁₋₃ alkyl is optionally substituted with 1 to 3 substituents independently selected from hydroxyl and amino. In certain embodiments, the compound of Formula (IV), (IVa) or (IVb), or a pharmaceutically acceptable salt thereof, wherein R¹² is C₁₋₂ alkyl, optionally substituted with 1 to 3 substituents independently selected from halogen, —OH, —NH₂, —NHC(O)—C₁₋₃ alkyl, —NHS(O)₂—C₁₋₃ alkyl, and C₁₋₃ haloalkyl. In certain embodiments, the compound of Formula (IV), (IVa) or (IVb), or a pharmaceutically acceptable salt thereof, wherein R¹² is methyl or ethyl, each optionally substituted with 1 or 2 substituents independently selected from halogen, —OH, —NH₂, —NHC(O)—C₁₋₃ alkyl, and C₁₋₃ haloalkyl. In certain embodiments, the compound of Formula (IV), (IVa) or (IVb), or a pharmaceutically acceptable salt thereof, wherein R¹² is methyl or ethyl, wherein the methyl or ethyl is substituted with 1 or 2 substituents independently selected from —OH and —NHC(O)CH₃. In certain embodiments, the compound of Formula (IV), (IVa) or (IVb), or a pharmaceutically acceptable salt thereof, wherein R¹² can be selected from CH₂OH, CH₂CH₂OH, CH(Me)OH, CH(CH₂F)OH, CH(CHF₂)OH, CH(CF₃)OH, CF₃, CH₂NH₂, CH₂NHC(O)Me, CH(CH₂F)NHC(O)Me, CH₂NHS(O)₂Me, C(O)NH₂, C(O)NHMe, C(O)NH—CH₂CH₂OH, C(O)NH—CH₂CH₂NH₂, C(O)NH-(pyridin-2-ylmethyl), imidazolyl, and triazolyl. In certain embodiments, the compound of Formula (IV), (IVa) or (IVb), or a pharmaceutically acceptable salt thereof, wherein R¹² can be selected from CH₂OH, CH(Me)OH, CH(CH₂F)OH, and CH₂NHC(O)Me. In certain embodiments, the compound of Formula (IV), (IVa) or (IVb), or a pharmaceutically acceptable salt thereof, wherein R¹² can be selected from CH₂OH, CH(Me)OH, and CH₂NHC(O)Me. In certain embodiments, the compound of Formula (IV), (IVa) or (IVb), or a pharmaceutically acceptable salt thereof, wherein R¹² is —CH₂OH or —CH₂NC(O)CH₃.

In certain embodiments, the compound of Formula (IV), (IVa) or (IVb), or a pharmaceutically acceptable salt thereof, wherein R¹² is C₁₋₂ alkyl substituted with —NR^{a}C(O)R^{b}, wherein each R^{a} and R^{b} is independently selected from the group consisting of hydrogen and C₁₋₃ alkyl, wherein each C₁₋₃ alkyl is optionally substituted with 1 to 3 substituents independently selected from hydroxyl and amino.

R¹³ of Formula (IV), (IVa) and (IVb) can be any suitable group selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur.

In certain embodiments, the compound of Formula (IV), (IVa) or (IVb), or a pharmaceutically acceptable salt thereof, is the compound wherein R¹³ is C₃₋₆ alkyl optionally substituted with 1 to 2 substituents independently selected from halogen and —OH. In certain embodiments, the compound of Formula (IV), (IVa) or (IVb), or a pharmaceutically acceptable salt thereof, is the compound wherein R¹³ is C₃₋₆ alkyl optionally substituted with 1 to 2 halogen substituents. In certain embodiments, the compound of Formula (IV), (IVa) or (IVb), or a pharmaceutically acceptable salt thereof, is the compound wherein R¹³ is C₃₋₆ alkyl. Representative C₃₋₆ alkyl groups for R¹³ include, but are not limited to, n-propyl, iso-propyl, n-butyl, sec-butyl, iso-butyl, tert-butyl, n-pentyl, tert-pentyl, neopentyl, isopentyl, sec-pentyl and 3-pentyl. In certain embodiments, the compound of Formula (IV), (IVa) or (IVb), or a pharmaceutically acceptable salt thereof, is the compound wherein R¹³ is propyl, butyl or pentyl. In certain embodiments, the compound of Formula (IV), (IVa) or (IVb), or a pharmaceutically acceptable salt thereof, is the compound wherein R¹³ is n-propyl, n-butyl or n-pentyl. In certain embodiments, the compound of Formula (IV), (IVa) or (IVb), or a pharmaceutically acceptable salt thereof, is the compound wherein R¹³ is propyl or butyl.

R²⁰ of Formula (IV), (IVa) and (IVb) can be any suitable group selected from halogen, C₁₋₆haloalkyl, CN, —NR^{a}R^{b}, S(O)₁₋₂R^{a}, and OR^{a}. In certain embodiments, each R²⁰ can independently be selected from halogen, CN, —NR^{a}R^{b}, and OR^{a}. In certain embodiments, each R²⁰ can independently be selected from halogen, CN, —NR^{a}R^{b}, and OR^{a}. In certain embodiments, each R²⁰ can independently be halogen. In certain embodiments, each R²⁰ can independently be selected from fluoro, chloro, bromo, CN, —NH₂, OH, OMe, and OEt. In certain embodiments, each R²⁰ can independently be selected from fluoro and chloro.

R^{a} and R^{b} of Formula (IV), (IVa) and (IVb) can each independently be any suitable group selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl. In certain embodiments, R^{a} and R^{b} can each independently be selected from hydrogen and C₁₋₃ alkyl, wherein each C₁₋₃ alkyl is optionally substituted with 1 to 3 substituents independently selected from halogen, hydroxyl, amino, and C₁₋₆ haloalkyl. In certain embodiments, R^{a} and R^{b} can each independently be selected from hydrogen and C₁₋₃ alkyl, wherein each C₁₋₃ alkyl is optionally substituted with 1 to 3 substituents independently selected from hydroxyl and amino. In certain embodiments, R^{a} and R^{b} can each independently be selected from hydrogen and C₁₋₃ alkyl, wherein each C₁₋₃ alkyl is optionally substituted with 1 substituent selected from hydroxyl and amino. In certain embodiments, R^{a} and R^{b} can each independently be selected from hydrogen and C₁₋₃ alkyl. In certain embodiments, R^{a} and R^{b} can each independently be selected from hydrogen, methyl, ethyl, propyl, butyl, CF₃, CH₂CF₃, CH₂CH₂CF₃, CH₂OH, CH₂CH₂OH, CH₂NH₂, and CH₂CH₂NH₂. In certain embodiments, R^{a} and R^{b} can each independently be selected from hydrogen, methyl, ethyl, CF₃, CH₂OH, CH₂CH₂OH, CH₂NH₂, and CH₂CH₂NH₂. In certain embodiments, R^{a} and R^{b} can each independently be selected from hydrogen, methyl, ethyl, CH₂CH₂OH, and CH₂CH₂NH₂. In certain embodiments, R^{a} and R^{b} can each independently be selected from hydrogen, methyl and ethyl. In certain embodiments, R^{a} and R^{b} can each independently be selected from hydrogen and methyl.

In certain embodiments, the compound of Formula (IV), (IVa) or (IVb), or a pharmaceutically acceptable salt thereof, is the compound wherein:

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;

R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R¹¹ is selected from the group consisting of hydrogen, C₁₋₂ alkyl, C₃₋₆ cycloalkyl, and C₁₋₃ haloalkyl;

R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

each R²⁰ is independently selected from the group consisting of halogen, CN, —NR^{a}R^{b}, and OR^{a}; and

each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IV), (IVa) or (IVb), or a pharmaceutically acceptable salt thereof, is the compound wherein:

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;

R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

R¹¹ is selected from the group consisting of C₁₋₂ alkyl, C₃₋₆ cycloalkyl, and C₁₋₃ haloalkyl;

R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, -NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

each R²⁰ is independently selected from the group consisting of halogen, CN, —NR^{a}R^{b}, and OR^{a}; and

each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IV), (IVa) or (IVb), or a pharmaceutically acceptable salt thereof, is the compound wherein:

R¹ is selected from the group consisting of hydrogen, halogen, C₁₋₆ alkyl, CN, and OR^{a}, wherein C₁₋₆ alkyl is optionally substituted with 1 to 5 R²⁰ groups;

R² is selected from the group consisting of hydrogen, halogen, C₁₋₆ alkyl, CN, and OR^{a}, wherein C₁₋₆ alkyl optionally substituted with 1 to 5 R²⁰ groups;

R³ is selected from the group consisting of hydrogen, halogen, C₁₋₆ alkyl, CN, and OR^{a}, wherein C₁₋₆ alkyl is optionally substituted with 1 to 5 R²⁰ groups;

R¹¹ is selected from the group consisting of hydrogen, C₁₋₂ alkyl, C₃₋₆ cycloalkyl, and C₁₋₃ haloalkyl;

R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

each R²⁰ is independently selected from the group consisting of halogen, CN, —NR^{a}R^{b}, and OR^{a}; and

each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IV), (IVa) or (IVb), or a pharmaceutically acceptable salt thereof, is the compound wherein:

R¹ is selected from the group consisting of hydrogen, halogen, C₁₋₆ alkyl, CN, and OR^{a}, wherein C₁₋₆ alkyl is optionally substituted with 1 to 5 R²⁰ groups;

R² is selected from the group consisting of hydrogen, halogen, C₁₋₆ alkyl, CN, and OR^{a}, wherein C₁₋₆ alkyl optionally substituted with 1 to 5 R²⁰ groups;

R³ is selected from the group consisting of hydrogen, halogen, C₁₋₆ alkyl, CN, and OR^{a}, wherein C₁₋₆ alkyl is optionally substituted with 1 to 5 R²⁰ groups;

R¹¹ is selected from the group consisting of C₁₋₂ alkyl, C₃₋₆ cycloalkyl, and C₁₋₃ haloalkyl;

R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂Ra, —S(O)₂NR^{a}R^{b}, NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

each R²⁰ is independently selected from the group consisting of halogen, CN, —NR^{a}R^{b}, and OR^{a}; and

each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IV), (IVa) or (IVb), or a pharmaceutically acceptable salt thereof, wherein R¹¹ is methyl or CF₃, R¹² is —CH₂OH, —CH(Me)OH or —CH₂NHC(O)CH₃, and R¹³ is selected from the group consisting of propyl, butyl and pentyl.

In certain embodiments, the compound of Formula (IV), (IVa) or (IVb), or a pharmaceutically acceptable salt thereof, wherein R¹¹ is methyl or CF₃, R¹² is —CH₂OH, —CH(Me)OH, CH₂NHCH(CH₃)(CF₃) or —CH₂NHC(O)CH₃, and R¹³ is selected from the group consisting of propyl, butyl and pentyl.

In certain embodiments, the compound of Formula (IV), (IVa) or (IVb), or a pharmaceutically acceptable salt thereof, wherein R¹¹ is methyl, R¹² is —CH₂OH or —CH₂NHC(O)CH₃, and R¹³ is selected from the group consisting of propyl and butyl.

In certain embodiments, the compound of Formula (IV), or a pharmaceutically acceptable salt thereof, wherein the moiety

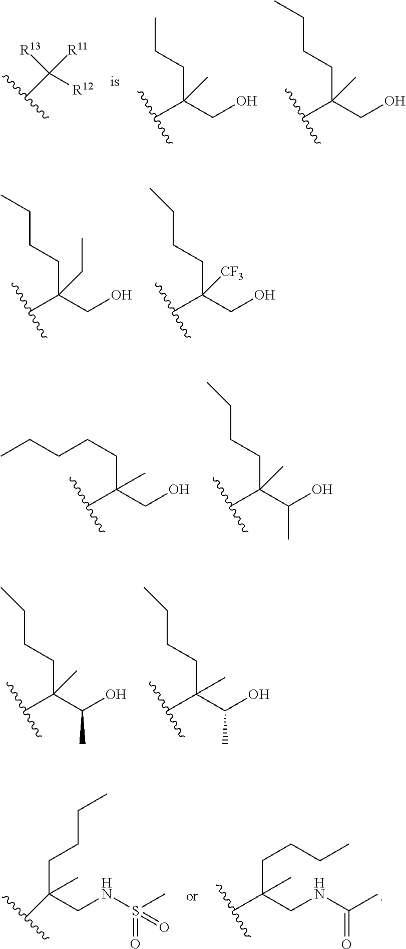

In certain embodiments, the compound of Formula (IV), or a pharmaceutically acceptable salt thereof, wherein the moiety

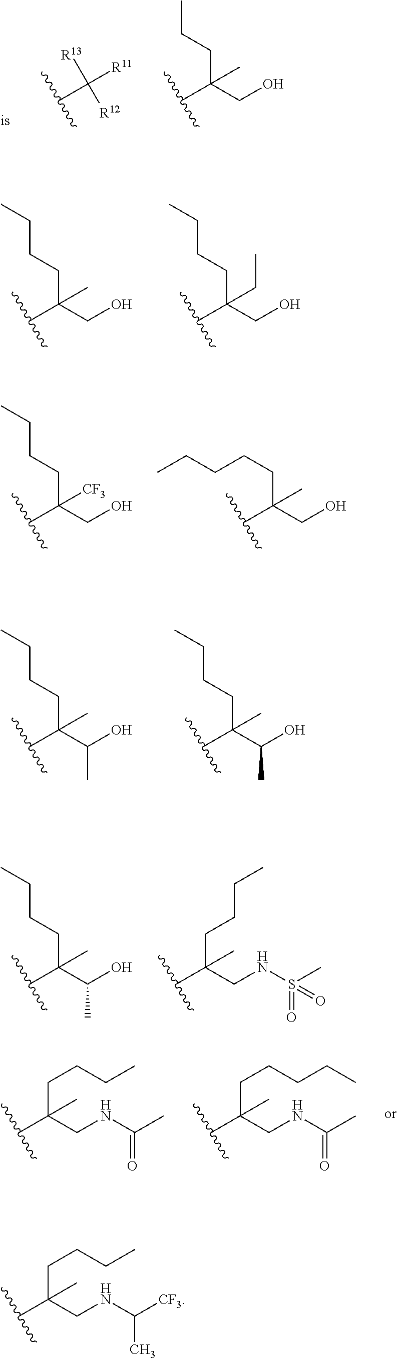

In certain embodiments, the compound of Formula (IV) or (IVa), or a pharmaceutically acceptable salt thereof, wherein the moiety

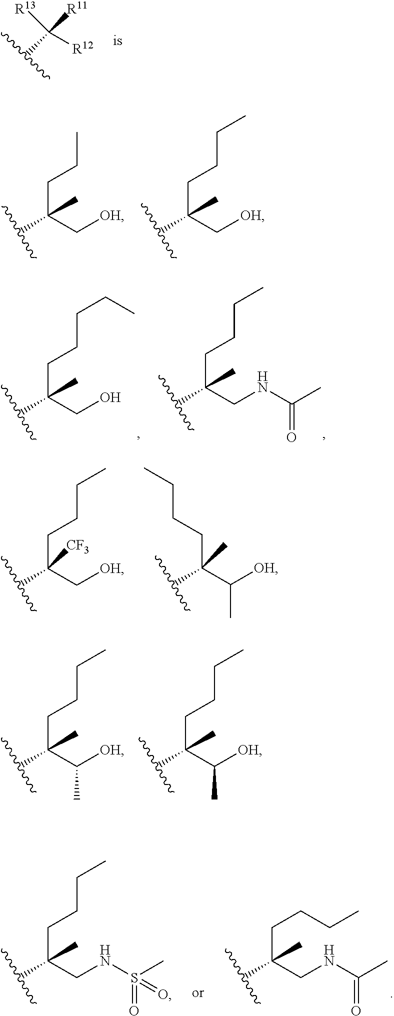

In certain embodiments, the compound of Formula (IV) or (IVa), or a pharmaceutically acceptable salt thereof, wherein the moiety

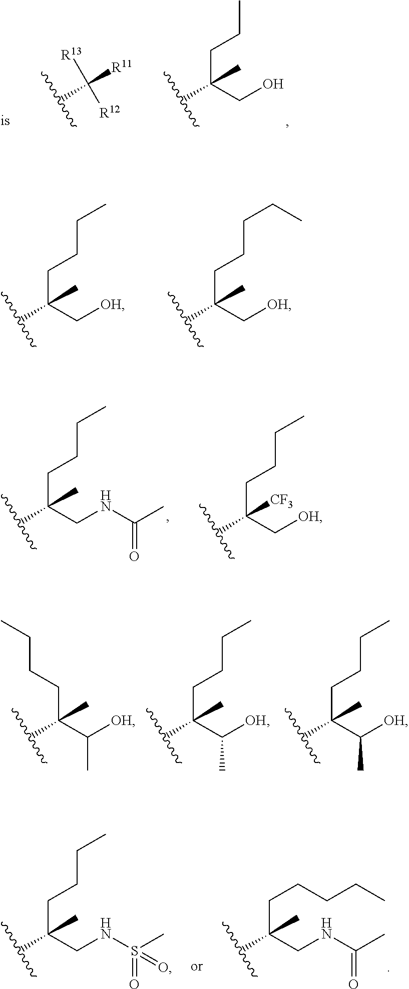

In certain embodiments, the compound of Formula (IV) or (IVa), or a pharmaceutically acceptable salt thereof, wherein the moiety

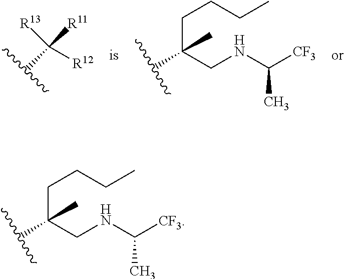

In certain embodiments, the compound of Formula (IV) or (IVa), or a pharmaceutically acceptable salt thereof, wherein the moiety

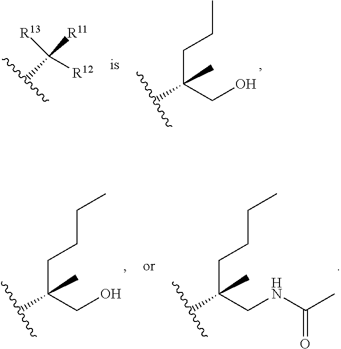

In certain embodiments, the compound of Formula (IV) or (IVa), or a pharmaceutically acceptable salt thereof, wherein the moiety

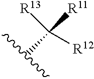

can also be drawn as the moiety

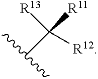

In certain embodiments, the compound of Formula (IV) or (IVb), or a pharmaceutically acceptable salt thereof, wherein the moiety

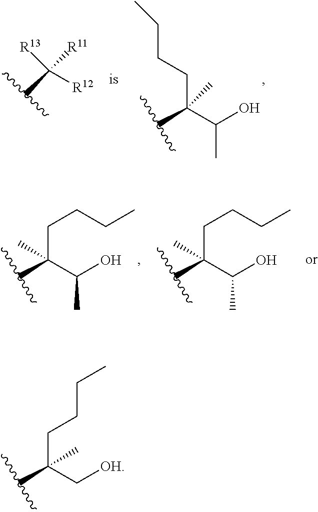

In certain embodiments, the compound of Formula (IV) or (IVb), or a pharmaceutically acceptable salt thereof, wherein the moiety

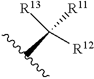

can also be drawn as the moiety

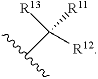

In certain embodiments, the compound of Formula (IV) or (IVa), or a pharmaceutically acceptable salt thereof, is a compound of Formula (IVc)

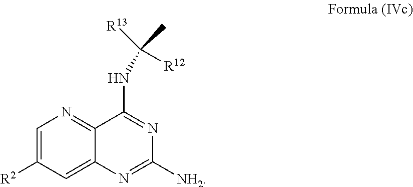

The R², R¹² and R¹³ groups of Formula (IVc) are as defined above for Formula (J), (I), (IV) or (IVa), or any combination thereof. For example, R² can be selected from hydrogen, halogen, C₁₋₃ alkyl, CN and OR^{a}, wherein C₁₋₃ alkyl is optionally substituted with 1 to 5 halogen groups, R¹² can be selected from C₁₋₂ alkyl, —C(O)NR^{a}R^{b}, and 5 membered heteroaryl having 1 to 3 nitrogen heteroatoms, wherein C₁₋₂ alkyl is optionally substituted with 1 to 5 substituents independently selected from halogen, —OH, —NR^{a}R^{b}, —NR^{a}C(O)R^{b}, —NR^{a}S(O)₂R^{b}, and C₁₋₃ haloalkyl, and R¹³ can be C₃₋₆ alkyl optionally substituted with 1 to 2 substituents independently selected from halogen and —OH. In certain embodiments, the compound of Formula (IV), (IVa), or (IVc), or a pharmaceutically acceptable salt thereof, is a compound wherein R² can be selected from hydrogen, methyl, ethyl, fluoro, chloro, bromo, CF₃, CN, OH, OMe, and OEt, and R¹² can be selected CH₂OH, CH₂CH₂OH, CH(Me)OH, CH(CH₂F)OH, CH(CHF₂)OH, CH(CF₃)OH, CF₃, CH₂NH₂, CH₂NHC(O)Me, CH(CH₂F)NHC(O)Me, CH₂NHS(O)₂Me, C(O)NH₂, C(O)NHMe, C(O)NH-CH₂CH₂OH, C(O)NH—CH₂CH₂NH₂, C(O)NH-(pyridin-2-ylmethyl), imidazolyl, and triazolyl, and R¹³ can be propyl, butyl or pentyl. In certain embodiments, the compound of Formula (IV), (IVa), or (IVc), or a pharmaceutically acceptable salt thereof, is a compound wherein R² can be selected from hydrogen, methyl, fluoro, and chloro, and R¹² can be selected CH₂OH, CH(Me)OH, CH(CH₂F)OH, and CH₂NHC(O)Me, and R¹³ can be propyl, butyl or pentyl. In certain embodiments, the compound of Formula (IV), (IVa), or (IVc), or a pharmaceutically acceptable salt thereof, is a compound wherein R² is hydrogen or fluoro, R¹² is —CH₂OH or —CH₂NHC(O)CH₃, and R¹³ is selected from propyl and butyl. In certain embodiments, the compound of Formula (IV), (IVa), or (IVc), or a pharmaceutically acceptable salt thereof, is a compound wherein R² is hydrogen, chloro, or fluoro, R¹² is —CH₂OH or —CH₂NHC(O)CH₃, and R¹³ is selected from butyl or pentyl.

In certain embodiments, the compound of Formula (IV) or (IVa), or a pharmaceutically acceptable salt thereof, is a compound of Formula (IVd)

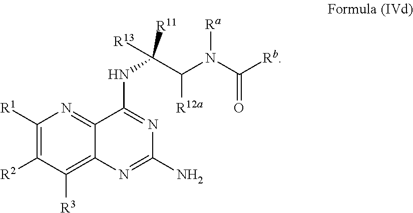

The R¹, R², R³, R¹¹, R¹³, R^{a} and R^{b} groups of Formula (IVd) can be as defined above for Formula (J), (I), (IV), or (IVa), or any combination thereof. R^{12a} can be any suitable group selected from hydrogen, C₁₋₂ alkyl and C₁₋₃ haloalkyl. In certain embodiments, the compound of Formula (IV), (IVa) or (IVd), or a pharmaceutically acceptable salt thereof, is a compound wherein R^{12a} can be selected from hydrogen, C₁₋₂ alkyl and C₁₋₃ haloalkyl. In certain embodiments, the compound of Formula (IV), (IVa) or (IVd), or a pharmaceutically acceptable salt thereof, is a compound wherein R^{12a} can be selected from hydrogen, methyl, ethyl and CF₃. In certain embodiments, the compound of Formula (IV), (IVa) or (IVd), or a pharmaceutically acceptable salt thereof, is a compound wherein R^{12a} can be hydrogen.

In certain embodiments, the compound of Formula (IVd), or a pharmaceutically acceptable salt thereof, is the compound wherein R¹ is selected from the group consisting of hydrogen, halogen, C₁₋₆ alkyl, CN, and OR^{a}, wherein C₁₋₆ alkyl is optionally substituted with 1 to 5 R²⁰ groups, R² is selected from the group consisting of hydrogen, halogen, C₁₋₆ alkyl, CN, and OR^{a}, wherein C₁₋₆ alkyl optionally substituted with 1 to 5 R²⁰ groups, R³ is selected from the group consisting of hydrogen, halogen, C₁₋₆ alkyl, CN, and OR^{a}, wherein C₁₋₆ alkyl is optionally substituted with 1 to 5 R²⁰ groups, R¹¹ is C₁₋₂ alkyl or CF₃, R^{12a} is selected from the group consisting of hydrogen, C₁₋₂ alkyl and C₁₋₃ haloalkyl, R¹³ is C₃₋₆ alkyl optionally substituted with 1 to 2 halogen substituents, each R²⁰ is independently selected from the group consisting of halogen, C₁₋₆haloalkyl, CN, —NR^{a}R^{b}, S(O)₁₋₂R^{a}, and OR^{a}, and each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IVd), or a pharmaceutically acceptable salt thereof, is the compound wherein R¹ is selected from the group consisting of hydrogen, halogen, and C₁₋₃ alkyl, R² is selected from the group consisting of hydrogen, halogen, and C₁₋₃ alkyl, R³ is selected from the group consisting of hydrogen, halogen, and C₁₋₃ alkyl, R¹¹ is C₁₋₂ alkyl or CF₃, R^{12a} is selected from the group consisting of hydrogen, C₁₋₂ alkyl and C₁₋₃ haloalkyl, R¹³ is C₃₋₆ alkyl optionally substituted with 1 to 2 halogen substituents, and each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IVd), or a pharmaceutically acceptable salt thereof, has the structure:

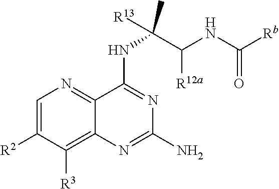

wherein R² is selected from the group consisting of hydrogen, methyl, fluoro, and chloro, R³ is selected from the group consisting of hydrogen and methyl, R^{12a} is selected from the group consisting of hydrogen, C₁₋₂ alkyl and C₁₋₃ haloalkyl, R¹³ is C₃₋₆ alkyl, and R^{b} is methyl or ethyl, each optionally substituted with hydroxyl or amino.

In certain embodiments, the compound of Formula (IVd), or a pharmaceutically acceptable salt thereof, has the structure:

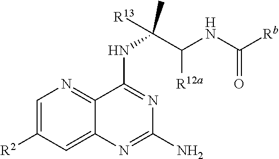

wherein R² is selected from the group consisting of hydrogen, methyl, fluoro, and chloro, R^{12a} is selected from the group consisting of hydrogen, C₁₋₂ alkyl and C₁₋₃ haloalkyl, R¹³ is C₃₋₆ alkyl, and R^{b} is methyl or ethyl, each optionally substituted with hydroxyl or amino. In certain embodiments, R² and R¹³ can be as defined above for Formula (J), (I), (IV), or (IVa), or any combination thereof.

In certain embodiments, the compound of Formula (IVd), or a pharmaceutically acceptable salt thereof, has the structure:

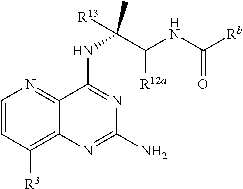

wherein R³ is selected from the group consisting of hydrogen and methyl, R^{12a} is selected from the group consisting of hydrogen, C₁₋₂ alkyl and C₁₋₃ haloalkyl, R¹³ is C₃₋₆ alkyl, and R^{b} is methyl or ethyl, each optionally substituted with hydroxyl or amino.

In certain embodiments, the compound of Formula (IVd), or a pharmaceutically acceptable salt thereof, has the structure:

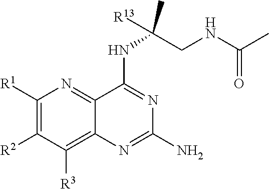

wherein R¹³ is C₃₋₆ alkyl. R¹, R² and R³ can be as defined above for Formula (J), (I), (IV), (IVa) or (IVd).

In certain embodiments, the compound of Formula (IVd), or a pharmaceutically acceptable salt thereof, has the structure:

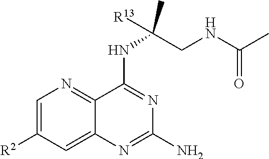

wherein R² is selected from the group consisting of hydrogen and F, and R¹³ is C₃₋₆ alkyl. In certain embodiments, R² and R¹³ can be as defined above for Formula (J), (I), (IV), or (IVa), or any combination thereof.

In certain embodiments, the compound of Formula (IVd), or a pharmaceutically acceptable salt thereof, has the structure:

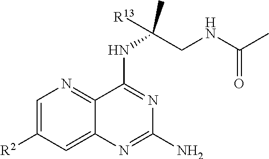

wherein R² is selected from the group consisting of hydrogen, Cl,. and F, and R¹³ is C₃₋₆ alkyl. In certain embodiments, R² and R¹³ can be as defined above for Formula (J), (I), (IV), or (IVa), or any combination thereof.

In certain embodiments, the compound of Formula (IVd), or a pharmaceutically acceptable salt thereof, has the structure:

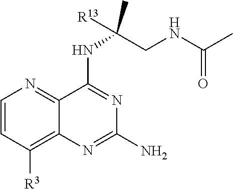

wherein R³ is selected from the group consisting of hydrogen and methyl, and R¹³ is C₃₋₆ alkyl.

In certain embodiments, the compound of Formula (J), (I), or (IV), is selected from:

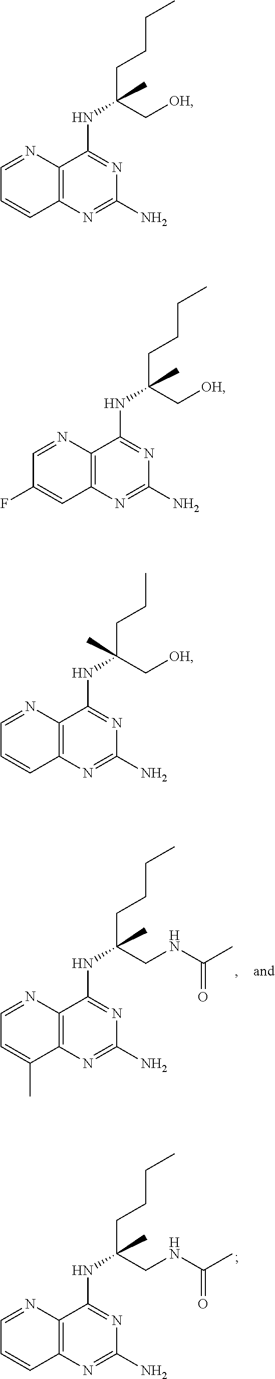

or a pharmaceutically acceptable salt thereof.

In certain embodiments, the compound of Formula (J), (I), or (IV), is selected from:

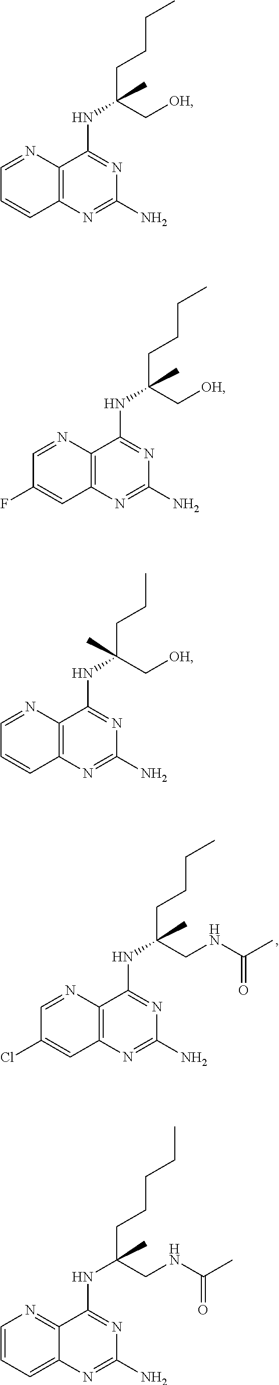

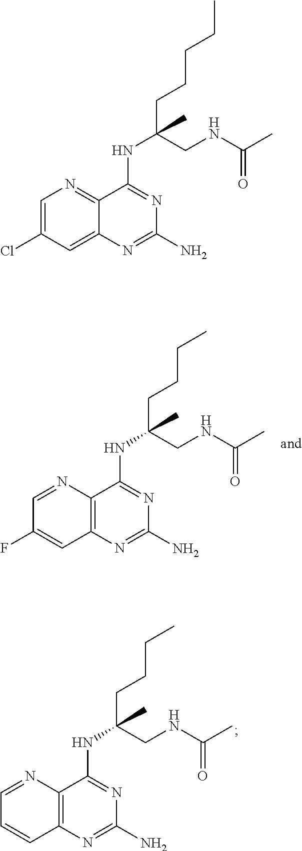

or a pharmaceutically acceptable salt thereof.

In certain embodiments, the compound of Formula (J), (I), or (IV), is:

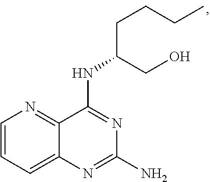 [SMILES: CCCC[C@H](CO)Nc1nc(N)nc2cccnc12]

or a pharmaceutically acceptable salt thereof.

In certain embodiments, the compound of Formula (J), (I), or (IV), or a pharmaceutically acceptable salt thereof, is a compound of the following formula:

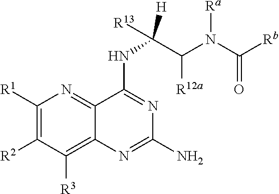

wherein R¹ is selected from the group consisting of hydrogen, halogen, C₁₋₆ alkyl, CN, and OR^{a}, wherein C₁₋₆ alkyl is optionally substituted with 1 to 5 R²⁰ groups, R² is selected from the group consisting of hydrogen, halogen, C₁₋₆ alkyl, CN, and OR^{a}, wherein C₁₋₆ alkyl optionally substituted with 1 to 5 R²⁰ groups, R³ is selected from the group consisting of hydrogen, halogen, C₁₋₆ alkyl, CN, and OR^{a}, wherein C₁₋₆ alkyl is optionally substituted with 1 to 5 R²⁰ groups, R^{12a} is selected from the group consisting of hydrogen, C₁₋₂ alkyl and C₁₋₃ haloalkyl, R¹³ is C₃₋₆ alkyl optionally substituted with 1 to 2 halogen substituents, each R²⁰ is independently selected from the group consisting of halogen, C₁₋₆haloalkyl, CN, —NR^{a}R^{b}, S(O)₁₋₂R^{a}, and OR^{a}, and each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (J), (I), or (IV), or a pharmaceutically acceptable salt thereof, is a compound of the following formula:

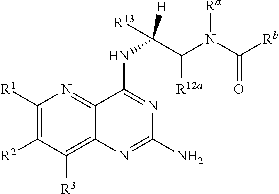

wherein R¹ is selected from the group consisting of hydrogen, halogen, and C₁₋₃ alkyl, R² is selected from the group consisting of hydrogen, halogen, and C₁₋₃ alkyl, R³ is selected from the group consisting of hydrogen, halogen, and C₁₋₃ alkyl, R^{12a} is selected from the group consisting of hydrogen, C₁₋₂ alkyl and C₁₋₃ haloalkyl, R¹³ is C₃₋₆ alkyl optionally substituted with 1 to 2 halogen substituents, and each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (J), (I), or (IV), or a pharmaceutically acceptable salt thereof, is a compound of the following formula:

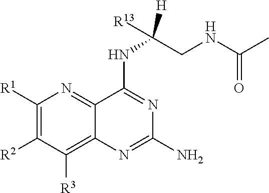

wherein R¹³ is C₃₋₆ alkyl. R¹, R² and R³ can be as defined above for Formula (J), (I), (IV), (IVa) or (IVd).

In certain embodiments of a compound of of Formula (J), (I), (II), (IIa), (IIb), (III), (IIIa), or (IIIb), R¹ is hydrogen, halogen, or C₁₋₆alkyl optionally substituted with 1 to 5 R²⁰ groups. In certain embodiments of a compound of Formula (J), (I), (II), (IIa), (IIb), (III), (IIIa), (IIIb), (IV), (IVa), (IVb), or (IVd), R¹ is hydrogen, halogen, or C₁₋₆alkyl optionally substituted with 1 to 5 R²⁰ groups.

In certain embodiments of a compound of of Formula (J), (I), (II), (IIa), (IIb), (III), (IIIa), or (IIIb), R¹ is hydrogen, halogen, or C₁₋₃alkyl optionally substituted with 1 to 5 halogens. In certain embodiments of a compound of Formula (J), (I), (II), (IIa), (IIb), (III), (IIIa), (IIIb), (IV), (IVa), (IVb) or (IVd), R¹ is hydrogen, halogen, or C₁₋₃alkyl optionally substituted with 1 to 5 halogens.

In certain embodiments of a compound of of Formula (J), (I), (II), (IIa), (IIb), (III), (IIIa), or (IIIb), R¹ is hydrogen, Cl, CH₃, or CF₃. In certain embodiments of a compound of Formula (J), (I), (II), (IIa), (IIb), (III), (IIIa), (IIIb), (IV), (IVa), (IVb) or (IVd), R¹ is hydrogen, Cl, CH₃, or CF₃.

In certain embodiments of a compound of of Formula (J), (I), (II), (IIa), (IIb), (III), (IIIa), or (IIIb), R² is hydrogen, halogen, CN, or C₁₋₆alkyl optionally substituted with 1 to 5 R²⁰ groups. In certain embodiments of a compound of Formula (J), (I), (II), (IIa), (IIb), (III), (IIIa), (IIIb), (IV), (IVa), (IVb), (IVc), or (IVd), R² is hydrogen, halogen, CN, or C₁₋₆alkyl optionally substituted with 1 to 5 R²⁰ groups.

In certain embodiments of a compound of of Formula (J), (I), (II), (IIa), (IIb), (III), (IIIa), or (IIIb), R² is hydrogen, halogen, CN or C₁₋₃alkyl optionally substituted with 1 to 5 halogens. In certain embodiments of a compound of Formula (J), (I), (II), (IIa), (IIb), (III), (IIIa), (IIIb), (IV), (IVa), (IVb), (IVc), or (IVd), R² is hydrogen, halogen, CN or C₁₋₃alkyl optionally substituted with 1 to 5 halogens.

In certain embodiments of a compound of of Formula (J), (I), (II), (IIa), (IIb), (III), (IIIa), or (IIIb), R² is hydrogen, CH₃, —CH₂CH₃, F, Br, CI, or CN. In certain embodiments of a compound of Formula (J), (I), (II), (IIa), (IIb), (III), (IIIa), (IIIb), (IV), (IVa), (IVb), (IVc), or (IVd), R² is hydrogen, CH₃, —CH₂CH₃, F, Br, Cl, or CN.

In certain embodiments of a compound of of Formula (J), (I), (II), (IIa), (IIb), (III), (IIIa), or (IIIb), R³ is hydrogen, halogen, or C₁₋₆alkyl optionally substituted with 1 to 5 R²⁰ groups. In certain embodiments of a compound of Formula (J), (I), (II), (IIa), (IIb), (III), (IIIa), (IIIb), (IV), (IVa), (IVb) or (IVd), R³ is hydrogen, halogen, or C₁₋₆alkyl optionally substituted with 1 to 5 R²⁰ groups.

In certain embodiments of a compound of of Formula (J), (I), (II), (IIa), (IIb), (III), (IIIa), or (IIIb), R³ is hydrogen, halogen, or C₁₋₃alkyl optionally substituted with 1 to 5 R²⁰ groups.

In certain embodiments of a compound of Formula (J), (I), (II), (IIa), (IIb), (III), (IIIa), (IIIb), (IV), (IVa), (IVb) or (IVd), R³ is hydrogen, halogen, or C₁₋₃alkyl optionally substituted with 1 to 5 R²⁰ groups.

In certain embodiments of a compound of of Formula (J), (I), (II), (IIa), (IIb), (III), (IIIa), or (IIIb), R³ is hydrogen, Cl, or CH₃. In certain embodiments of a compound of Formula (J), (I), (II), (IIa), (IIb), (III), (IIIa), (IIIb), (IV), (IVa), (IVb) or (IVd), R³ is hydrogen, Cl, or CH₃.

In certain embodiments of a compound of Formula (J), R¹⁰ is hydrogen, F, CI, or CH₃.

In certain embodiments of a compound of Formula (J), R¹⁰ is hydrogen.

In certain embodiments of a compound of of Formula (J), (I), (II), (IIa), (IIb), (III), (IIIa), or (IIIb), R¹, R², and R³ are hydrogen. In certain embodiments of a compound of Formula (J), (I), (II), (IIa), (IIb), (III), (IIIa), (IIIb), (IV), (IVa), (IVb), ((IVO, or (IVd), R¹, R², and R³ are hydrogen.

In certain embodiments of a compound of of Formula (J), (I), (II), (IIa), (IIb), (III), (IIIa), or (IIIb), R¹ and R³ are hydrogen and R² is F. In certain embodiments of a compound of Formula (J), (I), (II), (IIa), (IIb), (III), (IIIa), (IIIb), (IV), (IVa), (IVb), (IVc), or (IVd), R¹ and R³ are hydrogen and R² is F.

In certain embodiments of a compound of Formula (J), (I), (II), (IIa), (IIb), (III), (IIIa), or (IIIb), R¹ is hydrogen, halogen, or C₁₋₆alkyl optionally substituted with 1 to 5 R²⁰ groups.

In certain embodiments of a compound of Formula (J), (I), (II), (IIa), (IIb), (III), (IIIa), or (IIIb), R¹ is hydrogen, halogen, or C₁₋₃alkyl optionally substituted with 1 to 5 halogens.

In certain embodiments of a compound of Formula (J), (I), (II), (IIa), (IIb), (III), (IIIa), or (IIIb), R¹ is hydrogen, Cl, CH₃, or CF₃.

In certain embodiments of a compound of Formula (J), (I), (II), (IIa), (IIb), (III), (IIIa), or (IIIb), R² is hydrogen, halogen, CN, or C₁₋₆alkyl optionally substituted with 1 to 5 R²⁰ groups.

In certain embodiments of a compound of Formula (J), (I), (II), (IIa), (IIb), (III), (IIIa), or (IIIb), R² is hydrogen, halogen, CN or C₁₋₃alkyl optionally substituted with 1 to 5 halogens.

In certain embodiments of a compound of Formula (J), (I), (II), (IIa), (IIb), (III), (IIIa), or (IIIb), R² is hydrogen, CH₃, —CH₂CH₃, F, Br, Cl, or CN.

In certain embodiments of a compound of Formula (J), (I), (II), (IIa), (IIb), (III), (IIIa), or (IIIb), R³ is hydrogen, halogen, or C₁₋₆alkyl optionally substituted with 1 to 5 R²⁰ groups.

In certain embodiments of a compound of Formula (J), (I), (II), (IIa), (IIb), (III), (IIIa), or (IIIb), R³ is hydrogen, halogen, or C₁₋₃alkyl optionally substituted with 1 to 5 R²⁰ groups.

In certain embodiments of a compound of Formula (J), (I), (II), (IIa), (IIb), (III), (IIIa), or (IIIb), R³ is hydrogen, Cl, or CH₃.

In certain embodiments of a compound of Formula (J), R¹⁰ is hydrogen, F, Cl, or CH₃.

In certain embodiments of a compound of Formula (J), R¹⁰ is hydrogen.

In certain embodiments of a compound of Formula (J), (I), (II), (IIa), (IIb), (III), (IIIa), or (IIIb), R¹, R², and R³ are hydrogen.

In certain embodiments of a compound of Formula (J), (I), (II), (IIa), (IIb), (III), (IIIa), or (IIIb), R¹ and R³ are hydrogen and R² is F.

It is understood that each of the variables (e.g. R¹, R², R³, R⁴) may be combined with any other variables for Formula (J), (I), (II), (IIa) or (IIb) (e.g. R¹, R², R³, R⁴). Further, in instances describing a compound of Formula (J) or (I), it is understood that the variables also describe compounds of other formulae (e.g. Formula (II), (IIa), (IIb), (III), (IIIa), and (IIIb)) which fall within the scope of Formula (J) or (I).

It is understood that any variable for R¹ of Formula (J), (I), (II), (IIa), (IIb), (III), (IIIa), or (IIIb) may be combined with any variable of R⁴ in Formula (J), (I), (II), (IIa), (IIb), (III), (IIIa), or (IIIb), the same as if each and every combination were specifically and individually listed. For example, in one variation of Formula (J) or (I), R¹ is hydrogen, Cl, CH₃ or CF₃, and R⁴ is C₁₋₆ alkyl which is optionally substituted with 1 or 2 substituents independently selected from OH, CF₃,—C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl

It is understood that any variable for R² of Formula (J), (I), (II), (IIa), (IIb), (III), (IIIa), or (IIIb) may be combined with any variable of R⁴ in Formula (J), (I), (II), (IIa), (IIb), (III), (IIIa), or (IIIb), the same as if each and every combination were specifically and individually listed. For example, in one variation of Formula (J) or (I), R² is hydrogen, CH₃, —CH₂CH₃, F, Br, Cl, or CN, and R⁴ is C₁₋₆ alkyl which is optionally substituted with 1 or 2 substituents independently selected from OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl.

It is understood that any variable for R³ of Formula (J), (I), (II), (IIa), (IIb), (III), (IIIa), or (IIIb) may be combined with any variable of R⁴ in Formula (J), (I), (II), (IIa), (IIb), (III), (IIIa), or (IIIb), the same as if each and every combination were specifically and individually listed. For example, in one variation of Formula (J) or (I), R³ is hydrogen, Cl, or CH₃, and R⁴ is C₁₋₆ alkyl which is optionally substituted with 1 or 2 substituents independently selected from OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl.

In certain embodiments, the compound of Formula (J) or (I), or a pharmaceutically acceptable salt thereof, has one or more features selected from:
(a) R⁴ is C₁₋₆ alkyl which is optionally substituted with 1 or 2 substituents independently selected halogen, —OR^{a}, —C(O)OR^{a}, —C(O)NR^{a}R^{b}, —SR^{a}, C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups and wherein R^{a} and R^{b} are each independently hydrogen or C₁₋₄alkyl, wherein each C₁₋₄ alkyl is optionally substituted with —NH₂, OH, or pyridyl;
(b) R¹ is hydrogen, halogen, or C₁₋₆alkyl optionally substituted with 1 to 5 R²⁰ groups;
(c) R² is hydrogen, halogen, CN, or C₁₋₆alkyl optionally substituted with 1 to 5 R²⁰ groups; and
(d) R³ is hydrogen, halogen, or C₁₋₃alkyl optionally substituted with 1 to 5 R²⁰ groups.

In certain embodiments, the compound of Formula (J) or (I), or a pharmaceutically acceptable salt thereof has two or more features selected from (a)-(d), as listed above. In certain embodiments, the compound of Formula (J) or (I), or a pharmaceutically acceptable salt thereof has three or more features selected from (a)-(d), as listed above. In certain embodiments, the compound of Formula (J) or (I), or a pharmaceutically acceptable salt thereof has four features selected from (a)-(d), as listed above.

In certain embodiments, the compound of Formula (J) or (I), or a pharmaceutically acceptable salt thereof has one or more features selected from:
(e) R⁴ is C₁₋₆ alkyl which is optionally substituted with 1 or 2 substituents independently selected from OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl.
(f) R¹ is hydrogen, halogen, or C₁₋₃alkyl optionally substituted with 1 to 5 halogens;
(g) R² is hydrogen, halogen, CN or C₁₋₃alkyl optionally substituted with 1 to 5 halogens; and
(h) R³ is hydrogen, halogen, or C₁₋₃alkyl.

In certain embodiments, the compound of Formula (J) or (I), or a pharmaceutically acceptable salt thereof has two or more features selected from (e)-(h), as listed above. In certain embodiments, the compound of Formula (J) or (I), or a pharmaceutically acceptable salt thereof has three or more features selected from (e)-(h), as listed above. In certain embodiments, the compound of Formula (J) or (I), or a pharmaceutically acceptable salt thereof has two or more features selected from (e)-(h), as listed above.

In certain embodiments, the compound of Formula (J) or (I) is selected from:

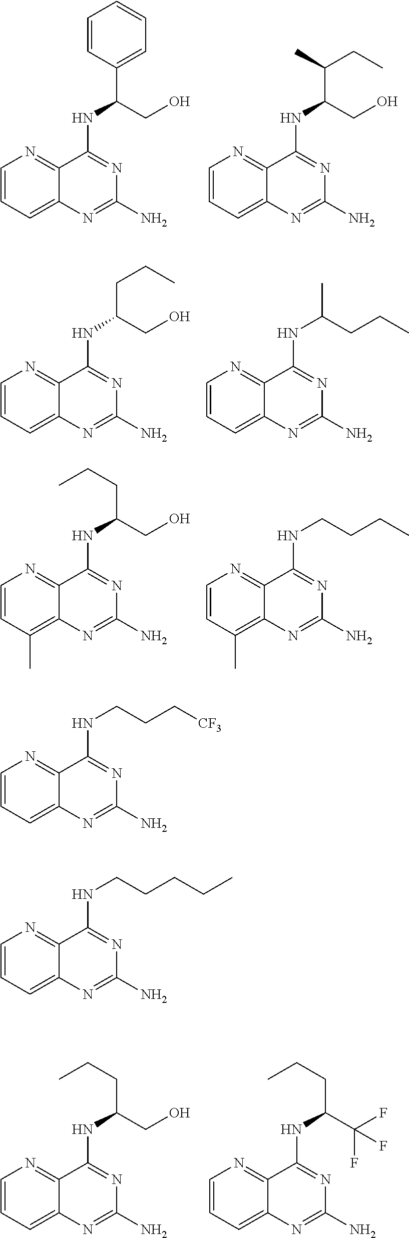

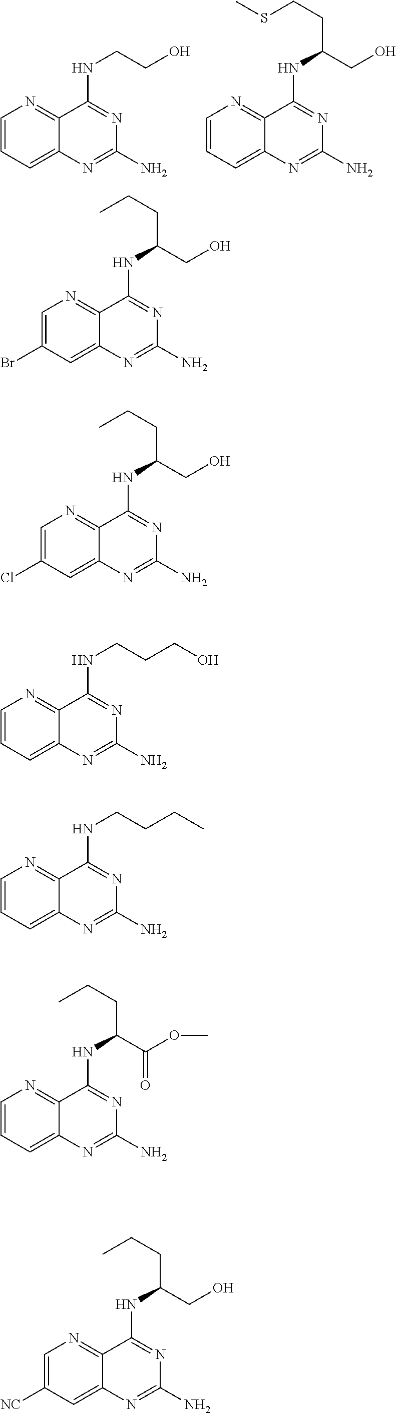

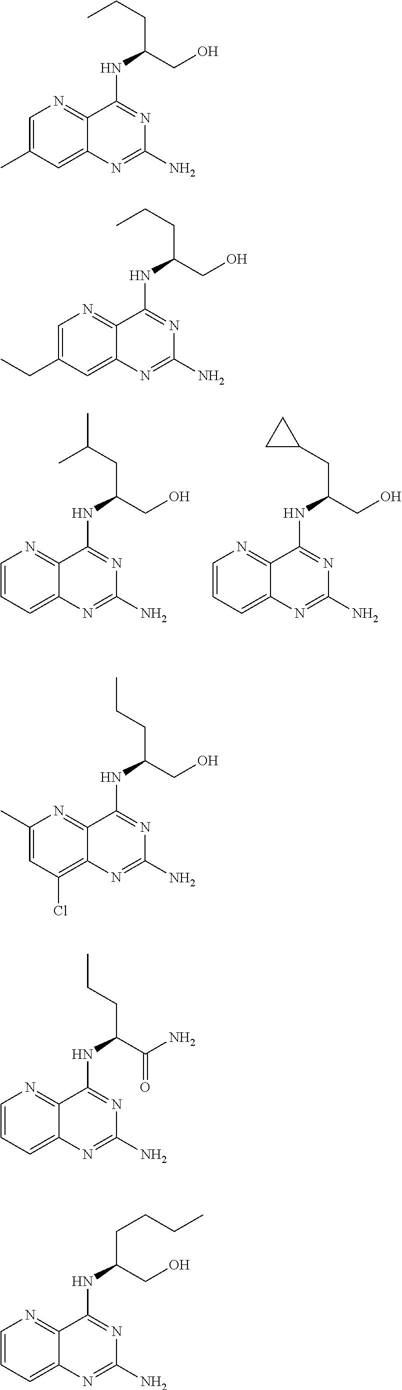

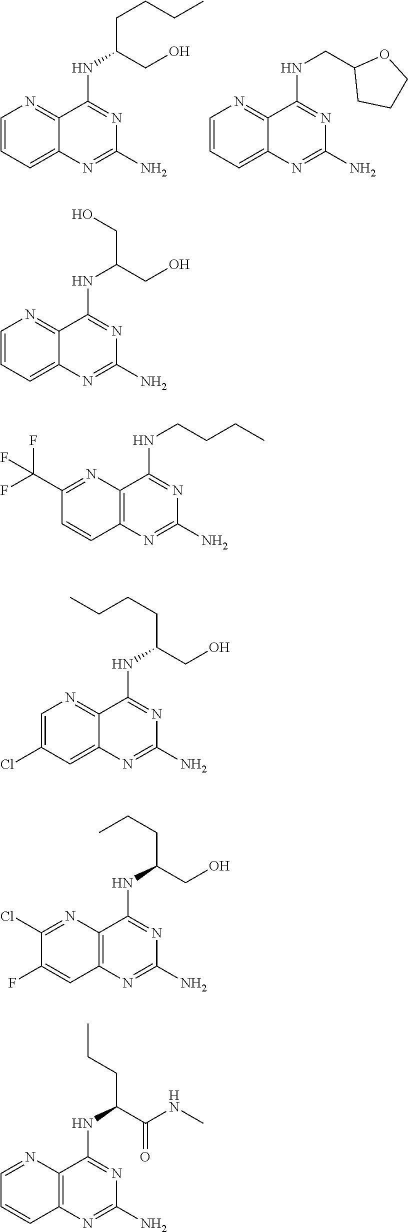

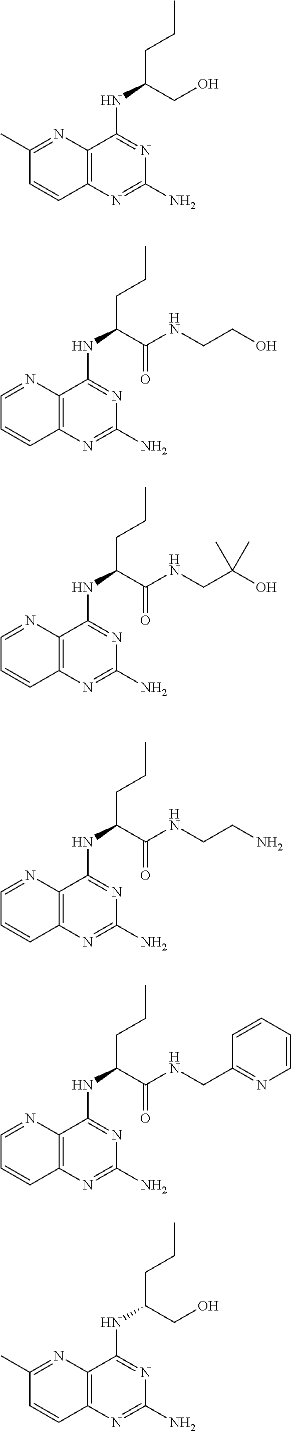

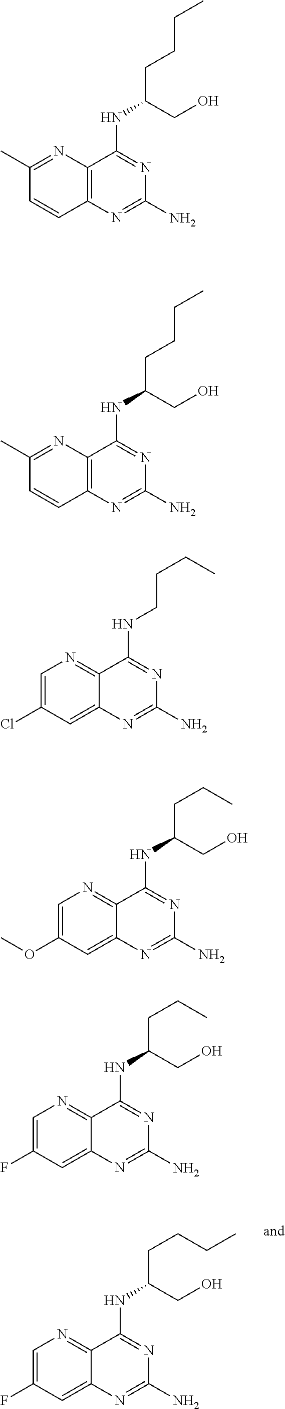

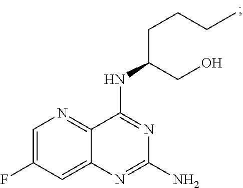 [SMILES: CCCC[C@@H](CO)Nc1nc(N)nc2cc(F)cnc12]

or a pharmaceutically acceptable salt thereof.

In certain embodiments, the compound of Formula (J) or (I) is selected from:

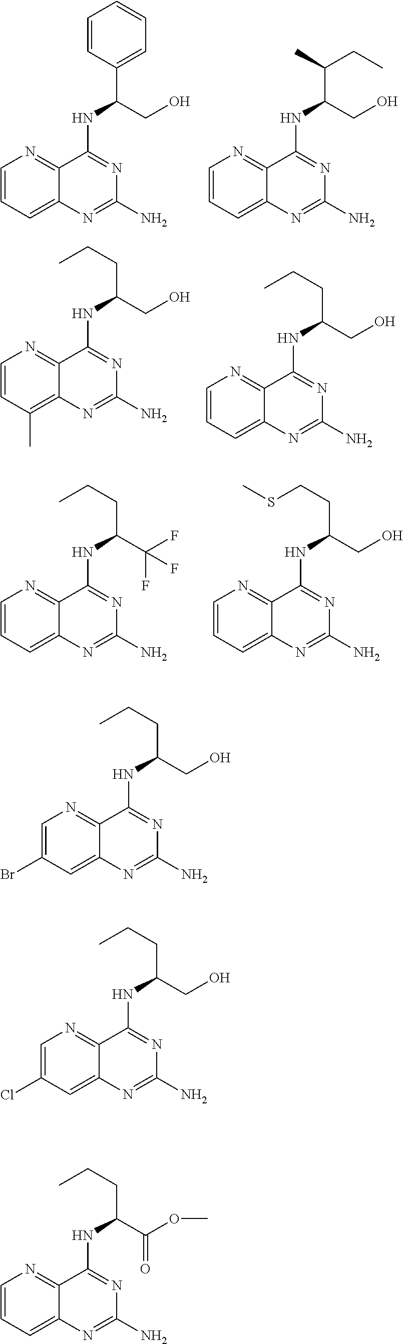

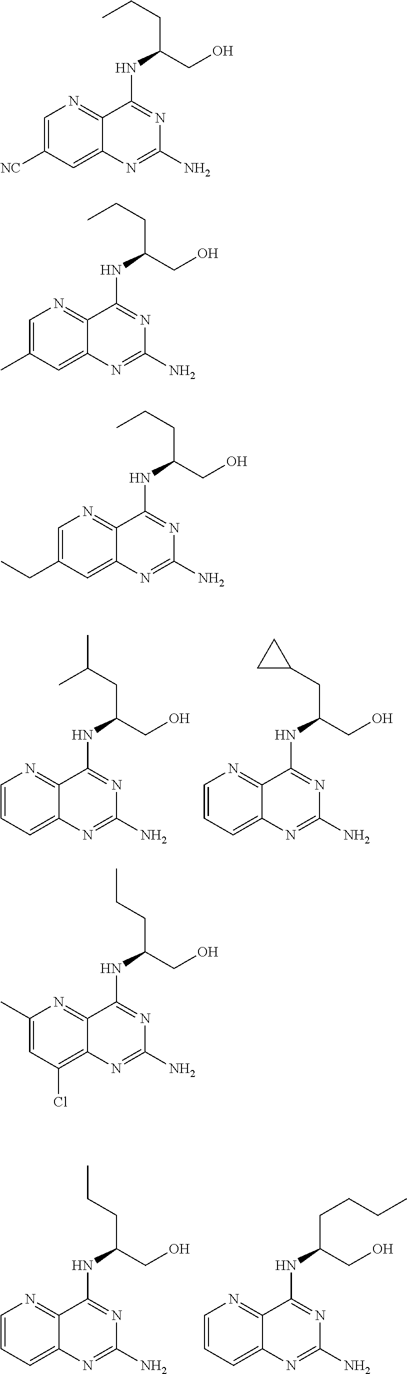

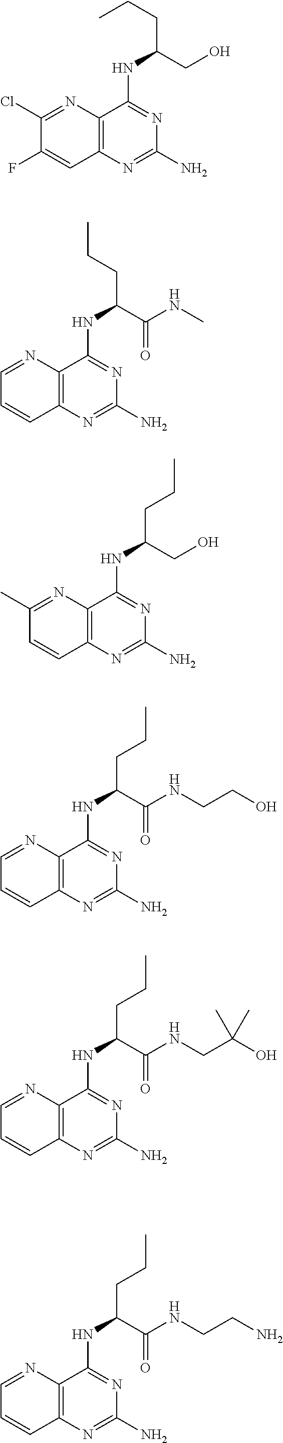

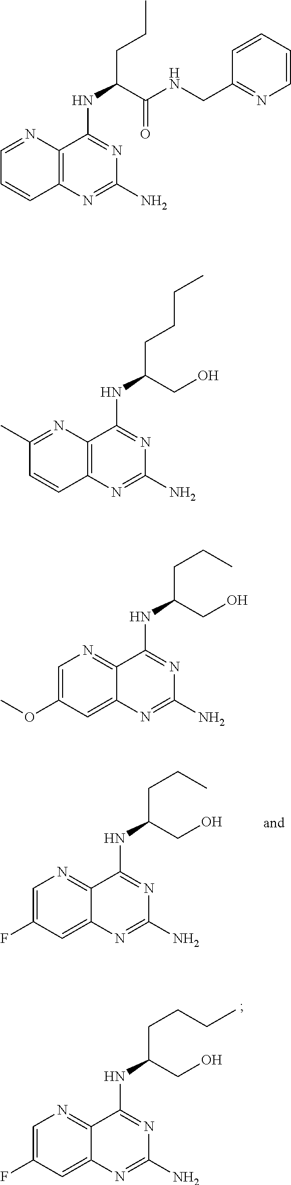

or a pharmaceutically acceptable salt thereof.

In certain embodiments, the compound of Formula (J) or (I) is selected from:

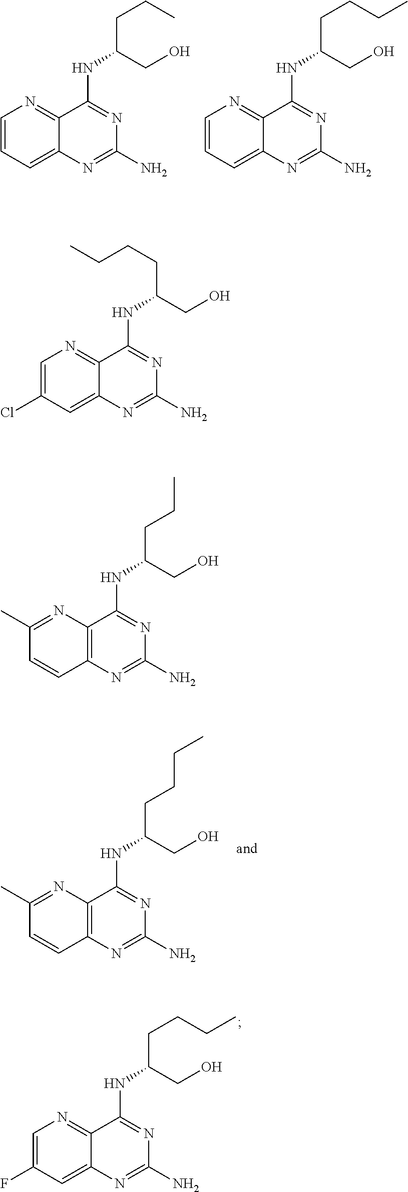 [SMILES: CCC[C@H](CO)Nc1nc(N)nc2cccnc12 ; CCCC[C@H](CO)Nc1nc(N)nc2cccnc12 ; CCCC[C@H](CO)Nc1nc(N)nc2cc(Cl)cnc12 ; CCC[C@H](CO)Nc1nc(N)nc2ccc(C)nc12 ; CCCC[C@H](CO)Nc1nc(N)nc2ccc(C)nc12 ; CCCC[C@H](CO)Nc1nc(N)nc2cc(F)cnc12]

or a pharmaceutically acceptable salt thereof.

In certain embodiments, the compound of Formula (J) is selected from:

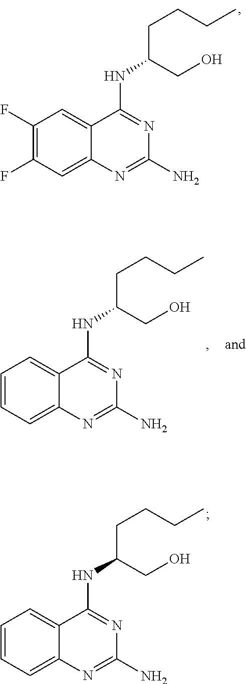 [SMILES: CCCC[C@H](CO)Nc1nc(N)nc2cc(F)c(F)cc12 ; CCCC[C@H](CO)Nc1nc(N)nc2ccccc12 ; CCCC[C@@H](CO)Nc1nc(N)nc2ccccc12]

or a pharmaceutically acceptable salt thereof.

In certain embodiments, the compound of Formula (J) or (I) is selected from:

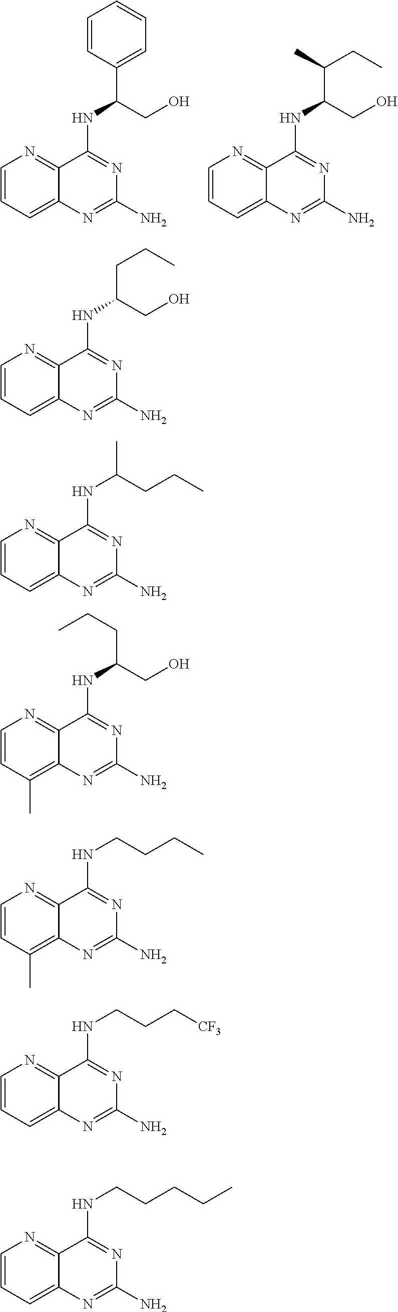

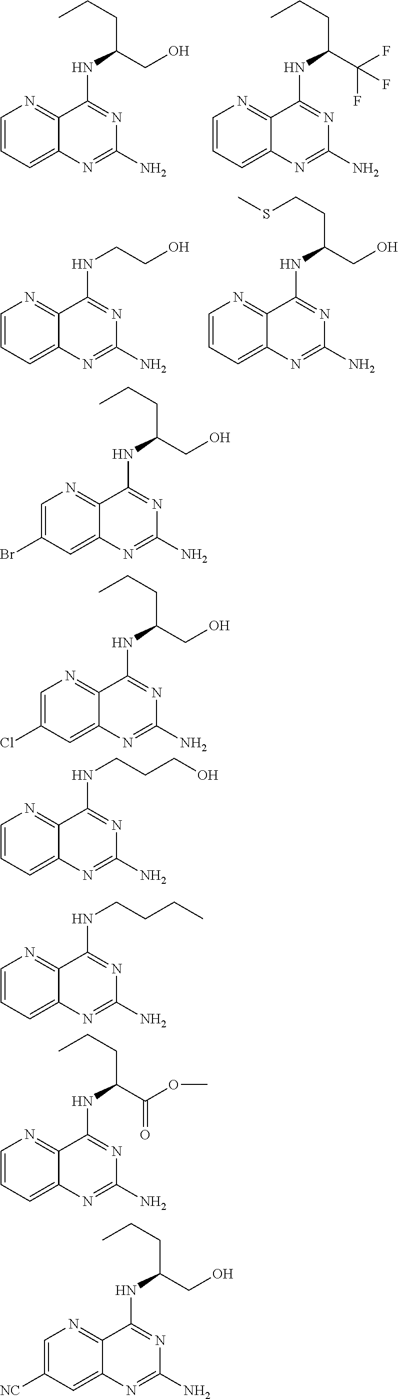

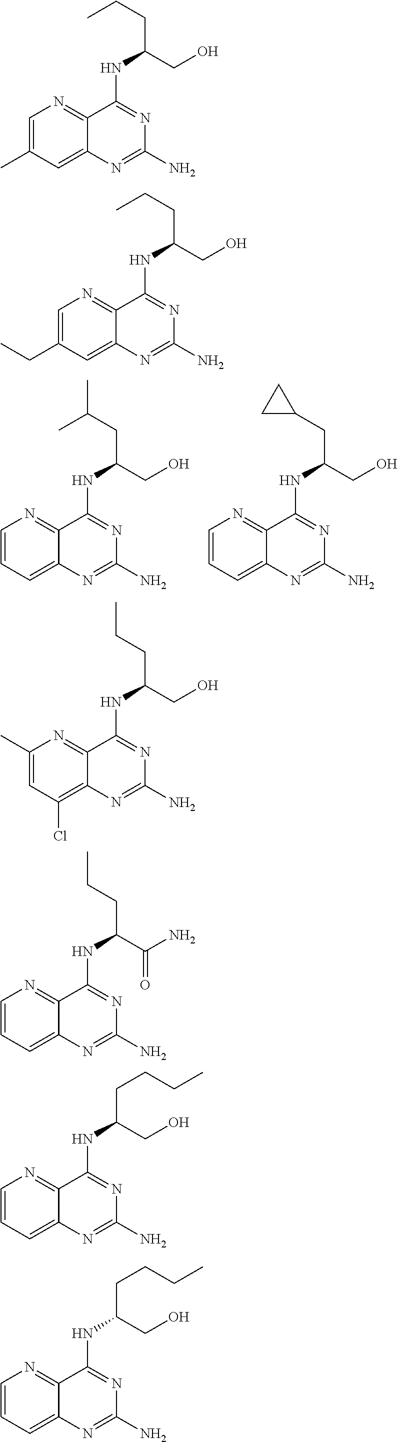

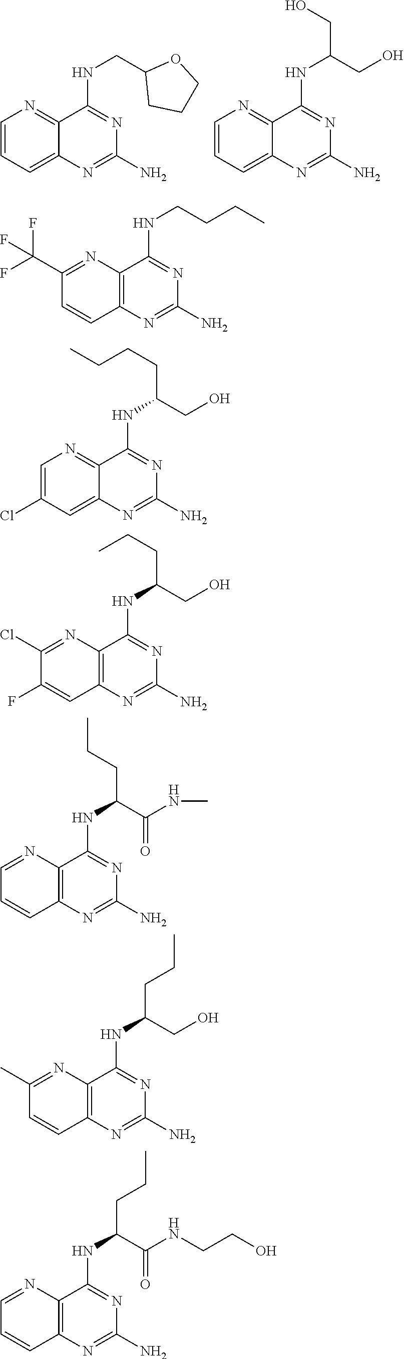

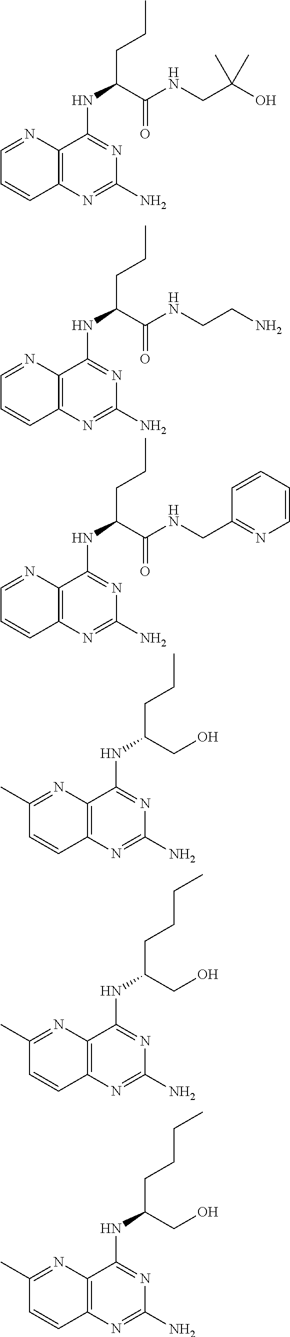

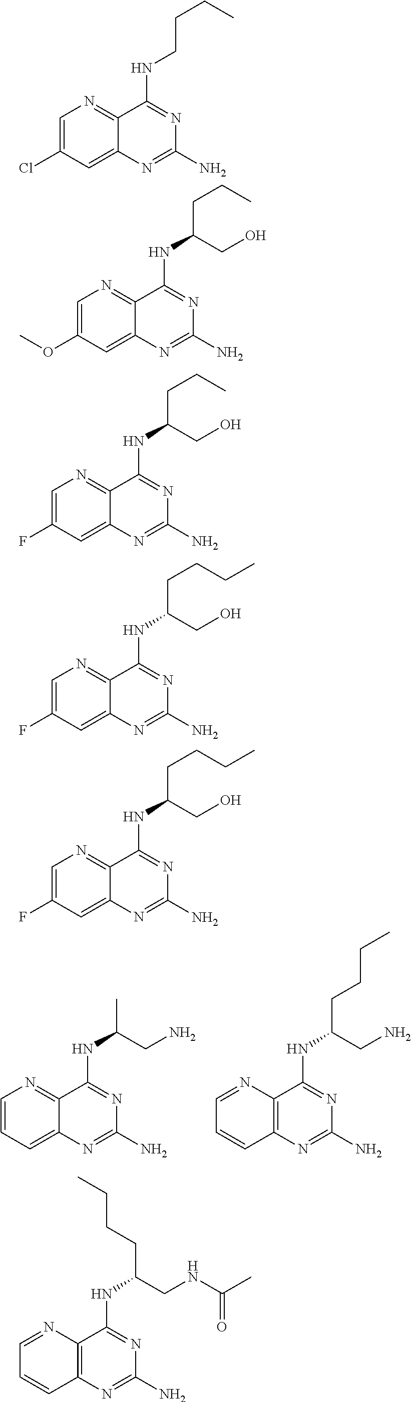

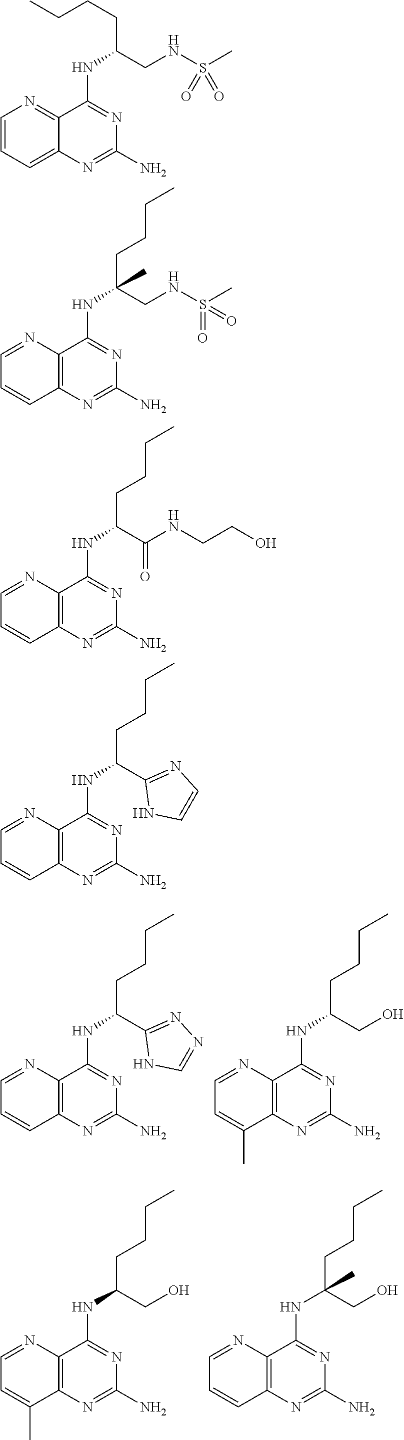

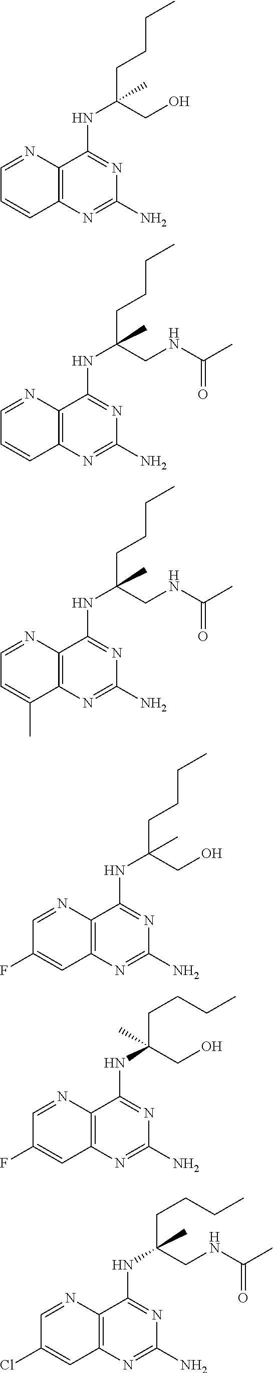

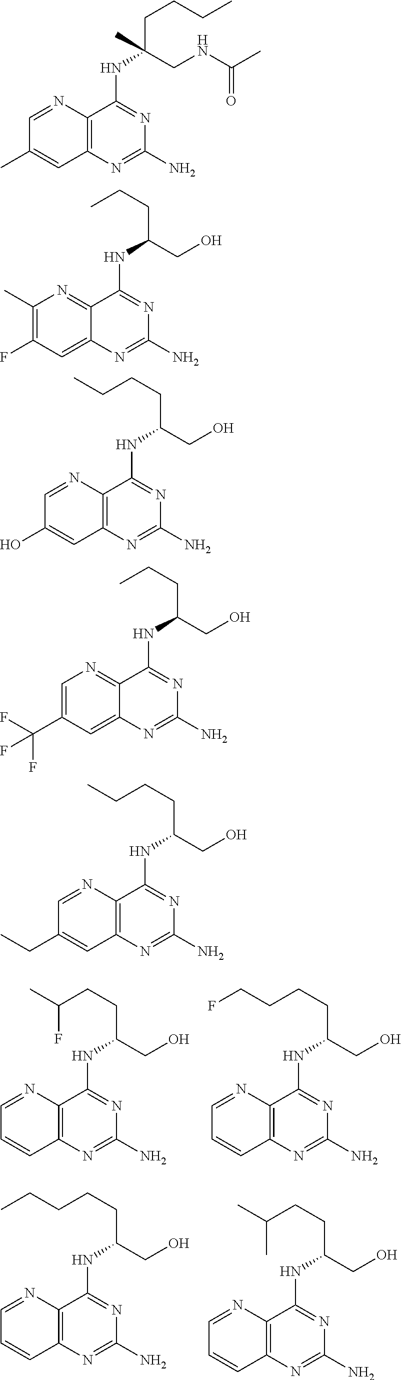

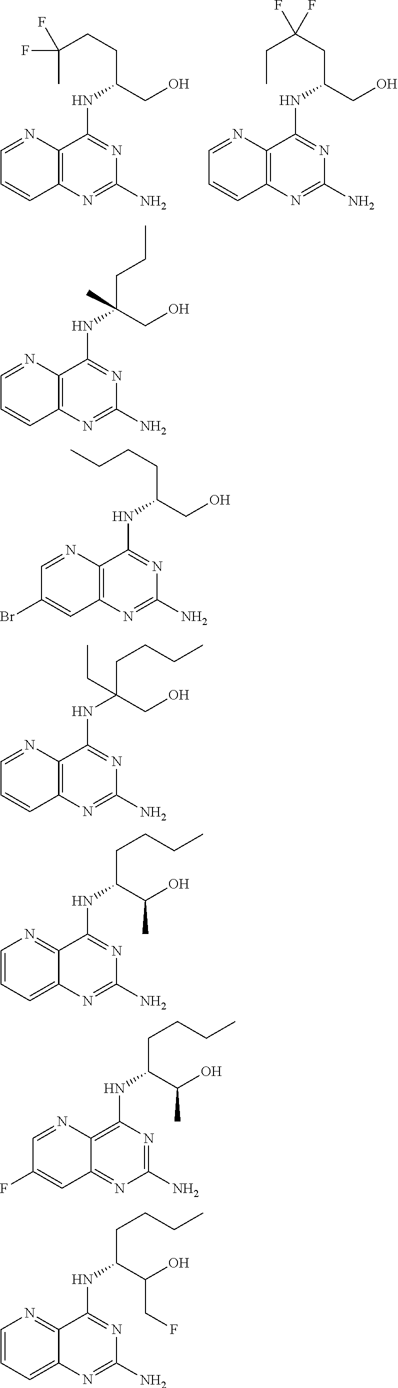

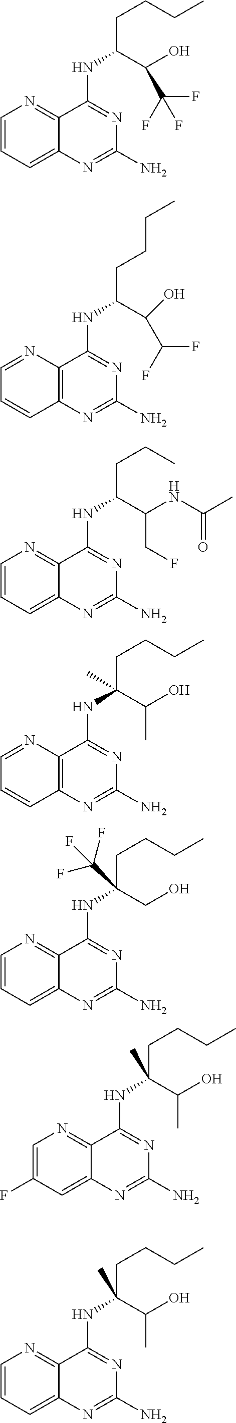

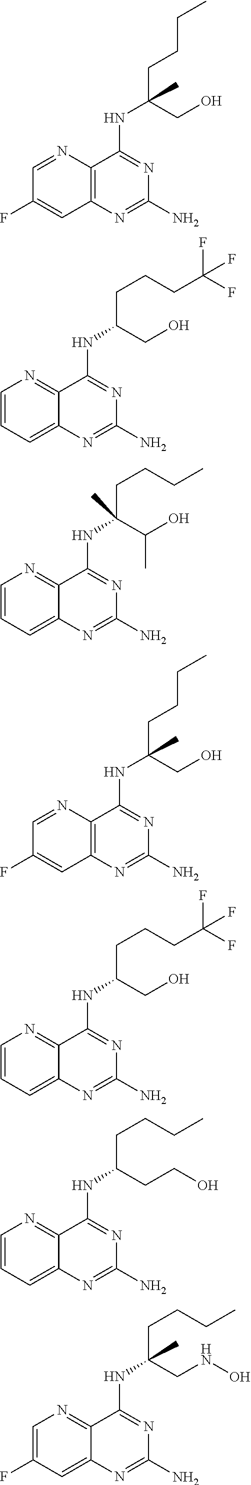

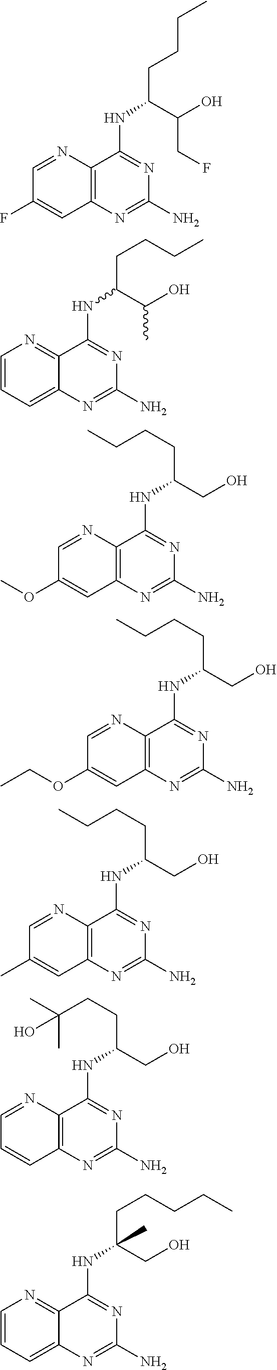

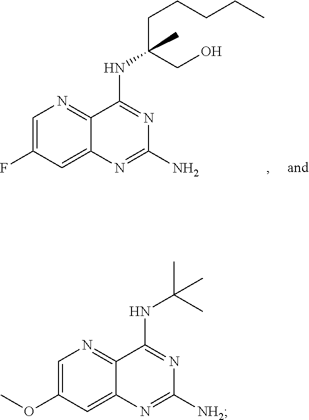

or a pharmaceutically acceptable salt thereof.

In certain embodiments, the compound of Formula (J), (I), (IV), or (IVa) is selected from:

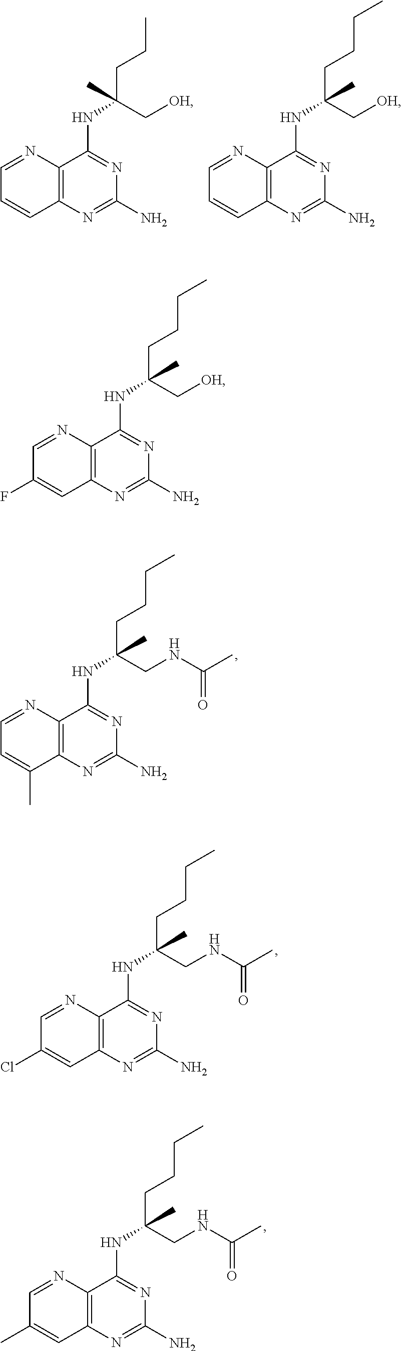

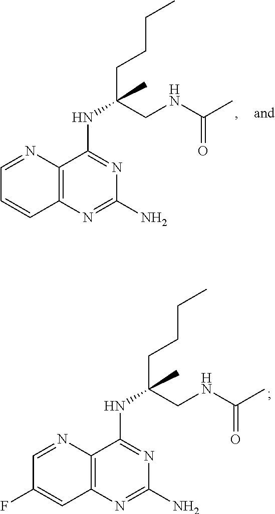

or a pharmaceutically acceptable salt thereof.

In certain embodiments, the compound of Formula (J), (I), (IV), or (IVa) is selected from:

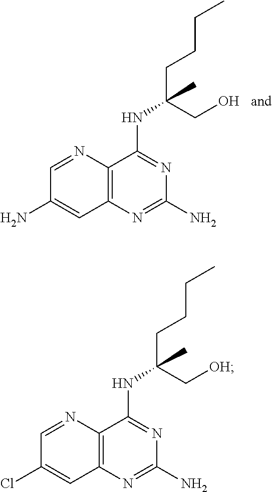

or a pharmaceutically acceptable salt thereof.

In certain embodiments, the compound of Formula (J), (I), (IV), or (IVa) is selected from:

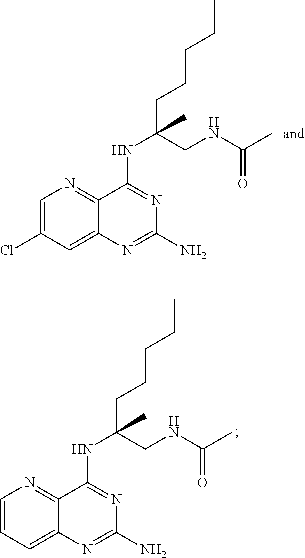 [SMILES: CCCCC[C@](C)(CNC(C)=O)Nc1nc(N)nc2cc(Cl)cnc12 ; "and" ; CCCCC[C@](C)(CNC(C)=O)Nc1nc(N)nc2cccnc12 ; ";"]

or a pharmaceutically acceptable salt thereof.

In certain embodiments, the compound of Formula (J), (I), (IV), or (IVa) is selected from:

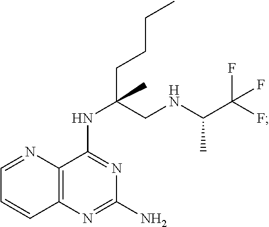 [SMILES: CCCC[C@](C)(CN[C@@H](C)C(F)(F)F)Nc1nc(N)nc2cccnc12]

or a pharmaceutically acceptable salt thereof.

In certain embodiments, the compound of Formula (J), (I), (IV), or (IVa) is selected from:

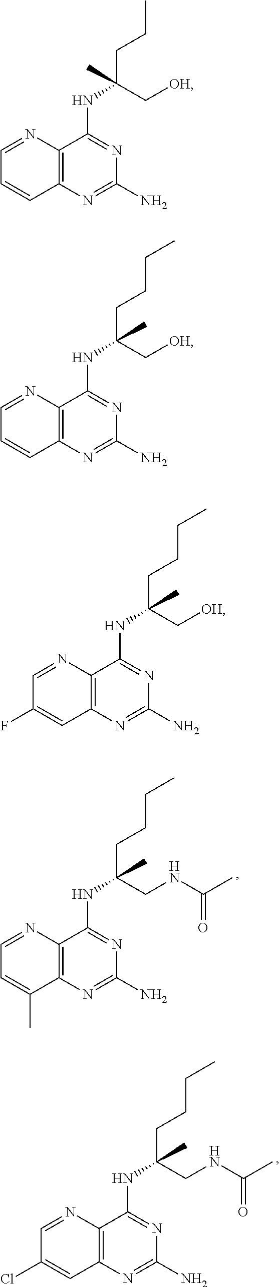

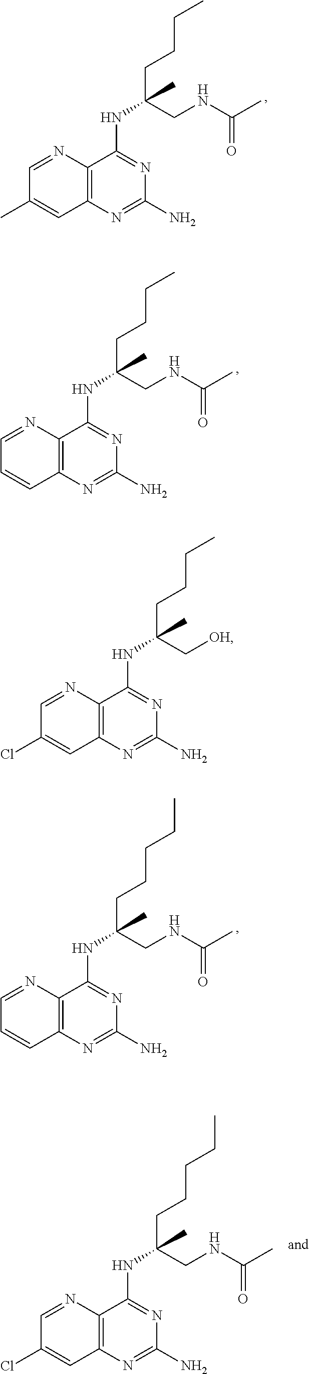

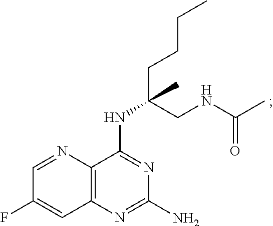

or a pharmaceutically acceptable salt thereof.

As used herein, “a compound of Formula (I)” includes compounds for Formula (II), (IIa), (IIb), (III), (IIIa), (IIIb), (IV), (IVa), (IVb), (IVc), or (IVd).

In certain embodiments, the present disclosure provides a pharmaceutical composition comprising a compound of the present disclosure (e.g. a compound of Formula (J), (I), (II), (IIa), (IIb), (III), (IIIa), (IIIb), (IV), (IVa), (IVb), (IVc), or (IVd)), or a pharmaceutically acceptable salt thereof, and a pharmaceutically acceptable excipient.

### Definitions

Unless stated otherwise, the following terms and phrases as used herein are intended to have the following meanings. The fact that a particular term or phrase is not specifically defined should not be correlated to indefiniteness or lacking clarity, but rather terms herein are used within their ordinary meaning. When trade names are used herein, applicants intend to independently include the tradename product and the active pharmaceutical ingredient(s) of the tradename product.

The acronym “HIV” refers to the human immunodeficiency virus that causes acquired immunodeficiency syndrome, “AIDS”.

The term “treating”, and grammatical equivalents thereof, when used in the context of treating a disease, means slowing or stopping the progression of a disease, or ameliorating at least one symptom of a disease, or ameliorating more than one symptom of a disease.

As used herein, “a compound of the invention”. “a compound described herein”, and “a compound of “Formula I”, as well as reference to a compound of each of the other formulas herein (e.g. Formula (J), (I), (II), (IIa), (IIb), (III), (IIIa), (IIIb), (IV), (IVa), (IVb), (IVc), or (IVd)), means a compound of the specified formula, structure, or chemical name, including alternative forms thereof such as, solvated forms, hydrated forms, esterified forms, or physiologically functional derivatives thereof. Compounds of the invention also include tautomeric forms thereof, e.g., tautomeric “enols” as described herein. Similarly, with respect to isolatable intermediates, the phrase “a compound of formula (number)” means a compound of that formula and alternative forms thereof.

The terms “combination antiretroviral therapy” (“cART”) refers to combinations or “cocktails” of antiretroviral medications used to treat human viral infections, including HIV infections. As used herein, the terms “combination antiretroviral therapy” and “cART include combinations and regimens often referred to as Highly Active Antiretroviral Therapy (HAART). HAART and cART combinations and regimens commonly include multiple, often three or more, drugs such as nucleoside or nucleotide reverse transcriptase inhibitors (NRTIs), non-nucleoside reverse transcriptase inhibitors (NNRTIs), protease inhibitors (PIs), integrase inhibitors, fusion inhibitors, CCR5 agonists, and/or integrase inhibitors.

The terms “chronic set point”, “set point in chronic HIV infection”, “viral load set point”, and “viral set point in chronic HIV infection” refer to the HIV viral load established in a patient's blood after infection or following the introduction of antiretroviral therapy or treatment, including combination antiretroviral therapy or treatment.

The terms “viral load” and “HIV viral load” refer to the level of HIV detectable in a the blood of an HIV infected human after HIV infection or following treatment with antiretroviral therapy, such as with cART or HAART treatment regimens. Viral load can be calculated by estimating the amount of virus in an involved body fluid. For example, it can be given in HIV RNA copies per millilitre of blood or blood plasma. An “undetectable” HIV viral load comprises a condition in which HIV RNA copies cannot be detected by standard viral load tests. An undetectable HIV viral load as used herein refers to a viral load of fewer than 50 HIV RNA copies per millilitre of blood or blood plasma. The term “viremia” refers to the measurable presence of virus or viral particles in circulation in a virally infected human. The term “transient viremia” refers to a brief, transitory, or temporary increase in the measurable presence of virus or viral particles in circulation in a virally infected human. Examples of transient HIV viremia include a period in which the HIV-1 RNA level in the blood or plasma of an HIV infected human which has been maintained for a period of time at a concentration of less than 50 copies of HIV-1 RNA per mL briefly, transitorily, or temporarily rises to a concentration of greater than 50 copies/mL, such as from 50 to 2,000 copies/mL, followed by a return to a concentration at, near, or below the initial viral concentration. In other embodiments, transient HIV viremia includes, without limitation, a period in which the HIV-1 RNA level in the blood or plasma of an HIV infected human which has been maintained for a period of time at a concentration of less than 40 copies of HIV-1 RNA per mL briefly, transitorily, or temporarily rises to a concentration of greater than 40 copies/mL, such as from 40 to 2,000 copies/mL. Transient, transitory, or temporary viremia may constitute a concentration of greater than 50 copies/mL after repeated testing of an “undetectable” HIV viral load of below 50 copies/mL for a designated period, such as one month, three months, six months, nine months, or one year. It may also constitute a concentration of greater than 50 copies/mL after repeated testing of an “undetectable” HIV viral load of below 50 copies/mL following a specificed number or series of tested concentrations of less than 50 copies of HIV-1 RNA per mL, as determined by a health care provider. In separate embodiments the number of consecutive tested concentrations of less than 50 copies may be 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, or 30 and may be for tests conducted, for instance, daily, weekly, biweekly, monthly, bimonthly, quarterly (every 3 months), biannually (twice per year), or annually (once per year).

The terms “virologic suppression” and “virologically suppressed” refer to a response to treatment in which the measurable level of viremia in a virally infected human is maintained at or below a desired level for a specified human or antiviral treatment or regimen. An example of HIV virologic suppression in an HIV-infected human may be the maintenance in the human of a measurable HIV viral load of less than 200 copies of HIV-1 RNA per mL of blood or plasma. Other examples of virologic suppression would be the maintenance in the human of a viral load of less than 100 copies/mL, less than 50 copies/ml, less than 40 copies/mL, less than 30 copies/mL, and less than 20 copies/mL.

The terms “latent HIV reservoir”, “ HIV latent reservoir”, “HIV reservoir”, “latent reservoir”, and “latent HIV infection” refer to a condition in which resting CD4+ T lymphocytes or other cells are infected with HIV but are not actively producing HIV. The presently inactive HIV infected cells are referred to as “latently infected cells”. Antiretroviral therapy (ART) can reduce the level of HIV in the blood to an undetectable level, while latent reservoirs of HIV continue to survive. When a latently infected cell is reactivated, the cell begins to produce HIV (HIV replication).

The term “regimen” refers to a systematic schedule of administering pharmaceutically effective agents to a patient in need thereof, such as a human in need thereof, to reach a therapeutic objective.

The terms “modulation”, “modulating” and “modulator” refer to the actions of an agent to agonize (activate or enhance) or antagonize (inhibit or diminish) the function of a biological target. Agonists or enhancers include those modulators which increase the activity of TLR3, TLR4, TLR7, TLR8, and/or TLR9 receptors. Within each method, combination, kit, use, composition, and regimen described herein utilizing or containing a TLR8 modulator or TLR8 modulating compound there is a separate embodiment in which the TLR8 modulator or TLR8 modulating compound is an agonist of TLR8. Methods for determining a particular compound modulates TLR8 are known to those of skill in the art, including measuring, for example, e.g. cytokine/chemokine induction, HIV activation, HIV-specific CD8 T cell function, anti-HIV Ab-mediated killing, etc.

The term “HIV antibody” refers to both non-neutralizing HIV antibodies and neutralizing HIV antibodies, including broadly neutralizing HIV antibodies. The terms “broadly neutralizing HIV-1 antibody” and “broadly neutralizing HIV-1 antibody” (bNAb) refer to neutralizing antibodies which neutralize multiple HIV-1 viral strains.

The acronyms “IL” and “IL-” refer to “interleukin”, such as the interleukins

The term “nucleoside sparing”, “nucleotide sparing”, and “nuc-sparing” refers to an antiretroviral combination, regimen, formulation, or therapy that does not utilize nucleoside or nucleotide pharmaceutical agents, such as nucleoside or nucleotide reverse transcriptase inhibitors (NRTIs)

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes without limitation pharmaceutically-acceptable acid addition salts and pharmaceutically-acceptable base addition salts. Lists of suitable salts are found in, for example, S. M. Birge et al., J. Pharm. Sci., 1977, 66, pp. 1-19.

The terms “mL” and “ml” refer to milliliter.

The terms “antiviral agent”, “antiretroviral agent”, “antiretroviral compound” refer to a compounds or agent used to treat an HIV infection in a human.

The terms “antiviral agent” and “antivirals” as used herein is intended to mean an agent that is effective to inhibit the formation and/or replication of a virus in a human, including but not limited to agents that interfere with either host or viral mechanisms necessary for the formation and/or replication of a virus in a human. The terms “antiviral agent” and “antivirals” include, for example, an HIV integrase catalytic site inhibitor selected from the group consisting: raltegravir (ISENTRESS®; Merck); elvitegravir (Gilead); soltegravir (GSK; ViiV); cabotegravir (GSK; ViiV) and dolutegravir; an HIV nucleoside or nucleotide reverse transcriptase inhibitor selected from the group consisting of: abacavir (ZIAGEN®; GSK); didanosine (VIDEX®; BMS); tenofovir disoproxil fumarate (VIREAD®; Gilead); tenofovir alafenamide (TAF); emtricitabine (EMTRIVA®; Gilead); lamivudine (EPIVIR®; GSK/Shire); stavudine (ZERIT®; BMS); zidovudine (RETROVIR®; GSK); abacavir (ZIAGEN, GSK), elvucitabine (Achillion); CMX-157 (Chimerix), and festinavir (BMS/Oncolys); an HIV non-nucleoside reverse transcriptase inhibitor selected from the group consisting of: nevirapine (VIRAMUNE®; BI); efavirenz (SUSTIVA®; BMS); etravirine (INTELENCE®; J&J); rilpivirine (TMC278, R278474; J&J); fosdevirine (GSK/ViiV); MK-1439 (Merck), and lersivirine (Pfizer/ViiV); an HIV protease inhibitor selected from the group consisting of: atazanavir (REYATAZ®; BMS); darunavir (PREZISTA®; J&J); indinavir (CRIXIVAN®; Merck); lopinavir (KALETRA®; Abbott); nelfinavir (VIRACEPT®; Pfizer); saquinavir (INVIRASE®; Hoffmann-LaRoche); tipranavir (APTIVUS®; BI); ritonavir (NORVIR®; Abbott); and fosamprenavir (LEXIVA®; GSK/Vertex); an HIV entry inhibitor selected from: maraviroc (SELZENTRY®; Pfizer); enfuvirtide (FUZEON®; Trimeris); and BMS-663068 (BMS); and an HIV maturation inhibitor selected from: bevirimat (Myriad Genetics), BMS-955176 (BMS), GSK2838232 (GSK/ViiV). A boosting agent, such as cobicistat or ritonavir, is included within the terms “antiviral agent” and “antivirals” when used in combination with one or more of the antiviral agents described herein.

The terms “effective amount”, “pharmaceutically effective amount”, and “therapeutically effective amount” refer to an amount that may be effective to elicit the desired biological or medical response, including the amount of a compound that, when administered to a subject for treating a disease, is sufficient to effect such treatment for the disease. The effective amount will vary depending on the compound, the disease and its severity and the age, weight, etc., of the subject to be treated. The effective amount can include a range of amounts. A pharmaceutically effective amount includes amounts of an agent which are effective when combined with other agents.

The terms “composition”, “pharmaceutical composition”, “formulation”, and “pharmaceutical formulation” refer to a composition comprising a pharmaceutically effective amount of a pharmaceutically active agent and at least one pharmaceutically acceptable excipient.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals

The terms “kit” and “pharmaceutical kit” refer to a commercial kit or package comprising, in one or more suitable containers, one or more pharmaceutical compositions and instructions for their use. Such kits may also be referred to by the terms “package” or “pharmaceutical package”.

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning. A prefix such as “Cᵤ₋ᵥ” or (Cᵤ-Cᵥ) indicates that the following group has from u to v carbon atoms, where u and v are integers. For example, “C₁₋₆alkyl” indicates that the alkyl group has from 1 to 6 carbon atoms.

“Alkyl” is a linear or branched saturated monovalent hydrocarbon. For example, an alkyl group can have 1 to 10 carbon atoms (i.e., (C₁₋₁₀)alkyl) or 1 to 8 carbon atoms (i.e., (C₁₋₆)alkyl) or 1 to 6 carbon atoms (i.e., (C₁₋₆ alkyl) or 1 to 4 carbon atoms (i.e., (C₁₋₄)alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃)_{.}

“Alkenyl” is a linear or branched monovalent hydrocarbon radical with at least one carbon-carbon double bond. For example, an alkenyl group can have 2 to 8 carbon atoms (i.e., C₂₋₈ alkenyl), or 2 to 6 carbon atoms (i.e., C₂₋₆ alkenyl) or 2 to 4 carbon atoms (i.e., C₂₋₄ alkenyl). Examples of suitable alkenyl groups include, but are not limited to, ethylene or vinyl (—CH═CH₂), allyl (—CH₂CH═CH₂), 5-hexenyl (—CH₂CH₂CH₂CH₂CH═CH₂), and 3-hexenyl (—CH₂CH₂CH═CHCH₂CH₂).

“Alkynyl” is a linear or branched monovalent hydrocarbon radical with at least one carbon-carbon triple bond. For example, an alkynyl group can have 2 to 8 carbon atoms (i.e., C₂₋₈ alkyne,) or 2 to 6 carbon atoms (i.e., C₂₋₆ alkynyl) or 2 to 4 carbon atoms (i.e., C₂₋₄ alkynyl). Examples of alkynyl groups include, but are not limited to, acetylenyl (—C≡CH), propargyl (—CH₂C≡CH), and —CH₂—C≡C—CH₃.

The term “halo”or “halogen” as used herein refers to fluoro (—F), chloro (—Cl), bromo (—Br) and iodo (—I).

The term “haloalkyl” as used herein refers to an alkyl as defined herein, wherein one or more hydrogen atoms of the alkyl are independently replaced by a halo substituent, which may be the same or different. For example, C₁-₆haloalkyl is a C₁₋₆alkyl wherein one or more of the hydrogen atoms of the C₁₋₆alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl.

The term “heteroalkyl” as used herein refers to an alkyl as defined herein, wherein one or more of the carbon atoms of the alkyl are replaced by an O, S, or NR^{q}, wherein each R^{q} is independently H or C₁₋₆alkyl. For example, C₁₋₈heteroalkyl intends a heteroalkyl of one to eight carbons wherein one or more carbon atoms is replaced by a heteroatom (e.g., O, S, NR^{q}, OH, SH or N(R^{q})₂), which may the same or different. Examples of heteroalkyls include but are not limited to methoxymethyl, ethoxymethyl, methoxy, 2-hydroxyethyl and N,N′-dimethylpropylamine. A heteroatom of a heteroalkyl may optionally be oxidized or alkylated. A heteroatom may be placed at any interior position of the heteroalkyl group or at a position at which the group is attached to the remainder of the molecule. Examples include, but are not limited to, —CH₂OCH₃, —CH₂CH₂NHCH₃, —CH₂CH₂N(CH₃)—CH₃, —CH₂SCH₂CH₃, —S(O)CH₃, —CH₂CH₂S(O)₂CH₃, —CHCHOCH₃, —CH₂CHNOCH₃, —CHCHN(CH₃)CH₃, —CH₂NHOCH₃ and —CH₂OS(CH₃)₃

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1, 2, 3, 4-tetrahydronaphthyl. Non-limiting examples of aryl groups include, but are not limited to, phenyl, indenyl, naphthyl, 1, 2, 3, 4-tetrahydronaphthyl, anthracenyl, and the like.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5 to 10 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclopenta[1,2-c]pyrazole.

The term “cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocyles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

The term “heterocyclyl” or “heterocycle” as used herein refers to a single saturated or partially unsaturated non-aromatic ring or a non-aromatic multiple ring system that has at least one heteroatom in the ring (i.e., at least one annular heteroatom selected from oxygen, nitrogen, and sulfur). Unless otherwise specified, a heterocyclyl group has from 5 to about 20 annular atoms, for example from 3 to 12 annular atoms, for example from 5 to 10 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring system can be connected to each other via fused, spiro and bridged bonds when allowed by valency requirements. Heterocycles include, but are not limited to, azetidine, aziridine, imidazolidine, morpholine, oxirane (epoxide), oxetane, piperazine, piperidine, pyrazolidine, piperidine, pyrrolidine, pyrrolidinone, tetrahydrofuran, tetrahydrothiophene, dihydropyridine, tetrahydropyridine, quinuclidine, N-bromopyrrolidine, N-chloropiperidine, and the like.

The term “oxo” as used herein refers to ═O.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The TLR8 compounds described herein may be prepared and/or formulated as pharmaceutically acceptable salts. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Wiliams and Wilkins, Philadelphia, Pa., 2006.

Provided are also compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds.

A “solvate” is formed by the interaction of a solvent and a compound. Solvates of salts of the compounds described herein are also provided. Hydrates of the compounds described herein are also provided.

A “prodrug” includes any compound that becomes a compound described herein when administered to a subject, e.g., upon metabolic processing of the prodrug.

One skilled in the art will recognize that substituents and other moieties of the compounds of the formulas herein should be selected in order to provide a compound which is sufficiently stable to provide a pharmaceutically useful compound which can be formulated into an acceptably stable pharmaceutical composition. Compounds of Formula J or I which have such stability are contemplated as falling within the scope of the present invention.

While not wishing to be bound by any one theory, the TLR8 modulating compounds described herein modulate TLR8 receptors as agonists. As is understood by those of skill in the art, modulators of TLR8 may, to some degree, modulate other toll-like receptors (e.g. TLR7). As such, in certain embodiments, the TLR8 modulating cmpounds described herein may also modulate TLR7 to a measureable degree. In certain embodiments, those compounds that modulate TLR8 to a higher degree than TLR7 are considered selective modulators of TLR8.

The definitions and substituents for various generic and subgeneric groups of the present compounds are described and illustrated herein. It should be understood by one skilled in the art that any combination of the definitions and substituents described above should not result in an inoperable species or compound. “Inoperable species or compounds” means compound structures that violates relevant scientific principles (such as, for example, a carbon atom connecting to more than four covalent bonds) or compounds too unstable to permit isolation and formulation into pharmaceutically acceptable dosage forms.

### Pharmaceutical Formulations

The TLR8 modulating compounds of the present disclosure are formulated with conventional excipients, which will be selected in accord with ordinary practice. Tablets will contain excipients, glidants, fillers, binders and the like. Aqueous formulations are prepared in sterile form, and when intended for delivery by other than oral administration generally will be isotonic. In certain embodiments, a liposomal formulation may be used, for example when the compounds are to be administered parenterally. All formulations will optionally contain excipients such as those set forth in the Handbook of Pharmaceutical Excipients (1986), herein incorporated by reference in its entirety. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations of the invention, both for veterinary and for human use, comprise at least one active ingredient, together with one or more acceptable excipients and optionally other therapeutic ingredients.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference in its entirety. Such methods include the step of bringing into association the active ingredient with the excipient which constitutes one or more accessory ingredients. In general the formulations are prepared by uniformly and intimately bringing into association the active ingredient with liquid excipients or finely divided solid excipients or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

Pharmaceutical formulations according to the present invention comprise one or more compounds of the invention together with one or more pharmaceutically acceptable excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The effective dose of an active ingredient depends at least on the nature of the condition being treated, toxicity, whether the compound is being used prophylactically (lower doses) or against an active disease or condition, the method of delivery, and the pharmaceutical formulation, and will be determined by the clinician using conventional dose escalation studies. The effective dose can be expected to be from about 0.0001 to about 10 mg/kg body weight per day, typically from about 0.001 to about 1 mg/kg body weight per day, more typically from about 0.01 to about 1 mg/kg body weight per day, even more typically from about 0.05 to about 0.5 mg/kg body weight per day. For example, the daily candidate dose for an adult human of approximately 70 kg body weight may range from about 0.05 mg to about 250 mg, or between about 1.0 mg and about 150 and may take the form of single or multiple doses.

In yet another embodiment, the present application discloses pharmaceutical compositions comprising a compound of Formula J or I or a pharmaceutically acceptable salt thereof, and a pharmaceutically acceptable excipient.

### Routes of Administration

One or more compounds of the invention (herein referred to as the active ingredients) are administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with for example the condition of the recipient. An advantage of certain compounds of this invention is that they are orally bioavailable and can be dosed orally.

### Combination Therapy

In one embodiment, the TLR8 modulating compounds described herein are used in combination with an additional active therapeutic ingredient or agent.

In one embodiment, combinations of one or more of the TLR8 modulating compounds described herein and additional active agents may be selected to treat patients with an HIV viral infection.

Combinations of the compounds are typically selected based on the condition to be treated, cross-reactivities of ingredients and pharmaco-properties of the combination. For example, when treating an infection (e.g., HIV), the compositions of the invention are combined with other active agents (such as those described herein).

Suitable active agents or ingredients which can be combined with the TLR8 modulating compounds described herein, or a salt thereof, can include TLR8 agonists selected from the group consisting of motolimod, 3M-051, 3M-052, MCT-465, IMO-4200, VTX-763, and VTX-1463 and mixtures thereof.

In addition, the TLR8 modulating compounds described herein may be employed in combination with other therapeutic agents for the treatment or prophylaxis of AIDS and/or one or more other diseases present in a human subject suffering from AIDS. The additional therapeutic agent(s) may be coformulated with one or more salts of the invention (e.g., coformulated in a tablet).

Examples of such additional therapeutic agents include agents that are effective for the treatment or prophylaxis of viral, parasitic or bacterial infections, or associated conditions, or for treatment of tumors or related conditions, include 3′-azido-3′-deoxythymidine (zidovudine, AZT), 2′-deoxy-3′-thiacytidine (3TC), 2′,3′-dideoxy-2′,3′-didehydroadenosine (D4A), 2′,3′-dideoxy-2′,3′-didehydrothymidine (D4T), carbovir (carbocyclic 2′,3′-dideoxy-2′,3′-didehydroguanosine), 3′-azido-2′,3′-dideoxpridine, 5-fluorothymidine, (E)-5-(2-bromovinyI)-2′-deoxyuridine (BVDU), 2-chlorodeoxyadenosine, 2-deoxycoformycin, 5-fluorouracil, 5-fluorouridine, 5-fluoro-2′-deoxyuridine, 5-trifluoromethyl-2′-deoxyuridine, 6-azauridine, 5-fluoroorotic acid, methotrexate, triacetyluridine, 1-(2′-deoxy-2′-fluoro-1-beta-arabinosyl)-5-iodocytidine (FIAC), tetrahydro-imidazo(4,5, 1-jk)-(1,4)-benzodiazepin-2(1H)-thione (TIBO), 2′-nor-cyclicGMP, 6-methoxypurine arabinoside (ara-M), 6-methoxypurine arabinoside 2′-O-valerate; cytosine arabinoside (ara-C), 2′,3′-dideoxynucleosides such as 2′,3′-dideoxycytidine (ddC), 2′,3′-dideoxyadenosine (ddA) and 2′,3′-dideoxyinosine (ddl); acyclic nucleosides such as acyclovir, penciclovir, famciclovir, ganciclovir, HPMPC, PMEA, PMEG, PMPA, PMPDAP, FPMPA, HPMPA, HPMPDAP, (2R, 5R)-9→tetrahydro-5-(phosphonomethoxy)-2-furanyladenine, (2R, 5R)-1→tetrahydro-5-(phosphonomethoxy)-2-furanylthymine; other antivirals including ribavirin (adenine arabinoside), 2-thio-6-azauridine, tubercidin, aurintricarboxylic acid, 3-deazaneoplanocin, neoplanocin, rimantidine, adamantine, and foscarnet (trisodium phosphonoformate); antibacterial agents including bactericidal fluoroquinolones (ciprofloxacin, pefloxacin and the like); aminoglycoside bactericidal antibiotics (streptomycin, gentamicin, amicacin and the like); beta-lactamase inhibitors (cephalosporins, penicillins and the like); other antibacterials including tetracycline, isoniazid, rifampin, cefoperazone, clarithromycin and azithromycin, antiparasite or antifungal agents including pentamidine (1,5-bis(4′-aminophenoxy)pentane), 9-deaza-inosine, sulfamethoxazole, sulfadiazine, quinapyramine, quinine, fluconazole, ketoconazole, itraconazole, Amphotericin B, 5-fluorocytosine, clotrimazole, hexadecylphosphocholine and nystatin; renal excretion inhibitors such as probenicid; nucleoside transport inhibitors such as dipyridamole, dilazep and nitrobenzylthioinosine, immunomodulators such as FK506, cyclosporin A, thymosin α-1; cytokines including TNF and TGF-β; interferons including IFN-α, IFN-β, and IFN-Y; interleukins including various interleukins, macrophage/granulocyte colony stimulating factors including GM-CSF, G-CSF, M-CSF, cytokine antagonists including anti-TNF antibodies, anti-interleukin antibodies, soluble interleukin receptors, protein kinase C inhibitors and the like. Examples of suitable active therapeutic agents or ingredients which can be combined with the TLR8 modulating compounds described herein, and which have activity against HIV, include:

1) HIV protease inhibitors, e.g., amprenavir, atazanavir, fosamprenavir, indinavir, lopinavir, ritonavir, lopinavir+ritonavir, nelfinavir, saquinavir, tipranavir, brecanavir, darunavir, TMC-126, mozenavir (DMP-450), JE-2147 (AG1776), AG1859, DG35, L-756423, R00334649, KNI-272, DPC-681, DPC-684, and GW640385X, DG17, PPL-100;

2) a HIV non-nucleoside inhibitor of reverse transcriptase, e.g., capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirine), BILR 355 BS, VRX 840773, UK-453,061, RDEA806, MK-1439;

3) a HIV nucleoside inhibitor of reverse transcriptase, e.g., zidovudine, emtricitabine, didanosine, stavudine, zalcitabine, lamivudine, abacavir, amdoxovir, elvucitabine, alovudine, MIV-210, racivir (-FTC), D-d4FC, phosphazide, fozivudine tidoxil, fosalvudine tidoxil, apricitibine (AVX754), amdoxovir, KP-1461, abacavir+lamivudine, abacavir+lamivudine+zidovudine, zidovudine+lamivudine;

4) a HIV nucleotide inhibitor of reverse transcriptase, e.g., tenofovir, tenofovir disoproxil fumarate+emtricitabine, tenofovir disoproxil fumarate+emtricitabine+efavirenz, and adefovir, CMX-157, and TAF;

5) a HIV integrase inhibitor, e.g., curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-707035, MK-2048, BA-011, BMS-538158, GSK364735C, GSK1265744 (GSK744, cabotegravir), elvitegravir, compounds disclosed in US 2014-0221356 (Gilead Sciences, Inc.) for example (2R,5S,13aR)-N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (2S,5R,13a5)-N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (1S,4R,12aR)-N-(2,4-difluorobenzyl)-7-hydroxy-6,8-dioxo-1,2,3,4,6,8,12,12a-octahydro-1,4-methanodipyrido[1,2-a:1′,Z-d]pyrazine-9-carboxamide, (1R,4S,12aR)-7-hydroxy-6,8-dioxo-N-(2,4,6-trifluorobenzyl)-1,2,3,4,6,8,12,12a-octahydro-1,4-methanodipyrido[1,2-a:1′,2′-d]pyrazine-9-carboxamide, (2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide, and (1R,4S,12aR)-N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide, US2015-0018298 (Gilead Sciences, Inc.) and US2015-0018359 (Gilead Sciences, Inc.), and dolutegravir;

6) HIV non-catalytic site, or allosteric, integrase inhibitors (NCINI) including, but not limited to, BI-224436, CX0516, CX05045, CX14442, compounds disclosed in US2014/0296228 (Boehringer Ingelheim), US 2013/0203747 (Boehringer Ingelheim), US 2013/0281433 (Gilead Sciences), US 2013/0281434 (Gilead Sciences) (Gilead Sciences), US 2014/0045818 (Gilead Sciences), US 2013/0203727 (Gilead Sciences), US 2013/0210801 (Gilead Sciences), and US 2015/0038549, each of which is incorporated by references in its entirety herein;

7) a gp41 inhibitor, e.g., enfuvirtide, sifuvirtide, FB006M, TRI-1144, SPC3, DES6, Locus gp41, CovX, and REP 9;

8) a CXCR4 inhibitor, e.g., AMD-070;

9) an entry inhibitor, e.g., SP01A, TNX-355, 9) a gp120 inhibitor, e.g., BMS-488043 and BlockAide/CR;

10) a G6PD and NADH-oxidase inhibitor, e.g., immunitin;

11) a CCRS inhibitor, e.g., aplaviroc, vicriviroc, INCB9471, PRO-140, INCB15050, PF-232798, CCR5mAb004, and maraviroc;

12) an interferon, e.g., pegylated rIFN-alpha 2b, pegylated rIFN-alpha 2a, rIFN-alpha 2b, IFN alpha-2b XL, rIFN-alpha 2a, consensus IFN alpha, infergen, rebif, locteron, AVI-005, PEG-infergen, pegylated IFN-beta, oral interferon alpha, feron, reaferon, intermax alpha, r-IFN-beta, infergen+actimmune, IFN-omega with DUROS, and albuferon;

13) ribavirin analogs, e.g., rebetol, copegus, levovirin, VX-497, and viramidine (taribavirin);

14) NS5a inhibitors, e.g., BMS-790052, GS-5885, GSK62336805, ACH-2928 AZD2836, AZD7295, BMS-790052, BMS-824393, GS-5885, PPI-1301, PPI-461, A-831 and A-689;

15) NS5b polymerase inhibitors, e.g., IDX-375, NM-283, valopicitabine, R1626, PSI-6130 (R1656), HIV-796, BILB 1941, MK-0608, NM-107, R7128, VCH-759, PF-868554, GSK625433, setrobuvir (ANA598), sofosbuvir, and XTL-2125;

16) NS3 protease inhibitors, e.g., SCH-503034 (SCH-7), VX-950 (Telaprevir), ITMN-191, and BILN-2065;

17) alpha-glucosidase 1 inhibitors, e.g., MX-3253 (celgosivir) and UT-231B;

18) hepatoprotectants, e.g., IDN-6556, ME 3738, MitoQ, and LB-84451;

19) non-nucleoside inhibitors of HIV, e.g., benzimidazole derivatives, benzo-1,2,4-thiadiazine derivatives, and phenylalanine derivatives;

20) other drugs for treating HIV, e.g., zadaxin, nitazoxanide (alinea), BIVN-401 (virostat), DEBIO-025, VGX-410C, EMZ-702, AVI 4065, bavituximab, oglufanide, PYN-17, KPE02003002, actilon (CPG-10101), KRN-7000, civacir, GI-5005, ANA-975 (isatoribine), XTL-6865, ANA 971, NOV-205, tarvacin, EHC-18, and NIM811;

21) pharmacokinetic enhancers, e.g., BAS-100 and SPI452;

22) RNAse H inhibitors, e.g., ODN-93 and ODN-112;

23) other anti-HIV agents, e.g., VGV-1, PA-457 (bevirimat), ampligen, HRG214, cytolin, polymun, VGX-410, KD247, AMZ 0026, CYT 99007, A-221 HIV, BAY 50-4798, MDX010 (iplimumab), PBS119, ALG889, and PA-1050040.

Additional agents for use in the methods herein include monoclonal antibodies that target, and small molecule inhibitors of, Arginase-1, adenosine deaminase, adenosine receptors, IL-4, IL-6 (such as siltuximab/Sylvant™), IL-10, IL-12, IL-15, IL-18, IL-21, C-Kit, stem cell factor (SCF), granulocyte-macrophage colony-stimulating factor (GM-CSF), transforming growth factor beta (TGF-β), vascular endothelial growth factor (VEGF), histone methyltransferases (HMT), glycogen synthase kinase 3 (GSK3),and CD32b.

Also useful are farnesyltransferase inhbitors, such as Lonafarnib (SCH66336, Sarasar™), Chaetomellic acid A, FPT Inhibitors I, II, and III, FTase Inhibitors I (CAS 149759-96-6) and II (CAS156707-43-6), FTI-276 trifluoroacetate salt, FTI-277 trifluoroacetate salt, FTI-2153, GGTI-297, Gingerol, Gliotoxin, L-744,832 Dihydrochloride, Manumycin A, Tipifarnib (R115777, Zarnestra), α-hydroxy Farnesyl Phosphonic Acid, BZA-5B, Manumycin A, hydroxyfarnesylphosphonic acid, butanoic acid, 2-[[(2S)-2-[(2S,3S)-2-[[(2R)-2-amino-3-mercaptopropyl]amino]-3-methylpentyl]oxy-1-oxo-3-phenylpropyl]amino]-4-(methylsulfonyl)-,1-methylethyl ester, (2S)-(9c1), BMS-214662, BMS-316810, dichlorobenzoprim (2,4-diamino-5-(4-(3,4-dichlorobenzylamino)-3-nitrophenyl)-6-ethylpyrimidine), B-581, B-956 (N-(8(R)-amino-2(S)-benzyl-5(S)-isopropyl-9-sulfanyl-3(Z),6(E)-nonadienoyl)-L-methionine), OSI-754, penny! alcohol (1-cyclohexene-1-methanol, 4-(1-methylethenyI)-, RPR-114334, Sch-48755, Sch-226374, (7,8-dichloro-5H-dibenzo(b,e)(1,4)diazepin-11-yl)-pyridin-3-ylmethylamine, J-104126, L-639749, L-731734 (pentanamide, 2-((2-((2-amino-3-mercaptopropyl)amino)-3-methylpentyl)amino)-3-methyl-N-(tetrahydro-2-oxo-3-furanyl)-, (3S-(3R*(2R*(2R*(S*),3S*),3R*)))-), L-744832 (butanoic acid, 2-((2-((2-((2-amino-3-mercaptopropyl)amino)-3-methylpentyl)oxy)-1-oxo-3-phenylpropyl)amino)-4-(methylsulfonyl)-, 1-methylethyl ester, (2S-(1(R*(R*)),2R*(S*),3R*))-), L-745631 (1-piperazinepropanethiol, β-amino-2-(2-methoxyethyl)-4-(1-naphthalenylcarbonyl)-, (8R,2S)-), N-acetyl-N-naphthylmethyl-2(S)-((1-(4-cyanobenzyI)-1H-imidazol-5-yl)acetyl)amino-3(S)-methylpentamine, (2alpha)-2-hydroxy-24,25-dihydroxylanost-8-en-3-one, UCF-1-C (2,4-decadienamide, N-(5-hydroxy-5-(74(2-hydroxy-5-oxo-1-cyclopenten-1-yl)amino-oxo-1,3,5-heptatrienyl)-2-oxo-7-oxabicyclo(4.1.0)hept-3-en-3-yl)-2,4,6-trimethyl-, (1S-(1alpha,3(2E,4E,6S*),5 alpha, 5(1E,3E,5E), 6 alpha))-), UCF-116-B, ARGLABIN (3H-oxireno[8,8a]azuleno[4,5-b]furan-8(4aH)-one, and 5,6,6a,7,9a,9b-hexahydro-1,4a-dimethyl-7-methylene-, (3aR,4aS,6aS,9aS,9bR)-).

Also useful in the methods and combinations herein are inhibitors of 26S proteasome, such as Lactacystin, Bortezomib (PS-341), ritonavir, MG-132 (Z-Leu-Leu-Leu-CHO), MG-115 (Z-LL-Nva-CHO), Proteasome Inhibitor I (Z-Ile-Giu(OtBu)-Ala-Leu-CHO), and Proteasome Inhibitor II (Z-LLF-CHO).

Useful inhibitors of E3 ubiquitin ligase include proTAME, RITA (5,5′-(2,5-Furandiyl)bis-2-thiophenemethanol), HLI 373 (5-[[3-Dimethylamino)propyl]amino]-3,10-dimethylpyrimido[4,5-b]quinoline-2,4(3H,10H)-dione dihydrochloride), Nutlin-3 ((±)-4-[4,5-Bis(4-chlorophenyI)-2-(2-isopropoxy-4-methoxy-phenyl)-4,5-dihydro-imidazole-1-carbonyl]-piperazin-2-one), SMER 3 (9H-Indeno[1,2-e][1,2,5]oxadiazolo[3,4-b]pyrazin-9-one), NSC 66811 (2-Methyl-7-[Phenyl(phenylamino)methyl]-8-quinolinol), TAME HCl (N²-[(4-Methylphenyl)sulfonyl]-L-arginine methyl ester hydrochloride), Heclin (N-(4-Acetylphenyl)-3-(5-ethyl-2-furanyl)-2-propenamide), PRT 4165 (2-(3-Pyridinylmethylene)-1H-Indene-1,3(2H)-dione), NAB 2 (N-[(2-Chlorophenyl)methyl]-1-(2,5-dimethylphenyl)-1H-benzimidazole-5-carboxamide), SP 141 (6-Methoxy-1-(1-naphthalenyl)-9H-pyrido[3,4-b]indole), SZL P1-41 (3-(2-Benzothiazolyl)-6-ethyl-7-hydroxy-8-(1-piperidinylmethyl)-4H-1-benzopyran-4-one), PTC 209 (N-(2,6-Dibromo-4-methoxyphenyl)-4-(2-methylimidazo[1,2-a]pyrimidin-3-yl)-2-thiazolamine), SKP C1 (2-[4-Bromo-2-[[4-oxo-3-(3-pyridinylmethyl)-2-thioxo-5-thiazolidinylidene]methyl]phenoxy]acetic acid), A01 ([4-[[4-Chloro-3-(trifluoromethyl)phenyl]sulfonyl]-1-piperazinyl][4-(5-methyl-1H-pyrazol-1-yl)phenyl]methanone), Apcin.

Useful agonists of protein kinase C (PKC) include midostaurin (PKC412, CGP41251, 4′-N-benzoyl staurosporine), ruboxistaurin (LY 333531 HCl, (9S)-9-[(Dimethylamino)methyl]-6,7,10,11-tetrahydro-9H,18H-5,21:12,17-dimethenodibenzo[e,k]pyrrolo[3,4-h][1,4,13]oxadiazacyclohexadecine-18,20(19H)-dione hydrochloride), sotrastaurin (AEB071), enzastaurin (LY317615 HCl), sotrastaurin (AEB071), CGP60474, chelerythrine chloride (HY-12048), Fasudil HCl (HY-10341, Go 6983 (HY-13689), and Zoledronic acid (CGP 42446). include phorbol esters, such as PMA, prostratin, and 12-deoxyphorbol 13-phenylacetate (DPP), and non-phorbol ester compounds including bryostatin compounds, including Bryostatin-1, diacylglycerol (DAG) analogs such as LMC03 and LMC07, including DAG lactones, such as HK654, HK434, HK602, and HK204, ingenol derivatives, including ITA, ingenol-3-hexanoate (IngB), and 1-3-A as well as ingot diterpenes, such as 8-methoxyingol 7,12-diacetate 3-phenylacetate, 8-methoxyingol 7,12-diacetate 3-phenylacetate (SJ23B), (5aS,7S,8aR,E)-1,1,4,7,10-pentamethyl-2-(((E)-2-methylbut-2-enoyhoxy)-9-oxo-1,1a,2,3,6,7,8,9,10,10a-decahydro-5a,8a-epoxycyclopenta[a]cyclopropa [e][10]annulene-6,10a-diyldiacetate, and gnidimacrin.

Again by way of example, the following list discloses exemplary HIV antivirals, with their corresponding U.S. Patent numbers, incorporated by reference with regard to the preparation of such antivirals, which can be combined in the present methods with the TLR8 modulating compounds described herein.

Exemplary HIV Antivirals and Patent Numbers

Examples of HIV antiviral agents useful in the combinations and methods herein include Ziagen (Abacavir sulfate, U.S. Pat. No. 5,034,394); Epzicom (Abacavir sulfate/lamivudine, U.S. Pat. No. 5,034,394); Hepsera (Adefovir dipivoxil, U.S. Pat. No. 4,724,233); Agenerase (Amprenavir, U.S. Pat. No. 5,646,180); Reyataz (Atazanavir sulfate, U.S. Pat. No. 5,849,911); Rescriptor (Delavirdine mesilate, U.S. Pat. No. 5,563,142); Hivid (Dideoxycytidine; Zalcitabine, U.S. Pat. No. 5,028,595); Videx (Dideoxyinosine; Didanosine, U.S. Pat. No. 4,861,759); Sustiva (Efavirenz, U.S. Pat. No. 5,519,021); Emtriva (Emtricitabine, U.S. Pat. No. 6,642,245)

Lexiva (Fosamprenavir calcium, U.S. Pat. No. 6,436,989); Virudin; Triapten; Foscavir (Foscarnet sodium, U.S. Pat. No. 6,476,009); Crixivan (Indinavir sulfate, U.S. Pat. No. 5,413,999); Epivir (Lamivudine, U.S. Pat. No. 5 047,407); Combivir (Lamivudine/Zidovudine, U.S. Pat. No. 4,724,232); Aluviran (Lopinavir); Kaletra (Lopinavir/ritonavir, U.S. Pat. No. 5,541,206); Viracept (Nelfinavir mesilate, U.S. Pat. No. 5,484,926);

Viramune (Nevirapine, U.S. Pat. No. 5,366,972); Norvir (Ritonavir, U.S. Pat. No. 5,541,206); Invirase; Fortovase (Saquinavir mesilate, U.S. Pat. No. 5,196,438); Zerit (Stavudine, U.S. Pat. No. 4,978,655); Truvada® (Tenofovir disoproxil fumarate/emtricitabine, U.S. Pat. No. 5,210,085); Viread® (tenofovir disoproxil fumarate)

Complera® (emtricitabine/rilpivirine/tenofovir disoproxil fumarate); Atripla® (efavirenz/emtricitabine/tenofovir disoproxil fumarate); Stribild® (elvitegravir 150 mg/cobicistat 150 mg/emtricitabine 200 mg/tenofovir disoproxil fumarate 300 mg); Aptivus (Tipranavir); emtricitabine/rilpivirine/tenofovir alafenamide; rilpivirine/tenofovir alafenamide; emtricitabine/tenofovir alafenamide; emtricitabine/rilpivirine/tenofovir alafenamide hemifumarate; rilpivirine/tenofovir alafenamide hemifumarate; emtricitabine/tenofovir alafenamide hemifumarate.

Retrovir (Zidovudine; Azidothymidine, U.S. Pat. No. 4,724,232); and Eviplera® (emtricitabine/rilpivirine/tenofovir disoproxil).

In yet another embodiment, the present application discloses pharmaceutical compositions comprising a TLR8 modulating compound described herein, or a pharmaceutically acceptable salt thereof, in combination with at least one additional active agent, and a pharmaceutically acceptable excipient. In yet another embodiment, the present application provides a combination pharmaceutical agent with two or more therapeutic agents in a unitary dosage form. Thus, it is also possible to combine any compound of the invention with one or more other active agents in a unitary dosage form.

The combination therapy may be administered as a simultaneous or sequential regimen. When administered sequentially, the combination may be administered in two or more administrations.

Co-administration of a compound of the invention with one or more other active agents generally refers to simultaneous or sequential administration of a compound of the invention and one or more other active agents, such that therapeutically effective amounts of the compound of the invention and one or more other active agents are both present in the body of the patient.

Co-administration includes administration of unit dosages of the compounds of the invention before or after administration of unit dosages of one or more other active agents, for example, administration of the compounds of the invention within seconds, minutes, or hours of the administration of one or more other active agents. For example, a unit dose of a compound of the invention can be administered first, followed within seconds or minutes by administration of a unit dose of one or more other active agents. Alternatively, a unit dose of one or more other active agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active agents. In other cases, it may be desirable to administer a unit dose of one or more other active agents first, followed, after a period of hours (e.g., 1-12 hours), by administration of a unit dose of a compound of the invention.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together.

### Methods of Treatment

As used herein, an “agonist” is a substance that stimulates its binding partner, typically a receptor. Stimulation is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Stimulation may be defined with respect to an increase in a particular effect or function that is induced by interaction of the agonist or partial agonist with a binding partner and can include allosteric effects.

As used herein, an “antagonist” is a substance that inhibits its binding partner, typically a receptor. Inhibition is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Inhibition may be defined with respect to a decrease in a particular effect or function that is induced by interaction of the antagonist with a binding partner, and can include allosteric effects.

As used herein, a “partial agonist” or a “partial antagonist” is a substance that provides a level of stimulation or inhibition, respectively, to its binding partner that is not fully or completely agonistic or antagonistic, respectively. It will be recognized that stimulation, and hence, inhibition is defined intrinsically for any substance or category of substances to be defined as agonists, antagonists, or partial agonists.

As used herein, “intrinsic activity” or “efficacy” relates to some measure of biological effectiveness of the binding partner complex. With regard to receptor pharmacology, the context in which intrinsic activity or efficacy should be defined will depend on the context of the binding partner (e.g., receptor/ligand) complex and the consideration of an activity relevant to a particular biological outcome. For example, in some circumstances, intrinsic activity may vary depending on the particular second messenger system involved. Where such contextually specific evaluations are relevant, and how they might be relevant in the context of the present invention, will be apparent to one of ordinary skill in the art.

As used herein, modulation of a receptor includes agonism, partial agonism, antagonism, partial antagonism, or inverse agonism of a receptor. A TLR8 modulating compound may also be referred to as a TLR8modulating agent, a TLR8 modulator, a compound which modulates TLR8 activity, or the like.

As will be appreciated by those skilled in the art, when treating a viral infection such as HIV, such treatment may be characterized in a variety of ways and measured by a variety of endpoints. The scope of the present invention is intended to encompass all such characterizations.

In one embodiment, the method can be used to modulate an immune response against multiple epitopes of a viral infection in a human. Induction of an immune response against viral infection can be assessed using any technique that is known by those of skill in the art for determining whether an immune response has occurred. Suitable methods of detecting an immune response for the present invention include, among others, detecting a decrease in viral load or antigen in a subjects serum, and detection of IFN-gamma-secreting antigen specific T cells. Additional methods include activation of immune cell subsets such as CD4, CD8, NK, monocytes, and or mDCs, monitoring general or cell-specific surface activation marekrs, production of cytokines or functional assays such antigen-specific and non-specific cell killing activities. In one embodiment, the detection of IFN-gamma-secreting antigen specific T cells is accomplished using an ELISPOT assay or FACS analysis.

A TLR8 modulating compound as described herein can be administered by any useful route and means, such as by oral or parenteral (e.g., intravenous) administration. Therapeutically effective amounts of a TLR8 modulating compound as described herein are from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 1mg to about 250 mg per day, or such as from about 10 mg to about 150 mg per day.

The frequency of dosage of a TLR8 modulating compound as described herein will be determined by the needs of the individual patient and can be, for example, once per day or twice, or more times, per day. Administration of a TLR8 modulating compound as described herein continues for as long as necessary to treat the HIV infection. For example, a TLR8 modulating compound as described herein can be administered to a human being infected with HIV for a period of from 20 days to 180 days or, for example, for a period of from 20 days to 90 days or, for example, for a period of from 30 days to 60 days.

Administration can be intermittent, with a period of one or more days during which a patient receives a daily dose of a TLR8 modulating compound as described herein, followed by a period of several or more days during which a patient does not receive a daily dose of a TLR8 modulating compound as described herein. For example, a patient can receive a dose of a TLR8 modulating compound as described herein every other day, or three times per week, once per week (every 7 days), once every other week (every 14 days), once per month, or once every other month. Again by way of example, a patient can receive a dose of a TLR8 modulating compound as described herein each day for a period of from 1 to 14 days, followed by a period of 7 to 30 days during which the patient does not receive a dose of a TLR8 modulating compound as described herein, followed by a subsequent period (e.g., from 1 to 14 days) during which the patient again receives a daily dose of a TLR8 modulating compound as described herein. For other examples, in separate embodiments, a patient can receive an initial single dose of a TLR8 modulating compound as described herein, followed by sequential doses every other day, or three times per week, once per week (every 7 days), once every other week (every 14 days), once per month, or once every other month. Alternating periods of administration of a TLR8 modulating compound as described herein, followed by non-administration of a TLR8 modulating compound as described herein, can be repeated as clinically required to treat the patient.

Each of the TLR8 modulating compounds of Formula (J), (I), (II), (IIa), (IIb), (III), (IIIa), (IIIb), (IV), (IVa), (IVb), (IVc), or (IVd), and Examples 1-48, are disclosed in U.S. Provisional Patent Applications No. 62/128,397, filed Mar. 4, 2015, and 62/250,403, filed Nov. 3, 2015, U.S. patent application Ser. No. 15/059,070, filed Mar. 2, 2016 and PCT Application No. PCT/US2016/020499, filed Mar. 2, 2016, and may be prepared according to methods described therein or by other methods known to those of skill in the art.

For reference. Scheme 1 shows a representative synthesis of the compounds of Formula (I) and (J). The methodology is compatible with a wide variety of functionalities.

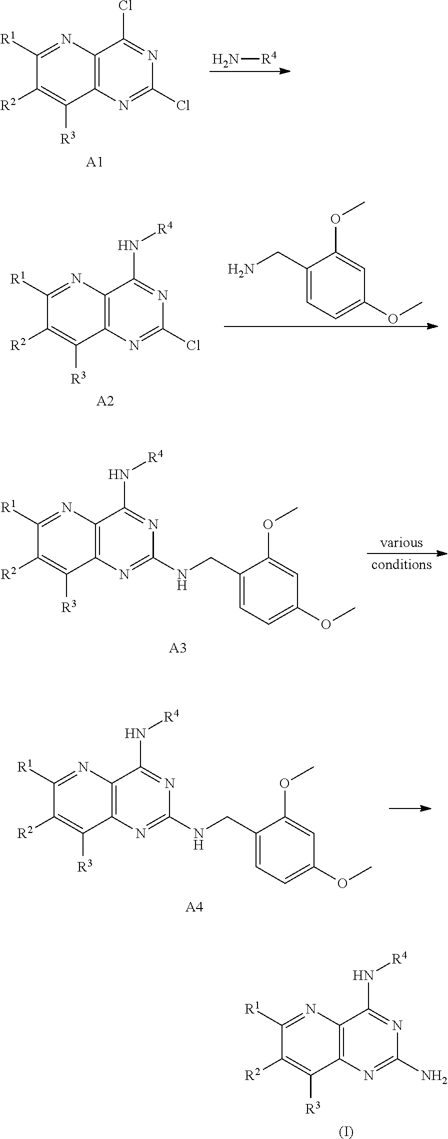

In Scheme 1, compounds of formula A1 (where R¹, R², and R³ are as defined herein or are suitably protected derivatives of R¹, R², and R³) are converted to the corresponding 4-amino, 2-chloro heterocycle by reaction with a nuclephilic amine in the presence of a suitable base (such as DIPEA) at room temperature. The compound of formula A2 is then treated with 2,4-dimethoxybenzylamine at elevated temperature resulting in a 2,4-diaminopyrimidine of formula A3. In cases where R¹, R², and R³ is a diversifiable chemical group such as Cl or Br, further replacement of R¹, R², and R³ by a variety of methods including cyanation, nucleophilic aromatic displacement, and metal catalyzed cross coupling reactions such as Suzuki couplings is carried out to provide products of formula A4. Treatment with a suitable acid (such as trifluoroacetic acid) leads to certain compounds of Formula (I) or (J)

Scheme 2 describes a general route which is used to prepare certain compounds of Formula (I) or (J).

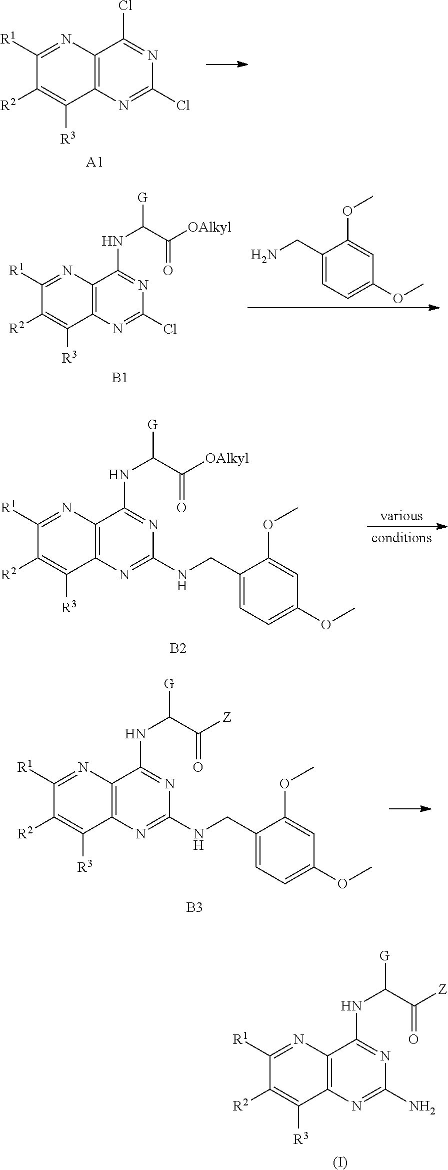

2,4-dichloro pyrido-pyrimidines of formula A1 (where R¹, R², and R³ are as defined herein or are suitably protected derivatives of R¹, R², and R³) are converted to the corresponding 4-amino,2-chloro heterocycle by reaction with an amino acid ester (such as L-norvaline methyl ester) in the presence of a suitable base (such as DIPEA) at room temperature to provide a compound of formula B1, where G is an the sidechain of the amino acid. The compound of formula B1 is then treated with 2,4-dimethoxybenzylamine in a microwave reactor at a suitable temperature (such as about 135° C.), resulting in a 2,4-diaminopyrimidine of formula B2. Hydrolysis of the ester group via treatment with a suitable base (such as aqueous KOH/THF) provides product of formula B3 where Z is hydroxyl. Further reaction of the resulting carboxylic acid leads to modification of Z via HATU-promoted amide formation with various amines. Protecting group removal with a suitable acid (such as trifluoroacetic acid) at room temperature then leads to certain compounds of Formula (J) or (I).

In certain instances, the above processes further involve the step of forming a salt of a compound of the present disclosure. Embodiments are directed to the other processes described herein; and to the product prepared by any of the processes described herein.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, 5^{th} edition, New York: Oxford University Press, 2009; Smith, March's Advanced Organic Chemistry: Reactions, Mechanisms, and Structure, 7^{th} edition, Wiley-Interscience, 2013.

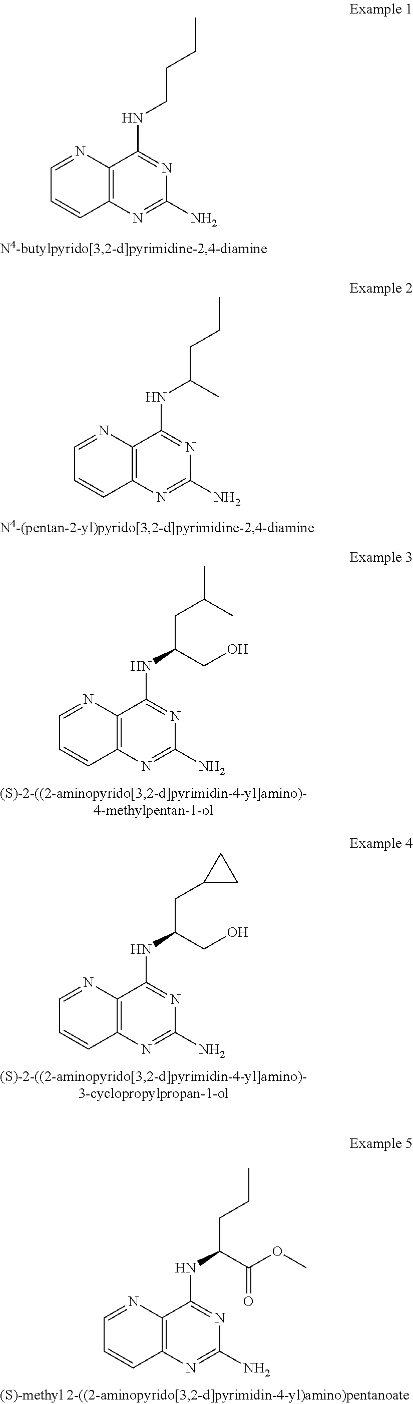 [SMILES: CCCCNc1nc(N)nc2cccnc12 ; CCCC(C)Nc1nc(N)nc2cccnc12 ; CC(C)C[C@@H](CO)Nc1nc(N)nc2cccnc12 ; Nc1nc(N[C@H](CO)CC2CC2)c2ncccc2n1 ; CCC[C@H](Nc1nc(N)nc2cccnc12)C(=O)OC]

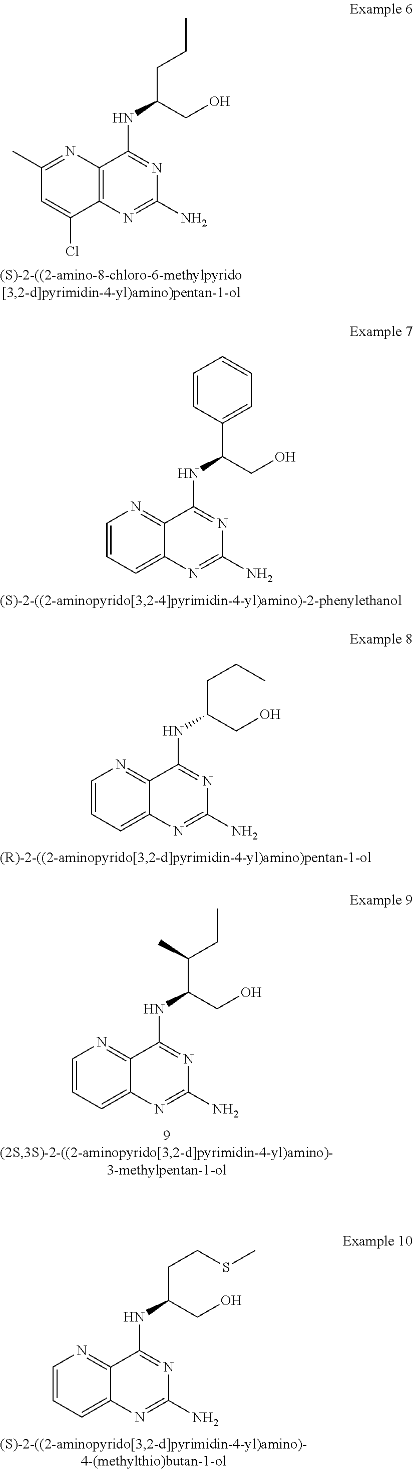 [SMILES: CCC[C@@H](CO)Nc1nc(N)nc2c(Cl)cc(C)nc12 ; Nc1nc(N[C@H](CO)c2ccccc2)c2ncccc2n1 ; CCC[C@H](CO)Nc1nc(N)nc2cccnc12 ; CC[C@H](C)[C@@H](CO)Nc1nc(N)nc2cccnc12 ; CSCC[C@@H](CO)Nc1nc(N)nc2cccnc12]

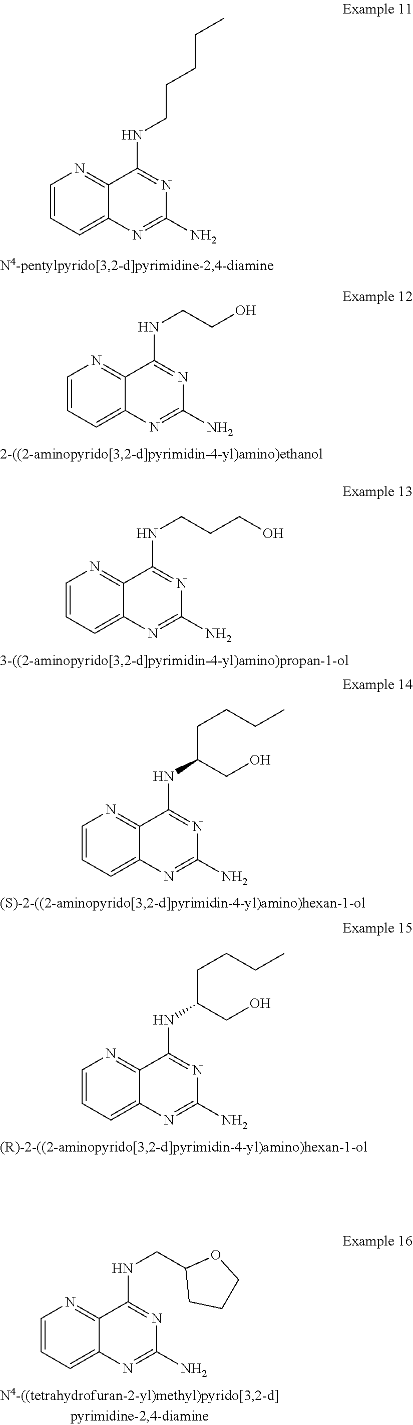 [SMILES: CCCCCNc1nc(N)nc2cccnc12 ; Nc1nc(NCCO)c2ncccc2n1 ; Nc1nc(NCCCO)c2ncccc2n1 ; CCCC[C@@H](CO)Nc1nc(N)nc2cccnc12 ; CCCC[C@H](CO)Nc1nc(N)nc2cccnc12 ; Nc1nc(NCC2CCCO2)c2ncccc2n1]

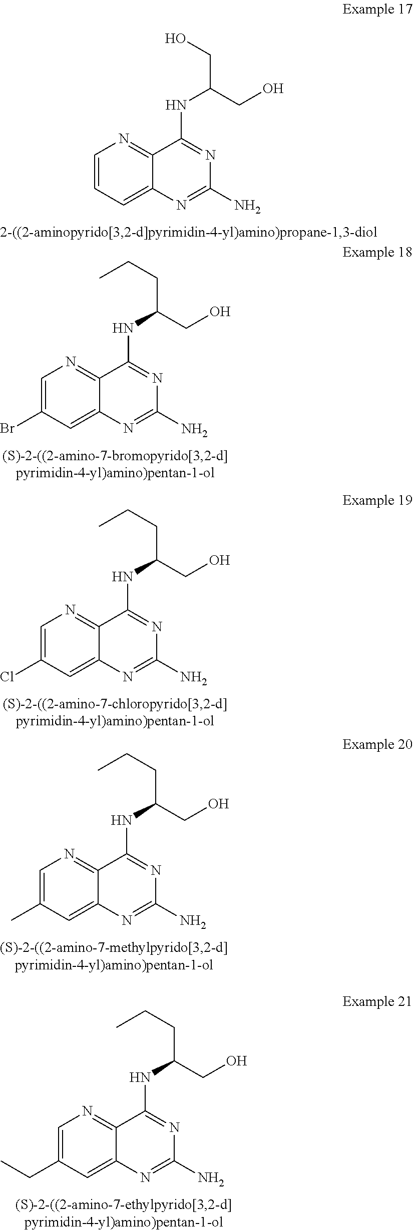 [SMILES: Nc1nc(NC(CO)CO)c2ncccc2n1 ; CCC[C@@H](CO)Nc1nc(N)nc2cc(Br)cnc12 ; CCC[C@@H](CO)Nc1nc(N)nc2cc(Cl)cnc12 ; CCC[C@@H](CO)Nc1nc(N)nc2cc(C)cnc12 ; CCC[C@@H](CO)Nc1nc(N)nc2cc(CC)cnc12]

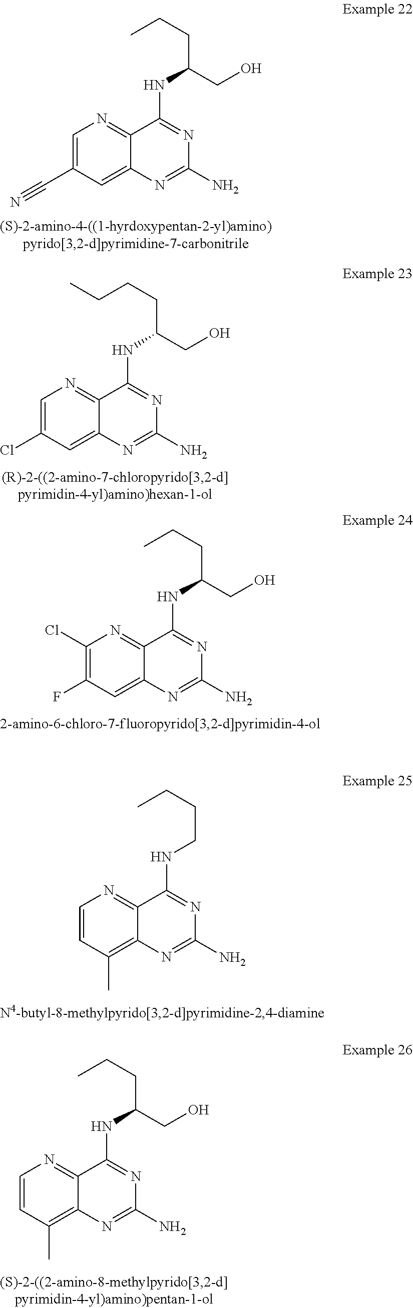 [SMILES: CCC[C@@H](CO)Nc1nc(N)nc2cc(C#N)cnc12 ; CCCC[C@H](CO)Nc1nc(N)nc2cc(Cl)cnc12 ; CCC[C@@H](CO)Nc1nc(N)nc2cc(F)c(Cl)nc12 ; CCCCNc1nc(N)nc2c(C)ccnc12 ; CCC[C@@H](CO)Nc1nc(N)nc2c(C)ccnc12]

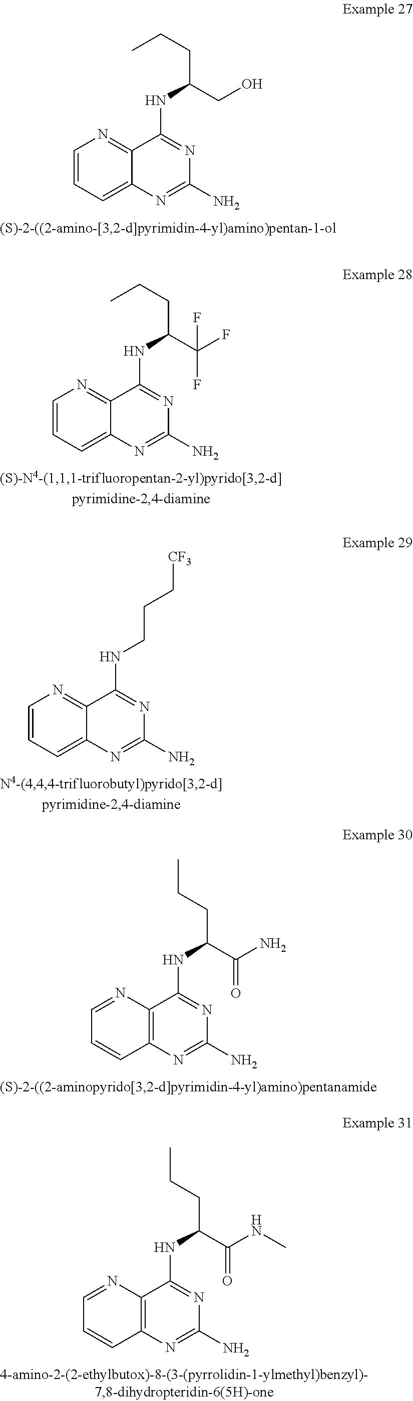 [SMILES: CCC[C@@H](CO)Nc1nc(N)nc2cccnc12 ; CCC[C@H](Nc1nc(N)nc2cccnc12)C(F)(F)F ; Nc1nc(NCCCC(F)(F)F)c2ncccc2n1 ; CCC[C@H](Nc1nc(N)nc2cccnc12)C(N)=O ; CCC[C@H](Nc1nc(N)nc2cccnc12)C(=O)NC]

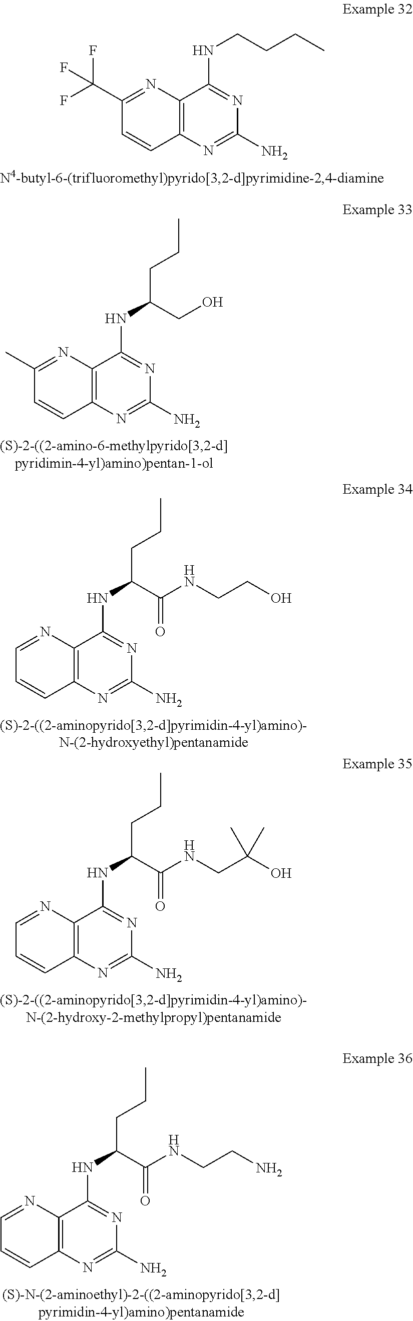

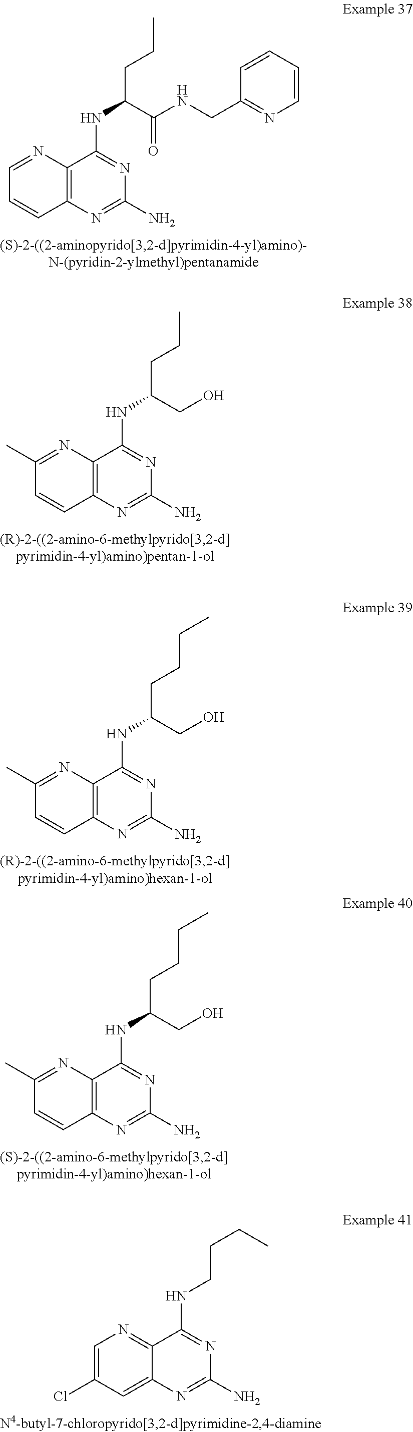

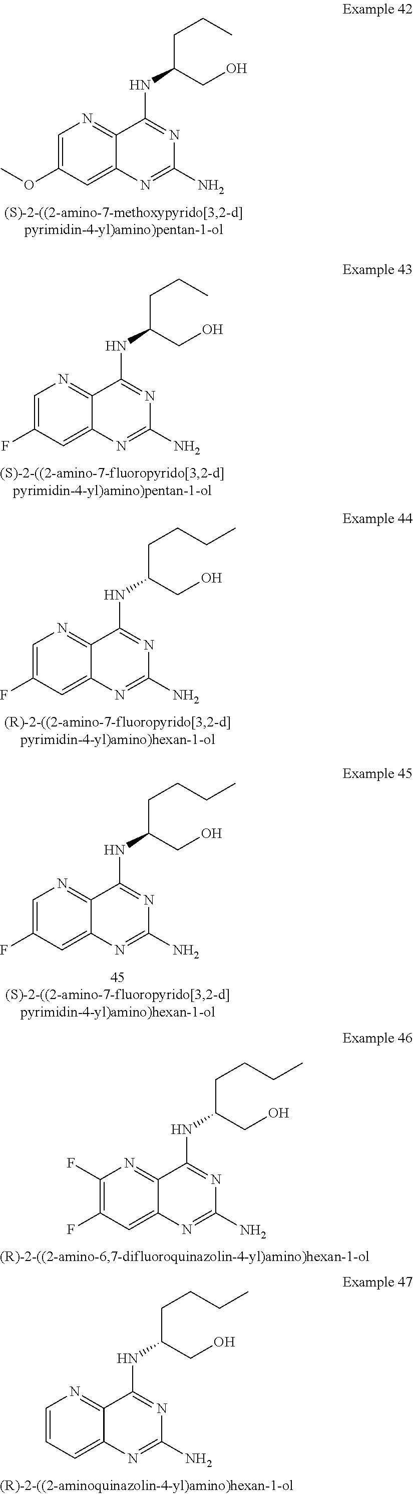 [SMILES: CCC[C@@H](CO)Nc1nc(N)nc2cc(OC)cnc12 ; CCC[C@@H](CO)Nc1nc(N)nc2cc(F)cnc12 ; CCCC[C@H](CO)Nc1nc(N)nc2cc(F)cnc12 ; CCCC[C@@H](CO)Nc1nc(N)nc2cc(F)cnc12 ; CCCC[C@H](CO)Nc1nc(N)nc2cc(F)c(F)nc12 ; CCCC[C@H](CO)Nc1nc(N)nc2cccnc12]

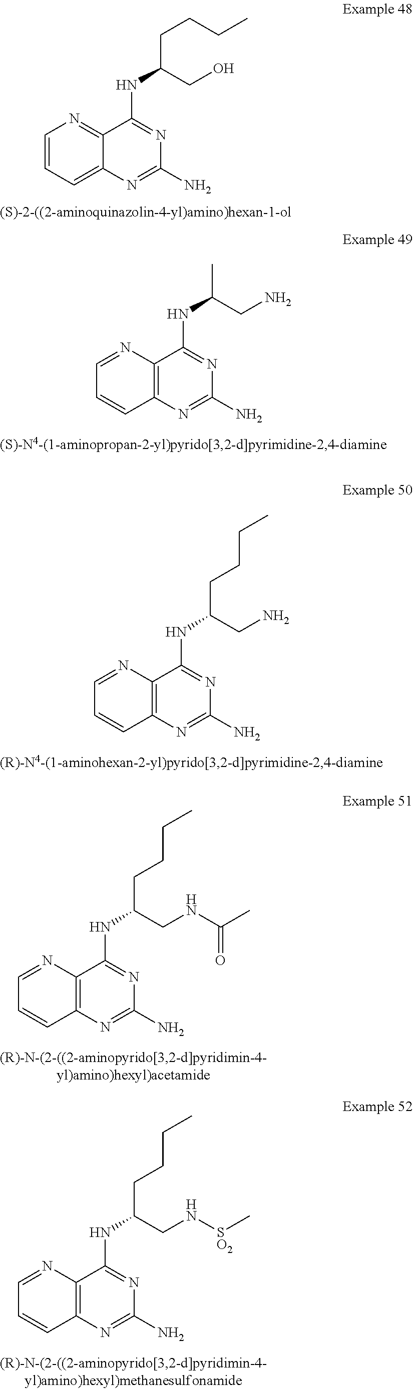 [SMILES: CCCC[C@@H](CO)Nc1nc(N)nc2cccnc12 ; C[C@@H](CN)Nc1nc(N)nc2cccnc12 ; CCCC[C@H](CN)Nc1nc(N)nc2cccnc12 ; CCCC[C@H](CNC(C)=O)Nc1nc(N)nc2cccnc12 ; CCCC[C@H](CNS(C)(=O)=O)Nc1nc(N)nc2cccnc12]

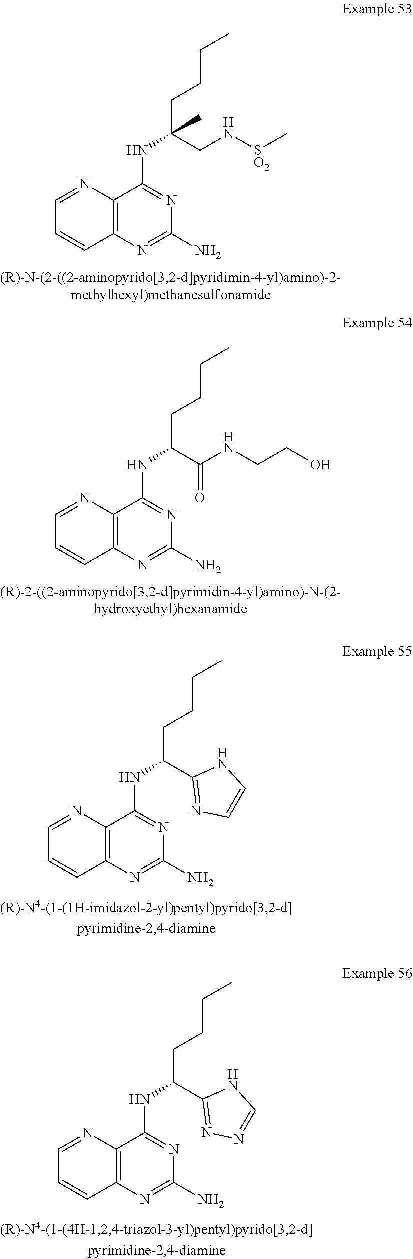

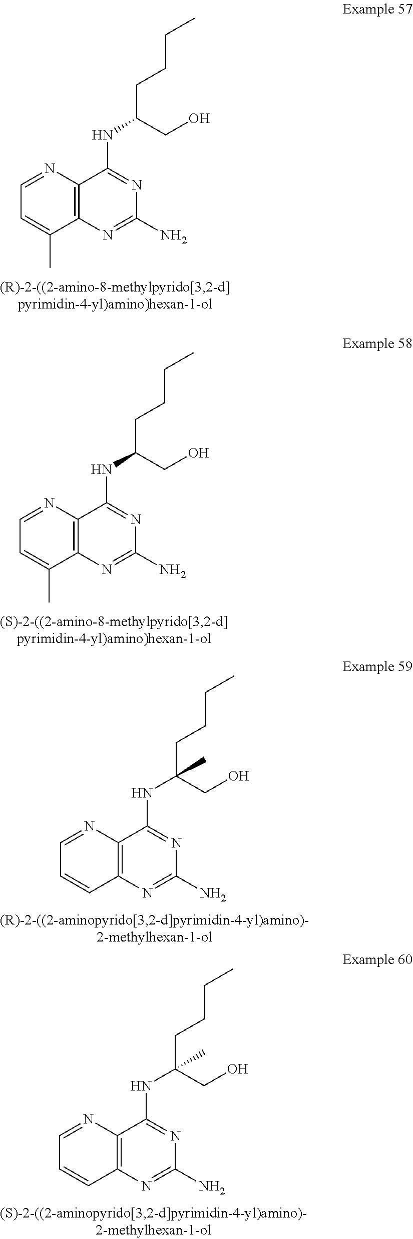 [SMILES: CCCC[C@H](CO)Nc1nc(N)nc2c(C)ccnc12 ; CCCC[C@@H](CO)Nc1nc(N)nc2c(C)ccnc12 ; CCCC[C@](C)(CO)Nc1nc(N)nc2cccnc12 ; CCCC[C@@](C)(CO)Nc1nc(N)nc2cccnc12]

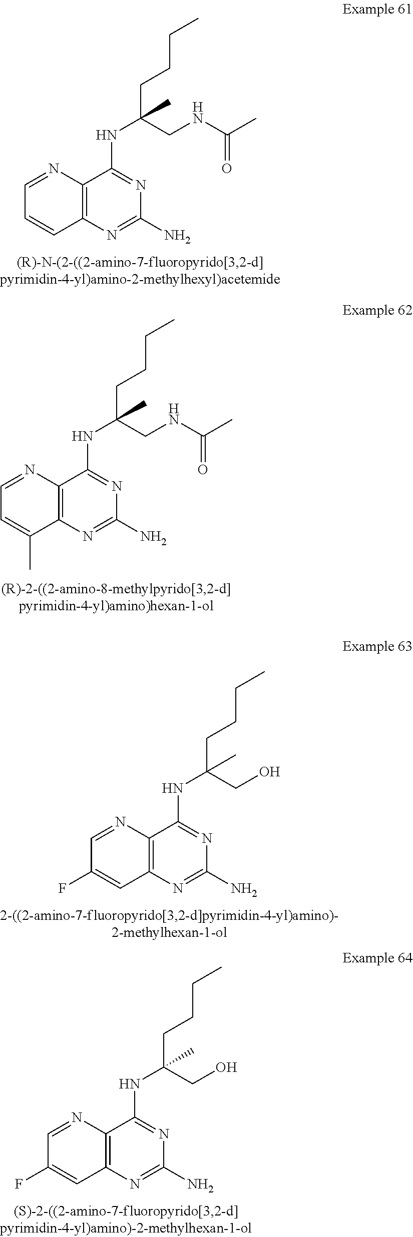 [SMILES: CCCC[C@](C)(CNC(C)=O)Nc1nc(N)nc2cccnc12 ; CCCC[C@](C)(CNC(C)=O)Nc1nc(N)nc2c(C)ccnc12 ; CCCCC(C)(CO)Nc1nc(N)nc2cc(F)cnc12 ; CCCC[C@@](C)(CO)Nc1nc(N)nc2cc(F)cnc12]

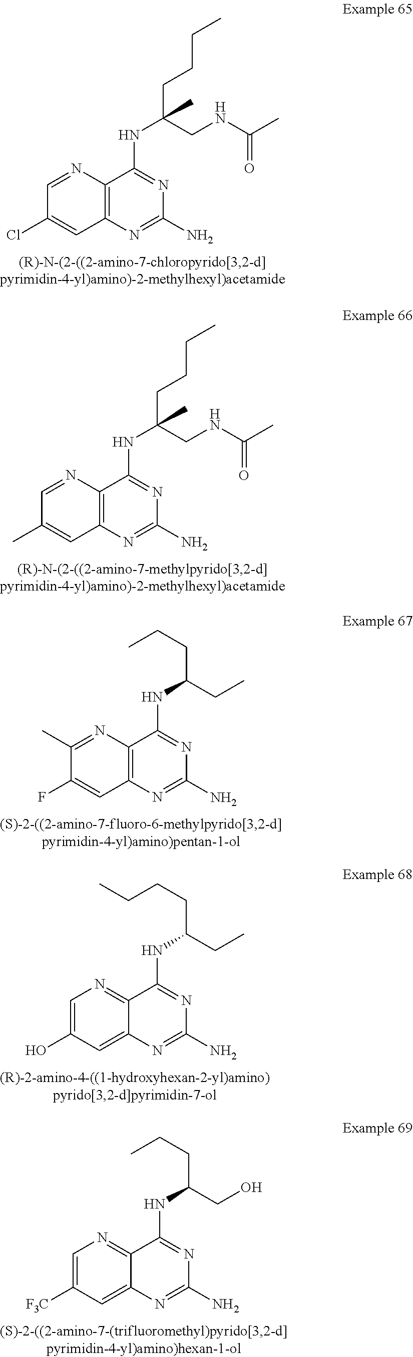

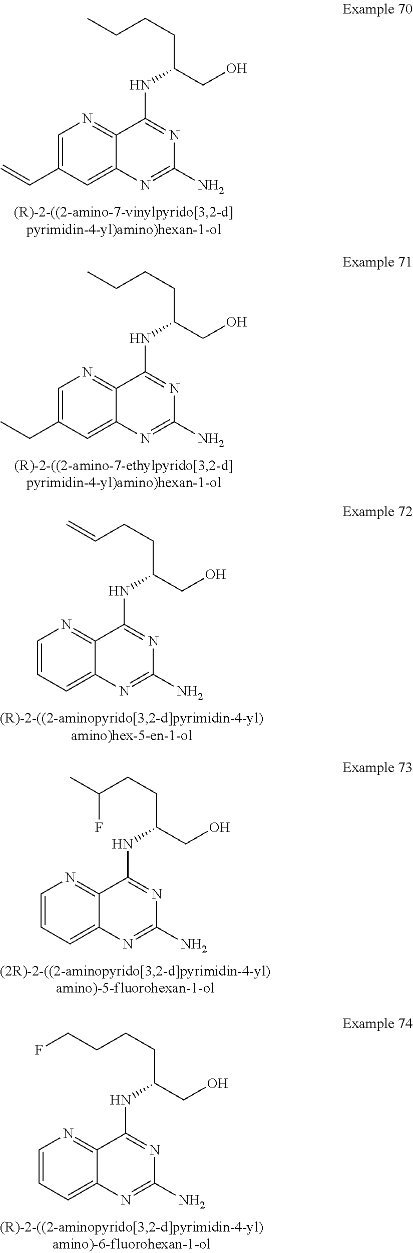 [SMILES: C=Cc1cnc2c(N[C@@H](CO)CCCC)nc(N)nc2c1 ; CCCC[C@H](CO)Nc1nc(N)nc2cc(CC)cnc12 ; C=CCC[C@H](CO)Nc1nc(N)nc2cccnc12 ; CC(F)CC[C@H](CO)Nc1nc(N)nc2cccnc12 ; Nc1nc(N[C@@H](CO)CCCCF)c2ncccc2n1]

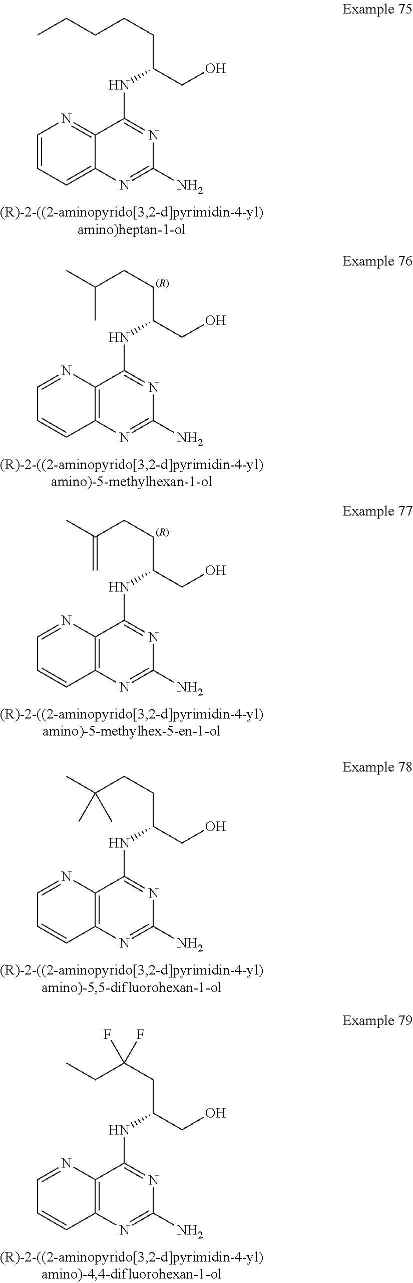

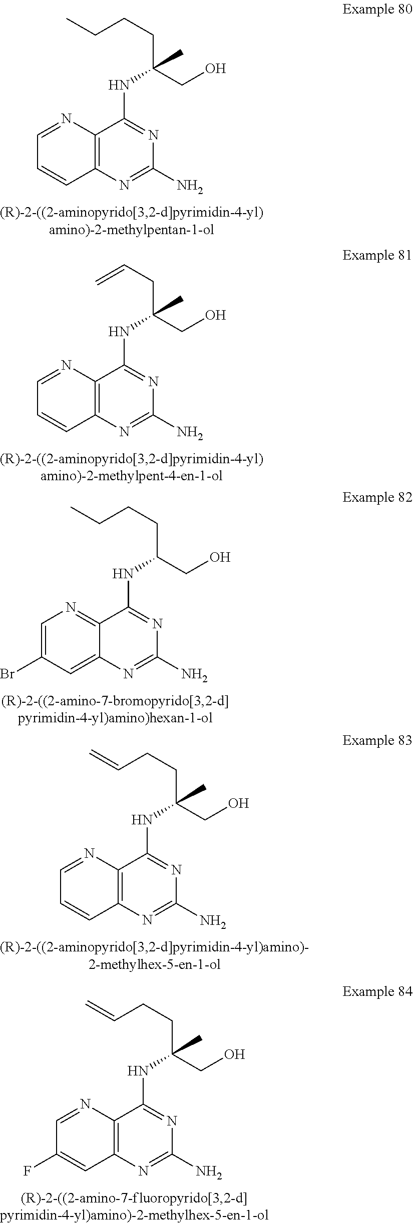

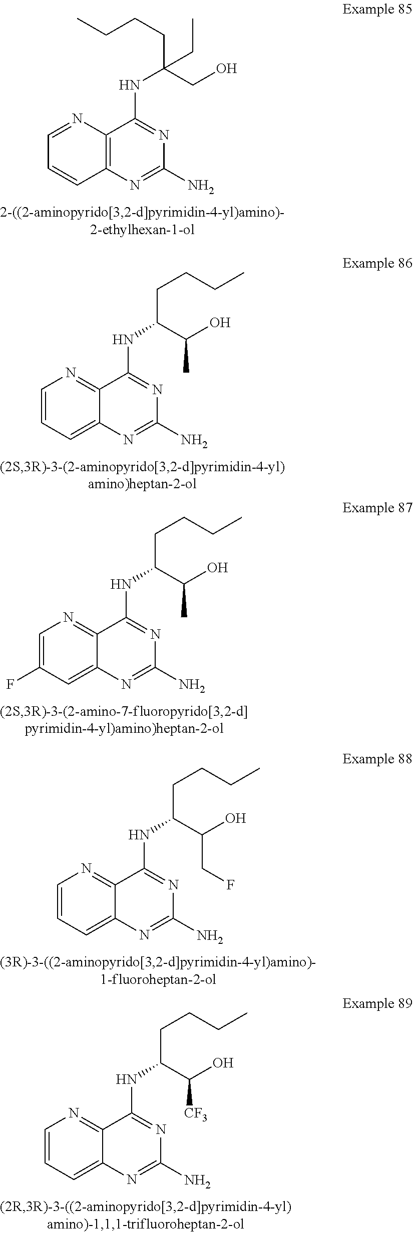 [SMILES: CCCCC(CC)(CO)Nc1nc(N)nc2cccnc12 ; CCCC[C@@H](Nc1nc(N)nc2cccnc12)[C@H](C)O ; CCCC[C@@H](Nc1nc(N)nc2cc(F)cnc12)[C@H](C)O ; CCCC[C@@H](Nc1nc(N)nc2cccnc12)C(O)CF ; CCCC[C@@H](Nc1nc(N)nc2cccnc12)[C@@H](O)C(F)(F)F]

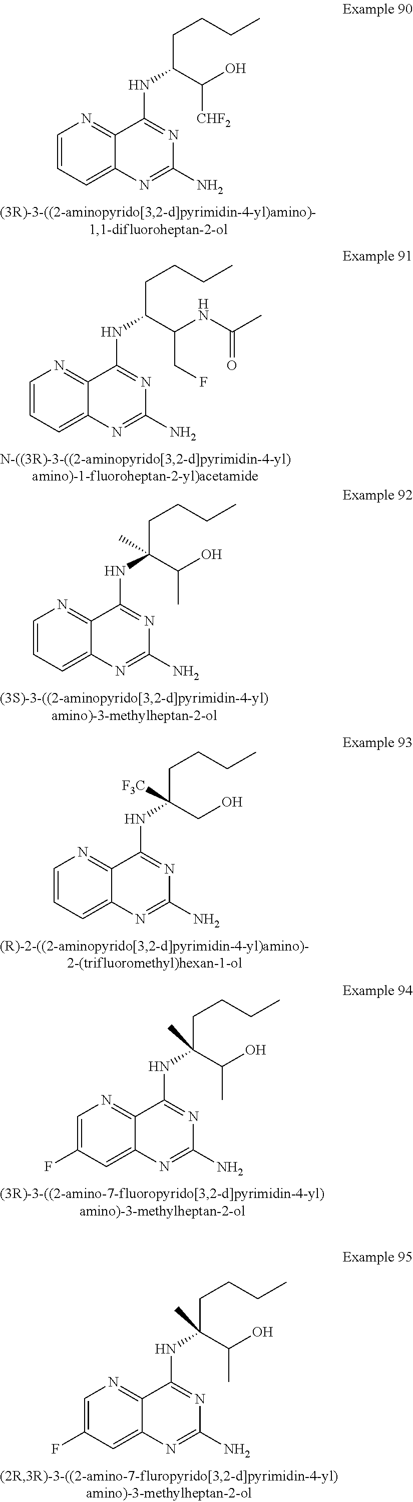 [SMILES: CCCC[C@@H](Nc1nc(N)nc2cccnc12)C(O)C(F)F ; CCCC[C@@H](Nc1nc(N)nc2cccnc12)C(CF)NC(C)=O ; CCCC[C@](C)(Nc1nc(N)nc2cccnc12)C(C)O ; CCCC[C@](CO)(Nc1nc(N)nc2cccnc12)C(F)(F)F ; CCCC[C@@](C)(Nc1nc(N)nc2cc(F)cnc12)C(C)O ; CCCC[C@@](C)(Nc1nc(N)nc2cc(F)cnc12)C(C)O]

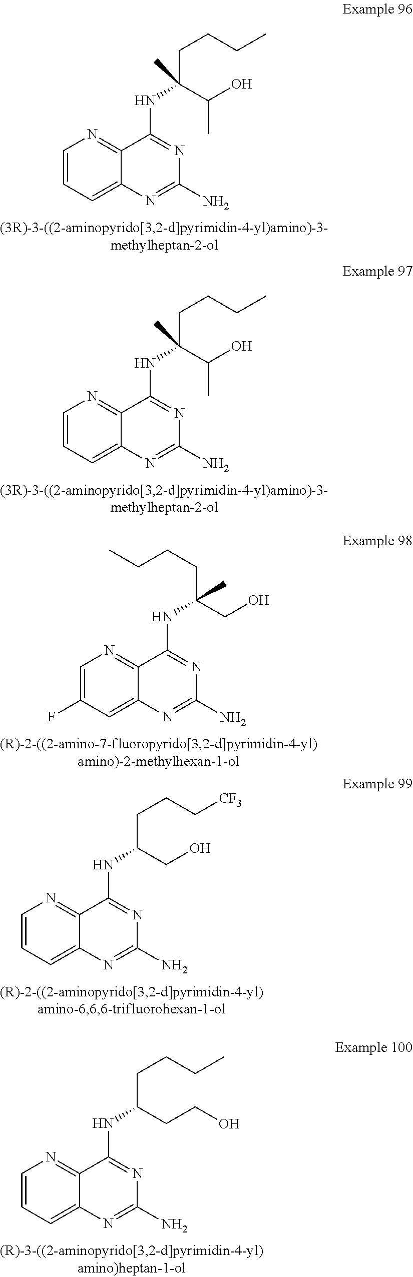

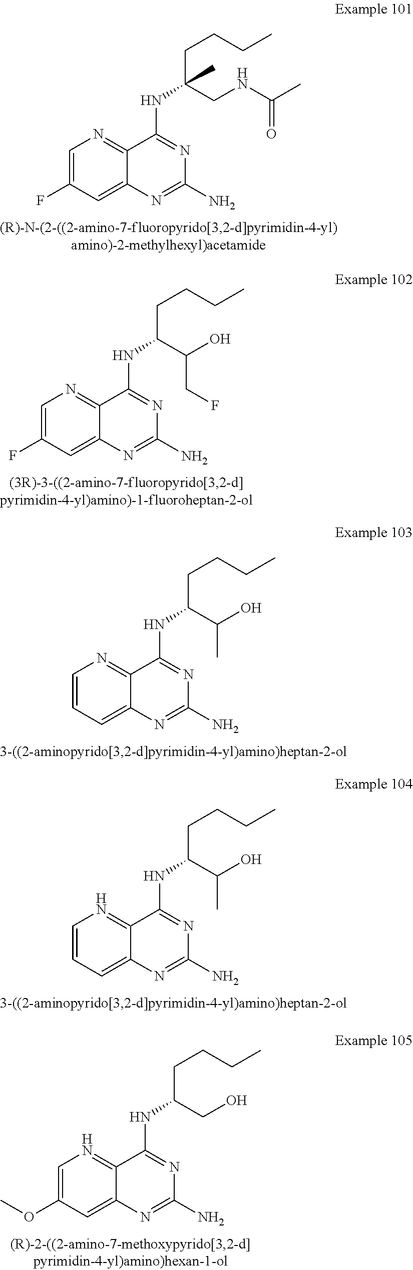

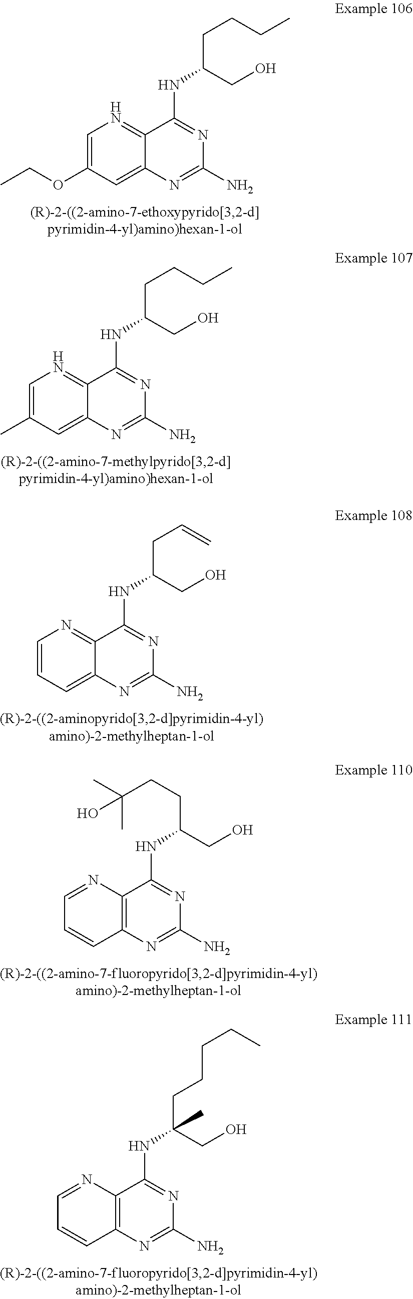

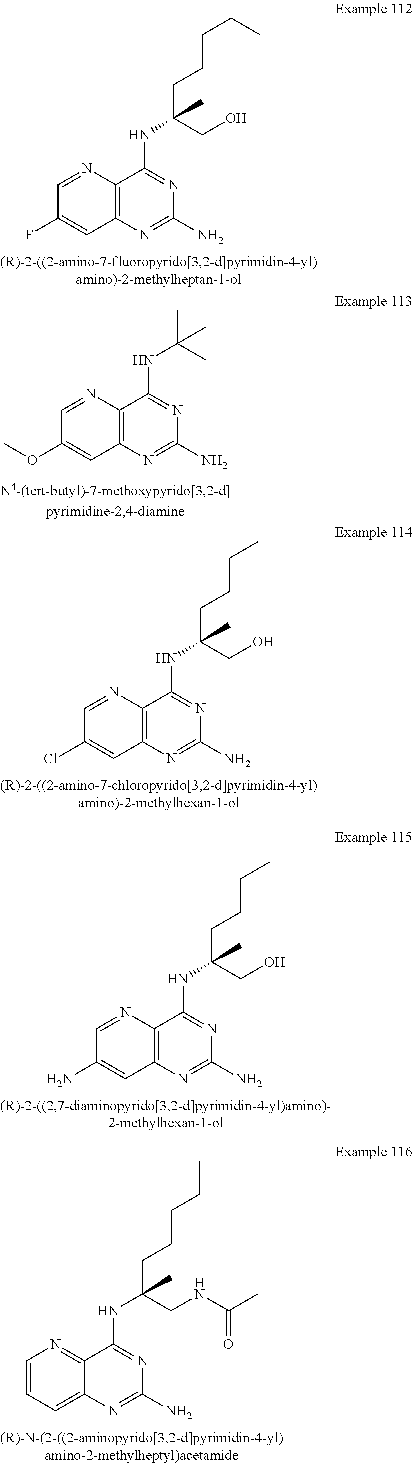

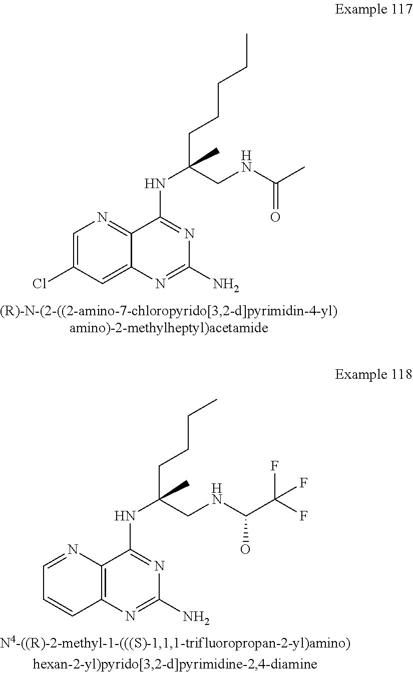 [SMILES: CCCCC[C@](C)(CNC(C)=O)Nc1nc(N)nc2cc(Cl)cnc12 ; CCCC[C@](C)(CN[C@@H](O)C(F)(F)F)Nc1nc(N)nc2cccnc12]

### Example 119

### (S)-2-((2-amino-6,7-difluoroquinazolin-4-yl)amino)pentan-1-ol

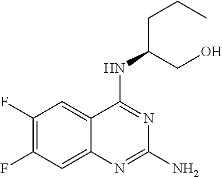 [SMILES: CCC[C@@H](CO)Nc1nc(N)nc2cc(F)c(F)cc12]

The compound of Example 119 can be prepared using the methods of PCT Application Publication No. WO2012/156498.

### Example 120

### 2-amino-N,N-dipropyl-8-(4-(pyrrolidine-1-carbonyl)phenyl)-3H-benzo[b]azepine-4-carboxamide

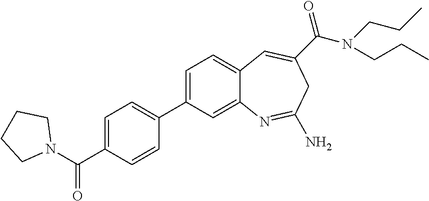 [SMILES: CCCN(CCC)C(=O)C1=Cc2ccc(-c3ccc(C(=O)N4CCCC4)cc3)cc2N=C(N)C1]

The compound of Example 120 can be prepared using the methods of U.S. Patent Application Publication No. 2008/0234251.

A pharmaceutically effective amount of a TLR8 modulating compound described herein, or a pharmaceutically acceptable salt thereof, includes individual doses of from about 0.1 mg to about 1000 mg, from about 1 mg to 500 mg, from about 1 mg to about 250 mg, and from about 1 mg to about 150mg, which may be delivered daily in one dose or in divided doses, such as once a month, once every two weeks, once a week (7 days),once a day, twice a day, three times a day, or four times a day. In certain embodiments, the individual dose is about 10 mg, 20 mg, 30 mg, 40 mg, 50 mg, 60 mg 70 mg, 80 mg, 90 mg, 100 mg, 110 mg, 120 mg, 130 mg, 140 mg, or 150 mg. Each of the methods of treatment, pharmaceutical combinations, and pharmaceutical compositions or formulations herein comprises further embodiments in which the pharmaceutically effective amount of the TLR8 modulating compound, including those of each of the formulas and specific examples herein, comprises in each separate embodiment one of the individual doses ranges listed in the prior sentences. Each of the methods of treatment, pharmaceutical combinations, and pharmaceutical compositions or formulations herein comprises further embodiments in which the pharmaceutically effective amount of the TLR8 modulating compound, including those of each of formulas and specific examples herein, comprises in each separate embodiment one of the individual doses listed in the prior sentences.

### Pharmaceutical Compositions

In one embodiment, the present application discloses pharmaceutical compositions comprising a TLR8 modulating compound as described herein, including a compound selected from the group of the compounds of Formula (J), (I), (II), (IIa), (IIb), (III), (IIIa), (IIIb), (IV), (IVa), (IVb), (IVc), or (IVd) and each of the individual compounds of the examples from Example 1 through Example 120, or a pharmaceutically acceptable salt thereof, in combination with at least one additional therapeutic agent selected from the group consisting of HIV protease inhibiting compounds, HIV non-nucleoside inhibitors of reverse transcriptase, HIV nucleoside or nucleotide inhibitors of reverse transcriptase, HIV nucleotide inhibitors of reverse transcriptase, HIV integrase strand transfer inhibitors, non-catalytic site integrase inhibitors, HIV gp120/41 inhibitors, CCR5 inhibitors, HIV capsid inhibitors, HIV Vif inhibitors, and combinations thereof, and a pharmaceutically acceptable excipient. Examples include nucleoside-sparing and nucleotide-sparing combinations.

In another embodiment, the present application provides pharmaceutical compositions comprising pharmaceutically effective amounts of a TLR8 modulating compound as described herein, or a pharmaceutically acceptable salt thereof, in combination with at least one additional therapeutic agent selected from the group consisting of amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, R00334649, KNI-272, DPC-681, DPC-684, GW640385X, DG17, PPL-100, DG35, AG 1859, capravirine, emivirine, delaviridine, efavirenz, nevirapine, (+) calanolide A, etravirine, doravirine (MK-1439), GW5634, DPC-083, DPC-961, DPC-963, MIV-150, TMC-120, TMC-278 (rilpivirine), BILR 355 BS, VRX 840773, UK-453061, RDEA806, zidovudine, emtricitabine, didanosine, stavudine, zalcitabine, lamivudine, abacavir, amdoxovir, elvucitabine, alovudine, MIV-210, Racivir (±-FTC), D-d4FC, phosphazide, fozivudine tidoxil, apricitibine AVX754, amdoxovir, KP-1461, and fosalvudine tidoxil (formerly HDP 99.0003),), tenofovir, tenofovir disoproxil fumarate, tenofovir alafenamide, CMX-157, adefovir dipivoxil, GS-9131, (2R,5S,13aR)-N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (2S,5R,13aS)-N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (1S,4R,12aR)-N-(2,4-difluorobenzyl)-7-hydroxy-6,8-dioxo-1,2,3,4,6,8,12,12a-octahydro-1,4-methanodipyrido[1,2-a:1′,Z-d]pyrazine-9-carboxamide, (1R,4S,12aR)-7-hydroxy-6,8-dioxo-N-(2,4,6-trifluorobenzyl)-1,2,3,4,6,8,12,12a-octahydro-1,4-methanodipyrido[1,2-a:1′,2′-d]pyrazine-9-carboxamide, (2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide, (1R,4S,12aR)-N-(2,4-difluorobenzyI)-7-hydroxy-6,8-dioxo-1,2,3,4,6,8,12,12a-octahydro-1,4-methanodipyrido[1,2-a:1′,Z-d]pyrazine-9-carboxamide, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, L-870810, MK-0518 (raltegravir), dolutegravir, elvitegravir, GSK1265744, BMS-538158, GSK364735C, BMS-707035, MK-2048, BA 011, enfuvirtide, sifuvirtide, FB006M, TRI-1144, AMD-070, SPO1A, BMS-488043, BMS-626529, BMS-663068, BlockAide/CR, immunitin, benzimidazole derivatives, benzo-1,2,4-thiadiazine derivatives, phenylalanine derivatives, aplaviroc, vicriviroc, and maraviroc, cenicriviroc (TBR-652), cyclosporine, FK-506, rapamycin, taxol, taxotere, clarithromycin, A-77003, A-80987, MK-639, saquinavir, VX-478, AG1343, DMP-323, XM-450, BILA 2011 BS, BILA 1096 BS, BILA 2185 BS, BMS 186,318, LB71262, SC-52151, SC-629 (N,N-dimethylglycyl-N-(2-hydroxy-3-(((4-methoxyphenyl)sulphonyl)(2-methylpropyl)amino)-1-(phenylmethyl)propyl)-3-methyl-L-valinamide), KNI-272, CGP 53437, CGP 57813 and U-103017 and a pharmaceutically acceptable excipient.

In still another embodiment, the present invention provides pharmaceutical compositions comprising pharmaceutically effective amounts of a TLR8 modulating compound as described herein, or a pharmaceutically acceptable salt thereof, in combination with two, three, four, five, or more additional therapeutic agents. For example, a TLR8 modulating compound as described herein, or a pharmaceutically acceptable salt thereof, is combined with two, three, four, five, or more additional therapeutic agents selected from the classes of HIV protease inhibitors, HIV non-nucleoside inhibitors of reverse transcriptase, HIV nucleoside inhibitors of reverse transcriptase, HIV nucleotide inhibitors of reverse transcriptase, HIV entry inhibitors and HIV integrase inhibitors. The two, three, four, five, or more additional therapeutic agents can be different therapeutic agents selected from the same class of therapeutic agents, or they can be selected from different classes of therapeutic agents. In a particular embodiment, the TLR8 modulating compound as described herein, or a pharmaceutically acceptable salt thereof, is combined with two, three, four, five, or more additional therapeutic agents selected from the classes of HIV protease inhibitors, HIV non-nucleoside inhibitors of reverse transcriptase, HIV nucleoside inhibitors of reverse transcriptase, HIV nucleotide inhibitors of reverse transcriptase, and HIV integrase inhibitors. In a still more particular embodiment, the pharmaceutical composition of the present invention comprises a compound selected from the group of compounds of Examples 1-50, or a pharmaceutically acceptable salt thereof, in combination with two, three, four, five, or more additional therapeutic agents selected from the classes of HIV protease inhibitors, HIV non-nucleoside inhibitors of reverse transcriptase, HIV nucleoside inhibitors of reverse transcriptase, HIV nucleotide inhibitors of reverse transcriptase, and HIV integrase inhibitors. For example, such combinations can comprise a compound selected from the group of compounds of Examples 1-50, or a pharmaceutically acceptable salt thereof in combination with two, three, four, five, or more additional therapeutic agents selected from the group consisting of tenofovir disoproxil fumarate (TDF), tenofovir alafenamide (TAF), tenofovir alafenamide hemifumarate, abacavir, abacavir sulfate, GS-9131, emtricitabine, lamuvidine, elvitegravir, efavirenz, atazanavir, (2R,5S,13aR)-N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (2S,5R,13aS)-N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (1S,4R,12aR)-N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide, (1R,4S,12aR)-7-hydroxy-6,8-dioxo-N-(2,4,6-trifluorobenzyl)-1,2,3,4,6,8,12,12a-octahydro-1,4-methanodipyrido[1,2-a:1′,2′-d]pyrazine-9-carboxamide, (2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide, (1R,4S,12aR)-N-(2,4-difluorobenzyl)-7-hydroxy-6,8-dioxo-1,2,3,4,6,8,12,12a-octahydro-1,4-methanodipyrido[1,2-a:1′,Z-d]pyrazine-9-carboxamide, darunavir, raltegravir, dolutegravir, GSK774, cobicistat, ritonavir, and rilpivirine (or pharmaceutically acceptable salts, solvates, and/or esters thereof).

Combinations and compositions herein include those comprising pharmaceutically effect amounts of TDF and emtricitabine, plus a third HIV therapeutic agent, as well TAF and emtricitabine, plus a third HIV therapeutic agent. Examples of HIV therapeutic agents that may be used with these combinations include HIV protease inhibitors (PIs), non-nucleoside reverse transcriptase inhibitors (NNRTIs), nucleoside reverse transcriptase inhibitors (NRTIs), Integrase Strand Transfer inhibitors (INSTIs), non-catalytic site integrase inhibitors (NCINIs), Capsid inhibitors, etc., listed herein.

Specific embodiments of ternary combinations which a) may be combined with a pharmaceutically acceptable excipient to prepare a pharmaceutical composition, or b) may be used in combination in each of the methods described herein, comprise, for example, pharmaceutically effective amounts of each of the compounds listed in the combinations below, or a pharmaceutically acceptable salt thereof:

Examples of antiviral agents that may be combined in the pharmaceutical compositions and regimens used in the methods described herein include TDF, TAF, emtricitabine (FTC), lamivudine (3TC), abacavir (ABC), zidovudine (AZT), efavirenz (EFV), rilpivirine (RPV), etravirine (ETV), atazanavir (ATV), atazanavir+ritonavir (ATV/r), atazanavir+cobicistat (ATV/COBI), darunavir (DRV), darunavir+ritonavir (DRV/r), darunavir+cobicistat (DRV/COBI), lopinavir (LPV), lopinavir+ritonavir (LPV/r), lopinavir+cobicistat (LPV/COBI), dolutegravir (DTG), raltegravir (RAL), elvitegravir (EVG), elvitegravir+ritonavir (EVG/r), elvitegravir+cobicistat (EVG/COBI), and maraviroc. As such, provided are separate combinations, each comprising a pharmaceutically effective amount of a TLR8 modulator, including those of each of the formulas and specific examples herein, or a pharmaceutically acceptable salt thereof, in combination with a pharmaceutically effective amount of each agent in the separate antiviral combinations of TDF/TAF, TDF/FTC, TDF/3TC, TDF/ABC, TDF/AZT, TDF/EFV, TDF/RPV, TDF/ETV, TDF/ATV, TDFATV/r, TDF/ATV/COBI, TDF/DRV, TDF/DRV/r. TDF/DRV/COBI, TDF/LPV, TDF/LPV/r, TDF/LPV/COBI, TDF/DTG, TDF/RAL, TDF/EVG, TDF/EVG/r, TDF/EVG/COBI, TDF/maraviroc, TAF/FTC, TAF/3TC, TAF/ABC, TAF/AZT, TAF/EFV, TAF/RPV, TAF/ETV, TAF/ATV,TAF/ATV/r, TAF/ATV/COBI, TAF/DRV, TAF/DRV/r, TAF/DRV/COBI, TAF/LPV, TAF/LPV/r, TAF/LPV/COBI, TAF/DTG, TAF/RAL, TAF/EVG, TAF/EVG/r, TAF/EVG/COBI, TAF/maraviroc, FTC/3TC, FTC/ABC, FTC/AZT, FTC/EFV, FTC/RPV, FTC/ETV, FTC/ATV, FTC/ATV/r, FTC/ATV/COBI, FTC/DRV, FTC/DRV/r, FTC/DRV/COBI, FTC/LPV, FTC/LPV/r, FTC/LPV/COBI, FTC/DTG, FTC/RAL, FTC/EVG, FTC/EVG/r, FTC/EVG/COBI, FTC/maraviroc, 3TC/ABC, 3TC/AZT, 3TC/EFV, 3TC/RPV, 3TC/ETV, 3TC/ATV, 3TC/ATV/r, 3TC/ATV/COBI, 3TC/DRV, 3TC/DRV/r, 3TC/DRV/COBI, 3TC/LPV, 3TC/LPV/r, 3TC/LPV/COBI, 3TC/DTG, 3TC/RAL, 3TC/EVG, 3TC/EVG/r, 3TC/EVG/COBI, 3TC/maraviroc, ABC/AZT, ABC/EFV, ABC/RPV, ABC/ETV, ABC/ATV, ABC/ATV/r, ABC/ATV/COBI, ABC/DRV, ABC/DRV/r, ABC/DRV/COBI, ABC/LPV, ABC/LPV/r, ABC/LPV/COBI, ABC/DTG, ABC/RAL, ABC/EVG, ABC/EVG/r, ABC/EVG/COBI, ABC/maraviroc, AZT/EFV, AZT/RPV, AZT/ETV, AZT/ATV, AZT/ATV/r, AZT/ATV/COBI, AZT/DRV, AZT/DRV/r, AZT/DRV/COBI, AZT/LPV, AZT/LPV/r, AZT/LPV/COBI, AZT/DTG, AZT/RAL, AZT/EVG, AZT/EVG/r, AZT/EVG/COBI, AZT/maraviroc, EFV/RPV, EFV/ETV, EFV/ATV, EFV/ATV/r, EFV/ATV/COBI, EFV/DRV, EFV/DRV/r, EFV/DRV/COBI, EFV/LPV,EFV/LPV/r, EFV/LPV/COBI, EFV/DTG, EFV/RAL, EFV/EVG,EFV/EVG/r, EFV/EVG/COBI, EFV/maraviroc, RPV/ETV, RPV/ATV, RPV/ATV/r, RPV/ATV/COBI, RPV/DRV, RPV/DRV/r, RPV/DRV/COBI, RPV/LPV, RPV/LPV/r, RPV/LPV/COBI, RPV/DTG, RPV/RAL, RPV/EVG, RPV/EVG/r, RPV/EVG/COBI, RPV/maraviroc, ETV/ATV, ETV/ATV/r, ETV/ATV/COBI, ETV/DRV, ETV/DRV/r, ETV/DRV/COBI, ETV/LPV, ETV/LPV/r, ETV/LPV/COBI, ETV/DTG, ETV/RAL, ETV/EVG, ETV/EVG/r, ETV/EVG/COBI, ETV/maraviroc, ATV/r, ATV/COBI, ATV/DRV, ATV/DRV/r, ATV/DRV/COBI, ATV/LPV, ATV/LPV/r, ATV/LPV/COBI, ATV/DTG, ATV/RAL, ATV/EVG, ATV/EVG/r, ATV/EVG/COBI, ATV/maraviroc, ATV/r/COBI, ATV/rDRV, ATV/rDRV/COBI, ATV/r/LPV, ATV/r/LPV, ATV/r/LPV/COBI, ATV/r/DTG, ATV/r/RAL, ATV/r/EVG, ATV/r/EVG, ATV/r/EVG/COBI, ATV/r/maraviroc, ATV/COBI/DRV, ATV/COB/IDRV/r, ATV/COBI/DRV, ATV/COBI/LPV, ATV/COBI/LPV/r, ATV/COBILPV/COBI, ATV/COBI/DTG, ATV/COBI/RAL, ATV/COBI/EVG, ATV/COBI/EVG/r, ATV/COBI/EVG, ATV/COBI/maraviroc, DRV/r, DRV/COBI, DRV/LPV, DRV/LPV/r, DRV/LPV/COBI, DRV/DTG, DRV/RAL, DRV/EVG, DRV/EVG/r, DRV/EVG/COBI, DRV/maraviroc, DRV/r, DRV/COBI, DRV/r/LPV, DRV/r/LPV/COBI, DRV/r/DTG, DRV/r/RAL, DRV/r/EVG, DRV/r/EVG/COBI, DRV/rmaraviroc, DRV/COBI/LPV, DRV/COB/ILPV/r, DRV/COBI/LPV/COBI, DRV/COBI/DTG, DRV/COBI/RAL, DRV/COBI/EVG, DRV/COBI/EVG/r, DRV/COBI/EVG/COBI, DRV/COBI/maraviroc, LPV/r, LPV/COBI, LPV/DTG, LPV/RAL, LPV/EVG, LPV/EVG/r, LPV/EVG/COBI, LPV/maraviroc, LPV/r/LPV/COBI, LPV/r/DTG, LPV/r/RAL, LPV/r/EVG, LPV/r/EVG/COBI, LPV/r/maraviroc, LPV/COBI/DTG, LPV/COBI/RAL, LPV/COBI/EVG, LPV/COBI/EVG/r, LPV/COBI/EVG, LPV/COBI/maraviroc, DTG/RAL, DTG/EVG, DTG/EVG/r, DTG/EVG/COBI, DTG/maraviroc, DTG/ABC/3TC, RAL/EVG, RAL/EVG/r, RAL/EVG/COBI, RAL/maraviroc, EVG/r, EVG/COBI, and EVG/maraviroc. Also provided are separate pharmaceutical compositions, the separate compositions each comprising a pharmaceutically acceptable excipient, a pharmaceutically effective amount of a TLR8 modulator, including in separate embodiments those of each of the formulas and specific examples herein, or a pharmaceutically acceptable salt thereof, and pharmaceutically effective amounts of each agent in the separate antiviral combinations listed in the preceding sentence. It is understood that the combination of an individual antiviral combination and an individual TLR8 modulator, along with a pharmaceutically acceptable excipient, comprises a separate pharmaceutical composition.

Specific embodiments of combinations which a) may be combined with a pharmaceutically acceptable excipient to prepare a pharmaceutical composition, or b) may be used in combination in each of the methods described herein, comprise the separate following examples, wherein “TLR8” refers to a TLR8 modulating compound, including each of those described herein. A specific example within each combination comprises the combination in which “TLR8” represents a compound of Formula (I). Specific examples within each combination comprises the combination in which “TLR8” represents a compound of Examples 1-50. Another specific example within each combination comprises the combination in which “TLR8” represents a compound of Examples 1-48. In each case, reference to a compound is understood to include the compound or a pharmaceutically acceptable salt thereof.

Combinations include TLR8/TDF/emtricitabine; TLR8/TAF/emtricitabine; TLR8/TDF/elvitegravir; TLR8/TAF/elvitegravir; TLR8/TDF/elvitegravir; TLR8/TAF/elvitegravir; TLR8/TDF/efavirenz; TLR8/TAF/efavirenz; TLR8/TDF/atazanavir; TLR8/TAF/atazanavir; TLR8/TDF/darunavir; TLR8/TAF/darunavir; TLR8/TDF/raltegravir; TLR8/TAF/raltegravir; TLR8/TDF/rilpivirine; TLR8/TAF/rilpivirine; TLR8/emtricitabine/elvitegravir; TLR8/emtricitabine/efavirenz; TLR8/emtricitabine/atazanavir; TLR8/emtricitabine/darunavir; TLR8/emtricitabine/raltegravir; TLR8/emtricitabine/rilpivirine; TLR8/elvitegravir/efavirenz; TLR8/elvitegravir/atazanavir; TLR8/elvitegravir/darunavir; TLR8/elvitegravir/raltegravir; TLR8/elvitegravir/rilpivirine; TLR8/efavirenz/atazanavir; TLR8/efavirenz/darunavir; TLR8/efavirenz/raltegravir; TLR8/efavirenz/rilpivirine; TLR8/atazanavir/darunavir; TLR8/atazanavir/raltegravir; TLR8/atazanavir/rilpivirine; TLR8/darunavir/raltegravir; TLR8/darunavir/rilpivirine; TLR8/raltegravir/rilpivirine; TLR8/darunavir/ritonavir; TLR8/GSK1265744/rilpivirine; and TLR8/abacavir/lamivudine.

Specific embodiments of quaternary combinations which a) may be combined with a pharmaceutically acceptable excipient to prepare a pharmaceutical composition, or b) may be used in combination in each of the methods described herein, comprise, for example: TLR8/TDF/emtricitabine/dolutegravir; TLR8/TAF/emtricitabine/dolutegravir; TLR8/TDF/emtricitabine/(2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide; TLR8/TAF/emtricitabine/2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide; TLR8/TDF/emtricitabine/elvitegravir; TLR8/TAF/emtricitabine/elvitegravir; TLR8/TDF/emtricitabine/efavirenz; TLR8/TAF/emtricitabine/efavirenz; TLR8/TDF/emtricitabine/atazanavir; TLR8/TAF/emtricitabine/atazanavir; TLR8/TDF/emtricitabine/darunavir; TLR8/TAF/emtricitabine/darunavir; TLR8/TDF/emtricitabine/raltegravir; TLR8/TAF/emtricitabine/raltegravir; TLR8/TDF/emtricitabine/rilpivirine; TLR8/TAF/emtricitabine/rilpivirine; TLR8/TDF/elvitegravir/efavirenz; TLR8/TAF/elvitegravir/efavirenz; TLR8/TDF/elvitegravir/atazanavir; TLR8/TAF/elvitegravir/atazanavir; TLR8/TDF/elvitegravir/darunavir; TLR8/TAF/elvitegravir/darunavir; TLR8/TDF/elvitegravir/raltegravir; TLR8/TAF/elvitegravir/raltegravir; TLR8/TDF/elvitegravir/rilpivirine; TLR8/TAF/elvitegravir/rilpivirine; TLR8/TDF/efavirenz/atazanavir; TLR8/TAF/efavirenz/atazanavir; TLR8/TDF/efavirenz/darunavir; TLR8/TAF/efavirenz/darunavir; TLR8/TDF/efavirenz/raltegravir; TLR8/TAF/efavirenz/raltegravir; TLR8/TDF/efavirenz/rilpivirine; TLR8/TAF/efavirenz/rilpivirine; TLR8/TDF/atazanavir/darunavir; TLR8/TAF/atazanavir/darunavir; TLR8/TDF/atazanavir/raltegravir; TLR8/TAF/atazanavir/raltegravir; TLR8/TDF/atazanavir/rilpivirine; TLR8/TAF/atazanavir/rilpivirine;TLR8/TDF/darunavir/raltegravir; TLR8/TAF/darunavir/raltegravir; TLR8/TDF/darunavir/rilpivirine; TLR8/TAF/darunavir/rilpivirine; TLR8/emtricitabine/elvitegravir/efavirenz; TLR8/emtricitabine/elvitegravir/atazanavir; TLR8/emtricitabine/elvitegravir/darunavir; TLR8/emtricitabine/elvitegravir/raltegravir; TLR8/emtricitabine/elvitegravir/rilpivirine; TLR8/emtricitabine/efavirenz/atazanavir; TLR8/emtricitabine/efavirenz/darunavir; TLR8/emtricitabine/efavirenz/raltegravir; TLR8/emtricitabine/efavirenz/rilpivirine; TLR8/emtricitabine/atazanavir/darunavir; TLR8/emtricitabine/atazanavir/raltegravir; TLR8/emtricitabine/atazanavir/rilpivirine; TLR8/emtricitabine/darunavir/raltegravir; TLR8I/emtricitabine/darunavir/rilpivirine; TLR8/emtricitabine/raltegravir/rilpivirine; TLR8/elvitegravir/efavirenz/atazanavir; TLR8/elvitegravir/efavirenz/darunavir; TLR8/elvitegravidefavirenz/raltegravir; TLR8/elvitegravir/efavirenz/rilpivirine; TLR8/elvitegravir/atazanavir/darunavir; TLR8/elvitegravir/atazanavir/raltegravir; TLR8/elvitegravir/raltegravir/rilpivirine; TLR8/efavirenz/atazanavir/darunavir; TLR8/efavirenz/atazanavir/raltegravir; TLR8/efavirenz/atazanavir/rilpivirine; TLR8/efavirenz/darunavir/raltegravir; TLR8/efavirenz/darunavir/rilpivirine; TLR8/efavirenz/raltegravir/rilpivirine; TLR8/atazanavir/darunavir/raltegravir; TLR8/atazanavir/darunavir/rilpivirine; TLR8/darunavir/raltegravir/rilpivirine; TLR8/dolutegravir/abacavir/lamivudine; TLR8/(2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide/abacavir/lamivudine; TLR8/raltegravir/darunavir; TLR8/raltegravir/ritonavir/darunavir; TLR8/raltegravir/cobicistat/darunavir; TLR8/raltegravir/atazanavir; TLR8/raltegravir/atazanavir/maraviroc; TLR8/raltegravir/maraviroc/etravirine; TLR8/raltegravir/maraviroc/rilpivirine; TLR8/maraviroc/darunavir/ritonavir; TLR8/maraviroc/darunavir/cobicistat; TLR8/raltegravir/darunavir/ritonavir/maraviroc; TLR8/raltegravir/darunavir/cobicistat/maraviroc; TLR8/raltegravir/darunavir/ritonavir/etravirine; TLR8/raltegravir/darunavir/cobicistat/etravirine; TLR8/atazanavir/ritonavir/efavirenz; TLR8/atazanavir/cobicistat/efavirenz; TLR8/raltegravir/etravirine; TLR8/ritonavir/lopinavir/raltegravir; TLR8/cobicistat/lopinavir/raltegravir; TLR8/ritonavir/darunavir/etravirine; TLR8/cobicistat/darunavir/etravirine; TLR8/ritonavir/lopinavir; and TLR8/ritonavir/lopinavir/maraviroc.

Additional specific embodiments comprise the combination of a) a pharmaceutically effective amount of a TLR8 modulating compound, including those of each of the formulas and specific examples herein, b) a pharmaceutically acceptable excipient, and c) a combination of five or more antiviral agents. These combinations may be used to prepare a pharmaceutical composition and/or may be used in combination in each of the methods described herein. Such combinations comprise, for example, a pharmaceutically effective amount of a TLR8 modulating compound, including those of each of the formulas and specific examples herein, including individual embodiments in each combination in which the TLR8 modulating compound is, respectively, a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, and the antiviral agents in each individual group of:
TDF/emtricitabine/atazanavir/ritonavir/maraviroc/raltegravir;
TAF/emtricitabine/atazanavir/ritonavir/maraviroc/raltegravir;
TDF/emtricitabine/atazanavir/cobicistat/maraviroc/raltegravir;
TAF/emtricitabine/atazanavir/cobicistat/maraviroc/raltegravir;
TDF/emtricitabine/atazanavir/ritonavir/maraviroc/dolutegravir;
TAF/emtricitabine/atazanavir/ritonavir/maraviroc/dolutegravir;
TDF/emtricitabine/atazanavir/cobicistat/maraviroc/dolutegravir;
TAF/emtricitabine/atazanavir/cobicistat/maraviroc/dolutegravir;
TDF/emtricitabine/darunavir/ritonavir/maraviroc/raltegravir;
TAF/emtricitabine/darunavir/ritonavir/maraviroc/raltegravir;
TDF/emtricitabine/darunavir/cobicistat/maraviroc/raltegravir;
TAF/emtricitabine/darunavir/cobicistat/maraviroc/raltegravir;
TDF/emtricitabine/darunavir/ritonavir/maraviroc/dolutegravir;
TAF/emtricitabine/darunavir/ritonavir/maraviroc/dolutegravir;
TDF/emtricitabine/darunavir/cobicistat/maraviroc/dolutegravir;
TAF/emtricitabine/darunavir/cobicistat/maraviroc/dolutegravir;
TDF/emtricitabine/efavirenz/ritonavir/lopinavir/maraviroc;
TAF/emtricitabine/efavirenz/ritonavir/lopinavir/maraviroc;
TDF/emtricitabine/efavirenz/cobicistat/lopinavir/maraviroc;
TAF/emtricitabine/efavirenz/cobicistat/lopinavir/maraviroc;
TDF/emtricitabine/cobicistat/lopinavir/m araviroc/raltegravir;
TAF/emtricitabine/cobicistat/lopinavir/maraviroc/raltegravir;
TDF/emtricitabine/ritonavir/lopinavir/maraviroc/raltegravir;
TAF/emtricitabine/ritonavir/lopinavir/maraviroc/raltegravir;
TDF/emtricitabine/cobicistat/lopinavir/maraviroc/dolutegravir;
TAF/emtricitabine/cobicistat/lopinavir/maraviroc/dolutegravir;
TDF/emtricitabine/ritonavir/lopinavir/maraviroc/dolutegravir;
TAF/emtricitabine/ritonavir/lopinavir/maraviroc/dolutegravir;
TDF/emtricitabine/cobicistat/fosamprenavir/maraviroc/raltegravir;
TAF/emtricitabine/cobicistat/fosamprenavir/maraviroc/raltegravir;
TDF/emtricitabine/ritonavir/fosamprenavir/maraviroc/raltegravir;
TAF/emtricitabine/ritonavir/fosamprenavir/maraviroc/raltegravir;
TDF/emtricitabine/cobicistat/fosamprenavir/maraviroc/dolutegravir;
TAF/emtricitabine/cobicistat/fosamprenavir/maraviroc/dolutegravir;
TDF/emtricitabine/ritonavir/fosamprenavir/maraviroc/dolutegravir; and
TAF/emtricitabine/ritonavir/fosamprenavir/maraviroc/dolutegravir.

In each of the combinations above, the specific agents in each combination may be administered in any pharmaceutically effective amount known in the art. In specific embodiments the agents are utilized in the combinations that include them in the following individual doses: tenofovir disoproxil fumarate (TDF) from about 250 mg to from about 350 mg/dose; TAF from about 5 mg to about 50 mg, emtricitabine from about 150 mg to about 250 mg/dose; elvitegravir, when administered in combination with a boosting agent such as cobicistat or ritonavir, from about 100 mg to about 200 mg/dose, and unboosted elvitegravir from about 800 mg to about1200 mg; efavirenz from about 500 mg to about 700 mg/dose; atazanavir from about 250 mg to about 350 mg/dose; darunavir from about 700 mg to about 900 mg/dose; raltegravir from about 350 mg to about 450 mg/dose; rilpivirine from about 20 mg to about 30 mg/dose (or from about 22.5 mg to about 32.5 mg/dose as rilpivirine HCL); ritonavir from about 50 mg to about 150 mg/dose; dolutegravir from about 30 mg to about 70 mg/dose, abacavir from about 500 mg to about 700 mg/dose, lamivudine from about 250 mg to about 350 mg/dose, GSK1265744 from about 10 mg to about 50 mg/dose, cobicistat from about 100 mg to 200 mg/dose, atazanavir from about 250 mg to about 350 mg/dose, maraviroc from about 100 mg to about 200 mg/dose, etravirine from about 100 mg to about 300 mg/dose, lopinavir from about 300 mg to about 500 mg/dose, and zidovudine from about 500 mg to about 750 mg/day.

In other specific embodiments the agents are utilized in the combinations that include them in the following individual doses: tenofovir disoproxil fumarate (TDF) from about 275 mg to about 325 mg/dose; TAF from about 5 mg to about 30 mg, emtricitabine from about 175 mg to about 225 mg/dose; elvitegravir from about 125 mg to about 175 mg/dose, when boosted by a boosting agent such as cobicistat or ritonavir; efavirenz from about 550 mg to about 650 mg/dose; atazanavir from about 275 mg to about 325 mg/dose; darunavir from about 750 mg to about 850 mg/dose; raltegravir from about 375 mg to about 425 mg/dose; rilpivirine from about 22 mg to about 28 mg/dose (or from about 24.5 mg to about 30.5 mg/dose as rilpivirine HCL); ritonavir from about 75 mg to about 125 mg/dose; dolutegravir from about 40 mg to about 60 mg/dose, abacavir from about 550 mg to about 650 mg/dose, lamivudine from about 275 mg to about 325 mg/dose, GSK1265744 from about 20 mg to about 40 mg/dose, cobicistat from about 125 mg to 175 mg/dose, atazanavir from about 275 mg to about 325 mg/dose, maraviroc from about 125 mg to about 175 mg/dose, etravirine from about 150 mg to about 250 mg/dose, lopinavir from about 350 mg to about 450 mg/dose, and zidovudine from about 550 mg to about 650 mg/day.

In further specific embodiments the agents are utilized in the combinations that include them in the following individual doses: tenofovir disoproxil fumarate (TDF) at about 300 mg/dose; TAF at about 25 mg/dose or at about 10 mg per dose in the presence of a boosting agent, such as cobicistat or ritonavir, emtricitabine at about 200 mg/dose; elvitegravir at about 150 mg/dose, when boosted by cobicistat or ritonavir; efavirenz at about 600 mg/dose; atazanavir at about 300 mg/dose; darunavir at about 800 mg/dose; raltegravir at about 400 mg/dose; rilpivirine at about 25 mg/dose (or at about 27.5 mg/dose as rilpivirine HCL); ritonavir at about 100 mg/dose; dolutegravir at about 50 mg/dose, abacavir at about 600 mg/dose, lamivudine at about 300 mg/dose, GSK1265744 at about 30 mg/dose, cobicistat at about 150 mg/dose, atazanavir at about 300 mg/dose, maraviroc at about 150 mg/dose, etravirine at about 200 mg/dose, lopinavir at about 400 mg/dose, and zidovudine at about 600 mg/day.

The TLR8 modulating compounds in the combinations above, including those of Formula (J), (I), (II), (IIa), (IIb), (III), (IIIa), (IIIb), (IV), (IVa), (IVb), (IVc), or (IVd), or each of the individual compounds of the examples from Example 1 through Example 120 or a pharmaceutically acceptable salt thereof, may be administered at a therapeutically effective amount as described herein.

It is understood that each of the dose ranges for the TLR8 modulating compounds can be combined in pharmaceutical compositions and pharmaceutical combinations and regiments with each of the doses for the other combination agents discussed above. For instance, the combination listed above as TLR8/TDF/emtricitabine/dolutegravir includes a therapeutically effective amount per dose of TLR8/from about 250 mg-350 mg per dose TDF/from about 150 mg to about 250 mg per dose emtricitabine/from about 30 mg to about 70 mg per dose dolutegravir; a therapeutically effective amount per dose of TLR8/from about 275 mg-325 mg per dose TDF/from about 175 mg to about 225 mg per dose emtricitabine/from about 40 mg to about 60 mg per dose dolutegravir; a therapeutically effective amount per dose of TLR8/300 mg per dose TDF/200 mg per dose emtricitabine/50 mg per dose dolutegravir; a therapeutically effective amount per dose of TLR8/from about 250 mg-350 mg per dose TDF/from about 150 mg to about 250 mg per dose emtricitabine/from about 30 mg to about 70 mg per dose dolutegravir; a therapeutically effective amount per dose of TLR8/from about 275 mg-325 mg per dose TDF/about 175 mg to about 225 mg per dose emtricitabine/from about 40 mg to about 60 mg per dose dolutegravir; a therapeutically effective amount per dose of TLR8/300 mg per dose TDF/200 mg per dose emtricitabine/50 mg per dose dolutegravir; a therapeutically effective amount per dose of TLR8/from about 250 mg-350 mg per dose TDF/from about 150 mg to about 250 mg per dose emtricitabine/from about 30 mg to about 70 mg per dose dolutegravir; a therapeutically effective amount per dose of TLR8/from about 275 mg-325 mg per dose TDF/from about 175 mg to about 225 mg per dose emtricitabine/from about 40 mg to about 60 mg per dose dolutegravir; a therapeutically effective amount per dose of TLR8/300 mg per dose TDF/200 mg per dose emtricitabine/50 mg per dose dolutegravir; a therapeutically effective amount per dose of TLR8/from about 250 mg-350 mg per dose TDF/from about 150 mg to about 250 mg per dose emtricitabine/from about 30 mg to about 70 mg per dose dolutegravir; a therapeutically effective amount per dose of TLR8/from about 275 mg-325 mg per dose TDF/from about 175 mg to about 225 mg per dose emtricitabine/from about 40 mg to about 60 mg per dose dolutegravir; and a therapeutically effective amount per dose of TLR8/300 mg per dose TDF/200 mg per dose emtricitabine/50 mg per dose dolutegravir. It is understood that each of the corresponding dose range combinations are included for each of the combinations of agents listed herein. It is understood that corresponding combinations are intended wherein, for each specific combination in this paragraph corresponding combinations exist wherein TLR8 includes compound of Formula (J), (I), (II), (IIa), (IIb), (III), (IIIa), (IIIb), (IV), (IVa), (IVb), (IVc), or (IVd), or each of the individual compounds of the examples from Example 1 through Example 120. It is also understood that the same breadth of intended combinations applies to each of the formulations listed above combining a TLR8 modulating compound with HIV agents and that embodiments exist wherein each of the combinations is used in the methods herein.

In yet another embodiment, the present application provides a combination pharmaceutical agent comprising:

a) a first pharmaceutical composition comprising a TLR8 modulating compound as described herein, or a pharmaceutically acceptable salt, solvate, or ester thereof; and

b) a second pharmaceutical composition comprising at least one additional therapeutic agent selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, HIV capsid inhibitors, interferons, immunomodulatory cytokines (IL-7, IL-15), ribavirin analogs, NS3 protease inhibitors, alpha-glucosidase 1 inhibitors, hepatoprotectants, nucleotide inhibitors of HCV, nucleoside inhibitors of HCV, non-nucleoside inhibitors of HCV, and other drugs for treating HCV, and combinations thereof. Forms of IL-15 useful in the methods herein include human native and recombinant IL-15, including the heterodimer hetIL-15, recombinant human IL-15 (rhIL15), and IL-15 fusion proteins

Within each of the embodiments herein that include “a pharmaceutically effective amount of a TLR8 modulating compound of or a pharmaceutically acceptable salt thereof”, including methods of treatment, pharmaceutical compositions, regimens, and kits, it is understood that separate further embodiments are contemplated wherein all other components or elements are as defined for the original embodiment and the “TLR8 modulating compound or a pharmaceutically acceptable salt thereof” is, in separate embodiments, a compound selected from each of the group of Formula (I), (II), (IIa), (IIb), (III), (IIIa), (IIIb), (IV), (IVa), (IVb), (IVc), and (IVd), and each of the individual compounds of the examples from Example 1 through Example 120, or a pharmaceutically acceptable salt thereof.

Inducing HIV Gene Expression in a Human Infected with HIV

Provided is a method of inducing HIV gene expression in a human infected with HIV, wherein active HIV gene expression in the human has been suppressed by administration of antiretroviral therapy, the method comprising administering to the human a pharmaceutically a pharmaceutically effective amount of a TLR8 modulating compound, or a pharmaceutically acceptable salt thereof.

Provided is a method of inducing HIV gene expression in a human infected with HIV, wherein active HIV gene expression in the human has been suppressed by administration of combination antiretroviral therapy, the method comprising administering to the human a pharmaceutically effective amount of a compound of Formula II, or a pharmaceutically acceptable salt thereof.

Provided is a method of inducing HIV gene expression in a human infected with HIV, the method comprising administration of combination antiretroviral therapy until active HIV replication is suppressed, followed by administering to the human a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Provided is a method of inducing HIV gene expression in an HIV-infected human undergoing combination antiretroviral therapy, the method comprising administering to the human a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Provided is a method of inducing HIV gene expression in HIV infected cells in a human, the method comprising administering to the human a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Also provided is a method of inducing HIV gene expression in HIV infected cells in a human, the method comprising administering to the human a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Also provided are separate methods of inducing HIV gene expression in HIV infected cells in a human, each of the separate methods comprising administering to the human a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

### Latent Reservoir

Provided is a method of inducing HIV gene expression in a latent HIV reservoir in a human infected with HIV, the method comprising administering to the human infected with HIV a pharmaceutically effective amount of a TLR8 modulating compound, or a pharmaceutically acceptable salt thereof.

Also provided is a method of inducing HIV gene expression in a latent HIV reservoir in a human infected with HIV, the method comprising administering to the human infected with HIV a pharmaceutically effective amount of a compound of Formula (Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Also provided are twenty separate methods of inducing HIV gene expression in HIV infected cells in a latent HIV reservoir in a human infected with HIV, each of the separate methods comprising administering to the human infected with HIV a pharmaceutically effective amount of a compound selected from one Formula (J), (I), (II), (IIa), (IIb), (III), (IIIa), (IIIb), (IV), (IVa), (IVb), (IVc), or (IVd), or each of the individual compounds of the examples from Example 1 through Example 120; or a pharmaceutically acceptable salt thereof. It is understood that one of such methods comprises administering to the human infected with HIV a pharmaceutically effective amount of Formula (J), or a pharmaceutically acceptable salt thereof, another method comprises administering to the human infected with HIV a pharmaceutically effective amount of Formula I, etc.

Also provided are separate methods of inducing HIV gene expression in HIV infected cells in a latent HIV reservoir in a human infected with HIV, each methods comprising administering to the human infected with HIV a pharmaceutically effective amount of a compound selected from one of Examples 1 through 50, or a pharmaceutically acceptable salt thereof.

Further embodiments for each of the methods, combinations, and pharmaceutical compositions described herein further comprise the addition of one or more latency-reversing agents (LRAs), also known as latency-reversing drugs (LRDs), such as: histone deacetylase inhibitors (such as romidepson), including hydroxamic acids (or hydroxamates) such as trichostatin A; cyclic tetrapeptides (such as trapoxin B) and the depsipeptides; benzamides; electrophilic ketones; aliphatic acid compounds such as phenylbutyrate and valproic acid, hydroxamic acids such as vorinostat (suberoylanilide hydroxamic acid—SAHA), belinostat, LAQ824, panobinostat, benzamides (e.g., entinostat (MS-275), C1994,mocetinostat, 4SC-202, abexinostat, ACTR, ACY-1215, AR-42, CG200745, CHR-2845, CHR-3996, CUDC-101, entinostat, GATA, givinostat, kevetrin, mocetinostat , panobinostat, resminostat, romidepsin, runx, SB939, sulforaphane, trichostatin A (TSA), trichostatin B, trichostatin C, trapoxin A, trapoxin B, chlamydocin, sodium salts of butyrate (sodium butyrate), butyric acid, sodium salts of phenylbutyrate, phenylbutyric acid, scriptaid, FR901228, depudecin, oxamflatin, pyroxamide, apicidin B, apicidin C, Helminthsporium carbonum toxin, 2-amino-8-oxo-9, 10-epoxy-decanoyl, 3-(4-aroyl-1H-pyrrol-2-yl)-N-hydroxy-2-propenamide, suberoylanilide hydroxamic acid, FK228 or m-carboxycinnamic acid bis-hydroxamide, ITF2357, MCT-1, MCT-3, NHC-51, and any of the histone deacetylase inhibitor compounds disclosed in Archin MN et al., AIDS 2009; 1799-806, which are incorporated herein by reference;

Akt pathway modulators such as disulfiram (Doyon et al, AIDS 2013 Jan. 14; 27(2):F7-F11);

methylation inhibitors, such as DNMTi, 5-aza-2′deoxycitidine (5-aza-dc), decitabine, DL-ethionine, D-methionine, 5-azacytidine, 5-aza-2′deoxycytidine, 5,6-dihydro-5-azacytidine, 5,6-dihydro-5-aza-2′deoxycytidine, 5-fluorocytidine, 5-fluoro-2′deoxycytidine, and short oligonucleotides containing 5-aza-2′deoxycytosine, 5,6-dihydro-5-aza-2′deoxycytosine, and 5-fiuoro-2′deoxycytosine, procainamide, Zebularine, and (−)-egallocatechin-3-gallate;

protein kinase C (PKC) modulators, such as indolactam, Ingenol and its derivative such as ingenol B, prostratin, bryostatin, rottlerin, isoquinoline sulfonamide H-7 and analogs thereof, 4-aminomethyl-I-[2,3-(di-n-decyloxy)n-propyl]-4-phenylpiperidine, phenothiazine agents, tamoxifen, quercetin, verapamil, adriamycin, polymyxin B, gangliosides, sangivamycin, retinal, staurosporine, aminoacridines, sphingosine and related sphingohpids, DAG lactones, such as HK654, HK434, HK602, and HK204;

cytokines or modulators of cytokines, such as TNF-α, TNF-β, IL-1, IL-6, IL-2, IL-4, IL-6, IL-7, IL-10, IL-15, IL-15SA (IL-15 super agonists, such as ALT-803), hetIL-15, Acrp30, AgRP, amphiregulin, angiopoietin-1, AXL, BDNF, bFGF, BLC, BMP-4, BMP-6, b-NGF, BTC, CCL28, Ck beta 8-1, CNTF, CTACK CTAC, Skinkine, Dtk, EGF, EGF-R, ENA-78, eotaxin, eotaxin-2, MPIF-2, eotaxin-3, MIP-4-alpha, Fas Fas/TNFRSF6/Apo-I/CD95, FGF-4, FGF-6, FGF-7, FGF-9, Flt-3 Ligand fms-like tyrosine kinase-3, FKN or FK, GCP-2, GCSF, GDNF Glial, GITR, GITR, GM-CSF, GRO, GRO-a, HCC-4, hematopoietic growth factor, hepatocyte growth factor, I-309, ICAM-1, ICAM-3, IFN-γ, IGFBP-1, IGFBP-2, IGFBP-3, IGFBP-4, IGFBP-6, IGF-I, IGF-I SR, IL-Ia, IL-Iβ, IL-1, IL-1 R4, ST2, IL-3, IL-4, IL-5, IL-6, IL-8, IL-10, IL-11, IL-12 p40, IL-12p70, IL-13, IL-16, IL-17, IL-21, 1-TAC, alpha chemoattractant, lymphotactin, MCP-1, MCP-2, MCP-3, MCP-4, M-CSF, MDC, MIF, MIG, MIP-1, MIP-Iβ, MIP-Iδ, MIP-3a, MIP-3β, MSP-a, NAP-2, NT-3, NT-4, osteoprotegerin, oncostatin M, PARC, PDGF, P1GF, RANTES, SCF, SDF-1, soluble glycoprotein 130, soluble TNF receptor I, soluble TNF receptor II, TARC, TECK, TGF-beta 1, TGF-beta 3, TIMP-1, TIMP-2, TNF-a, TNF-β, thrombopoietin, TRAIL R3, TRAIL R4, uPAR, VEGF and VEGF-D;

modulators of AV6, HIV-1-reacting protein factor (HRF), Quinolin-8-ol, dactinomycin, aclarubicin, cytarabine, PKC412, englarin A, oxaliplatin, 1-cinnamoyl-3,11-dihydroxymeliacarpin (CDM), nordihydroguaiaretic acid (NDGA), and curcumin (Cur);

proteasome inhibitors, such as bortezomib, carfilzomib, oprozomib, and ixazomib;

histone methyltransferace inhibitors, such as inhibitors of Suv39H1 (e.g. chaetocin), inhibitors of EZH2 (e.g. GSK343, E7438, and GSK-126) and inhibitors ofG9a (e.g. BIX-01249 and UNC-0638);

BRD4 inhibitors, such as JQ1 ([(R,S)-4-(4-Chlorophenyl)-2,3,9-trimethyl-6H-1-thia-5,7,8,9a-tetraaza-cyclopenta[e]azulen-6-yl]-acetic acid tert-butyl ester), GSK525762 (IBET or (S)-2-(6-(4-chlorophenyl)-8-methoxy-1-methyl-4H-benzo[f][1,2,4]triazolo[4,3-a][1,4]diazepin-4-yl)-N-ethylacetamide), OTX015 (HY15743 -(6S)-4-(4-chlorophenyl)-N-(4-hydroxyphenyl)-2,3,9-trimethyl-6H-thieno[3,2-f][1,2,4]triazolo[4,3-a][1,4]diazepine-6-acetamide), CPI-0610, and Ten-010; and

recombinant HIV Tat protein.

### Method of Inducing Transient HIV Viremia

Also provided is a method for inducing transient HIV-1 viremia in a virologically suppressed human infected with HIV-1, the method comprising administering to the human a pharmaceutically effective amount of a TLR8 modulating compound, or a pharmaceutically acceptable salt thereof.

Also provided is a method for inducing transient HIV-1 viremia in a virologically suppressed human infected with HIV-1, wherein the virologically suppressed human infected with HIV is receiving a combination antiretroviral therapy, the method comprising administering to the human a pharmaceutically effective amount of a TLR8 modulating compound, or a pharmaceutically acceptable salt thereof.

Also provided is a method for inducing transient HIV-1 viremia in a virologically suppressed human infected with HIV-1, wherein the virologically suppressed human infected with HIV is receiving a combination antiretroviral therapy and has a plasma HIV-1 RNA concentration of less than 50 copies of HIV-1 RNA per mL, the method comprising administering to the human a pharmaceutically effective amount of a TLR8 modulating compound, or a pharmaceutically acceptable salt thereof, to increase the plasma HIV-1 RNA concentration in the human to a concentration of greater than 50 copies of HIV-1 RNA per mL. A further embodiment comprises this method in which the plasma HIV-1 RNA concentration in the human is raised to a concentration of from 50 copies of HIV-1 RNA per mL to at least 500 copies of HIV-1 RNA per mL. A further embodiment comprises this method in which the plasma HIV-1 RNA concentration in the human is raised to a concentration of from 50 copies of HIV-1 RNA per mL to at least 1,000 copies of HIV-1 RNA per mL. A further embodiment comprises this method in which the plasma HIV-1 RNA concentration in the human is raised to a concentration of from 50 copies of HIV-1 RNA per mL to at least 2,000 copies of HIV-1 RNA per mL.

Also provided is a method for inducing transient HIV-1 viremia in a virologically suppressed human infected with HIV-1, wherein the virologically suppressed human infected with HIV is receiving a combination antiretroviral therapy and has maintained a plasma HIV-1 RNA concentration of less than 50 copies of HIV-1 RNA per mL for a period of at least three months, the method comprising administering to the human a pharmaceutically effective amount of a TLR8 modulating compound, or a pharmaceutically acceptable salt thereof, to increase the plasma HIV-1 RNA concentration in the human to a concentration of greater than 50 copies of HIV-1 RNA per mL. A further embodiment comprises this method in which the plasma HIV-1 RNA concentration in the human is raised to a concentration of from 50 copies of HIV-1 RNA per mL or below to at least 500 copies of HIV-1 RNA per mL. A further embodiment comprises this method in which the plasma HIV-1 RNA concentration in the human is raised to a concentration of from 50 copies of HIV-1 RNA per mL or below to at least 1,000 copies of HIV-1 RNA per mL. A further embodiment comprises this method in which the plasma HIV-1 RNA concentration in the human is raised to a concentration of from 50 copies of HIV-1 RNA per mL or below to at least 2,000 copies of HIV-1 RNA per mL.

Also provided is a method for inducing transient HIV-1 viremia in a virologically suppressed human infected with HIV-1, wherein the virologically suppressed human infected with HIV is receiving a combination antiretroviral therapy and has maintained a plasma HIV-1 RNA concentration of less than 50 copies of HIV-1 RNA per mL for a period of at least six months, the method comprising administering to the human a pharmaceutically effective amount of a TLR8 modulating compound, or a pharmaceutically acceptable salt thereof, to increase the plasma HIV-1 RNA concentration in the human to a concentration of greater than 50 copies of HIV-1 RNA per mL. A further embodiment comprises this method in which the plasma HIV-1 RNA concentration in the human is raised to a concentration of from 50 copies of HIV-1 RNA per mL to at least 1,000 copies of HIV-1 RNA per mL. A further embodiment comprises this method in which the plasma HIV-1 RNA concentration in the human is raised to a concentration of from 50 copies of HIV-1 RNA per mL to at least 2,000 copies of HIV-1 RNA per mL. Additional separate embodiments exist for these methods in which the viral concentration of 50 copies of HIV-1 RNA per mL or less has been maintained in the human infected with HIV-1 for at least a) one month, b) two months, c) three months, d) four months, e) five months, f) six months, g) seven months, h) eight months, i) nine months, j) ten months, k) eleven months, and I) twelve months. Additional separate embodiments exist for these methods in which the viral concentration of 50 copies of HIV-1 RNA per mL or less has been maintained in the human infected with HIV-1 for a period of a) from about one month to about three months, b) from about two months to about three months, c) from about three months to about six months, d) from about six months to about 9 months, e) from about six months to about one year, f) from about nine months to about one year, g) from about ten months to about one year, h) from about one year to about one year and three months, i) from about one year to about one year and six months, j) from about one year to about one year and nine months, and k) from about one year to about two years.

Within methods for inducing transient HIV-1 viremia in a virologically suppressed human infected with H1V-1above there are separate additional embodiments wherein the TLR8 modulating compound comprises a pharmaceutically effective amount of a TLR8 modulating compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Separate further embodiments within each of the methods for inducing transient HIV-1 viremia in a virologically suppressed human infected with HIV-lcomprise the method wherein the combination antiretroviral therapy is selected from each of the combinations of antiretroviral agents listed herein, wherein the use of each separate combination of antiretroviral agents comprises a separate embodiment.

As one example, provided is a method for inducing transient HIV-1 viremia in a virologically suppressed human infected with HIV-1, wherein the virologically suppressed human infected with HIV is receiving a combination antiretroviral therapy, wherein the combination antiretroviral therapy comprises a pharmaceutically effective amount of TDF, a pharmaceutically effective amount of emtricitabine, and a pharmaceutically effective amount of dolutegravir, the method comprising administering to the human a pharmaceutically effective amount of a TLR8 modulating compound, or a pharmaceutically acceptable salt thereof.

Additional separate methods comprise those above in which the combination antiretroviral therapy comprises individually the separate combinations listed in each of Tables A, B, C, D, E, F, G, H, I, and J is administered to the human infected with HIV-1 in combination with a pharmaceutically effective amount of a TLR8 modulator. In certain embodiments, the TLR modulator is a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120,

Also provided is a method for inducing transient HIV-1 viremia in a virologically suppressed human infected with HIV-1, wherein the virologically suppressed human infected with HIV is receiving a combination antiretroviral therapy, wherein the combination antiretroviral therapy comprises a pharmaceutically effective amount of TDF or TAF, a pharmaceutically effective amount of elvitegravir, a pharmaceutically effective amount of cobicistat, and a pharmaceutically effective amount of emtricitabine, the method comprising administering to the human a pharmaceutically effective amount a TLR8 modulating compound, or a pharmaceutically acceptable salt thereof.

### Enhancing Gene Expression in a Human/Latent Reservoir

Provided is a method of enhancing HIV gene expression in HIV infected cells in a human infected with HIV, the method comprising administering to the human infected with HIV a pharmaceutically effective amount of a TLR8 modulating compound, or a pharmaceutically acceptable salt thereof.

Also provided is a method of enhancing HIV gene expression in HIV infected cells in a human infected with HIV, the method comprising administering to the human infected with HIV a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Also provided is a method of enhancing HIV gene expression in HIV infected cells in a latent HIV reservoir in a human infected with HIV, the method comprising administering to the human infected with HIV a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Also provided is a method of enhancing HIV gene expression in HIV infected T-cells in a human infected with HIV, the method comprising administering to the human infected with HIV a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120or a pharmaceutically acceptable salt thereof.

Also provided are separate methods of a) enhancing HIV gene expression in HIV infected cells in a human infected with HIV; b) enhancing HIV gene expression in HIV infected cells in a latent HIV reservoir in a human infected with HIV; and c) enhancing HIV gene expression in HIV infected T-cells in a human infected with HIV, each of the methods individually comprising administering to the human infected with HIV a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120,or a pharmaceutically acceptable salt thereof. It is understood that one of such methods comprises administering to the human infected with HIV a pharmaceutically effective amount of Formula I, or a pharmaceutically acceptable salt thereof, another method comprises administering to the human infected with HIV a pharmaceutically effective amount of Formula 1(a), etc.

Also provided are separate methods of enhancing HIV gene expression in HIV infected cells in a human infected with HIV, each of the separate methods comprising administering to the human infected with HIV a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Also provided is a method of enhancing HIV gene expression in HIV infected cells in a latent HIV reservoir in a human infected with HIV, the method comprising administering to the human infected with HIV a pharmaceutically effective amount of a TLR8 modulating compound, or a pharmaceutically acceptable salt thereof.

Also provided is a method of enhancing HIV gene expression in HIV infected cells in a latent HIV reservoir in a human infected with HIV, the method comprising administering to the human infected with HIV a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

Also provided is a method of enhancing HIV gene expression in HIV infected cells in a latent HIV reservoir in a human infected with HIV, the method comprising administering to the human infected with HIV a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120,or a pharmaceutically acceptable salt thereof. It is understood that one of such methods comprises administering to the human infected with HIV a pharmaceutically effective amount of Formula (J), or a pharmaceutically acceptable salt thereof, another method comprises administering to the human infected with HIV a pharmaceutically effective amount of Formula (I), etc.

Also provided are separate methods of enhancing HIV gene expression in HIV infected cells in a latent HIV reservoir in a human infected with HIV, each of the separate methods comprising administering to the human infected with HIV a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof.

### Method of Treatment of HIV Infections

Provided is a method of treating an HIV infection in a human, the method comprising administering to the human a pharmaceutically effective amount of a TLR8 modulating compound, or a pharmaceutically acceptable salt thereof.

Also provided is a method of treating an HIV infection in a human, the method comprising administering to a human in need thereof a pharmaceutically effective amount of a TLR8 modulating compound, or a pharmaceutically acceptable salt thereof, sufficient to lower the level of HIV detected in the human's blood or plasma from a first level to a second level, the second level comprising a lower concentration of HIV in the human's blood or plasma than the concentration of HIV in the human's blood or plasma in the first level.

Within each of the methods of treating an HIV infection in a human herein comprising administering to a human in need thereof a pharmaceutically effective amount of a TLR8 modulating compound, or a pharmaceutically acceptable salt thereof, there is a further embodiment comprising the method wherein the second level of concentration of HIV in the human's blood or plasma comprises a viral load (VL) in plasma of less than 50 copies of HIV RNA/ml. Additional separate embodiments within each of the methods comprises the method described wherein the level of HIV in the human's blood or plasma in the second level comprises a viral load (VL) in plasma of a) less than 40 copies of HIV RNA/ml; b) less than 30 copies of HIV RNA/ml; c) less than 20 copies of HIV RNA/ml; d) less than 10 copies of HIV RNA/ml; e) less than 5 copies of HIV RNA/ml; f) less than 3 copies of HIV RNA/ml; less than 1 copy of HIV RNA/ml; and less than 0.5 copies of HIV RNA/ml.

Within each of the methods of treating an HIV infection above there are separate additional embodiments wherein the TLR8 modulating compound comprises a pharmaceutically effective amount of a TLR8 modulating compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120; or a pharmaceutically acceptable salt thereof.

### HIV Treatment Combining Antiretroviral Agents and a TLR8 Modulator

Provided is a method of treatment of an HIV-1 infection in a human, comprising administering to the human a pharmaceutically effective amount of a TLR8 modulating compound or a pharmaceutically acceptable salt thereof and a combination antiretroviral therapy. In certain embodiments, the TLR8 modulating compound or a pharmaceutically acceptable salt thereof is administered prior to administration of the combination antiretroviral therapy.

Within the method of treatment of an HIV-1 infection in a human above there are separate additional embodiments wherein the TLR8 modulating compound comprises a pharmaceutically effective amount of a TLR8 modulating compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120; or a pharmaceutically acceptable salt thereof.

Within the method of treatment of an HIV-1 infection in a human above are further embodiments wherein the combination antiretroviral therapy is selected from each of the combinations of antiretroviral agents listed herein, wherein the use of each separate combination of antiretroviral agents comprises a separate embodiment. Provided is a method of treatment of an HIV-1 infection in a virologically suppressed human infected with HIV-1, wherein the virologically suppressed human infected with HIV is receiving a combination antiretroviral therapy, the method comprising administering to the human a pharmaceutically effective amount of a TLR8 modulating compound, or a pharmaceutically acceptable salt thereof.

Within the method of treatment of an HIV-1 infection in a virologically suppressed human infected with HIV-1 above there are separate additional embodiments wherein the TLR8 modulating compound comprises a pharmaceutically effective amount of a TLR8 modulating compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120; or a pharmaceutically acceptable salt thereof.

Separate further embodiments within each of the methods above for treatment of an HIV-1 infection in a virologically suppressed human infected with HIV-1 comprise the method wherein the combination antiretroviral therapy is selected from each of the combinations of antiretroviral agents listed herein, wherein the use of each separate combination of antiretroviral agents comprises a separate embodiment.

As one example, provided is a method of treatment of an HIV-1 infection in a virologically suppressed human infected with HIV-1, wherein the virologically suppressed human infected with HIV is receiving a combination antiretroviral therapy, wherein the combination antiretroviral therapy comprises a pharmaceutically effective amount of TDF, a pharmaceutically effective amount of emtricitabine, and a pharmaceutically effective amount of dolutegravir, the method comprising administering to the human a pharmaceutically effective amount of the TLR8 modulating compound, or a pharmaceutically acceptable salt thereof.

Additional separate methods comprise those above in which the combination antiretroviral therapy comprises individually the separate combinations listed in in each of Tables A, B, C, D, E, F, G, H, I, and J is administered to the human infected with HIV-1 in combination with a pharmaceutically effective amount of a TLR8 modulating compound.

Also provided is a method of treatment of an HIV-1 infection in a virologically suppressed human infected with HIV-1, wherein the virologically suppressed human infected with HIV is receiving a combination antiretroviral therapy, wherein the combination antiretroviral therapy comprises a pharmaceutically effective amount of TDF or TAF, a pharmaceutically effective amount of elvitegravir, a pharmaceutically effective amount of cobicistat, and a pharmaceutically effective amount of emtricitabine, the method comprising administering to the human a pharmaceutically effective amount of the TLR8 modulating compound, or a pharmaceutically acceptable salt thereof.

Additional separate methods comprise those above in which the combination antiretroviral therapy comprises individually the separate combinations listed in Combination Antiretroviral Tables A, B, C, D, E, F, G, H, I, and J is administered to the human infected with HIV-1 in combination with a pharmaceutically effective amount of a TLR8 modulating compound.

Also provided is a method of treating an HIV infection in a human, the method comprising:
a) administering to a human in need thereof a pharmaceutically effective amount of an antiretroviral agent sufficient to lower the level of HIV detected in the human's blood or plasma from a first level to a second level, the second level comprising a lower concentration of HIV in the human's blood or plasma than the concentration of HIV in the human's blood or plasma in the first level; and
b) administering to the human a pharmaceutically effective amount of a TLR8 modulating compound, or a pharmaceutically acceptable salt thereof.

Also provided is a method of treating an HIV infection in a human, the method comprising:
a) administering to a human in need thereof a pharmaceutically effective amount of a combination antiretroviral therapy regimen sufficient to lower the level of HIV detected in the human's blood or plasma from a first level to a second level, the second level comprising a lower concentration of HIV in the human's blood or plasma than the concentration of HIV in the human's blood or plasma in the first level; and
b) administering to the human a pharmaceutically effective amount of a TLR8 modulating compound, or a pharmaceutically acceptable salt thereof.

Also provided is a method of treating an HIV infection in a human, the method comprising:
a) administering to a human in need thereof a pharmaceutically effective amount of a combination antiretroviral therapy regimen sufficient to lower the level of HIV detected in the human's blood or plasma from a first level to a second level, the second level comprising a lower concentration of HIV in the human's blood or plasma than the concentration of HIV in the human's blood or plasma in the first level; and
b) administering to the human a pharmaceutically effective amount of a compound of Formula (I), or a pharmaceutically acceptable salt thereof.

Provided is a method of treating an HIV infection in a human, the method comprising:
a) administering to a human in need thereof a pharmaceutically effective amount of a combination antiretroviral therapy regimen sufficient to lower the level of HIV detected in the human's blood or plasma to a specified level; and
b) administering to the human a pharmaceutically effective amount of a compound of Formula (I), or a pharmaceutically acceptable salt thereof.

Provided is a method of treating an HIV infection in a human, the method comprising:
a) a first step of administering to a human in need thereof a pharmaceutically effective amount of a combination antiretroviral therapy regimen sufficient to lower the level of HIV in the human's blood or plasma to below a detectable level; and
b) a second step following the first step, the second step comprising administering to the human a pharmaceutically effective amount of a combination antiretroviral therapy regimen and a pharmaceutically effective amount of a compound of Formula (I), or a pharmaceutically acceptable salt thereof.

Provided is a method of treating an HIV infection in a human, the method comprising:
a) a first step of administering to a human in need thereof a pharmaceutically effective amount of a combination antiretroviral therapy regimen sufficient to lower the level of HIV in the human's plasma to below 50 copies of HIV RNA/ml; and
b) a second step following the first step, the second step comprising administering to the human a pharmaceutically effective amount of a combination antiretroviral therapy regimen and a pharmaceutically effective amount of a compound of Formula (I), or a pharmaceutically acceptable salt thereof.

Provided is a method of treating an HIV infection in a human, the method comprising:
a) a first step of administering to a human in need thereof a pharmaceutically effective amount of a combination antiretroviral therapy regimen sufficient to lower the level of HIV in the human's blood or plasma to below a detectable level; and
b) a second step following the first step, the second step comprising administering to the human a pharmaceutically effective amount of a combination antiretroviral therapy regimen and a pharmaceutically effective amount of a TLR8 modulating compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120; or a pharmaceutically acceptable salt thereof.

Within each of the methods herein of treating an HIV infection in a human, the method comprising a first step of administering to a human in need thereof a pharmaceutically effective amount of an antiretroviral agent or a combination antiretroviral therapy (cART) regimen sufficient to lower the level of HIV in the human's blood or plasma to below a detectable level, followed by a second step following the first step, the second step comprising administering to the human a pharmaceutically effective amount of a combination antiretroviral therapy regimen and a pharmaceutically effective amount of a TLR8 modulating compound, there is a further embodiment in which the antiretroviral agent or cART regimen and the TLR8 modulating compound are both administered daily to the human. Within each of these methods herein of treating an HIV infection in a human there are further embodiments in which the antiretroviral agent or cART regimen is administered daily to the human and the TLR8 modulating compound is administered less than daily. Separate additional embodiments within each of these methods of treating an HIV infection in a human comprise administering antiretroviral agent or the cART regimen to the human daily and administering the TLR8 modulating compound to the human once or twice every other day or once or twice every third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty first, twenty second, twenty third, twenty fourth, twenty fifth, twenty sixth, twenty seventh, twenty eight, twenty ninth, thirtieth, forty fifth, or sixtieth day.

Also provided are separate methods within the method above, each utilizing in the second step a separate compound of one or more of the group of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof. One of such methods is as described above wherein, in the second step, the human is administered a pharmaceutically effective amount of Formula (J), or a pharmaceutically acceptable salt thereof, another method comprises administering to the human infected with HIV a pharmaceutically effective amount of Formula (I), etc.

As an example, provided is a method of treating an HIV infection in a human, the method comprising:
a) a first step of administering to a human in need thereof a pharmaceutically effective amount of an antiretroviral agent or a combination antiretroviral therapy regimen sufficient to lower the level of HIV in the human's blood or plasma to below a detectable level; and
b) a second step following the first step, the second step comprising administering to the human a pharmaceutically effective amount of an antiretroviral agent or a combination antiretroviral therapy regimen and a pharmaceutically effective amount of the TLR8 modulating compound, or a pharmaceutically acceptable salt thereof.

Within each of the methods of treating an HIV infection in a human herein wherein the first step comprises administering to a human in need thereof a pharmaceutically effective amount of an antiretroviral agent or a combination antiretroviral therapy regimen sufficient to lower the level of HIV in the human's blood or plasma to below a detectable level, there is a further embodiment comprising the method wherein the level of HIV in the human's blood or plasma below a detectable level comprises a viral load (VL) in plasma of less than 50 copies of HIV RNA/ml. Additional separate embodiments within each of the methods comprises the method described wherein the level of HIV in the human's blood or plasma below a detectable level comprises a viral load (VL) in plasma of a) less than 40 copies of HIV RNA/ml; b) less than 30 copies of HIV RNA/ml; c) less than 20 copies of HIV RNA/ml; d) less than 10 copies of HIV RNA/ml; e) less than 5 copies of HIV RNA/ml; f) less than 3 copies of HIV RNA/ml; less than 1 copy of HIV RNA/ml; and less than 0.5 copies of HIV RNA/ml.

Non-limiting assays useful in determining the concentration of HIV RNA in blood or plasma include the COBAS® AMPLICOR HIV-1 MONITOR Test, v1.5 (quantification of HIV-1 RNA from 50 to 750,000 copies/mL), COBASO AmpliPrep/COBASO TaqMan® HIV-1 Test, v2.0 (quantitates HIV-1 RNA from 20-10,000,000 copies/mL), the Abbott RealTime HIV-1 assay (quantitation of HIV-1 in human plasma from 40 to 10,000,000 copies/mL), or ultra-sensitive single copy quantitative PCR assays (SCA, iSCA, or gSCA). Other useful assays include the VERSANT® HIV-1 RNA 1.0 Assay (kPCR), the NucliSENS EasyQ® HIV-1 v2.0 assay, and the APTIMA® HIV-1 RNA Qualitative Assay.

Combination antiretroviral therapies and compositions which are included for use in each of the methods herein include the marketed products:
a) STRIBILD® tablets (elvitegravir 150 mg, cobicistat 150 mg, emtricitabine 200 mg, tenofovir disoproxil fumarate 300 mg) (Gilead Sciences, Inc.);
b) TRUVADA® tablets (emtricitabine 200 mg, tenofovir disoproxil fumarate 300 mg) (Gilead Sciences, Inc.);
c) ATRIPLA® tablets (efavirenz 600 mg, emtricitabine 200 mg, tenofovir disoproxil fumarate 300 mg) (Gilead Sciences, Inc.);
d) COMPLERA® tablets (200 mg emtricitabine, 25 mg rilpivirine, 300 mg of tenofovir disoproxil fumarate) (Gilead Sciences, Inc.);
e) EPZICOM® tablets (Eq. 600 mg base abacavir sulfate, 300 mg lamivudine);
f) COMBIVIR® tablets (150 mg lamivudine, 300 mg zidovudine (GlaxoSmithKline);
g) TIVICAY® tablets (50 mg dolutegravir)
h) TRIUMEQ® tablets (abacavir 600 mg, 50 mg dolutegravir, 300 mg lamivudine) and
i) TRIVIR® tablets (Eq. 300 mg base abacavir sulfate, 150 mg lamivudine, 300 mg zidovudine).

Also included for use in each of the methods herein is an antiretroviral combination of:
a) a pharmaceutically effective amount of elvitegravir;
b) a pharmaceutically effective amount of cobicistat;
c) a pharmaceutically effective amount of emtricitabine; and
d) a pharmaceutically effective amount of tenofovir alafenamide, or a pharmaceutically acceptable salt thereof.

Also included for use in each of the methods herein is an antiretroviral combination of:
a) a pharmaceutically effective amount of emtricitabine; and
b) a pharmaceutically effective amount of tenofovir alafenamide, or a pharmaceutically acceptable salt thereof.

Also included for use in each of the methods herein is an antiretroviral combination of:
a) a pharmaceutically effective amount of rilpivirine;
b) a pharmaceutically effective amount of emtricitabine; and
c) a pharmaceutically effective amount of tenofovir alafenamide, or a pharmaceutically acceptable salt thereof.

Also included for use in each of the methods herein is an antiretroviral combination of:
a) (2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide
b) a pharmaceutically effective amount of emtricitabine; and
c) a pharmaceutically effective amount of tenofovir alafenamide, or a pharmaceutically acceptable salt thereof.

Also included for use in each of the methods herein is an antiretroviral combination of:
a) a pharmaceutically effective amount of dolutegravir;
b) a pharmaceutically effective amount of cobicistat;
c) a pharmaceutically effective amount of emtricitabine; and
d) a pharmaceutically effective amount of tenofovir alafenamide, or a pharmaceutically acceptable salt thereof.

Also included for use in each of the methods herein is an antiretroviral combination of:
a) a pharmaceutically effective amount of atazanavir, or a pharmaceutically acceptable salt thereof, such as atazanavir sulfate; and
b) a pharmaceutically effective amount of cobicistat.

Also included for use in each of the methods herein is an antiretroviral combination of a) a pharmaceutically effective amount of TDF, b) a pharmaceutically effective amount of emtricitabine, and c) a pharmaceutically effective amount of a compound selected from the group of efavirenz, rilpivirine, elvitegravir, efavirenzatazanavir, darunavir, dolutegravir, raltegravir, and tipranavir.

Also included for use in each of the methods herein is an antiretroviral combination of a) a pharmaceutically effective amount of TAF, b) a pharmaceutically effective amount of emtricitabine, and c) a pharmaceutically effective amount of a compound selected from the group of efavirenz, rilpivirine, elvitegravir, efavirenz, atazanavir, darunavir, raltegravir, dolutegravir, and tipranavir.

Also provided is a method of eliminating an HIV infection in a human, the method comprising:
a) administering to a human in need thereof a pharmaceutically effective amount of a TLR8 modulating compound, or a pharmaceutically acceptable salt thereof

Provided is a method of eliminating an HIV infection in a human, the method comprising:
a) administering to a human in need thereof a pharmaceutically effective amount of a compound of Formula (I), or a pharmaceutically acceptable salt thereof; and
b) administering to the human a pharmaceutically effective amount of one or more antiretroviral agents.

Also provided is a method of eliminating an HIV infection in a human, the method comprising:
a) administering to a human in need thereof a pharmaceutically effective amount of a combination antiretroviral therapy; and
b) administering to a human in need thereof a pharmaceutically effective amount of a TLR8 modulating compound, or a pharmaceutically acceptable salt thereof.

Also provided is a method of eliminating an HIV infection in a human, the method comprising:
c) administering to a human in need thereof a pharmaceutically effective amount of a combination antiretroviral therapy; and
d) administering to a human in need thereof a pharmaceutically effective amount of a compound of Formula (I), or a pharmaceutically acceptable salt thereof.

Within each of the methods of eliminating an HIV infection in a human described herein there are further separate embodiments in which the TLR8 modulating compound is selected from the group comprising Formula (J), (I), (II), (IIa), (Ilb), (III), (IIIa), (IIIb), (IV), (IVa), (IVb), (IVc), or (IVd), or each of the individual compounds of the examples from Example 1 through Example 120; or a pharmaceutically acceptable salt thereof.

Within each of the methods herein of eliminating an HIV infection in a human, the method comprising administering to a human in need thereof a pharmaceutically effective amount of an antiretroviral agent or a combination antiretroviral therapy (cART) regimen and administering to the human a pharmaceutically effective amount of a combination antiretroviral therapy regimen and a pharmaceutically effective amount of a TLR8 modulating compound, there is a further embodiment in which the antiretroviral agent or cART regimen and the TLR8 modulating compound are both administered daily to the human. Within each of these methods herein of treating an HIV infection in a human there are further embodiments in which the antiretroviral agent or cART regimen is administered daily to the human and the TLR8 modulating compound is administered less than daily. Separate additional embodiments within each of these methods of treating an HIV infection in a human comprise administering the antiretroviral agent or the cART regimen to the human daily and administering the TLR8 modulating compound to the human once or twice every other day or once or twice every third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty first, twenty second, twenty third, twenty fourth, twenty fifth, twenty sixth, twenty seventh, twenty eight, twenty ninth, thirtieth, forty fifth, or sixtieth day.

cART Combinations and Regimens

Each of the methods of treatment above may utilize combinations of antiretroviral compounds, or a pharmaceutically acceptable salt thereof. Examples of combinations of specific dose ranges of antiretroviral agents that may be used in these methods of treatment are included in the tables below. It is understood that in the practice of the methods of treatment herein, the antiretroviral agents listed for each combination may be administered together in a single pharmaceutical composition or in divided forms, such as a single tablet or oral solution per agent or in different pharmaceutical compositions combining different groups of the agents. The amounts of each agent listed are intended to be a daily dosage of each agent, though the daily dosage may be administered to the human in need thereof in the present methods of treatment as a single dose of each agent per day or it may be divided and administered in multiple doses per day, such as dividing the daily dose into two, three, or four divided doses to be administered in a twice daily, three times daily, or four times daily regimen.

Combinations of the agents listed in each of the Pharmaceutical Composition Tables below may be used in each of the methods herein. It is understood that for each of the individual methods discussed herein there are separate methods in which each of the pharmaceutical combinations listed in the Pharmaceutical Composition Tables below are used in the each of the individual methods. For instance, provided in Combination Antiretroviral Table A are eight separate methods of treating an HIV infection in a human, as described above, comprising administering to a human infected with HIV the combinations of the pharmaceutical agents listed as Combination Examples A-1, A-2, A-3, A-4, A-5. A-6, A-7, and A-8 in combination with a pharmaceutically effective amount of TLR8 modulating compound, as described herein.

### Combination Antiretroviral Table A

Antiviral combinations and regimens for use in the methods herein comprising elvitegravir, cobicistat, emtricitibine, and TDF or TAF include:

| Comb. | | | | | |
|---|---|---|---|---|---|
| Ex. | elvitegravir | cobicistat | emtricitabine | TDF | TAF |
| A-1 | 100 mg to | 100 mg to | 150 mg to | 250 mg to 350 mg | 0 mg |
| | 200 mg | 200 mg | 250 mg | | |
| A-2 | 125 mg to | 125 mg to | 175 mg to | 275 mg to 325 mg | 0 mg |
| | 175 mg | 175 mg | 225 mg | | |
| A-3 | 145 mg to | 145 mg to | 190 mg to | 290 mg to 310 mg | 0 mg |
| | 155 mg | 155 mg | 210 mg | | |
| A-4 | 150 mg | 150 mg | 200 mg | 300 mg | 0 mg |
| A-5 | 100 mg to | 100 mg to | 150 mg to | 0 mg | 5 mg to 30 mg |
| | 200 mg | 200 mg | 250 mg | | |
| A-6 | 125 mg to | 125 mg to | 175 mg to | 0 mg | 5 mg to 30 mg |
| | 175 mg | 175 mg | 225 mg | | |
| A-7 | 145 mg to | 145 mg to | 190 mg to | 0 mg | 5 mg to 30 mg |
| | 155 mg | 155 mg | 210 mg | | |
| A-8 | 150 mg | 150 mg | 200 mg | 0 mg | 25 mg |
| A-9 | 150 mg | 150 mg | 200 mg | 0 mg | 10 mg |

### Combination Antiretroviral Table B

Antiviral combinations and regimens for use in the methods herein comprising emtricitibine and TDF or TAF include:

| Combination | | | |
|---|---|---|---|
| Example | emtricitabine | TDF | TAF |
| B-1 | 150 mg to 250 mg | 250 mg to 350 mg | 0 mg |
| B-2 | 175 mg to 225 mg | 275 mg to 325 mg | 0 mg |
| B-3 | 200 mg | 300 mg | 0 mg |
| B-4 | 150 mg to 250 mg | 0 mg | 5 mg to 30 mg |
| B-5 | 175 mg to 225 mg | 0 mg | 5 mg to 30 mg |
| B-6 | 175 mg to 225 mg | 0 mg | 25 mg |
| B-7 | 200 mg | 0 mg | 25 mg |

### Combination Antiretroviral Table C

Antiviral combinations and regimens for use in the methods herein comprising emtricitibine, TDF or TAF, and raltegravir include:

| Comb. | | | | |
|---|---|---|---|---|
| Example | emtricitabine | TDF | raltegravir | TAF |
| C-1 | 150 mg to 250 mg | 250 mg to 350 mg | 350 mg to 450 mg | 0 mg |
| C-2 | 175 mg to 225 mg | 275 mg to 325 mg | 375 mg to 425 mg | 0 mg |
| C-3 | 200 mg | 300 mg | 400 mg | 0 mg |
| C-4 | 150 mg to 250 mg | 0 mg | 350 mg to 450 mg | 5 mg to 30 mg |
| C-5 | 175 mg to 225 mg | 0 mg | 375 mg to 425 mg | 5 mg to 30 mg |
| C-6 | 175 mg to 225 mg | 0 mg | 375 mg to 425 mg | 25 mg |
| C-7 | 200 mg | 0 mg | 400 mg | 25 mg |

### Combination Antiretroviral Table D

Antiviral combinations and regimens for use in the methods herein comprising emtricitibine, TDF or TAF, and dolutegravir include:

| Comb. | | | | |
|---|---|---|---|---|
| Ex. | emtricitabine | TDF | dolutegravir | TAF |
| D-1 | 150 mg to 250 mg | 250 mg to 350 mg | 30 mg to 70 mg | 0 mg |
| D-2 | 150 mg to 250 mg | 250 mg to 350 mg | 40 mg to 60 mg | 0 mg |
| D-3 | 175 mg to 225 mg | 275 mg to 325 mg | 40 mg to 60 mg | 0 mg |
| D-4 | 190 mg to 210 mg | 290 mg to 310 mg | 45 mg to 55 mg | 0 mg |
| D-5 | 200 mg | 300 mg | 50 mg | 0 mg |
| D-6 | 150 mg to 250 mg | 0 mg | 30 mg to 70 mg | 5 mg to 30 mg |
| D-7 | 150 mg to 250 mg | 0 mg | 40 mg to 60 mg | 5 mg to 30 mg |
| D-8 | 175 mg to 225 mg | 0 mg | 40 mg to 60 mg | 5 mg to 30 mg |
| D-9 | 190 mg to 210 mg | 0 mg | 45 mg to 55 mg | 5 mg to 30 mg |
| D-10 | 200 mg | 0 mg | 50 mg | 25 mg |

### Combination Antiretroviral Table E

Antiviral combinations and regimens for use in the methods herein comprising rilpivirine HCl, emtricitibine, and TDF or TAF include:

| Comb. | | | | |
|---|---|---|---|---|
| Ex. | Rilpivirine HCl | emtricitabine | TDF | TAF |
| E-1 | 20 mg to 30 mg | 150 mg to 250 mg | 250 mg to 350 mg | 0 mg |
| E-2 | 22 mg to 28 mg | 175 mg to 225 mg | 275 mg to 325 mg | 0 mg |
| E-3 | 27.5 mg | 200 mg | 300 | 0 mg |
| E-4 | 20 mg to 30 mg | 150 mg to 250 mg | 0 mg | 5 mg to 30 mg |
| E-5 | 22 mg to 28 mg | 175 mg to 225 mg | 0 mg | 5 mg to 30 mg |
| E-6 | 27.5 mg | 200 mg | 0 mg | 25 mg |

### Combination Antiretroviral Table F

Antiviral combinations and regimens for use in the methods herein comprising efavirenz, emtricitibine, and TDF or TAF include:

| Comb. | | | | |
|---|---|---|---|---|
| Ex. | efavirenz | emtricitabine | TDF | TAF |
| F-1 | 500 mg to 700 mg | 150 mg to 250 mg | 150 mg to 250 mg | 0 mg |
| F-2 | 550 mg to 650 mg | 175 mg to 225 mg | 175 mg to 225 mg | 0 mg |
| F-3 | 575 mg to 625 mg | 175 mg to 225 mg | 175 mg to 225 mg | 0 mg |
| F-4 | 600 mg | 200 mg | 200 mg | 0 mg |
| F-5 | 500 mg to 700 mg | 150 mg to 250 mg | 0 mg | 5 mg to 30 mg |
| F-6 | 550 mg to 650 mg | 175 mg to 225 mg | 0 mg | 5 mg to 30 mg |
| F-7 | 575 mg to 625 mg | 175 mg to 225 mg | 0 mg | 5 mg to 30 mg |
| F-8 | 575 mg to 625 mg | 175 mg to 225 mg | 0 mg | 25 mg |
| F-9 | 600 mg | 200 mg | 0 mg | 25 mg |

### Combination Antiretroviral Table G

Antiviral combinations and regimens for use in the methods herein comprising elvitregravir, emtricitibine, and TAF, with and without cobicistat, include:

| Combination | | | | |
|---|---|---|---|---|
| Example | elvitegravir | emtricitabine | cobicistat | TAF |
| G-1 | 100 mg to 200 mg | 150 mg to 250 mg | 100 mg to 200 mg | 5 mg to 50 mg |
| G-2 | 600 mg to 1200 mg | 150 mg to 250 mg | 0 mg | 5 mg to 50 mg |
| G-3 | 100 mg to 200 mg | 150 mg to 250 mg | 100 mg to 200 mg | 5 mg to 30 mg |
| G-4 | 600 mg to 1200 mg | 150 mg to 250 mg | 0 mg | 5 mg to 30 mg |
| G-5 | 125 mg to 175 mg | 175 mg to 225 mg | 125 mg to 175 mg | 5 mg to 30 mg |
| G-6 | 700 mg to 1200 mg | 175 mg to 225 mg | 0 mg | 5 mg to 30 mg |
| G-7 | 125 mg to 175 mg | 175 mg to 225 mg | 125 mg to 175 mg | 5 mg to 15 mg |
| G-8 | 700 mg to 1200 mg | 175 mg to 225 mg | 0 mg | 5 mg to 15 mg |
| G-9 | 125 mg to 175 mg | 175 mg to 225 mg | 125 mg to 175 mg | 20 mg to 30 mg |
| G-10 | 700 mg to 1200 mg | 175 mg to 225 mg | 0 mg | 20 mg to 30 mg |
| G-11 | 150 mg | 200 mg | 150 mg | 5 mg to 30 mg |
| G-12 | 800 mg to 1200 mg | 200 mg | 0 mg | 5 mg to 30 mg |
| G-13 | 150 mg | 200 mg | 150 mg | 5 mg to 15 mg |
| G-14 | 800 mg to 1200 mg 150 | 200 mg | 0 mg | 5 mg to 15 mg |
| G-15 | 150 mg | 200 mg | 150 mg | 20 mg to 30 mg |
| G-16 | 800 mg to 1200 mg 150 | 200 mg | 0 mg | 20 mg to 30 mg |
| G-17 | 150 mg | 200 mg | 150 mg | 25 mg |
| G-18 | 800 mg to 1200 mg 150 | 200 mg | 0 mg | 25 mg |
| G-19 | 150 mg | 200 mg | 150 mg | 10 mg |
| G-20 | 800 mg to 1200 mg 150 | 200 mg | 0 mg | 10 mg |

### Combination Antiretroviral Table H

Antiviral combinations and regimens for use in the methods herein comprising atazanavir sulfate and cobicistat include:

| Combination | | |
|---|---|---|
| Example | atazanavir sulfate | cobicistat |
| H-1 | 250 mg to 350 mg | 100 mg to 200 mg |
| H-2 | 275 mg to 325 mg | 125 mg to 175 mg |
| H-3 | 290 mg to 310 mg | 140 mg to 160 mg |
| H-4 | 300 mg | 150 mg |

### Combination Antiretroviral Table I

Antiviral combinations and regimens for use in the methods herein comprising abacavir (such as administered as abacavir sulfate), lamivudine, and, optionally, dolutegravir include:

| Comb. | | | |
|---|---|---|---|
| Ex. | abacavir | lamivudine | dolutegravir |
| I-1 | 500 mg to 700 mg | 250 mg to 350 mg | 0 mg |
| I-2 | 275 mg to 325 mg | 125 mg to 175 mg | 0 mg |
| I-3 | 290 mg to 310 mg | 140 mg to 160 mg | 0 mg |
| I-4 | 300 mg | 150 mg | 0 mg |
| I-1 | 500 mg to 700 mg | 250 mg to 350 mg | 25 mg to 75 mg |
| I-2 | 275 mg to 325 mg | 125 mg to 175 mg | 40 mg to 60 mg |
| I-3 | 290 mg to 310 mg | 140 mg to 160 mg | 45 mg to 55 mg |
| I-4 | 300 mg | 150 mg | 50 mg |

### Combination Antiretroviral Table J

Antiviral combinations and regimens for use in the methods herein comprising darunavir (such as administered as a Prezista® tablet or oral solution) and ritonavir or cobicistat include:

| Combi- | | | |
|---|---|---|---|
| nation | | | |
| Ex- | | | |
| ample | darunavir | ritonavir | cobicistat |
| J-1 | 50 mg to 1000 mg | 50 mg to 150 mg | 0 mg |
| J-2 | 50 mg to 1000 mg | 0 mg | 50 mg to 200 mg |
| J-3 | 500 mg to 900 mg | 50 mg to 150 mg | 0 mg |
| J-4 | 500 mg to 900 mg | 0 mg | 50 mg to 200 mg |
| J-5 | 500 mg to 700 mg | 75 mg to 125 mg | 0 mg |
| J-6 | 500 mg to 700 mg | 0 mg | 75 mg to 175 mg |
| J-7 | 600 mg | 100 mg | 0 mg |
| J-9 | 800 mg | 100 mg | 0 mg |
| J-10 | 600 mg | 0 mg | 50 mg to 150 mg |
| J-11 | 800 mg | 0 mg | 100 mg to 200 mg |
| J-12 | 600 mg | 0 mg | 100 mg |
| J-13 | 800 mg | 0 mg | 150 mg |

### Lowering Viremia and Chronic Set Point of HIV Viral Load

Provided is a method of reducing HIV viremia in a human infected with HIV, the method comprising administering to the human a pharmaceutically effective amount of a TLR8 modulating compound, or a pharmaceutically effective amount thereof.

Also provided is a method of reducing HIV viremia in a human infected with HIV, wherein the human infected with HIV is receiving treatment with one or more antiviral agents, the method comprising administering to the human infected with HIV a pharmaceutically effective amount of a TLR8 modulating compound, or a pharmaceutically effective amount thereof.

Provided is a method of lowering the chronic set point of HIV viral load in a human infected with HIV, the method comprising administering to the human a pharmaceutically effective amount of a TLR8 modulating compound, or a pharmaceutically effective amount thereof.

Also provided is a method of lowering the chronic set point of HIV viral load in a human infected with HIV, the method comprising administering to the human a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically effective amount thereof.

Also provided is a method of lowering the chronic set point of HIV viral load in an HIV infected human receiving combination antiretroviral therapy, the method comprising administering to the HIV infected human receiving combination antiretroviral therapy a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically effective amount thereof.

Further provided is a method of lowering the chronic set point of HIV viral load in an HIV infected human receiving highly active antiretroviral therapy, the method comprising administering to the HIV infected human receiving highly active antiretroviral therapy a pharmaceutically effective amount of a compound of Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically effective amount thereof.

Also provided are separate methods of reducing HIV viremia in a human infected with HIV and of lowering the chronic set point of HIV viral load in a human infected with HIV, each of the separate methods comprising administering to the human infected with HIV a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120; or a pharmaceutically acceptable salt thereof.

Provided is a method of lowering the chronic set point of HIV viral load in a human infected with HIV, the method comprising:
a) a first step of administering to a human in need thereof a pharmaceutically effective amount of a combination antiretroviral therapy regimen sufficient to lower the level of HIV in the human's blood or plasma to below a detectable level; and
b) a second step following the first step, the second step comprising administering to the human a pharmaceutically effective amount of a combination antiretroviral therapy regimen and a pharmaceutically effective amount of a TLR8 modulating compound or a pharmaceutically acceptable salt thereof.

Also provided is a method of lowering the chronic set point of HIV viral load in a human infected with HIV, the method comprising:
a) a first step of administering to a human in need thereof a pharmaceutically effective amount of a combination antiretroviral therapy regimen sufficient to lower the chronic set point of HIV in the human's blood or plasma to a first level of less than 50 copies of HIV-1 RNA/ml of plasma; and
b) a second step following the first step, the second step comprising administering to the human a pharmaceutically effective amount of a combination antiretroviral therapy regimen and a pharmaceutically effective amount of a TLR8 modulating compound or a pharmaceutically acceptable salt thereof, to lower the chronic set point of HIV in the human's blood or plasma to a second level, the second level being less than the first level.

Provided is a method of lowering the chronic set point of HIV viral load in a human infected with HIV, the method comprising:
a) a first step of administering to a human in need thereof a pharmaceutically effective amount of a combination antiretroviral therapy regimen sufficient to lower the level of HIV in the human's blood or plasma to below a detectable level; and
b) a second step following the first step, the second step comprising administering to the human a pharmaceutically effective amount of a combination antiretroviral therapy regimen and a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120; or a pharmaceutically acceptable salt thereof.

Also provided is a method of lowering the chronic set point of HIV viral load in a human infected with HIV, the method comprising:
a) a first step of administering to a human in need thereof a pharmaceutically effective amount of a combination antiretroviral therapy regimen sufficient to lower the chronic set point of HIV in the human's blood or plasma to a first level of less than 50 copies of HIV-1 RNA/ml of plasma; and
b) a second step following the first step, the second step comprising administering to the human a pharmaceutically effective amount of a combination antiretroviral therapy regimen and a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120 or a pharmaceutically acceptable salt thereof, to lower the chronic set point of HIV in the human's blood or plasma to a second level, the second level being less than the first level.

Nine additional separate embodiments within each of the methods described above wherein in one embodiment each, respectively, the designated method of lowering the chronic set point of HIV viral load in a human infected with HIV comprises the method described wherein the second level of the chronic set point of HIV in the human's blood or plasma is a concentration in the human's plasma of a) less than 40 copies of HIV-1 RNA/ml of plasma; b) less than 30 copies of HIV-1 RNA/ml of plasma; c) less than 20 copies of HIV-1 RNA/ml of plasma; d) less than 10 copies of HIV-1 RNA/ml of plasma; e) less than 5 copies of HIV-1 RNA/ml of plasma; f) less than 3 copies of HIV-1 RNA/ml of plasma; g) less than 1 copy of HIV-1 RNA/ml of plasma; h) less than 0.5 copies of HIV-1 RNA/ml of plasma; i) less than 0.3 copies of HIV-1 RNA/ml of plasma; and j) less than 0.1 copies of HIV-1 RNA/ml of plasma.

Also provided are separate embodiments within each of the methods of lowering the chronic set point of HIV viral load in a human infected with HIV, above, each comprising in the second step administering to the human a separate compound of one of the group Formula (J), (I), (II), (IIa), (IIb), (III), (IIIa), (IIIb), (IV), (IVa), (IVb), (IVc), or (IVd), or each of the individual compounds of the examples from Example 1 through Example 120, or a pharmaceutically acceptable salt thereof. One of such methods is as described above wherein, in the second step, the human is administered a pharmaceutically effective amount of Formula (J), or a pharmaceutically acceptable salt thereof, another method comprises administering to the human infected with HIV a pharmaceutically effective amount of Formula (I), etc.

In each of the methods listed above for lowering the chronic set point of HIV viral load in a human infected with HIV there is a further embodiment in which the detectable level in the first step is a concentration in the human's blood plasma of less than 50 copies of HIV-1 RNA/mL.

### Enhancing Immune Activity and Increasing HIV Gene Expression

Provided is a method of enhancing immune cell activity and increasing HIV gene expression in a human infected with HIV, the method comprising administering to the human infected with HIV a pharmaceutically effective amount of a TLR8 modulating compound, or a pharmaceutically acceptable salt thereof.

Also provided is a method of enhancing immune cell activity and increasing HIV gene expression in a human infected with HIV, the method comprising administering to the human infected with HIV a pharmaceutically effective amount of a compound of Formula I, or a pharmaceutically acceptable salt thereof.

Also provided is a method of enhancing immune cell activity and increasing HIV gene expression in a human infected with HIV, the method comprising administering to the human infected with HIV a pharmaceutically effective amount of a compound selected from one of Formula (J), (I), (II), (IIa), (IIb), (III), (IIIa), (IIIb), (IV), (IVa), (IVb), (IVc), or (IVd), or each of the individual compounds of the examples from Example 1 through Example 120 or a pharmaceutically acceptable salt thereof. It is understood that one of such methods comprises administering to the human infected with HIV a pharmaceutically effective amount of Formula I, or a pharmaceutically acceptable salt thereof, another method comprises administering to the human infected with HIV a pharmaceutically effective amount of Formula 1(a), etc. Also provided are separate methods of enhancing immune cell activity and increasing HIV gene expression in a human infected with HIV, each of the separate methods comprising administering to the human infected with HIV a pharmaceutically effective amount of a compound selected from one of Formula (J), (1), (II), (IIa), (IIb), (III), (IIIa), (IIIa), (IV), (IVa), (IVb), (IVc), or (IVd), or each of the individual compounds of the examples from Example 1 through Example 120or a pharmaceutically acceptable salt thereof.

Within each of the methods of enhancing immune cell activity and increasing HIV gene expression in a human infected with HIV there are further separate embodiments wherein the immune cell activity is, respectively, in each of the further embodiments one of the activities selected from the group of a) plasmacytoid dendritic cell (pDC) activity, b) B-cell activity; c) T-cell activity, d) CD4 T-cell activity, e) CD8 T-cell activity, and f) natural killer (NK) cell activity, g) invariant NK T cell activity, h) monocyte/macrophage activity, i) myeloid dendritic cell (mDC) activity

### Enhancing Antiviral Efficacy

Also provided is a method of enhancing the efficacy of an antiviral agent in a human infected with HIV, the method comprising administering to the human infected with HIV a pharmaceutically effective amount of a TLR8 modulating compound and a pharmaceutically effective amount of an antiviral agent.

Also provided is a method of enhancing the efficacy of two or more antiviral agents in a human infected with HIV, the method comprising administering to the human infected with HIV a pharmaceutically effective amount of a TLR8 modulating compound and a pharmaceutically effective amount of each of the two or more antiviral agents.

Separate embodiments within the method of enhancing the efficacy of an antiviral agent in a human infected with HIV comprise the method wherein the TLR8 modulating compound is a compound of Formula (I), or a pharmaceutically acceptable salt thereof.

Also provided is a method of enhancing the efficacy of an antiviral agent in a human infected with HIV, the method comprising administering to the human infected with HIV a pharmaceutically effective amount of a TLR8 modulating compound and a pharmaceutically effective amount of an antiviral agent and a pharmaceutically effective amount of cobicistat. Separate embodiments within the method of enhancing the efficacy of an antiviral agent in a human infected with HIV comprise the method wherein the TLR8 modulating compound is a compound of Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof.

Also provided is a method of enhancing the efficacy of an antiviral agent in a human infected with HIV, the method comprising administering to the human infected with HIV a pharmaceutically effective amount of a TLR8 modulating compound, a pharmaceutically effective amount of an antiviral agent, and a pharmaceutically effective amount of ritonavir. Separate embodiments within the method of enhancing the efficacy of an antiviral agent in a human infected with HIV comprise the method wherein the TLR8 modulating compound is a compound of Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof.

Additional separate embodiments of the methods above of enhancing the efficacy of an antiviral agent in a human infected with HIV, comprise the method wherein the compound of Formula II, or a pharmaceutically acceptable salt thereof, is selected from the group of Formula (J), (I), (II), (IIa), (IIb), (III), (IIIa), (IIIa), (IV), (IVa), (IVb), (IVc), or (IVd), or each of the individual compounds of the examples from Example 1 through Example 120or a pharmaceutically acceptable salt thereof.

Enhancing the efficacy of an antiviral agent refers the achievement of greater antiviral activity in a human infected with HIV from administration of the antiviral agent and a TLR8 modulating compound than would be achieved by administration of the same dosage or regimen of the antiviral agent alone. Enhancing the efficacy of an antiviral agent includes achieving a lower viral set point or a lower viral load in the human infected with HIV by administration of the antiviral agent and a TLR8 modulating compound than would be achieved by administration of the same dosage or regimen of the antiviral agent alone, as well as achieving a desired viral set point or viral load in the human through the administration of a lower dose of the antiviral agent. Enhancing the efficacy of an antiviral agent also includes achieving elimination of HIV infection in the human infected with HIV. TLR8 modulating compounds may be used in the methods herein to enhance the efficacy of combination antiviral agents, including those listed in Tables A through J.

### HIV Vaccines

Provided is a method of treating an HIV infection in a human, the method comprising:
a) administering to a human in need thereof a pharmaceutically effective amount of a TLR8 modulating compound, or a pharmaceutically acceptable salt thereof; and
b) administering to the human a pharmaceutically effective amount of an HIV vaccine.

TLR8 modulating compounds described herein may also be administered in the methods herein in combination with an HIV vaccine, such as a peptide vaccines, recombinant subunit protein vaccines (including subunit proteins gp120, gp140, and gp160, live vector vaccines encoding HIV-1 antigens, such as those selected from the group of gag, pol, env, nef, rev, tat, vif, vpr, vpu, and antigenic proteins, variants and fusion proteins thereof), inactivated vaccines, modified envelope vaccines, replicons (including Venezuelan equine encephalitis (VEE), Semliki forest virus (SFV), adenovirus-associated virus (AAV), including self-complementary adeno-associated virus (scAAV), vesicular stomatitis virus (VSV), and human papillomavirus (HPV) replicon systems), DNA vaccines, vaccine combinations, and virus-like particle vaccines (pseudovirion vaccines). Recombinant HIV vaccines may be produced using vaccine viral vector platforms known in the art, including those developed from Adenoviridae, Poxviridae, Herpesviridae, or Adeno-associated viruses, as well as cytomegalovirus, carynpox, rubella, poliovirus, Venezuelan equine encephalitis virus, lentivirus, salmonella, bacilli Calmete-Guerin (BCG), and Sendai vectors.

Examples of HIV vaccines for use with the methods herein include ALVAC-HIV MN120TMG (vCP205), rgp120, monomeric gp120, trimeric gp120, gp120 monomer+gp120 trimer, MN rgp120/HIV-1 and GNE8 rgp120/HIV-1, ALVAC-HIV (vCP1521), ALVAC+gp120/MF59, ALVAC-HIV MN120TMG (vCP205), ALVAC(2)120(B,MN)GNP (vCP1452), ALVAC(1)120(B,MN)GNP (vCP1433), ALVAC-HIV+AIDSVAX® B/E, ALVAC VIH 1433, AIDSVAX BIB, AIDSVAX B/E, tgAAC09 (a Gag-PR-RT AAV HIV vaccine), Ad35, Ad35-GRIN/ENV, Ad35-GRIN, Ad35-ENV, the SeV-G(NP) vaccine, EN41-FPA2 HIV, EN41-UGR7C, Ad4-EnvC150, GSK 692342, GSK 732461, GSK 732462, MRKAd5 HIV-1 Gag, MRKAd5 HIV-1 gag/pol/nef, JS7 DNA, pGA2/JS7, Sub C gp140, trimeric gp140, trimeric gp140 +monomeric gp120, trimeric gp140+trimeric gp120, trimeric gp140+monomeric gp120+trimeric gp120, TBC-M4, MVA-nef, rMVA-HIV (env/gag [TBC-M358], tat/rev/nef-RT [TBC-M335], rFPV-HIV (env/gag [TBC-F357], tat/rev/nef-RT [TBC-F349], TBC-3B, ADVAX e/g+ADVAX p/N-t (ADVAX), MVA-C+gp140/MF59, DNA-C, DNA-C2, MVA-C, MVA HIV-B (MVATG17401), MVA-mBN120B, MF59, MTP-PE/MF59, DNA-C2+MVA-C, DNA-C2+MVA-C+gp140/MF59, NYVAC, NYVAC-B/rAd5, rAd5/NYVAC-B NYVAC-HIV-PT1, NYVAC-HIV-PT4, DNA+NYVAC+gp120, NYVAC+gp120, Ad26, Ad26.ENVA.01 (rAd26), MVA, Ad26/MVA, HIV gp41, HIV gp41 monomer, HIV gp41 trimer, gp120, gp140, gp160, PENNVAX®-B HIV Vaccine, PENNVAX-G DNA, Salmonella typhi CVD 908-HIV-1 LAI gp 120 (VVG 203), HIV-1MN, rgp120/HIV-1MN, VRC4302, VRC-HIVDNA016-00-VP, VRC-HIVDNA009-00-VP, VRC-HIVDNA009-00-VP, VRC-HIVADV014-00-VP, gp160 MN/LAI-2, VRC-HIVADV027-00-VP, VRC-HIVADV038-00-VP, VRC-HIVDNA044-00-VP, VRC-HIVDNA016-00-VP, VRC rAd5 vaccine (rAd5 gag-pot/env A/B/C), HIV-v, LIPO-4, LIPO-5, LIPO-6T, Modified Vaccinia Ankara (MVA) Vectored HIV-1 (ADMVA), CTL MEP/RC529-SE/GM-CSF (CTL MEP), AVX101, REMUNE® HIV-1 immunogen, HIV p24/MF59, HIV-1 p24(gag), HIV SF2 gp120/MF59, rgp120/HIV-1 SF-2 (gp120), rgp120/HIV-1 SF-2, MVA-CMDR, SCBaL/M9, DNA Nat-B env, NYVAC Nat-B env, DNA CON-S env, NYVAC CON-S env, DNA Mosaic env, NYVAC Mosaic env, rAd5 env A, rAd5 env B, rAd5 env C, rAd5 gag-pot, GENEVAX-HIV (APL 400-003), rMVA-HIV (rMVA-HIV env/gag+rMVA-HIV tat/rev/nef-RT), rFPV-HIV (rFPV-HIV env/gag+rFPV-HIV tat/rev/nef-RT), HIV-1 gag DNA plus IL-12 DNA adjuvant, DNA-HIV-PT123, DNA HIVIS, HIVIS 03 DNA, MVA-CMDR, EnvDNA, PolyEnvl, EnvPro, SAAVI DNA-C2, SAAVI MVA-C, HIV-1 C4-V3 Polyvalent Peptide, EP HIV-1043, EP HIV-1090, HIV-MAG, CN54gp140, CN54gp140/GLA-AF, HIV DNA plasmid/recombinant fowlpox vector, HIV62B, MVA/HIV62, pGA2/JS7 DNA/MVA/HIV62, VSV-Indiana HIV gag, MRKAd5 (Clade B), Clade B gag DNA/PLG, MRKAd5 HIV-1 gag/pol/nef, env DNA/PLG, GEO-D03 DNA, Trivalent MRKAd5 HIV-1 gag/pol/nef, HIVAC-1e, MVA.HIVconsv, pSG2.HIVconsv DNA, Electroporated pSG2.HIVconsv, pHIS-HIV-AE, rAAV1-PG9DP, Ad5.ENVA.48 HIV-1, Ad26.ENVA.01 HIV-1, NefTat, gp120W61D, Profectus HIV MAG pDNA, pGA2/JS2 Plasmid DNA, ChAdV63.HIVconsv, HIV gp120/NefTat/ASO2A, rgp120/HIV-1111B, rgp120/HIV-1MN Monovalent Octameric V3 Peptide Vaccine, HIV-1 C4-V3 Polyvalent Peptide Vaccine, HIV-1 Gag-Pol DNA (APL 400-047), AFO-18, NYVAC-C, UBI HIV-1 MN PND peptide immunogen, UBI microparticulate monovalent HIV-1 MN branched peptide, HIV p17/p24:Ty-VLP, A244 rgp120/HIV-1, Env 2-3, MTP-PE/MF59, P3C541b Lipopeptide, rAd5 Gag-Pol Env A/B/C, rAd5 Gag-Pol, Ad4-H5-VTN, EP-1233, MVA-mBN32, rVSV, pGA2/JS7 DNA, MVA/HIV62, pGA2/JS7 (JS7)DNA, MVA62B, HIV-1 Tat/delta-V2 Env combined, HIV-1 delta-V2 Env, GTU-multiHIV B, E1M184V peptide, VCR-HIVDNA006-00-VP, HIV LFn-p24, VAC-3S, MYM-V101, DCVax-001, DCVax plus poly-ICLC, Vacc-4x, TUTI-16, gp120/ASO2A, gp120/nef/tat/SIV nef/ASO2A, nef/tat/SIV nef/ASO2A, gp120/nef/tat/SIV nef, nef/tat/SIV nef/AS06, VICHREPOL, Ad35-ENVA, Ad5HVR48.ENVA.01, ADVAX e/g, ADVAX p/n-t, Cervico-vaginal CN54gp140-hsp70 conjugate vaccine (TL01), DNA (Gag, Pot, and Env genes from HIV-1 CN54)+Tiantian vaccinia vector, HIV-1 CN54 gag, HIV-1 CN54 pot, HIV-1 CN54 env, MV1-F4-CT1, MVA.HIVA, MVA HIV-B, rAd35, and rVSV_{IN} HIV-1 Gag vaccines, and combinations thereof.

Examples of HIV vaccines that may also be used in the present methods and useful vectors for preparing them include those disclosed in US 2008/0199493 A1 (Picker et al.), US 2013/0142823 (Picker et al.), US20040223977 (Diamond), WO2014039840 (Levy), WO2014026033 (Yamamoto), WO2013182660 (Sorensen et al.), WO2013110818 (Brander et al.), WO2013110790 (Bomsel et al.), WO2013059442 (Song et al.), WO2012156750 (Davis et al.), WO2012137072 (Andrieu et al.), WO2012116142 (Podack et al.), US20120107910 (Liu et al.), WO2012018856 (Rautsola et al.), US20120021000 (Opendra et al.), US20110305749 (Ryttergaard et al.), WO2011117408 (Bourguignon et al.), US20130195904A1 (August et al.), US20110159025 (Littman et al.), US20110123485 (Desrosiers et al.), US20110311585A1 (Berman), US20110159025A1 (Littman et al.), US20110014221 (Kang et al.), US20120263720A1 (Gronvold et al.), US20100304483 (Abulafia-Lapid), US20100215695 (Yu), US20100135994 (Banchereau et al.), US20120045472A1 (Harrison et al.), US20110195083A1 (Anglister et al.), U.S. Pat. No. 7,612,173B2 (Albrecht et al.), US20080199493A1 (Picker et al.), and U.S. Pat. No. 7,364,744B2 (Hovanessian et al.), US20150132332 (Shao et al.), WO2015073291 (Weiner et al.), WO2015048512 (Haynes et al.), WO2015001128 (Benarous et al.), US20140302080 (Barouch et al.), (WO2014039840 (Levy et al.), WO2014026033 (Yamamoto et al.), WO2015007337 (Hoie et al.), US20150132255 (Birger et al.), US20150050310 (Brander et al.), and US20150004190 (Bomsel et al.), the contents of each of which are incorporated herein by reference.

Also useful in the methods and combinations with the vaccines and methods described herein are agents that provide adjuvant activity to a vaccine, such as agonists of TLR3, TLR4, TLR7, TLR9, NOD-1/2 (NOD-like receptors), and RIG-I (RIG-I-like receptors).

Also provided is a method of enhancing the efficacy of an HIV vaccine, the method comprising administering to a human in need thereof a pharmaceutically effective amount of an HIV vaccine and a pharmaceutically effective amount of a TLR8 modulating compound. One method of enhancing the efficacy of an HIV vaccine comprises a first step of administering to a human in need thereof a pharmaceutically effective amount of a TLR8 modulating compound and a second step of administering to the human in need thereof a pharmaceutically effective amount of an HIV vaccine. Another method of enhancing the efficacy of an HIV vaccine comprises a first step of administering to a human in need thereof a pharmaceutically effective amount of an HIV vaccine and a second step of administering to the human in need thereof a pharmaceutically effective amount of a TLR8 modulating compound. Specific separate embodiments within each of these methods of enhancing efficacy of an HIV vaccine comprise the method indicated wherein the TLR8 modulating compound is a compound selected from Formula (J), (I), (II), (IIa), (IIb), (III), (IIIa), (IIIa), (IV), (IVa), (IVb), (IVc), or (IVd), or each of the individual compounds of the examples from Example 1 through Example 120; or a pharmaceutically acceptable salt thereof. Non-limiting examples of HIV vaccines for use in these methods include those described herein.

### HIV Antibodies

Provided is a method of treating an HIV infection in a human, the method comprising:
a) administering to a human in need thereof a pharmaceutically effective amount of a TLR8 modulating compound, or a pharmaceutically acceptable salt thereof; and
b) administering to the human a pharmaceutically effective amount of an HIV antibody.

Also provided is a method of treating an HIV infection in a human, the method comprising:
a) administering to a human in need thereof a pharmaceutically effective amount of a TLR8 modulating compound, or a pharmaceutically acceptable salt thereof; and
b) administering to the human a pharmaceutically effective amount of two or more HIV antibodies.

Provided is a method of treating an HIV infection in a human, the method comprising:
a) administering to a human in need thereof a pharmaceutically effective amount of a compound of Formula (I), or a pharmaceutically acceptable salt thereof; and
b) administering to the human a pharmaceutically effective amount of an HIV antibody.

Also provided is a method of treating an HIV infection in a human, the method comprising:
a) administering to a human in need thereof a pharmaceutically effective amount of a compound of Formula (I), or a pharmaceutically acceptable salt thereof; and
b) administering to the human a pharmaceutically effective amount of two or more HIV antibodies.

Also provided are further separate embodiments, each comprising the method of treating an HIV infection in a human through administration of a compound of Formula (I) and an HIV antibody, as just described, wherein in each of the separate embodiments the compound of Formula (I) is one compound selected from the group of Formula (I), (II), (IIa), (IIb), (III), (IIIa), (IIIa), (IV), (IVa), (IVb), (IVc), (IVd) or each of the individual compounds of the examples from Example 1 through Example 118; or a pharmaceutically acceptable salt thereof.

For each of the methods described herein comprising administering to a human in need thereof a pharmaceutically effective amount of a TLR8 modulating compound, and a pharmaceutically effective amount of an HIV antibody, there are further embodiments directed to the sequence of administering each agent.

In one embodiment within each method the TLR8 modulating compound and the HIV antibody may be administered to the human together, such as each being administered in the same day. Pharmaceutically effective amounts of each agent can be administered on a specified regimen, such as once weekly, once every other week, once every three weeks, once per month, etc. In another embodiment within each method the initial doses of the TLR8 modulating compound and the HIV antibody may be administered to the human together, with subsequent administrations being at staggered time points. For instance, following an initial dose of each agent, the TLR8 compound could be administered to the human every day or in 2-, 3-, 4-, 5-, 6-, 7-, 8-, 9-, 10-, 11, 12-, 13-, 14-, or 15 day intervals, wherein the HIV antibody is administered once per week, twice per month, monthly, etc.

In another embodiment within each method the TLR8 modulating compound may be administered in an initial administration, with the HIV antibody being administered to the human in a subsequent administration, such as 1, 2, 3, 4, 5, 6, 7, 8, 9, 10, 11, 12, 13, 14, or 15 days following administration of the TLR8 modulating compound. In another embodiment within each method the HIV antibody may be administered in an initial administration, with the TLR8 modulating compound being administered to the human in a subsequent administration, such as 1, 2, 3, 4, 5, 6, 7, 8, 9, 10, 11, 12, 13, 14, or 15 days following administration of the TLR8 modulating compound.

Similar regimens of administration are understood for the methods described herein comprising administering to a human in need thereof a pharmaceutically effective amount of a TLR8 modulating compound, including those of Formula (J), (I), (II), (IIa), (IIb), (III), (IIIa), (IIIa), (IV), (IVa), (IVb), (IVc), or (IVd), or each of the individual compounds of the examples from Example 1 through Example 120, etc., a pharmaceutically effective amount of an HIV antibody, and a combination antiretroviral therapy there are further embodiments directed to the sequence of administering each agent. For instance, in instances in which the human in need thereof is already being administered an antiretroviral combination therapy, such as a cART or HAART regimen, the TLR8 modulating compound and the HIV antibody may be added to the ongoing antiretroviral combination therapy using any of the regimens described for them above. In additional embodiments within each method, the TLR8 modulating compound may be administered as the initial agent, followed by subsequent administrations of the agents of the combination antiretroviral therapy and the HIV antibody. In additional embodiments within each method, the TLR8 modulating compound and the HIV antibody may be administered to the human in need thereof in one of the regimens described for them above and the agents of the combination antiretroviral therapy may be administered at a later point in time.

Provided is a method of treating an HIV infection in a human, the method comprising:
a) administering to a human in need thereof a pharmaceutically effective amount of a combination antiretroviral therapy;
b) administering to a human in need thereof a pharmaceutically effective amount of a TLR8 modulating compound, or a pharmaceutically acceptable salt thereof; and
c) administering to the human a pharmaceutically effective amount of an HIV antibody.

Provided is a method of treating an HIV infection in a human, the method comprising:
a) administering to a human in need thereof a pharmaceutically effective amount of a combination antiretroviral therapy;
b) administering to a human in need thereof a pharmaceutically effective amount of a compound of Formula (I) or a pharmaceutically acceptable salt thereof; and
c) administering to the human a pharmaceutically effective amount of an HIV antibody.

Also provided are further separate embodiments, each comprising the method of treating an HIV infection in a human through administration of a combination antiretroviral therapy, a compound of Formula (I), and an HIV antibody, as just described, wherein in each of the separate embodiments the compound of Formula (I) is one compound selected from the group of Formula (I), (II), (IIa), (IIb), (III), (IIIa), (IIIa), (IV), (IVa), (IVb), (IVc), (IVd)or each of the individual compounds of the examples from Example 1 through Example 118; or a pharmaceutically acceptable salt thereof.

Also provided is a method of treating an HIV infection in a human, the method comprising:
a) administering to a human in need thereof a pharmaceutically effective amount of an antiretroviral agent;
b) administering to a human in need thereof a pharmaceutically effective amount of a TLR8 modulating compound, or a pharmaceutically acceptable salt thereof; and
c) administering to the human a pharmaceutically effective amount of an HIV antibody.

Also provided is a method of treating an HIV infection in a human, the method comprising:
a) administering to a human in need thereof a pharmaceutically effective amount of a combination antiretroviral therapy;
b) administering to a human in need thereof a pharmaceutically effective amount of a compound of Formula (I), or a pharmaceutically acceptable salt thereof; and
c) administering to the human a pharmaceutically effective amount of an HIV antibody.

Provided is a method of treating an HIV infection in a human, the method comprising:
a) administering to a human in need thereof a pharmaceutically effective amount of a combination antiretroviral therapy regimen sufficient to lower the level of HIV detected in the human's blood or plasma to a specified level; and
b) administering to a human in need thereof a pharmaceutically effective amount of a compound of Formula (I), or a pharmaceutically acceptable salt thereof; and
c) administering to the human a pharmaceutically effective amount of an HIV antibody.

Also provided are twenty further separate embodiments, each comprising the method of treating an HIV infection in a human through administration of a combination antiretroviral therapy regimen sufficient to lower the level of HIV detected in the human's blood or plasma to a specified level, a compound of Formula (I), and an HIV antibody, as just described, wherein in each of the separate embodiments the compound of Formula II is one compound selected from the group of Formula (II), (IIa), (IIb), (III), (IIIa), (IIIa), (IV), (IVa), (IVb), (IVc), (IVd), or each of the individual compounds of the examples from Example 1 through Example 118; or a pharmaceutically acceptable salt thereof.

HIV antibodies useful in the methods herein include:

CD4-binding site-directed antibodies, including those that bind to the CD4 binding site on gp120 such as VRC01, VRCO2, VRC03, VRC04, VR0007, b12, HJ16, NIH45-46, 3BNC60, BNC62, 3BNC117, 12A12, 12A21, 12A30, VRC-PG04, VRC-CH30, VRC-CH31, VRC-CH32, VRC-CH33, VRC-CH34, VRC-PG04, VRC-PG04b, 8ANC131, 8ANC37, 8ANC134, CH103, CH104, CH105, CH106, 3BNC117, 3BNC60, NIH45, NIH46, 12A12, 12A21, 8ANC131, 8ANC134, 1NC9, 162530, 7B2, and A32;

Gp-120 variable region 1 and variable region 2 (V1/V2)-directed antibodies, such as PG9, PG16, CH01-04, PGT141, PGT142, PGT143, PGT144, PGT145, and CAP256-VRC26;

Glycan V3-directed antibodies, such as the PGT121 series of antibodies, including PGT121, PGT122, PGT123, PGT 124, PGT 125, PGT126, PGT127, PGT128, PGT130, PGT131, PGT-132, PGT135, PGT136, and PGT137, as well as 2G12;

membrane-proximal external region (MPER)-directed antibodies, such as the 2F5, Z13, 4E10, 10E8, PGT150 series of antibodies, M66.6, CAP206-CH12, and 10E81. PG and PGT antibodies are described in WO 2010/107939 and WO 2012/030904.

Additional antibodies for use with the methods herein include PGT-138, PGT-139, PGT-133, PGT-134, PGT-135, PGT-136, PGT-137, PGT-141, PGT-142, PGT-143, PGT-144, PGT-145, PGT-151, PGT-152, PGT-153, PGT-154, PGT-155, PGT-156, PGT-157, and PGT-158.

Additional antibodies for use with the methods herein include bi-specific antibodies. Such bi-specific antibodies will have at least one variable region recognizing a portion of the HIV virus, e.g., gp120 or gp41. In certain embodiments, the bi-specific antibodies include a second variable region recognizing a memory cell surface, such as CD3 or CD4. Exemplary bi-specific antibodies include but are not limited to those inducing the redirected CD8 T cell-dependent lysis of HIV infected cells such as those recognizing HIV gp120/41 envelope (arm A) and CD3 receptor (arm B) as described in WO2013163427 A1. Furthermore, the bi-specific antibodies may include additional platforms such as BiTEs (Amgen), DARTs (Macrogenics), Duobodies (GenMab) as well as other platforms (Xencor, Sanofi, etc.). Additional examples of bispecific antibodies may include those inducing redirected NK cell-mediated lysis of HIV infected cells such as those recognizing HIV gp120/41 envelope (arm A) and NKG2D receptor (arm B) based on Affimed platform.

Additional antibodies for use with the methods herein include bi-specific antibodies such as those inducing the redirected CD8 T cell-dependent lysis of HIV infected cells such as those recognizing HIV gp120/41 envelope (arm A) and CD3 receptor (arm B) as described in W02013163427 Al. Furthermore, the bi-specific antibodies may include additional platforms such as BiTEs (Amgen), DARTs (Macrogenics), Duobodies (GenMab) as well as other platforms (Xencor, Sanofi, etc.). Additional examples of bispecific antibodies may include those inducing redirected NK cell-mediated lysis of HIV infected cells such as those recognizing HIV gp120/41 envelope (arm A) and NKG2D receptor (arm B) based on Affimed platform.

The antibody delivery method may include any form of injection of pharmaceutically formulated antibody or a persistent expression in tissues, eg intramuscularly such as that described in Balazs, Nature (2011), 481 (7379), pp. 81-4 and in Balazs, Nat Med. (2014), 20(3), pp. 296-300.

### Immunomodulatory Antibodies and Small Molecule Agents

Specific antibodies for use include also immunomodulatory monoclonal antibodies:
inhibitory anti-PD-1 mAbs such as Nivolimumab (BMS-936558 or MDX1106), Pembrolizumab (MK-3475); inhibitory anti-PD-L1 mAbs such as BMS-936559. MPDL3280A , MEDI4736, MSB0010718C, and MDX1105-01; inhibitory anti-CTLA-4 mAbs, such as Ipilimumab, and Tremilimumab;
inhibitory anti-Tim3 mAbs, such as those from Tesaro, Inc.; inhibitory anti-LAG-3 mAbs, such as BMS-986016, IMP321; inhibitory anti-KIR mAbs, such as Lirilumab (IPH2102/BMS-986015);
stimulatory anti-CD27 mAbs, such as CDX-1127; stimulatory anti-CD40 mAbs, such CP-870,893, and BMS-986090; inhibitory anti-CD47 mAbs, such as those seen in Tseng et al, Proc Natl Acad Sci USA. Jul. 2, 2013; 110(27): 11103-11108; stimulatory anti-CD134 (OX40) mAbs, such as MEDI-6469 or those seen in WO-2009079335, and WO-2006121810;
Stimulatory anti-CD137 mAbs, such as BMS-663513; PF-05082566; additional antibodies against immunomodulatory receptors such as TIGIT, BTLA and others as listed in Chen and Flies, Nat. Rev. Immunol. 13, 227-42 (2013); and nucleic acid encoding fusion proteins that prevent or inhibit HIV infection, administered by themselves or via a vector, such as a VEE, SFV, AAV, scAAV, or HPV vector, including those described in U.S. 2011/0305670A1 (Farzan), such as the eCD4-Ig, eCD4-Ig.A, eCD4-Ig.B, CD4-Ig, E1-Ig, E2-Ig, E3-Ig, e3-CD4-Ig, e4-CD4-Ig, and CCR5mim-Ig, including AAV-expressed eCD4-Ig and scAAV-expressed eCD4-Ig.

Small molecule immunomodulatory agents to used in combination with TLR8 modulating compounds include for example indole oxygenase inhibitors (also known as inhbitors of IDO, IDO, indoleamine-2,3-dioxygenase, indoleamine dioxygenase-1, or indoleamine-pyrrole 2,3-dioxygenase), such as 1-methyl-D-tryptophan, NLG919, epacadostat (INCB24360), F-001287, resminostat (4SC-201), SN-35837, NLG-919, GDC-0919, and indoximod, PI3K delta inhibitors such as Idelalisib, GS-9820, and GS-9901, and other TLR8 agonist such as VTX-1463 or VTX-2337.

In certain embodiments, the methods described herein comprise the further step of administering to the human in need thereof a pharmaceutically effective amount of an immunomodulatory monoclonal antibody or immunomodulatory small molecule agent. In certain embodiments, the immunomodulatory monoclonal antibody is an inhibitory anti-PD-1 monoclonal antibody or inhibitory anti-PD-L1 monoclonal antibody. In certain embodiments, the inhibitory anti-PD-1 monoclonal antibody is Nivolimumab, Pembrolizumab, BMS-936559, MPDL3280A, MED14736, MSB0010718C, or MDX1105-01. In certain embodiments, the immunomodulatory small molecule agent is an IDO inhibitor. In certain embodiments, the immunomodulatory small molecule agent is 1-methyl-D-tryptophan, NLG919, epacadostat, F-001287, resminostat, SN-35837, NLG-919, GDC-0919, or indoximod.

Provided is a method of eliminating an HIV infection in a human, the method comprising:
a) administering to a human in need thereof a pharmaceutically effective amount of a TLR8 modulating compound, or a pharmaceutically acceptable salt thereof; and
b) administering to the human a pharmaceutically effective amount of an HIV antibody or small molecule immunomodulatory agent.

Provided is a method of eliminating an HIV infection in a human, the method comprising:
a) administering to a human in need thereof a pharmaceutically effective amount of a compound of Formula (I), or a pharmaceutically acceptable salt thereof; and
b) administering to the human a pharmaceutically effective amount of an HIV antibody or small molecule immunomodulatory agent.

Also provided is a method of eliminating an HIV infection in a human, the method comprising:
a) administering to a human in need thereof a pharmaceutically effective amount of a combination antiretroviral therapy;
b) administering to a human in need thereof a pharmaceutically effective amount of a compound of Formula (I), or a pharmaceutically acceptable salt thereof; and
c) administering to the human a pharmaceutically effective amount of an HIV antibody or small molecule immunomodulatory agent.

Also provided are separate embodiments comprising the use of a pharmaceutically effective amount of a TLR8 modulating compound as described herein, or a pharmaceutically acceptable salt thereof, for one or more of:
a) use in treating an HIV infection in a human;
b) use in treating an HIV infection in a virologically suppressed human;
c) use in inducing HIV gene expression in a human infected with HIV;
d) use in inducing HIV gene expression in a human infected with HIV wherein active HIV gene expression in the human has been suppressed by administration of antiretroviral therapy;
e) use in inducing HIV gene expression in a latent HIV reservoir in a human infected with HIV;
f) use in enhancing HIV gene expression in HIV infected cells in a human infected with HIV;
g) use in lowering the chronic set point of HIV viral load in a human infected with HIV;
h) use in inducing transient HIV-1 viremia in a virologically suppressed human infected with HIV-1;
i) use in reducing HIV viremia in a human infected with HIV;
j) use in enhancing immune cell activity and increasing HIV gene expression in a human infected with HIV;
k) use in enhancing the efficacy of an antiviral agent in a human infected with HIV;
l) use in inducing transient HIV-1 viremia in a virologically suppressed human infected with HIV-1;
m) use in enhancing the efficacy of an HIV vaccine; or
n) use in eliminating an HIV infection in a human.

Also provided are separate embodiments comprising the use of a pharmaceutically effective amount of a TLR8 modulating compound as described herein, or a pharmaceutically acceptable salt thereof, for one or more of:
a) the treatment of an HIV infection in a human;
b) the treatment of an HIV infection in a virologically suppressed human;
c) the induction of HIV gene expression in a human infected with HIV;
d) the induction of HIV gene expression in a human infected with HIV wherein active HIV gene expression in the human has been suppressed by administration of antiretroviral therapy;
e) the induction of HIV gene expression in a latent HIV reservoir in a human infected with HIV;
f) the enhancement of HIV gene expression in HIV infected cells in a human infected with HIV;
g) lowering the chronic set point of HIV viral load in a human infected with HIV;
h) the induction of transient HIV-1 viremia in a virologically suppressed human infected with HIV-1;
i) for reducing HIV viremia in a human infected with HIV;
j) the enhancement of immune cell activity and increase of HIV gene expression in a human infected with HIV;
k) the enhancement of the efficacy of an antiviral agent in a human infected with HIV;
l) use in inducing transient HIV-1 viremia in a virologically suppressed human infected with HIV-1;
m) the enhancement of the efficacy of an HIV vaccine; or
n) the elimination of an HIV infection in a human.

### Pharmaceutical Compositions

Provided herein are pharmaceutical compositions that may be used in the methods discussed above.

Provided is a pharmaceutical composition comprising:
a) a pharmaceutically effective amount of a combination antiretroviral therapy;
b) a pharmaceutically effective amount of a TLR8 modulating compound, or a pharmaceutically acceptable salt thereof; and
c) a pharmaceutically acceptable excipient.

Provided herein are pharmaceutical compositions that may be used in the methods discussed above.

Provided is a pharmaceutical composition comprising:
a) a pharmaceutically effective amount of a combination antiretroviral therapy;
b) a pharmaceutically effective amount of a compound of Formula (I), or a pharmaceutically acceptable salt thereof; and
c) a pharmaceutically acceptable excipient.

Also provided are twenty further separate embodiments, each comprising pharmaceutical compositions, as just defined, wherein the compound of Formula (I) is a compound of Formula (II), (IIa), (IIb), (III), (IIIa), (IIIa), (IV), (IVa), (IVb), (IVc), (IVd), or each of the individual compounds of the examples from Example 1 through Example 118; or a pharmaceutically acceptable salt thereof. Additionally, as previously disclosed, a TLR8 modulating compound includes any compound of Formula (J), (I), (II), (IIa), (IIb), (III), (IIIa), (IIIa), (IV), (IVa), (IVb), (IVc), or (IVd), or each of the individual compounds of the examples from Example 1 through Example 120, in separate embodiments.

### Elvitedravir/cobicistat/emtricitabine/TDF or TAF/TLR8 Modulator Combinations

Pharmaceutically effective amounts of the TLR8 modulating compounds, including those of Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof, can be combined with a pharmaceutically effective amounts of elvitegravir, cobicistat, emtricitabine, and tenofovir disoproxil fumarate (TDF) or tenofovir alafenamide (TAF) for use in the methods of treatment discussed herein. For instance, as separate dosage forms, a pharmaceutically effective dose of the TLR8 modulating compounds may be combined in a treatment regimen with a STRIBILD® tablet (Gilead Sciences, Inc.) containing 150 mg elvitegravir, 150 mg cobicistat, 200 mg emtricitabine, and 300 mg tenofovir disoproxil fumarate.

Provided is a pharmaceutical composition comprising:

a) a pharmaceutically effective amount of elvitegravir;

b) a pharmaceutically effective amount of cobicistat;

c) a pharmaceutically effective amount of emtricitabine;

d) a pharmaceutically effective amount of tenofovir disoproxil fumarate;

e) a pharmaceutically effective amount of a TLR8 modulating compound, or a pharmaceutically acceptable salt thereof; and

f) a pharmaceutically acceptable excipient.

Provided is a pharmaceutical composition comprising:

a) a pharmaceutically effective amount of elvitegravir;

b) a pharmaceutically effective amount of cobicistat;

c) a pharmaceutically effective amount of emtricitabine;

d) a pharmaceutically effective amount of tenofovir disoproxil fumarate;

e) a pharmaceutically effective amount of a compound of Formula (I), or a pharmaceutically acceptable salt thereof; and

f) a pharmaceutically acceptable excipient.

Also provided is a pharmaceutical composition comprising:

a) a pharmaceutically effective amount of elvitegravir;

b) a pharmaceutically effective amount of cobicistat;

c) a pharmaceutically effective amount of emtricitabine;

d) a pharmaceutically effective amount of tenofovir alafenamide;

e) a pharmaceutically effective amount of a TLR8 modulating compound, or a pharmaceutically acceptable salt thereof; and

f) a pharmaceutically acceptable excipient.

Also provided is a pharmaceutical composition comprising:

a) a pharmaceutically effective amount of elvitegravir;

b) a pharmaceutically effective amount of cobicistat;

c) a pharmaceutically effective amount of emtricitabine;

d) a pharmaceutically effective amount of tenofovir alafenamide;

e) a pharmaceutically effective amount of a compound of Formula (I), or a pharmaceutically acceptable salt thereof; and

f) a pharmaceutically acceptable excipient.

Provided herein is a series of lists of combinations of antiviral agents and TLR8 modulating compounds in ranges of doses and/or specific doses. Each indicated combination is an embodiment, with each embodiment providing a pharmaceutical composition comprising the pharmaceutically effective amounts of the combined antiviral agents and TLR8 modulating compounds, or a pharmaceutically acceptable salt thereof, alone or combined with one or more pharmaceutically acceptable carriers or excipients.

Each such individual combination of ranges of doses and/or specific doses also provides a pharmaceutically effective amount of the antiviral agents and TLR8 modulating compounds that may be used in each of the methods described herein. Each such individual combination of ranges of doses and/or specific doses described herein administered to a human in need thereof in each of the individual methods described herein comprises a separate embodiment for the method in question. For instance, the use of combination A-1 with the method described for treating an HIV infection, above provides a method of treating an HIV infection in a human comprising:
a) administering to a human in need thereof from 100 mg to 200 mg elvitegravir, 100 mg to 200 mg cobicistat, and from 250 mg to 350 mg TDF to lower the level of HIV detected in the human's blood or plasma from a first level to a second level, the second level comprising a lower concentration of HIV in the human's blood or plasma than the concentration of HIV in the human's blood or plasma in the first level; and
b) administering to the human a pharmaceutically effective amount of a compound of Formula (I), or a pharmaceutically acceptable salt thereof.
   For each of these separate methods there are further embodiments directed to the sequence of administering each agent.

In one embodiment within each method the TLR8 modulating compound (TLR8 modulating agent) and the antiviral agent or agents may be administered to the human together, such as each being administered in the same day. Pharmaceutically effective amounts of each agent can be administered on a specified regimen, such once daily, twice daily, once weekly, once every two weeks, once every three weeks, once per month, once every two months, etc. In another embodiment within each method the initial doses of the TLR8 modulating compound and the antiviral agent or agents may be administered to the human together, with subsequent administrations being at staggered time points. For instance, following an initial dose of each agent, the TLR8 compound could be administered to the human every day or in 2-, 3-, 4-, 5-, 6-, 7-, 8-, 9-, 10-, 11, 12-, 13-, 14-, or 15-day intervals, wherein the HIV antibody is administered once per week, twice per month, monthly, etc., as can each of the individual antiviral agents.

In another embodiment within each method the TLR8 modulating compound may be administered in an initial administration, with the antiviral agent or agents being administered to the human in a subsequent administration, such as 1, 2, 3, 4, 5, 6, 7, 8, 9, 10, 11, 12, 13, 14, or 15 days following administration of the TLR8 modulating compound. In another embodiment within each method the antiviral agents may be administered in an initial administration, with the TLR8 modulating compound being administered to the human in a subsequent administration, such as 1, 2, 3, 4, 5, 6, 7, 8, 9, 10, 11, 12, 13, 14, or 15 days following administration of the TLR8 modulating compound. In another embodiment within each method administration of the TLR8 modulating compound may be added to an existing antiviral agent regimen.

The TLR8 modulating agent may being administered to the human daily with the antiviral agents or, in conjunction with daily antiviral agent administrations, the subsequent administration of TLR8 modulating agent may follow a staggered regimen, such as every 1, 2, 3, 4, 5, 6, 7, 8, 9, 10, 11, 12, 13, 14, or 15 days. In addition, administration of an antiviral agent or agents to a human infected with HIV may be added in a regimen following administration of the TLR8 modulating agent. For instance, the TLR8 modulating agent (compound) may be administered in a single dose, in a series of once or twice daily doses, or in a series of doses staggered across a period of time, followed by administration to the human of a regimen of an antiviral agent or agents.

Provided are examples separate pharmaceutical compositions and combinations, below, wherein each composition comprises a pharmaceutically acceptable excipient and the amounts of elvitegravir, cobicistat, emtricitabine, tenofovir disoproxil fumarate (TDF) or tenofovir alafenamide (TAF) as described in Table A, and a TLR8 modulating compound as described herein,. The pharmaceutical compositions comprise the pharmaceutically effective amounts of each agent in the composition and a pharmaceutically acceptable excipient. The pharmaceutical combinations in each of the pharmaceutical compositions herein may be utilized together in the methods of treatment described herein, with the listed pharmaceutical agents of each composition being administered to a human in need thereof as a single pharmaceutical composition, such as a tablet or oral liquid, or the agents may be administered separately or in any of the possible combinations. For instance, a pharmaceutically effective amount of a compound of Formula (I), may be administered to a human in need thereof in a first tablet in conjunction with the administration of a second tablet containing the remaining agents of the combination, such as a STRIBILD® tablet.

Also provided is a pharmaceutical kit comprising:
1) a series of daily doses of a single pharmaceutical composition comprising:
   a. a pharmaceutically effective amount of elvitegravir;
   b. a pharmaceutically effective amount of TDF;
   c. a pharmaceutically effective amount of a TLR8 modulating compound, or a pharmaceutically acceptable salt thereof;
   d. a pharmaceutically effective amount of cobicistat;
   e. a pharmaceutically effective amount of emtricitabine; and
   f. a pharmaceutically acceptable excipient; and
2) directions for the administration of the daily doses of the pharmaceutical composition.

Also provided is a pharmaceutical kit comprising:
1) a series of daily doses of a single pharmaceutical composition comprising:
   a. a pharmaceutically effective amount of elvitegravir;
   b. a pharmaceutically effective amount of TAF;
   c. a pharmaceutically effective amount of a TLR8 modulating compound , or a pharmaceutically acceptable salt thereof;
   d. a pharmaceutically effective amount of cobicistat;
   e. a pharmaceutically effective amount of emtricitabine; and
   f. a pharmaceutically acceptable excipient; and
2) directions for the administration of the daily doses of the pharmaceutical composition.

Further provided is a pharmaceutical kit comprising:
1) a series of doses of a first pharmaceutical composition comprising:
   a) a pharmaceutically effective amount of elvitegravir;
   b) a pharmaceutically effective amount of cobicistat
   c) a pharmaceutically effective amount of TDF;
   d) a pharmaceutically effective amount of emtricitabine; and
   e) a pharmaceutically acceptable excipient; and
2) a series of doses of a second pharmaceutical composition comprising a pharmaceutically effective amount of a TLR8 modulating compound, or a pharmaceutically acceptable salt thereof and a pharmaceutically acceptable excipient; and
3) directions for the administration of the doses of the first and second pharmaceutical composition; wherein the first and second pharmaceutical compositions are both administered once daily.

Another embodiment comprises the kit immediately above wherein the first and second pharmaceutical compositions are both administered twice daily.

Further provided is a pharmaceutical kit comprising:
1) a series of doses of a first pharmaceutical composition comprising:
   a) a pharmaceutically effective amount of elvitegravir;
   b) a pharmaceutically effective amount of cobicistat
   c) a pharmaceutically effective amount of TAF;
   d) a pharmaceutically effective amount of emtricitabine; and
   e) a pharmaceutically acceptable excipient; and
2) a series of doses of a second pharmaceutical composition comprising a pharmaceutically effective amount of a TLR8 modulating compound, or a pharmaceutically acceptable salt thereof and a pharmaceutically acceptable excipient; and
3) directions for the administration of the doses of the first and second pharmaceutical composition; wherein the first and second pharmaceutical compositions are both administered once daily.

Another embodiment within each of the kits above comprises the kit wherein the first and second pharmaceutical compositions are both administered twice daily.

Another embodiment within each of the kits above comprises the kit wherein the first pharmaceutical composition is administered twice daily and the second pharmaceutical composition is administered less than daily. Further embodiments comprise those wherein the first pharmaceutical composition is administered daily and the second pharmaceutical composition is administered, respectively, every other day, or every 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, or 30^{th} day.

Within the first embodiment of the pharmaceutical kit above comprising pharmaceutically effective amounts of elvitegravir, cobicistat, emtricitabine, tenofovir disoproxil fumarate, and a compound of Formula (I), there is a further embodiment comprising the kit, as described, wherein the first pharmaceutical composition comprises 150 mg of elvitegravir, 150 mg of cobicistat, 200 mg of emtricitabine, and 300 mg of tenofovir disoproxil fumarate, and the second pharmaceutical composition comprises a pharmaceutically effective amount of a compound of Formula (I), or a pharmaceutically acceptable salt thereof.

Within the embodiment of the pharmaceutical kit above, there is a further embodiment comprising the kit, as described, wherein the first pharmaceutical composition comprises 150 mg of elvitegravir, 150 mg of cobicistat, 200 mg of emtricitabine, and 300 mg of tenofovir disoproxil fumarate, and the second pharmaceutical composition comprises a pharmaceutically effective amount of a compound of Formula (I), or a pharmaceutically acceptable salt thereof.

Within the second embodiment of the pharmaceutical kit above comprising pharmaceutically effective amounts of elvitegravir, cobicistat, emtricitabine, TAF, and a compound of Formula (I), there is a further embodiment comprising the kit, as described, wherein the first pharmaceutical composition comprises 150 mg of elvitegravir, 150 mg of cobicistat, 200 mg of emtricitabine, and 10 mg of TAF, and the second pharmaceutical composition comprises a pharmaceutically effective amount of a compound of Formula (I) or a pharmaceutically acceptable salt thereof.

Within the embodiment of the pharmaceutical kit above, there is a further embodiment comprising the kit, as described, wherein the first pharmaceutical composition comprises 150 mg of elvitegravir, 150 mg of cobicistat, 200 mg of emtricitabine, and 10 mg of TAF, and the second pharmaceutical composition comprises a pharmaceutically effective amount of a compound of Formula (I) or a pharmaceutically acceptable salt thereof.

Combinations of emtricitabine/TDF/TLR8 or emtricitabine/TAF/TLR8 Modulators

Also provided is a pharmaceutical composition comprising:
a) a pharmaceutically effective amount of emtricitabine;
b) a pharmaceutically effective amount of tenofovir disoproxil fumarate;
c) a pharmaceutically effective amount of TLR8 modulating compound, or a pharmaceutically acceptable salt thereof; and
d) a pharmaceutically acceptable excipient.

Also provided is a pharmaceutical composition comprising:
a) a pharmaceutically effective amount of emtricitabine;
b) a pharmaceutically effective amount of TAF;
c) a pharmaceutically effective amount of TLR8 modulating compound, or a pharmaceutically acceptable salt thereof; and
d) a pharmaceutically acceptable excipient.
   Combinations of emtricitabine/TDF/TLR8 Modulators/ralteciravir

Provided are a pharmaceutical combination and a composition, each comprising:
a) a pharmaceutically effective amount of emtricitabine;
b) a pharmaceutically effective amount of tenofovir disoproxil fumarate;
c) a pharmaceutically effective amount of a TLR8 modulating compound, or a pharmaceutically acceptable salt thereof;
d) a pharmaceutically effective amount of an integrase strand transfer inhibitor; and
e) a pharmaceutically acceptable excipient.

Provided are a pharmaceutical combination and a composition, each comprising:
a) a pharmaceutically effective amount of emtricitabine;
b) a pharmaceutically effective amount of tenofovir disoproxil fumarate;
c) a pharmaceutically effective amount of a compound of Formula (I), or a pharmaceutically acceptable salt thereof;
d) a pharmaceutically effective amount of an integrase strand transfer inhibitor; and
e) a pharmaceutically acceptable excipient.

Provided are a pharmaceutical combination and a composition, each comprising:
a) a pharmaceutically effective amount of emtricitabine;
b) a pharmaceutically effective amount of tenofovir alafenamide;
c) a pharmaceutically effective amount of a TLR8 modulating compound, or a pharmaceutically acceptable salt thereof;
d) a pharmaceutically effective amount of an integrase strand transfer inhibitor; and
e) a pharmaceutically acceptable excipient.

Provided are a pharmaceutical combination and a composition, each comprising:
a) a pharmaceutically effective amount of emtricitabine;
b) a pharmaceutically effective amount of tenofovir alafenamide;
c) a pharmaceutically effective amount of a compound of Formula (I), or a pharmaceutically acceptable salt thereof;
d) a pharmaceutically effective amount of an integrase strand transfer inhibitor; and
e) a pharmaceutically acceptable excipient.

Also are a pharmaceutical combination and a composition, each comprising:
a) a pharmaceutically effective amount of emtricitabine;
b) a pharmaceutically effective amount of tenofovir disoproxil fumarate;
c) a pharmaceutically effective amount of a TLR8 modulating compound, or a pharmaceutically acceptable salt thereof;
d) a pharmaceutically effective amount of raltegravir; and
e) a pharmaceutically acceptable excipient.

Also provided are a pharmaceutical combination and a composition, each comprising:
a pharmaceutically effective amount of emtricitabine;
a) a pharmaceutically effective amount of tenofovir disoproxil fumarate;
b) a pharmaceutically effective amount of a compound of Formula (I), or a pharmaceutically acceptable salt thereof;
c) a pharmaceutically effective amount of raltegravir; and
d) a pharmaceutically acceptable excipient.

Also provided are a pharmaceutical combination and a composition, each comprising:
a pharmaceutically effective amount of emtricitabine;
b) a pharmaceutically effective amount of tenofovir alafenamide;
c) a pharmaceutically effective amount of a TLR8 modulating compound, or a pharmaceutically acceptable salt thereof;

d) a pharmaceutically effective amount of raltegravir; and
e) a pharmaceutically acceptable excipient.

Also provided are a pharmaceutical combination and a composition, each comprising:
a) a pharmaceutically effective amount of emtricitabine;
b) a pharmaceutically effective amount of tenofovir alafenamide;
c) a pharmaceutically effective amount of a compound of Formula (I), or a pharmaceutically acceptable salt thereof;
d) a pharmaceutically effective amount of raltegravir; and
e) a pharmaceutically acceptable excipient.

Also provided is a pharmaceutical kit, the kit comprising:
1) a series of daily doses of a single pharmaceutical composition comprising:
   a. a pharmaceutically effective amount of emtricitabine;
   b. a pharmaceutically effective amount of tenofovir disoproxil fumarate;
   c. a pharmaceutically effective amount of a compound of Formula (I), or a pharmaceutically acceptable salt thereof;
   d. a pharmaceutically effective amount of raltegravir; and
   e. a pharmaceutically acceptable excipient; and
2) directions for the administration of the daily doses of the pharmaceutical composition.

Also provided are separate pharmaceutical kits, as just described, wherein the pharmaceutical composition comprises, in each of the separate pharmaceutical kits, one of the pharmaceutical compositions described above having raltegravir as a component or element.

Further provided is a pharmaceutical kit, the kit comprising:
1) a series of doses of a first pharmaceutical composition comprising:
   a) a pharmaceutically effective amount of emtricitabine;
   b) a pharmaceutically effective amount of tenofovir disoproxil fumarate;
   c) a pharmaceutically effective amount of a compound of Formula (I), or a pharmaceutically acceptable salt thereof;
   d) a pharmaceutically acceptable excipient; and
2) a series of doses of a second pharmaceutical composition comprising a pharmaceutically effective amount of raltegravir and a pharmaceutically acceptable excipient; and
3) directions for the administration of the doses of the first and second pharmaceutical composition; wherein the first and second pharmaceutical compositions are both administered once daily.

Further provided is a pharmaceutical kit, the kit comprising:
1. a series of daily doses of a first pharmaceutical composition comprising:
   b) a pharmaceutically effective amount of emtricitabine;
   c) a pharmaceutically effective amount of tenofovir disoproxil fumarate;
   d) a pharmaceutically effective amount of a compound of Formula (I), or a pharmaceutically acceptable salt thereof;
   e) a pharmaceutically acceptable excipient; and
2) a series of daily doses of a second pharmaceutical composition comprising a pharmaceutically effective amount of raltegravir and a pharmaceutically acceptable excipient; and
3) directions for the administration of the daily doses of the first and second pharmaceutical composition; wherein the first and second pharmaceutical compositions are both administered twice daily.

Further provided is a pharmaceutical kit, the kit comprising:
1) a series of doses of a first pharmaceutical composition comprising:
   a) a pharmaceutically effective amount of emtricitabine;
   b) a pharmaceutically effective amount of tenofovir disoproxil fumarate;
   c) a pharmaceutically effective amount of a compound of Formula (I), or a pharmaceutically acceptable salt thereof;
   d) a pharmaceutically acceptable excipient; and
2) a series of doses of a second pharmaceutical composition comprising a pharmaceutically effective amount of raltegravir and a pharmaceutically acceptable excipient; and
3) directions for the administration of the doses of the first and second pharmaceutical composition; wherein the first pharmaceutical composition is administered once daily and second pharmaceutical composition is administered twice daily.

Further provided is a pharmaceutical kit, the kit comprising:
1) a series of doses of a first pharmaceutical composition comprising:
   a) a pharmaceutically effective amount of emtricitabine;
   b) a pharmaceutically effective amount of tenofovir disoproxil fumarate;
   c) a pharmaceutically acceptable excipient; and
2) a series of doses of a second pharmaceutical composition comprising a pharmaceutically effective amount of raltegravir and a pharmaceutically acceptable excipient; and
3) a series of doses of a third pharmaceutical composition comprising a pharmaceutically effective amount of a compound of Formula II, or a pharmaceutically acceptable salt thereof; and directions for the administration of the doses of the first and second pharmaceutical composition; wherein each of the first pharmaceutical composition, the second pharmaceutical composition, and the third pharmaceutical composition is administered once daily.

Within the embodiment of the pharmaceutical kit immediately above, there is a further embodiment comprising the kit, as described, wherein the second pharmaceutical composition comprises from 10 mg to 500 mg of raltegravir. Within the embodiment of the pharmaceutical kit immediately above, there is a further embodiment comprising the kit, as described, wherein the second pharmaceutical composition comprises from 300 mg to 500 mg of raltegravir. Within the embodiment of the pharmaceutical kit immediately above, there is a another embodiment comprising the kit, as described, wherein the second pharmaceutical composition comprises from 350 mg to 450 mg of raltegravir. Within the embodiment of the pharmaceutical kit immediately above, there is another embodiment comprising the kit, as described, wherein the second pharmaceutical composition comprises 400 mg of raltegravir.

Further provided is a pharmaceutical kit, the kit comprising:
1) a series of doses of a first pharmaceutical composition comprising:
   a) a pharmaceutically effective amount of emtricitabine;
   b) a pharmaceutically effective amount of tenofovir disoproxil fumarate;
   c) a pharmaceutically acceptable excipient; and
2) a series of doses of a second pharmaceutical composition comprising a pharmaceutically effective amount of raltegravir and a pharmaceutically acceptable excipient; and
3) a series of doses of a third pharmaceutical composition comprising a pharmaceutically effective amount of a compound of Formula (I), or a pharmaceutically acceptable salt thereof; and
4) directions for the administration of the doses of the first and second pharmaceutical composition; wherein the first pharmaceutical composition and third pharmaceutical composition are each administered once daily and the second pharmaceutical composition is administered twice daily.

Within the embodiment of the pharmaceutical kit above, there is a further embodiment comprising the kit, as described, wherein the first pharmaceutical composition comprises 200 mg of emtricitabine and 300 mg of tenofovir disoproxil fumarate, the second pharmaceutical composition comprises from 10 mg to 500 mg of raltegravir, and the third pharmaceutical composition comprises a pharmaceutically acceptable amound of a compound of Formula (I), or a pharmaceutically acceptable salt thereof.

Within the embodiment of the pharmaceutical kit above, there is a further embodiment comprising the kit, as described, wherein the first pharmaceutical composition comprises

200 mg of emtricitabine and 300 mg of tenofovir disoproxil fumarate, the second pharmaceutical composition comprises from 15 mg to 35 mg of raltegravir, and the third pharmaceutical composition comprises a pharmaceutically acceptable amound of a compound of Formula (I), or a pharmaceutically acceptable salt thereof.

Within the embodiment of the pharmaceutical kit above, there is a further embodiment comprising the kit, as described, wherein the first pharmaceutical composition comprises

200 mg of emtricitabine and 300 mg of tenofovir disoproxil fumarate, the second pharmaceutical composition comprises from 90 mg to 110 mg of raltegravir, and the third pharmaceutical composition comprises a pharmaceutically acceptable amound of a compound of Formula (I), ora pharmaceutically acceptable salt thereof.

Within the embodiment of the pharmaceutical kit above, there is a further embodiment comprising the kit, as described, wherein the first pharmaceutical composition comprises

200 mg of emtricitabine and 300 mg of tenofovir disoproxil fumarate, the second pharmaceutical composition comprises from 375 mg to 425 mg of raltegravir, and the third pharmaceutical composition comprises a pharmaceutically effective amount of a compound of Formula (I), or a pharmaceutically acceptable salt thereof.

Combinations of emtricitabine/TDF/TLR8 Modulators/dolutegravir

Pharmaceutically effective amounts of the TLR8 modulating compounds, including those of Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof, can be combined with pharmaceutically effective amounts of emtricitabine, TDF or TAF, and dolutegravir for use in the methods of treatment discussed herein. For instance, as separate dosage forms, a pharmaceutically effective dose of the TLR8 modulating compounds may be combined in a treatment regimen with a TRUVADA® tablet (200 mg of emtricitabine and 300 mg of tenofovir disoproxil fumarate), which is available from Gilead Sciences, and a TIVICAY® tablet (50 mg dolutegravir), which is available from GlaxoSmithKline.

Also provided is a pharmaceutical composition comprising:
a) a pharmaceutically effective amount of emtricitabine;
b) a pharmaceutically effective amount of tenofovir disoproxil fumarate;
c) a pharmaceutically effective amount of a TLR8 modulating compound, or a pharmaceutically acceptable salt thereof;
d) a pharmaceutically effective amount of dolutegravir; and
e) a pharmaceutically acceptable excipient.

Also provided is a pharmaceutical composition comprising:
a) a pharmaceutically effective amount of emtricitabine;
b) a pharmaceutically effective amount of tenofovir alafenamide;
c) a pharmaceutically effective amount of a TLR8 modulating compound, or a pharmaceutically acceptable salt thereof;
d) a pharmaceutically effective amount of dolutegravir; and
e) a pharmaceutically acceptable excipient.

Also provided is a pharmaceutical composition comprising:
a) a pharmaceutically effective amount of emtricitabine;
b) a pharmaceutically effective amount of tenofovir disoproxil fumarate;
c) a pharmaceutically effective amount of a compound of Formula (I), or a pharmaceutically acceptable salt thereof;
d) a pharmaceutically effective amount of dolutegravir; and
e) a pharmaceutically acceptable excipient.

Also provided is a pharmaceutical composition comprising:
a) a pharmaceutically effective amount of emtricitabine;
b) a pharmaceutically effective amount of tenofovir alafenamide;
c) a pharmaceutically effective amount of a compound of Formula (I), or a pharmaceutically acceptable salt thereof;
d) a pharmaceutically effective amount of dolutegravir; and
e) a pharmaceutically acceptable excipient.

Also provided is a pharmaceutical kit, the kit comprising:
1) a series of daily doses of a single pharmaceutical composition comprising:
   a) a pharmaceutically effective amount of emtricitabine;
   b) a pharmaceutically effective amount of tenofovir disoproxil fumarate;
   c) a pharmaceutically effective amount of a TLR8 modulating compound, or a pharmaceutically acceptable salt thereof;
   d) a pharmaceutically effective amount of dolutegravir; and
   e) a pharmaceutically acceptable excipient; and
2) directions for the administration of the daily doses of the pharmaceutical composition.

Also provided is a pharmaceutical kit, the kit comprising:
3) a series of daily doses of a single pharmaceutical composition comprising:
   f) a pharmaceutically effective amount of emtricitabine;
   g) a pharmaceutically effective amount of tenofovir disoproxil fumarate;
   h) a pharmaceutically effective amount of a TLR8 modulating compound, or a pharmaceutically acceptable salt thereof;
   i) a pharmaceutically effective amount of dolutegravir; and
   j) a pharmaceutically acceptable excipient; and
4) directions for the administration of the daily doses of the pharmaceutical composition.

Also provided are separate pharmaceutical kits, as just described, wherein the pharmaceutical composition comprises, in each of the separate pharmaceutical kits, one of the pharmaceutical compositions described above having dolutegravir as a component or element.

Further provided is a pharmaceutical kit, the kit comprising:
1) a series of doses of a first pharmaceutical composition comprising:
   a) a pharmaceutically effective amount of emtricitabine;
   b) a pharmaceutically effective amount of tenofovir disoproxil fumarate;
   c) a pharmaceutically effective amount of a TLR8 modulating compound, or a pharmaceutically acceptable salt thereof;
2) a pharmaceutically acceptable excipient; and
3) a series of doses of a second pharmaceutical composition comprising a pharmaceutically effective amount of dolutegravir and a pharmaceutically acceptable excipient; and
4) directions for the administration of the doses of the first and second pharmaceutical composition; wherein the first and second pharmaceutical compositions are both administered once daily.

Further provided is a pharmaceutical kit, the kit comprising:

a series of daily doses of a first pharmaceutical composition comprising:
a) a pharmaceutically effective amount of emtricitabine;
b) a pharmaceutically effective amount of tenofovir disoproxil fumarate;
c) a pharmaceutically effective amount of a TLR8 modulating compound, or a pharmaceutically acceptable salt thereof;
d) a pharmaceutically acceptable excipient; and

a series of daily doses of a second pharmaceutical composition comprising a pharmaceutically effective amount of dolutegravir and a pharmaceutically acceptable excipient; and

directions for the administration of the daily doses of the first and second pharmaceutical composition; wherein the first and second pharmaceutical compositions are both administered twice daily.

Further provided is a pharmaceutical kit, the kit comprising:

a series of doses of a first pharmaceutical composition comprising:
a) a pharmaceutically effective amount of emtricitabine;
b) a pharmaceutically effective amount of tenofovir disoproxil fumarate;
c) a pharmaceutically effective amount of a TLR8 modulating compound, or a pharmaceutically acceptable salt thereof;
d) a pharmaceutically acceptable excipient; and

a series of doses of a second pharmaceutical composition comprising a pharmaceutically effective amount of dolutegravir and a pharmaceutically acceptable excipient; and directions for the administration of the doses of the first and second pharmaceutical composition; wherein the first pharmaceutical composition is administered once daily and second pharmaceutical composition is administered twice daily.

Further provided is a pharmaceutical kit, the kit comprising:
1) a series of doses of a first pharmaceutical composition comprising:
   a) a pharmaceutically effective amount of emtricitabine;
   b) a pharmaceutically effective amount of tenofovir disoproxil fumarate;
   c) a pharmaceutically acceptable excipient; and
2) a series of doses of a second pharmaceutical composition comprising a pharmaceutically effective amount of dolutegravir and a pharmaceutically acceptable excipient; and
3) a series of doses of a third pharmaceutical composition comprising a pharmaceutically effective amount of a TLR8 modulating compound, or a pharmaceutically acceptable salt thereof; and
4) directions for the administration of the doses of the first and second pharmaceutical composition; wherein each of the first pharmaceutical composition, the second pharmaceutical composition, and the third pharmaceutical composition is administered once daily.

Within the embodiment of the pharmaceutical kit immediately above, there is a further embodiment comprising the kit, as described, wherein the second pharmaceutical composition comprises from 30 mg to 70 mg of dolutegravir. Within the embodiment of the pharmaceutical kit immediately above, there is a further embodiment comprising the kit, as described, wherein the second pharmaceutical composition comprises from 40 mg to 60 mg of dolutegravir. Within the embodiment of the pharmaceutical kit immediately above, there is a another embodiment comprising the kit, as described, wherein the second pharmaceutical composition comprises from 45 mg to 55 mg of dolutegravir. Within the embodiment of the pharmaceutical kit immediately above, there is a another embodiment comprising the kit, as described, wherein the second pharmaceutical composition comprises 50 mg of dolutegravir.

Further provided is a pharmaceutical kit, the kit comprising:

a series of doses of a first pharmaceutical composition comprising:

1) a pharmaceutically effective amount of emtricitabine; 2) a pharmaceutically effective amount of tenofovir disoproxil fumarate; 3) a pharmaceutically acceptable excipient; and

a series of doses of a second pharmaceutical composition comprising a pharmaceutically effective amount of dolutegravir and a pharmaceutically acceptable excipient; and a series of doses of a third pharmaceutical composition comprising a pharmaceutically effective amount of a TLR8 modulating compound, or a pharmaceutically acceptable salt thereof; and

directions for the administration of the doses of the first and second pharmaceutical composition; wherein the first pharmaceutical composition and third pharmaceutical composition are each administered once daily and the second pharmaceutical composition is administered twice daily.

Within the embodiment of the pharmaceutical kit above, there is a further embodiment comprising the kit, as described, wherein the first pharmaceutical composition comprises

200 mg of emtricitabine and 300 mg of tenofovir disoproxil fumarate, the second pharmaceutical composition comprises from 30 mg to 70 mg of dolutegravir, and the third pharmaceutical composition comprises a pharmaceutically effective amount of a TLR8 modulating compound, or a pharmaceutically acceptable salt thereof.

Within the embodiment of the pharmaceutical kit above, there is a further embodiment comprising the kit, as described, wherein the first pharmaceutical composition comprises

200 mg of emtricitabine and 300 mg of tenofovir disoproxil fumarate, the second pharmaceutical composition comprises from 40 mg to 60 mg of dolutegravir, and the third pharmaceutical composition comprises a pharmaceutically effective amount of a TLR8 modulating compound, or a pharmaceutically acceptable salt thereof.

Within the embodiment of the pharmaceutical kit above, there is a further embodiment comprising the kit, as described, wherein the first pharmaceutical composition comprises

200 mg of emtricitabine and 300 mg of tenofovir disoproxil fumarate, the second pharmaceutical composition comprises from 45 mg to 55 mg of dolutegravir and the third pharmaceutical composition comprises a pharmaceutically effective amount of a TLR8 modulating compound, or a pharmaceutically acceptable salt thereof.

Within the embodiment of the pharmaceutical kit above, there is a further embodiment comprising the kit, as described, wherein the first pharmaceutical composition comprises

200 mg of emtricitabine and 300 mg of tenofovir disoproxil fumarate, the second pharmaceutical composition comprises 50 mg of dolutegravir, and the third pharmaceutical composition comprises a pharmaceutically effective amount of a TLR8 modulating compound, or a pharmaceutically acceptable salt thereof.

It is understood that each description of the kits provided for herein containing a TLR8 modulating compound includes separate individual kits wherein the TLR8 modulating compound is of each Formula and compound example disclosed herein.

### Compositions Comprising TDF or TAF and a TLR8 Modulator

Pharmaceutically effective amounts of the TLR8 modulating compounds, Formula (J), (I), (II), (IIa), (I lb), (III), (IIIa), (IIIa), (IV), (IVa), (IVb), (IVc), or (IVd), or each of the individual compounds of the examples from Example 1 through Example 120, or a pharmaceutically acceptable salt thereof, can be combined with a pharmaceutically effective amount of tenofovir disoproxil fumarate (TDF) for use in the methods of treatment discussed herein. For instance, as separate dosage forms, a pharmaceutically effective dose of the TLR8 modulating compounds may be combined in a treatment regimen with a Viread® TDF tablet, which are available from Gilead Sciences, Inc. in 150 mg, 200 mg, 250 mg, and 300 mg strengths.

Provided is a pharmaceutical composition comprising:
a) a pharmaceutically effective amount of tenofovir disoproxil fumarate;
b) a pharmaceutically effective amount of a TLR8 modulating compound, or a pharmaceutically acceptable salt thereof; and
c) a pharmaceutically acceptable excipient.

Provided is a pharmaceutical composition comprising:
a) a pharmaceutically effective amount of tenofovir disoproxil fumarate;
b) a pharmaceutically effective amount of a compound of Formula (I), or a pharmaceutically acceptable salt thereof; and
c) a pharmaceutically acceptable excipient.

Provided is a pharmaceutical composition comprising:
a) a pharmaceutically effective amount of TAF;
b) a pharmaceutically effective amount of a TLR8 modulating compound, or a pharmaceutically acceptable salt thereof; and
c) a pharmaceutically acceptable excipient.

Provided is a pharmaceutical composition comprising:
a) a pharmaceutically effective amount of TAF;
b) a pharmaceutically effective amount of a compound of Formula (I), or a pharmaceutically acceptable salt thereof; and
c) a pharmaceutically acceptable excipient.

Specific pharmaceutical compositions and combinations comprising a pharmaceutically acceptable excipient and the individual amounts of TDF or TAF, and b) a pharmaceutically effective amount of a TLR8 Modulating Compound or a pharmaceutically acceptable salt thereof, include the specific amounts of TDF and TAF as listed below

```
TDF
25 mg to 350 mg
30 mg to 50 mg
75 mg to 125 mg
125 mg to 175 mg
175 mg to 225 mg
275 mg to 325 mg
  40 mg
  50 mg
  75 mg
100 mg
150 mg
200 mg
300 mg
50 mg to 350 mg
50 mg to 300 mg
50 mg to 250 mg
```

TAF
5 mg to 35 mg
10 mg to 30 mg
10 mg to 20 mg
20 mg to 30 mg
5 mg to 15 mg
15 mg to 25 mg
10 mg
15 mg
20 mg
25 mg
30 mg
35 mg
5 mg to 35 mg
1 mg to 25 mg
1 mg to 20 mg
1 mg to 15 mg

Also provided is a pharmaceutical kit, the kit comprising:
1) a series of daily doses of a single pharmaceutical composition comprising:
   a) a pharmaceutically effective amount of tenofovir disoproxil fumarate;
   b) a pharmaceutically effective amount of a TLR8 modulating compound, or a pharmaceutically acceptable salt thereof; and
   c) a pharmaceutically acceptable excipient; and
2) directions for the administration of the daily doses of the pharmaceutical composition.

Also provided is a pharmaceutical kit, the kit comprising:
a) a series of daily doses of a single pharmaceutical composition comprising a pharmaceutically effective amount of tenofovir disoproxil fumarate for daily administration;
b) a series of doses of a pharmaceutically effective amount of a TLR8 modulating compound for less than daily administration, or a pharmaceutically acceptable salt thereof; and
c) and directions for the administration of the daily doses of tenofovir disoproxil fumarate and the less than daily administration of the doses of the TLR8 modulating compound.

Also provided is a pharmaceutical kit, the kit comprising:
1) a series of daily doses of a single pharmaceutical composition comprising:
   a) a pharmaceutically effective amount of tenofovir disoproxil fumarate;
   b) a pharmaceutically effective amount of a compound of Formula (I), or a pharmaceutically acceptable salt thereof; and
   c) a pharmaceutically acceptable excipient; and
2) directions for the administration of the daily doses of the pharmaceutical composition.

Provided are a series of individual pharmaceutical kits as just described wherein each individual kit is provided comprises a pharmaceutically acceptable excipient, one of the pharmaceutically effective amounts of tenofovir disoproxil fumarate referenced above and a pharmaceutically effective amount of a compound of Formula (I), or a pharmaceutically acceptable salt thereof.

Further provided is a pharmaceutical kit, the kit comprising a series of doses of a first pharmaceutical composition comprising a pharmaceutically effective amount of tenofovir disoproxil fumarate and a pharmaceutically acceptable excipient; a series of doses of a second pharmaceutical composition comprising a pharmaceutically effective amount of a compound of Formula (I), or a pharmaceutically acceptable salt thereof; and a pharmaceutically acceptable excipient; and directions for the administration of the doses of the first and second pharmaceutical composition; wherein the first and second pharmaceutical compositions are both administered once daily. Further provided is a pharmaceutical kit, the kit comprising the series of doses of the first pharmaceutical composition, the second pharmaceutical composition and the directions just described, wherein the first and second pharmaceutical compositions are both administered twice daily. Also provided is a pharmaceutical kit, the kit comprising the series of doses of the first pharmaceutical composition, the second pharmaceutical composition and the directions just described, wherein the first pharmaceutical composition is administered once daily and the second pharmaceutical compositions is administered twice daily.

Combinations of rilpivirine/emtricitabine/TDF/TLR8 Modulators

Pharmaceutically effective amounts of the TLR8 modulating compounds, including those Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof, can be combined with pharmaceutically effective amounts of emtricitabine, rilpivirine, and tenofovir disoproxil fumarate (TDF) for use in the methods of treatment discussed herein. For instance, as separate dosage forms, a pharmaceutically effective dose of the TLR8 modulating compounds may be combined in a treatment regimen with a COMPLERA ® tablet, which is available from Gilead Sciences, Inc. and contain 200 mg of emtricitabine, 25 mg rilpivirine, and 300 mg of TDF.

Also provided is a pharmaceutical composition comprising:
a) a pharmaceutically effective amount of rilpivirine, or a pharmaceutically acceptable salt thereof;
b) a pharmaceutically effective amount of emtricitabine;
c) a pharmaceutically effective amount of tenofovir disoproxil fumarate;
d) a pharmaceutically effective amount of a TLR8 modulaitng compound, or a pharmaceutically acceptable salt thereof; and
e) a pharmaceutically acceptable excipient.

Also provided is a pharmaceutical composition comprising:
a) a pharmaceutically effective amount of rilpivirine HCI;
b) a pharmaceutically effective amount of emtricitabine;
c) a pharmaceutically effective amount of tenofovir disoproxil fumarate;
d) a pharmaceutically effective amount of a compound of Formula (I), or a pharmaceutically acceptable salt thereof; and
e) a pharmaceutically acceptable excipient.

Also provided are separate pharmaceutical compositions, each comprising 1) a pharmaceutically effective amount of rilpivirine; 2) a pharmaceutically effective amount of emtricitabine; 3) a pharmaceutically effective amount of tenofovir disoproxil fumarate; 4) a pharmaceutically acceptable excipient; and 5) a pharmaceutically effective amount of a TLR8 modulating compound selected from one of the group of Formula (J), (I), (II), (Ila), (Ilb), (III), (IIIa), (IIIb), (IV), (IVa), (IVb), (IVc), or (IVd), or each of the individual compounds of the examples from Example 1 through Example 120; or a pharmaceutically acceptable salt thereof. Each of the separate pharmaceutical compositions comprises one formula, for instance, one embodiment comprises 1) a pharmaceutically effective amount of rilpivirine or a pharmaceutically acceptable salt thereof; 2) a pharmaceutically effective amount of emtricitabine; 3) a pharmaceutically effective amount of tenofovir disoproxil fumarate; 4) a pharmaceutically acceptable excipient; and 5) a pharmaceutically effective amount of a compound of Formula (J), or a pharmaceutically acceptable salt thereof, another comprises 1) a pharmaceutically effective amount of rilpivirine or a pharmaceutically acceptable salt thereof; 2) a pharmaceutically effective amount of emtricitabine; 3) a pharmaceutically effective amount of tenofovir disoproxil fumarate; 4) a pharmaceutically acceptable excipient; and 5) a pharmaceutically effective amount of a compound of Formula (I)or a pharmaceutically acceptable salt thereof, etc.

Combinations of efavirenz/emtricitabine/TDF or TAF/TLR8 Modulators

Pharmaceutically effective amounts of the TLR8 modulating compounds, including those of Formula (J), (I), (II), (IIa), (IIb), (III), (IIIa), (IIIa), (IV), (IVa), (IVb), (IVc), or (IVd), or each of the individual compounds of the examples from Example 1 through Example 120or a pharmaceutically acceptable salt thereof, can be combined with pharmaceutically effective amounts of efavirenz, emtricitabine, and tenofovir disoproxil fumarate (TDF) for use in the methods of treatment discussed herein. For instance, as separate dosage forms, a pharmaceutically effective dose of a TLR8 modulating compound, or pharmaceutically acceptable salt thereof, may be combined in a treatment regimen with an ATRIPLA® tablet (600 mg efavirenz, 200 mg emtricitabine and 300 mg tenofovir disoproxil fumarate), which is available from Gilead Sciences, Inc.

Also provided is a pharmaceutical composition comprising:
a) a pharmaceutically effective amount of efavirenz;
b) a pharmaceutically effective amount of emtricitabine;
c) a pharmaceutically effective amount of tenofovir disoproxil fumarate;
d) a pharmaceutically effective amount of a TLR8 modulating compound, or a pharmaceutically acceptable salt thereof; and
e) a pharmaceutically acceptable excipient.

Also provided is a pharmaceutical composition comprising:
a) a pharmaceutically effective amount of efavirenz;
b) a pharmaceutically effective amount of emtricitabine;
c) a pharmaceutically effective amount of tenofovir disoproxil fumarate;
d) a pharmaceutically effective amount of a compound of Formula (I), or a pharmaceutically acceptable salt thereof; and
e) a pharmaceutically acceptable excipient.

Also provided is a pharmaceutical composition comprising:
a) a pharmaceutically effective amount of efavirenz;
b) a pharmaceutically effective amount of emtricitabine;
c) a pharmaceutically effective amount of TAF;
d) a pharmaceutically effective amount of a TLR8 modulating compound, or a pharmaceutically acceptable salt thereof; and
e) a pharmaceutically acceptable excipient.

Also provided is a pharmaceutical composition comprising:
f) a pharmaceutically effective amount of efavirenz;
g) a pharmaceutically effective amount of emtricitabine;
h) a pharmaceutically effective amount of TAF;
i) a pharmaceutically effective amount of a compound of Formula (I), or a pharmaceutically acceptable salt thereof; and
j) a pharmaceutically acceptable excipient.

Also provided are separate pharmaceutical compositions, each comprising 1) a pharmaceutically effective amount of efavirenz; 2) a pharmaceutically effective amount of emtricitabine; 3) a pharmaceutically effective amount of tenofovir disoproxil fumarate; 4) a pharmaceutically acceptable excipient; and 5) a pharmaceutically effective amount of a compound selected from one of the group of Formula (J), (I), (II), (IIa), (IIb), (III), (IIIa), (IIIa), (IV), (IVa), (IVb), (IVc), or (IVd), or each of the individual compounds of the examples from Example 1 through Example 120; or a pharmaceutically acceptable salt thereof. Each of the separate pharmaceutical compositions comprises one formula, for instance, one embodiment comprises 1) a pharmaceutically effective amount of efavirenz; 2) a pharmaceutically effective amount of emtricitabine; 3) a pharmaceutically effective amount of tenofovir disoproxil fumarate; 4) a pharmaceutically acceptable excipient; and 5) a pharmaceutically effective amount of a compound of Formula (J), or a pharmaceutically acceptable salt thereof, another comprises 1) a pharmaceutically effective amount of efavirenz; 2) a pharmaceutically effective amount of emtricitabine; 3) a pharmaceutically effective amount of tenofovir disoproxil fumarate; 4) a pharmaceutically acceptable excipient; and 5) a pharmaceutically effective amount of a compound of Formula (I) or a pharmaceutically acceptable salt thereof, etc.

Combinations of elvitegravir/cobicistat/emtricitabine/TAF/TLR8 Modulators

Pharmaceutically effective amounts of the TLR8 modulating compounds, including compounds of Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof,can be combined with pharmaceutically effective amounts of elvitegravir, cobicistat, emtricitabine, and TAF for use in the methods of treatment discussed herein. For instance, as separate dosage forms, a pharmaceutically effective dose of a TLR8 modulating compound, or pharmaceutically acceptable salt thereof, may be combined in a treatment regimen of 150 mg elvitegravir, 150 mg cobicistat, 200 mg emtricitabine, and 300 mg tenofovir disoproxil fumarate, such as with a STRIBILD® tablet available from Gilead Sciences, Inc.

Also provided is a pharmaceutical composition comprising:
a) a pharmaceutically effective amount of elvitegravir;
b) a pharmaceutically effective amount of cobicistat;
c) a pharmaceutically effective amount of emtricitabine;
d) a pharmaceutically effective amount of tenofovir alafenamide (TAF), or a pharmaceutically acceptable salt thereof;
e) a pharmaceutically effective amount of a TLR8 modulating compound, or a pharmaceutically acceptable salt thereof; and
f) a pharmaceutically acceptable excipient.

Also provided is a pharmaceutical composition comprising:
a) a pharmaceutically effective amount of elvitegravir;
b) a pharmaceutically effective amount of cobicistat;
c) a pharmaceutically effective amount of emtricitabine;
d) a pharmaceutically effective amount of tenofovir alafenamide (TAF), or a pharmaceutically acceptable salt thereof;
e) a pharmaceutically effective amount of a compound of Formula (I), or a pharmaceutically acceptable salt thereof; and
f) a pharmaceutically acceptable excipient.

Also provided are additional separate pharmaceutical compositions, each comprising 1) a pharmaceutically effective amount of elvitegravir; 2) a pharmaceutically effective amount of emtricitabine; 3) a pharmaceutically effective amount of cobicistat; 4) a pharmaceutically effective amount of tenofovir alafenamide; 5) a pharmaceutically acceptable excipient; and 6) a pharmaceutically effective amount of a compound selected from one of the group of Formula (J), (I), (II), (IIa), (IIb), (III), (IIIa), (IIIa), (IV), (IVa), (IVb), (IVc), or (IVd), or each of the individual compounds of the examples from Example 1 through Example 120; ora pharmaceutically acceptable salt thereof. Each of the separate pharmaceutical compositions comprises one formula, for instance, one embodiment comprises 1) a pharmaceutically effective amount of elvitegravir; 2) a pharmaceutically effective amount of emtricitabine; 3) a pharmaceutically effective amount of cobicistat; 4) a pharmaceutically effective amount of tenofovir alafenamide; 5) a pharmaceutically acceptable excipient; and 6) a pharmaceutically effective amount of a compound of Formula (J), or a pharmaceutically acceptable salt thereof, another comprises 1) a pharmaceutically effective amount of elvitegravir; 2) a pharmaceutically effective amount of emtricitabine; 3) a pharmaceutically effective amount of cobicistat; 4) a pharmaceutically effective amount of tenofovir alafenamide; 5) a pharmaceutically acceptable excipient; and 6) a pharmaceutically effective amount of a compound of Formula (I), or a pharmaceutically acceptable salt thereof, etc.

Also provided are pharmaceutical combinations and compositions comprising a pharmaceutically effective amount of a TLR8 modulating compound, a pharmaceutically effective amount of TAF, a pharmaceutically effective amount of emtricitabine, and a pharmaceutically effective amount of one or more antiviral agents selected from the group of:

non-nucleoside reverse transcriptase inhibitors, such as etravirine, delaviridine, efavirenz, and nevirapine, and pharmaceutically acceptable salts thereof;

nucleoside reverse transcriptase inhibitors, such as lamivudine, zidovudine, emtricitabine, abacavir, zalcitabine, TDF, and stavudine, and pharmaceutically acceptable salts thereof;

protease inhibitors, such as amprenavir, tipranavir, indinavir, saquinavir, lopinovir, ritonavir, fosamprenavir, darunivir, atazanavir, and nelfinavir, and pharmaceutically acceptable salts thereof;

CCR5 antagonists, such as maraviroc and enfuvirtide, and pharmaceutically acceptable salts thereof;

HIV integrase strand transfer inhibitors, such as raltegravir, and pharmaceutically acceptable salts thereof;

non-catalytic site integrase inhibitors, such as B1224436;and

capsid inhibitors.

Within each of the pharmaceutical compositions listed herein which include the component “tenofovir alafenamide, or a pharmaceutically acceptable salt thereof” or “TAF” there is a further embodiment in which that component comprises tenofovir alafenamide fumarate in the amount indicated for “tenofovir alafenamide, or a pharmaceutically acceptable salt thereof” and all other components or elements are as listed for the specific composition. Within each of the pharmaceutical compositions listed herein which include the component “tenofovir alafenamide, or a pharmaceutically acceptable salt thereof” there is a further embodiment in which that component comprises tenofovir alafenamide hemifumarate in the amount indicated for “tenofovir alafenamide, or a pharmaceutically acceptable salt thereof” and all other components or elements are as listed for the specific composition.

Also provided are pharmaceutical combinations and compositions comprising a pharmaceutically effective amount of a TLR8 modulating compound, a pharmaceutically effective amount of TDF, a pharmaceutically effective amount of emtricitabine, and a pharmaceutically effective amount of one or more antiviral agents selected from the group of:

non-nucleoside reverse transcriptase inhibitors, such as etravirine, delaviridine, efavirenz, and nevirapine, and pharmaceutically acceptable salts thereof;

nucleoside reverse transcriptase inhibitors, such as lamivudine, zidovudine, emtricitabine, abacavir, zalcitabine, TAF, and stavudine, and pharmaceutically acceptable salts thereof;

protease inhibitors, such as amprenavir, tipranavir, indinavir, saquinavir, lopinovir, ritonavir, fosamprenavir, darunivir, atazanavir, and nelfinavir, and pharmaceutically acceptable salts thereof;

CCR5 antagonists, such as maraviroc and enfuvirtide, and pharmaceutically acceptable salts thereof;

HIV integrase strand transfer inhibitors, such as raltegravir, and pharmaceutically acceptable salts thereof;

non-catalytic site integrase inhibitors, such as B1224436;and

capsid inhibitors.

Combination of atazanavir sulfate, cobicistat, and a TLR8 Modulator

Pharmaceutically effective amounts of the TLR8 modulating compounds, including those of Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof, can be combined with pharmaceutically effective amounts of atazanavir sulfate and cobicistat for use in the methods of treatment discussed herein. For instance, as separate dosage forms, a pharmaceutically effective dose of a TLR8 modulating compound, or pharmaceutically acceptable salt thereof, may be combined in a treatment regimen with a pharmaceutically effective dose of cobicistat and a REYATAZ® 150 mg, 200mg, or 300 mg atazanavir sulfate capsule, which are available from Bristol-Meyers Squibb Co. As another example, a combined dosage unit, such as a tablet or capsule, comprising a pharmaceutically effective amount of cobicistat and a pharmaceutically effective amount of a TLR8 modulating compound, or pharmaceutically acceptable salt thereof, may be administered to a human in need thereof in coordination with administration of a pharmaceutically effective dose of atazanavir or atazanavir sulfate, such as seen in the 150 mg, 200 mg, or 300 mg REYATAZ® capsules atazanavir or atazanavir sulfate.

Also provided is a pharmaceutical composition comprising:
a) a pharmaceutically effective amount of atazanavir, or a pharmaceutically acceptable salt thereof;
b) a pharmaceutically effective amount of cobicistat;
c) a pharmaceutically effective amount of a TLR8 modulating compound; and
d) a pharmaceutically acceptable excipient.

Also provided is a pharmaceutical composition comprising:
a) a pharmaceutically effective amount of atazanavir sulfate;
b) a pharmaceutically effective amount of cobicistat;
c) a pharmaceutically effective amount of a compound of Formula (I) or a pharmaceutically acceptable salt thereof; and
d) a pharmaceutically acceptable excipient.

Also provided is a pharmaceutical composition comprising:
a) a pharmaceutically effective amount of cobicistat;
b) a pharmaceutically effective amount of a TLR8 modulating compound; and pharmaceutically acceptable excipient.

Also provided is a pharmaceutical composition comprising:
a) a pharmaceutically effective amount of cobicistat;
b) a pharmaceutically effective amount of a compound of Formula (I) or a pharmaceutically acceptable salt thereof; and
c) a pharmaceutically acceptable excipient.

Also provided is a pharmaceutical composition comprising:
a) a pharmaceutically effective amount of ritonavir;
b) a pharmaceutically effective amount of a TLR8 modulating compound; and
c) a pharmaceutically acceptable excipient.

Also provided is a pharmaceutical composition comprising:
a) a pharmaceutically effective amount of ritonavir;
b) a pharmaceutically effective amount of a compound of Formula (I) or a pharmaceutically acceptable salt thereof; and
c) a pharmaceutically acceptable excipient.

Also provided are separate pharmaceutical compositions, each comprising 1) a pharmaceutically effective amount of atazanavir, or a pharmaceutically acceptable salt thereof; 2) a pharmaceutically effective amount of cobicistat; 3) a pharmaceutically acceptable excipient; and 4) a pharmaceutically effective amount of a compound selected from one of the group of Formula (J), (I), (II), (IIa), (IIb), (III), (IIIa), (IIIa), (IV), (IVa), (IVb), (IVc), or (IVd), or each of the individual compounds of the examples from Example 1 through Example 120or a pharmaceutically acceptable salt thereof. Each of the separate pharmaceutical compositions comprises one formula, for instance, one embodiment comprises 1) a pharmaceutically effective amount of atazanavir, or a pharmaceutically acceptable salt thereof; 2) a pharmaceutically effective amount of cobicistat; 3) a pharmaceutically acceptable excipient; and 4) a pharmaceutically effective amount of a compound selected from one of the group of Formula (J), or a pharmaceutically acceptable salt thereof, another comprises 1) a pharmaceutically effective amount of atazanavir, or a pharmaceutically acceptable salt thereof; 2) a pharmaceutically effective amount of cobicistat; 3) a pharmaceutically acceptable excipient; and 4) a pharmaceutically effective amount of a compound selected from one of the group of Formula (I) or a pharmaceutically acceptable salt thereof, etc.

Combinations of abacavir/lamivudine/dolutegravir/TLR8 Modulators

Pharmaceutically effective amounts of the TLR8 modulating compounds, including those of Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof,can be combined with pharmaceutically effective amounts of abacavir, lamivudine, and dolutegravir for use in the methods of treatment discussed herein. For instance, as separate dosage forms, a pharmaceutically effective dose of a TLR8 modulating compound, or pharmaceutically acceptable salt thereof, may be combined in a treatment regimen of 300 mg abacavir, 150 mg lamivudine, and 50 mg dolutegravir, such as with a TRIUMEQ® tablet available from ViiV Healthcare. abacavir, lamivudine, and dolutegravir

Also provided is a pharmaceutical composition comprising:
a) a pharmaceutically effective amount of abacavir;
b) a pharmaceutically effective amount of lamivudine;
c) a pharmaceutically effective amount of dolutegravir;
d) a pharmaceutically effective amount of a TLR8 modulating compound, or a pharmaceutically acceptable salt thereof; and
e) a pharmaceutically acceptable excipient.

Also provided is a pharmaceutical composition comprising:
a) a pharmaceutically effective amount of abacavir;
b) a pharmaceutically effective amount of lamuvidine;
c) a pharmaceutically effective amount of dolutegravir;
d) a pharmaceutically effective amount of a compound of Formula (I), or a pharmaceutically acceptable salt thereof; and
e) a pharmaceutically acceptable excipient.

Also provided are additional separate pharmaceutical compositions, each comprising 1) a pharmaceutically effective amount of abacavir; 2) a pharmaceutically effective amount of lamivudine; 3) a pharmaceutically effective amount of dolutegravir; 4) a pharmaceutically acceptable excipient; and 5) a pharmaceutically effective amount of a compound selected from one of the group of Formula (J), (I), (II), (IIa), (IIb), (III), (IIIa), (IIIa), (IV), (IVa), (IVb), (IVc), or (IVd), or each of the individual compounds of the examples from Example 1 through Example 120; or a pharmaceutically acceptable salt thereof. Each of the separate pharmaceutical compositions comprises one formula, for instance, one embodiment comprises 1) a pharmaceutically effective amount of abacavir; 2) a pharmaceutically effective amount of lamivudine; 3) a pharmaceutically effective amount of dolutegravir; 4) a pharmaceutically acceptable excipient; and 5) a pharmaceutically effective amount of a compound of Formula (J), or a pharmaceutically acceptable salt thereof, another comprises 1) a pharmaceutically effective amount of abacavir; 2) a pharmaceutically effective amount of lamivudine; 3) a pharmaceutically effective amount of dolutegravir; 4) a pharmaceutically acceptable excipient; and 5) a pharmaceutically effective amount of a compound of Formula (I) or a pharmaceutically acceptable salt thereof, etc.

Combinations of darunavir/ritonavir/cobicistat/TLR8 Modulators

Pharmaceutically effective amounts of the TLR8 modulating compounds, including thos of Formula (J), (I), (II), (IIa), (IIb), (III), (IIIa), (IIIa), (IV), (IVa), (IVb), (IVc), or (IVd), or each of the individual compounds of the examples from Example 1 through Example 120, or a pharmaceutically acceptable salt thereof,can be combined with pharmaceutically effective amounts of darunavir and one of ritonavir or dolutegravir for use in the methods of treatment discussed herein. darunavir, ritonavir, and dolutegravirAlso provided is a pharmaceutical composition comprising a pharmaceutically effective amount of darunavir and one of ritonavir or dolutegravir, and a pharmaceutically excipient.

Also provided is a pharmaceutical composition comprising:
a) a pharmaceutically effective amount of darunavir;
b) a pharmaceutically effective amount of ritonavir or cobicistat;
c) a pharmaceutically effective amount of a TLR8 modulating compound, or a pharmaceutically acceptable salt thereof; and
d) a pharmaceutically acceptable excipient.

Also provided is a pharmaceutical composition comprising:
a) a pharmaceutically effective amount of darunavir;
b) a pharmaceutically effective amount of ritonavir or cobicistat;
c) a pharmaceutically effective amount of a compound of Formula (I), or a pharmaceutically acceptable salt thereof; and
d) a pharmaceutically acceptable excipient.

Also provided are additional separate pharmaceutical compositions, each comprising 1) a pharmaceutically effective amount of darunavir; 2) a pharmaceutically effective amount of ritonavir or cobiscistat; 3) a pharmaceutically acceptable excipient; and 4) a pharmaceutically effective amount of a compound selected from one of the group of Formula (J), (I), (II), (IIa), (IIb), (III), (IIIa), (IIIa), (IV), (IVa), (IVb), (IVc), or (IVd), or each of the individual compounds of the examples from Example 1 through Example 120; ora pharmaceutically acceptable salt thereof. Each of the separate pharmaceutical compositions comprises one formula, for instance, one embodiment comprises 1) a pharmaceutically effective amount of darunavir; 2) a pharmaceutically effective amount of ritonavir or cobiscistat; 3) a pharmaceutically acceptable excipient; and 4) a pharmaceutically effective amount of a compound of Formula (J), or a pharmaceutically acceptable salt thereof, another comprises 1) a pharmaceutically effective amount of darunavir; 2) a pharmaceutically effective amount of ritonavir or cobiscistat; 3) a pharmaceutically acceptable excipient; and 4)) a pharmaceutically effective amount of a compound of Formula (I) or a pharmaceutically acceptable salt thereof, etc.

### Cobicistat or Ritonovir

Also provided are the specific pharmaceutical compositions and combinations which comprises a pharmaceutically acceptable excipient and the individual pharmaceutically effective amounts of cobicistat or ritonavir and a pharmaceutically effective amount of a TLR8 Modulating Compound or a pharmaceutically acceptable salt thereof.

In certain embodiments, the amount of cobicsitat is from 100 mg to 200 mg; 125 mg to 175 mg, 140 mg to 175 mg, or 150 mg. In certain embodiments, the amout of ritonavir is from 50 mg to 800 mg, from 75 mg to 450 mg, from 250 mg to 450 mg, from 75 mg to 250 mg, from 50 mg to 150 mg, from 25 to 75 mg, or 100 mg.

A pharmaceutically effective amount of a TLR8 modulating compound, including each of those described herein, can also be combined in dosing regimens and pharmaceutical compositions with a pharmaceutically effective amount of a protease inhibitor known in the art, or a pharmaceutically acceptable salt thereof.

Provided is a pharmaceutical composition comprising:

a) a pharmaceutically effective amount of a protease inhibitor;

b) a pharmaceutically effective amount of a TLR8 modulating compound; and

c) a pharmaceutically acceptable excipient.

For instance, combinations useful in pharmaceutical regimens and pharmaceutical compositions comprise:
a) a pharmaceutically effective amount of a protease inhibitor, or a pharmaceutically acceptable salt thereof, selected from the group of atazanavir, darunavir, indinavir, lopinavir, nelfinavir, saquinavir, tpranavir, fosamprenavir, ritonavir, amprenavir, and telaprevir; and
b) a pharmaceutically effective amount of a TLR8 modulating compound, or a pharmaceutically acceptable salt thereof, selected from the group of a compound of Formula (J), (I), (II), (IIa), (IIb), (III), (IIIa), (IIIa), (IV), (IVa), (IVb), (IVc), or (IVd), or each of the individual compounds of the examples from Example 1 through Example 120, motolimod, 3M-051, 3M-052, MCT-465, IMO-4200, VTX-763, VTX-1463

wherein the separate pharmaceutical regimens comprise a pharmaceutically effective amount of each of the protease inhibitors in group a) above, individually, combined with a pharmaceutically effective amount of each TLR8 modulating compound, represented by a specific compound, agent, or formula of group b), individually, and the separate pharmaceutical compositions each comprise a pharmaceutically acceptable excipient, a pharmaceutically effective amount of each of the protease inhibitors in group a) above, individually, and a pharmaceutically effective amount of each TLR8 modulating compound, represented by a specific compound, agent, or formula of group b), individually. It is understood that the combinations of the eleven protease inhibitors in group a), individually, with the one hundred and forty one TLR8 modulating compounds of group b), represented by a specific compound, agent, or formula, provides separate combinations of one protease inhibitor and one TLR8 modulating compound each.

Also provided are the specific pharmaceutical compositions and combinations which comprises a pharmaceutically acceptable excipient and the individual pharmaceutically effective amounts of a protease inhibitor and a pharmaceutically effective amount of a TLR8 Modulating Compound or a pharmaceutically acceptable salt thereof.

In certain embodiments the protease inhibitor is atazanavir. In certain embodiments, atazanavir is present in an amount of 100 mg to 500 mg, 300 mg, or 400 mg. In certain embodiments the protease inhibitor is darunavir. In certain embodiments, darunavir is present in an amount of 50 mg to 1000 mg, 50 mg to 800 mg, 800 mg, 600 mg, 150 mg, or 75 mg. . In certain embodiments the protease inhibitor is indinavir. In certain embodiments, indinavir is present in an amount of 100 mg to 1000 mg, 200 mg to 800 mg, 400 mg , or 800 mg. . In certain embodiments the protease inhibitor is lopinavir. In certain embodiments, lopinavir is present in an amount of 100 mg to 1000 mg, 200 mg to 800 mg, 200 mg, 400 mg , or 800 mg.

In certain embodiments the protease inhibitor is nelfinavir. In certain embodiments, nelfinavir is present in an amount of 100 mg to 2500 mg, 250 mg to 2500 mg, 250 mg , or 625 mg. In certain embodiments the protease inhibitor is saquinavir. In certain embodiments, saquinavir is present in an amount of 200 mg to 1500 mg, 500 mg to 1000 mg, 200 mg , or 500 mg. In certain embodiments the protease inhibitor is tipranavir. In certain embodiments, tipranavir is present in an amount of 50 mg to 500 mg, 100 mg to 500 mg, 100 mg , or 250 mg. In certain embodiments the protease inhibitor is fosamprenavir. In certain embodiments, fosamprenavir is present in an amount of 500 mg to 1500 mg, 500 mg, or 700 mg. In certain embodiments the protease inhibitor is ritonavir. In certain embodiments, ritonavir is present in an amount of 100 mg to 1000 mg, 200 mg to 600 mg, 50 mg , or 100 mg. In certain embodiments the protease inhibitor is amprenavir. In certain embodiments, amprenavir is present in an amount of 100 mg to 1500 mg, 500 mg to 1300 mg, 100 mg , or 150 mg.

Another embodiment comprises a kit comprising a pharmaceutically effective amount of an antiviral agent, or a pharmaceutically acceptable salt thereof, a pharmaceutically effective amount of a TLR8 modulating compound, or a pharmaceutically acceptable salt thereof, and directions for the administration of the antiviral agent and the TLR8 modulating compound. A further embodiment comprises the kit just described wherein the antiviral agent and the TLR8 modulating compound are present in the same pharmaceutical composition. Another embodiment comprises the kit just described wherein the antiviral agent and the TLR8 modulating compound are present in separate pharmaceutical compositions.

Each of the kits described herein may further comprise a container and a label or a package insert on or associated with the container. The term “package insert” refers to instructions customarily included in commercial packages of therapeutic products providing relevant information, such as indications, usage, dosages, administration, contraindications and/or warnings associated with the therapeutic products.

Separate embodiments also comprise individual kits each comprising pharmaceutical compositions comprising the pharmaceutically effective amounts of the one or more antiviral agents and the TLR8 modulator described for each of the individual combinations described herein, and directions for their administration. It is understood that each listed combination appears in a separate kit. A further set of separate embodiments comprises the kits just described wherein the one or more antiviral agents and the TLR8 modulating compound are present in the same pharmaceutical composition. Another set of embodiments comprises the kits just described wherein the one or more antiviral agents of each combination are in one pharmaceutical composition and the TLR8 modulating compound is present in a separate pharmaceutical composition. Another set of embodiments for those combinations with three or more antiviral agents comprises the kits just described wherein two or more antiviral agents of each combination are in one pharmaceutical composition, at least one antiviral agent is in a separate pharmaceutical composition, and the TLR8 modulating compound is present in another separate pharmaceutical composition. A further set of embodiments comprises the kits just described wherein each of the antiviral agents of each combination are in a separate pharmaceutical composition, with one antiviral agent per composition, and the TLR8 modulating compound is present in another separate pharmaceutical composition.

Also provided are separate embodiments comprising the use of the pharmaceutically effective amounts of each of the antiviral compounds as listed herein (e.g.the amount in Tables A-J and listed throughout this disclosure) and TLR8 modulating compounds. It is understood that each individual combination in the tables represents a separate embodiment for use in the treatment of an HIV infection in a human. Also provided are separate embodiments comprising the use of the pharmaceutically effective amounts of each of the antiviral compounds and TLR8 modulating compounds in each of individual combinations listed above for one or more of:
a) use in treating an HIV infection in a virologically suppressed human;
b) use in inducing HIV of inducing HIV gene expression in a human infected with HIV;
c) use in inducing HIV gene expression in a human infected with HIV wherein active HIV gene expression in the human has been suppressed by administration of antiretroviral therapy;
d) use in inducing HIV gene expression in a latent HIV reservoir in a human infected with HIV;
e) use in enhancing HIV gene expression in HIV infected cells in a human infected with HIV;
f) use in lowering the chronic set point of HIV viral load in a human infected with HIV;
g) use in inducing transient HIV-1 viremia in a virologically suppressed human infected with HIV-1;
h) use in reducing HIV viremia in a human infected with HIV;
i) use in enhancing immune cell activity and increasing HIV gene expression in a human infected with HIV;
j) use in enhancing the efficacy of an antiviral agent in a human infected with HIV;
k) use in inducing transient HIV-1 viremia in a virologically suppressed human infected with HIV-1;
l) use in enhancing the efficacy of an HIV vaccine; and/or
m) use in eliminating an HIV infection in a human.

Also provided are separate embodiments comprising the use of the pharmaceutically effective amounts of each of the antiviral compounds and TLR8 modulating compounds in each of individual combinations listed above for the manufacture of a medicament for one or more of:
a) treating an HIV infection in a virologically suppressed human;
b) inducing HIV of inducing HIV gene expression in a human infected with HIV;
c) inducing HIV gene expression in a human infected with HIV wherein active HIV gene expression in the human has been suppressed by administration of antiretroviral therapy;
d) inducing HIV gene expression in a latent HIV reservoir in a human infected with HIV;
e) enhancing HIV gene expression in HIV infected cells in a human infected with HIV;
f) lowering the chronic set point of HIV viral load in a human infected with HIV;
g) inducing transient HIV-1 viremia in a virologically suppressed human infected with HIV-1;
h) reducing HIV viremia in a human infected with HIV;
i) enhancing immune cell activity and increasing HIV gene expression in a human infected with HIV;
j) enhancing the efficacy of an antiviral agent in a human infected with HIV;
k) use in inducing transient HIV-1 viremia in a virologically suppressed human infected with HIV-1;
l) enhancing the efficacy of an HIV vaccine; and/or
m) eliminating an HIV infection in a human.

Further separate embodiments for each of the kits described herein comprises the individual kit as described wherein
a) each of the pharmaceutical compositions comprising the one or more antiviral agents and the TLR8 modulator are each provided for daily dosing;
b) each of the pharmaceutical compositions comprising antiviral agents are provided for once daily administration and the pharmaceutical composition comprising the TLR8 modulator are provided for less than daily administration; and
c) each of the pharmaceutical compositions comprising antiviral agents are provided for once or twice daily administration and the pharmaceutical composition comprising the TLR8 modulator are provided for less than daily administration.

For each of the kits in which the pharmaceutical compositions comprising antiviral agents are provided for once or twice daily administration and the pharmaceutical composition comprising the TLR8 modulator are provided for less than daily administration, the pharmaceutical composition comprising the TLR8 modulator may be for administration every other day or every 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} day. Another embodiment for each of such kits comprises:
a) a number of daily dose compositions, such as an oral tablet or capsule, each daily dose composition comprising a pharmaceutically effective amount of at least one antiviral agent;
b) a number of less than daily dose compositions, such as an oral tablet or capsule, each daily dose composition comprising a pharmaceutically effective amount of at least one antiviral agent and a pharmaceutically effective amount of a TLR8 modulator; and
c) directions for the administration of the daily dose compositions and the less than daily dose compositions.

It is understood that in such kits there would be an equal number of daily dose compositions and less than daily dose compositions in kits wherein the TLR8 modulator is intended for every other day administration, there would be twice as many daily dose compositions as less than daily dose compositions in kits wherein the TLR8 modulator is intended for every third day administration, etc. Such kits may further include means for containing the compositions for scheduled administration, such as a cycle pack or a blister pack into which the individual compositions are contained in separate sections according to the order of their scheduled administration.

Also provided is a kit comprising:
a) a pharmaceutically effective amount of a TLR8 modulating compound;
b) a pharmaceutically effective amount of an HIV antibody; and
c) directions for the administration of the TLR8 modulating compound and the HIV antibody.

Also provided is a kit comprising:
a) a pharmaceutically effective amount of a TLR8 modulating compound;
b) a pharmaceutically effective amount of an HIV vaccine; and
c) directions for the administration of the TLR8 modulating compound and the HIV vaccine.

Also provided is a kit comprising:
a) a pharmaceutically effective amount of a TLR8 modulating compound;
b) a pharmaceutically effective amount of a latency reversing agent; and
c) directions for the administration of the TLR8 modulating compound and the latency reversing agent.

Provided are separate embodiments comprising:

the use of a TLR8 modulating compound and an antiretroviral agent, or a pharmaceutically acceptable salt thereof, in the preparation of a medicament for treating an HIV infection in a human; or

the use of a TLR8 modulating compound of Formula (I) and an antiretroviral agent, or a pharmaceutically acceptable salt thereof, in the preparation of a medicament for treating an HIV infection in a human.

Also provided are separate embodiments comprising:

the use of a TLR8 modulating compound and a latency-reversing agent, or a pharmaceutically acceptable salt thereof, in the preparation of a medicament for treating an HIV infection in a human; or

the use of a TLR8 modulating compound of Formula (I) and a latency-reversing agent, or a pharmaceutically acceptable salt thereof, in the preparation of a medicament for treating an HIV infection in a human.

Provided are separate embodiments comprising:

the use of a TLR8 modulating compound, or a pharmaceutically acceptable salt thereof, in the preparation of a medicament for enhancing the efficacy of an antiviral agent; or

the use of a TLR8 modulating compound of Formula (I), or a pharmaceutically acceptable salt thereof, in the preparation of a medicament for enhancing the efficacy of an antiviral agent.

Provided are separate embodiments comprising:

the use of a TLR8 modulating compound, or a pharmaceutically acceptable salt thereof, in the preparation of a medicament for enhancing the efficacy of an HIV vaccine; or

the use of a TLR8 modulating compound of Formula (I), or a pharmaceutically acceptable salt thereof, in the preparation of a medicament for enhancing the efficacy of an HIV vaccine.

Provided are separate embodiments comprising:

the use of a TLR8 modulating compound, or a pharmaceutically acceptable salt thereof, in the preparation of a medicament for eliminating an HIV infection in a human; or

the use of a TLR8 modulating compound of Formula (I), or a pharmaceutically acceptable salt thereof, in the preparation of a medicament for eliminating an HIV infection in a human.

Provided are separate embodiments comprising:

the use of a TLR8 modulating compound, or a pharmaceutically acceptable salt thereof, in the preparation of a medicament for inducing HIV gene expression in a human infected with HIV; or

the use of a TLR8 modulating compound of Formula (I), or a pharmaceutically acceptable salt thereof, in the preparation of a medicament for inducing HIV gene expression in a human infected with HIV.

Provided are separate embodiments comprising:

the use of a TLR8 modulating compound, or a pharmaceutically acceptable salt thereof, in the preparation of a medicament for enhancing HIV gene expression in HIV infected cells in a human infected with HIV; or

the use of a TLR8 modulating compound of Formula (I), or a pharmaceutically acceptable salt thereof, in the preparation of a medicament for enhancing HIV gene expression in HIV infected cells in a human infected with HIV.

Provided are separate embodiments comprising:

the use of a TLR8 modulating compound, or a pharmaceutically acceptable salt thereof, in the preparation of a medicament for inducing transient HIV-1 viremia in a virologically suppressed human infected with HIV-1; or

the use of a TLR8 modulating compound of Formula (I), or a pharmaceutically acceptable salt thereof, in the preparation of a medicament inducing transient HIV-1 viremia in a virologically suppressed human infected with HIV-1.

Provided are separate embodiments comprising:

the use of a TLR8 modulating compound, or a pharmaceutically acceptable salt thereof, in the preparation of a medicament for the treatment of an HIV-1 infection in a virologically suppressed human infected with HIV-1; or

the use of a TLR8 modulating compound of Formula (I), or a pharmaceutically acceptable salt thereof, in the preparation of a medicament for the treatment of an HIV-1 infection in a virologically suppressed human infected with HIV-1.

### BIOLOGICAL EXAMPLES

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### Example 121

### Induced HIV-1 Expression in PBMC Cultures from HIV-1 Infected Subjects on cART Treated with TLR8 Agonists

To assess the ability to activate HIV-1 expression in PBMC cultures, leukapheresis samples were obtained from 12 HIV-1 infected subjects on cART and virally suppressed with plasma HIV RNA<50 copies/mL for ≥1 year. Ficoll-purified PBMCs were cultured and treated with TLR8 modulating compounds of Example 15, Example 119, and Example 120, at concentrations from 10 nM to 2,500 nM, or with dimethyl sulfoxide (DMSO, vehicle control) for 4 days. In some experiments, IL-15, the TLR8 modulating compound of Example 15 or the combination of IL-15 and Example 15 were used at the indicated concentrations (Table 4). The cultures were maintained in the presence of antivirals (elvitegravir and efavirenz at 100 nM each) to prevent viral spread and amplification in order to measure the quantity of virus produced (latency reversal) by the TLR8 modulating compounds. At the end of the incubation period, cell-free culture supernatants were harvested and HIV-1 RNA levels were quantified by the COBAS® AmpliPrep/COBAS®TaqMan HIV-1 Test, v2.0 (Roche).

In the absence of other activation stimulus, treatment with Example 15 activated HIV-1 expression and virus production two-fold or higher compared to DMSO in PBMCs from 9 out of 12 (75%) subjects tested at concentrations of 10, 100, and/or 1,000 nM. The HIV fold-activation range in the PBMCs from these subjects was 2.2 to 20.1-fold, and the HIV fold-activation geometric mean was 4.4-fold (Table 1). Treatment with Example 119 activated HIV-1 expression two-fold or higher over the DMSO control in PBMC cultures from 5 out of 5 (100%) subjects tested at concentrations of 156, 625, and/or 2,500 nM. The HIV fold-activation range in the PBMCs from these subjects was 2.2 to 5.2-fold, and the HIV fold-activation geometric mean was 4.1-fold (Table 2). Treatment with Example 120 activated HIV-1 expression two-fold or higher compared to the DMSO control in PBMC cultures from 4 out of 5 (80%) subjects tested at concentrations of 156, 625, and/or 2,500 nM. The HIV fold-activation range in these subjects was 2.0 to 17.0-fold, and the HIV fold-activation geometric mean was 7.5-fold (Table 3). In each table, fold-activation is calculated as a ratio of HIV-1 RNA copies per ml of cell culture supernatants from TLR8 agonist treated samples and DMSO treated controls. Geometric mean fold-activation from three independent incubation samples for each condition of each subject's PBMCs. Values in bold indicate ≥2-fold HIV activation.

To assess if the latency reversal induced by TLR8 agonism could further potentiate latency reversal by a separate mechanisms, we treated cells as described above with IL-15, Example 15, or a combination of IL-15 and Example 15 (Table 4). In PBMCs from these patients, treatment with 1000 nM Example 15 led to a 2.1-fold increase in HIV RNA and treatment with 1 nM IL-15 led to a 2.9-fold increase in HIV RNA. Treatment with the same concentrations of these 2 compounds together led to a 12-fold increase in HIV RNA. In PBMC cultures from every subject treated, the combination led to greater HIV RNA production than was seen after treatment with either agent alone.

Overall, these data demonstrate the in vitro induction of HIV-1 expression by the TLR8 modulating agents and show that these agents can effectively increase HIV activation induced by IL-15.

TABLE 1
Activation of HIV-1 Expression by TLR8 Agonist Example 15
in PBMC (Cultures from HIV-1 Infected Subjects on cART

| | | Example 15 Treatment (nM) | | | |
|---|---|---|---|---|---|
| | Subject ID | | | | 10, 100, |
| | (N = 12) | 10 | 100 | 1,000 | or 1000 |
| Fold HIV-1 | 172 | 20.1 | 10.1 | 1.6 | |
| Activation^{a} | 173 | 0.8 | 1.3 | 5.7 | |
| | 174 | 2.6 | 3.8 | 2.7 | |
| | 175 | 2.2 | 6.6 | 2.4 | |
| | 176 | 4.7 | 2.2 | 3.3 | |
| | 177 | 1.2 | 1.5 | 1.2 | |
| | 178 | 1.9 | 1.0 | 4.7 | |
| | 179 | 1.3 | 0.5 | 0.3 | |
| | 180 | 0.3 | 0.2 | 3.4 | |
| | 181 | 0.5 | 0.1 | 0.3 | |
| | 182 | 1.0 | 0.7 | 3.0 | |
| | 183 | 4.1 | 1.8 | 1.3 | |
| | Geometric Mean | 4.6 | 4.8 | 3.4 | 4.4 |
| | with ≥2-Fold- | | | | |
| | Activation | | | | |
| | Range with ≥2- | 2.2-20.1 | 2.2-10.1 | 2.4-5.7 | 2.2-20.1 |
| | Fold-Activation | | | | |

| | | | | |
|---|---|---|---|---|
| N with ≥2-Fold-Activation (%) | 5 of 12 | 4 of 12 | 7 of 12 | 9 of 12 |
| | (42) | (33) | (58) | (75) |

TABLE 2
Activation of HIV-1 Expression by TLR8 Agonist of Example
119 in PBMC Cultures from HIV-1 Infected Subjects on cART

| | | Example 119 Treatment (nM) | | | |
|---|---|---|---|---|---|
| | | | | | 156, |
| | Subject ID | | | | 625, or |
| | (N = 5) | 156 | 625 | 2,500 | 2500 |
| Fold HIV-1 | 151 | 0.4 | 0.5 | 2.2 | |
| Activation^{a} | 152 | 3.6 | 3.4 | 4.1 | |
| | 153 | 4.9 | 4.1 | 4.2 | |
| | 154 | n.d.^{b} | 4.8 | 3.2 | |
| | 155 | n.d. | 0.3 | 5.2 | |
| | Geometric Mean | 4.2 | 4.1 | 3.6 | 4.1 |
| | with ≥2-Fold- | | | | |
| | Activation | | | | |
| | Range with ≥2- | 3.6-4.9 | 3.4-4.8 | 2.2-5.2 | 2.2-5.2 |
| | Fold-Activation | | | | |

| | | | | |
|---|---|---|---|---|
| N with ≥2-Fold-Activation (%) | 2 of 3 | 3 of 5 | 5 of 5 | 5 of 5 |
| | (67) | (60) | (100) | (100) |

TABLE 3
Activation of HIV-1 Expression by TLR8 Agonist of Example
120 in PBMC Cultures from HIV-1 Infected Subjects on cART

| | | Example 120 Treatment (nM) | | | |
|---|---|---|---|---|---|
| | | | | | 156, |
| | Subject ID | | | | 625, or |
| | (N = 5) | 156 | 625 | 2,500 | 2500 |
| Fold HIV-1 | 151 | 0.5 | 0.4 | 2.9 | |
| Activation^{a} | 152 | 9.0 | 15.2 | 10.2 | |
| | 153 | 17.0 | 14.0 | 4.0 | |
| | 154 | n.d.^{b} | 2.0 | 5.0 | |
| | 155 | n.d. | 0.9 | 1.2 | |
| | Geometric Mean | 12.4 | 7.5 | 4.8 | 7.5 |
| | with ≥2-Fold- | | | | |
| | Activation | | | | |
| | Range with ≥2- | 9.0-17.0 | 2.0-14.0 | 2.9-10.2 | 2.0-17.0 |
| | Fold-Activation | | | | |

| | | | | |
|---|---|---|---|---|
| N with ≥2-Fold-Activation (%) | 2 of 3 | 3 of 5 | 4 of 5 | 4 of 5 |
| | (67) | (60) | (80) | (80) |

TABLE 4
Activation of HIV-1 Expression by TLR8 Agonist
of Example 15 and/or IL-15 in PBMC Cultures
from HIV-1 Infected Subjects on cART

| | | Example Treatment (nM) | | |
|---|---|---|---|---|
| | Subject ID | Example 15 | IL-15 | Example 15 + |
| | (N = 6) | 1000 | 1 | IL-15 |
| Fold HIV-1 | 205 | 1.9 | 1.4 | 2.4 |
| Activation^{a} | 206 | 0.6 | 1.6 | 3.7 |
| | 207 | 4.7 | 2.6 | 6.4 |
| | 208 | 0.8 | 3.3 | 17.6 |
| | 209 | 9.7 | 4.4 | 343.1 |
| | 210 | 2.0 | 6.3 | 8.1 |
| | Geometric Mean | 2.1 | 2.9 | 12 |

| N with ≥2-Fold-Activation (%) | 3 of 6 | 4 of 6 | 6 of 6 |
|---|---|---|---|
| | 50 | 66 | 100 |

### Example 122

### Induced Cytokines and Chemokines in PBMC Cultures from HIV-1 Infected Subjects on cART Treated with TLR8 Agonist Example 15

To further characterize the immune modulatory effect of TLR8 agonists in cells from HIV-infected subjects, the production of secreted cytokines and chemokines was measured in PBMCs from 4 HIV-infected subjects on suppressive cART (anonymized participant IDs 180-183). PBMCs were purified, as described in Example 121, and immediately added to tissue culture plates pre-equilibrated at 37° C. with media containing DMSO (vehicle control) or with the TLR8 modulating compound Example 15 at concentrations ranging from 1.0 nM to 10.0 μM. On day 2 after treatment, cell-free culture supernatants were collected and frozen at −80 C for subsequent analysis. Frozen supernatants were thawed at room temperature, and the indicated cytokines and chemokines were quantified with a custom multiplexed xMAP Luminex® assay following the manufacturer's instructions (Thermo Fisher Scientific Inc., Grand Island, N.Y., custom-designed kit).

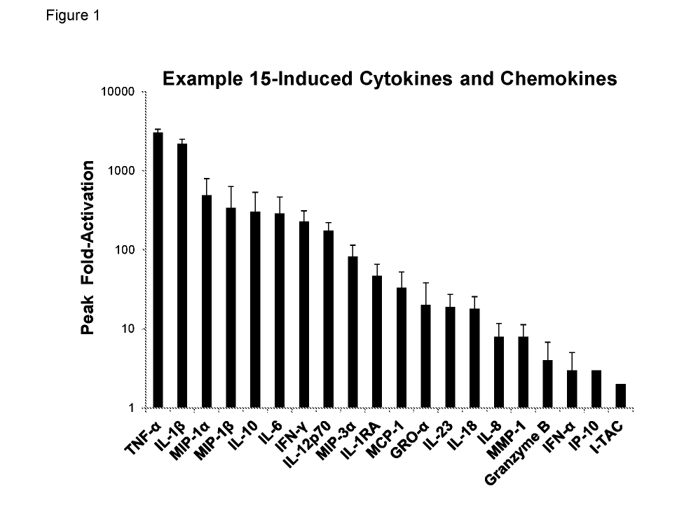

In the absence of other activation stimuli, treatment with the TLR8 agonist Example 15 activated peak levels of multiple cytokines and chemokines ≥2-fold compared to DMSO control in PBMC cultures from each subject tested (FIG. 1). Peak absolute levels of cytokines induced were achieved with Example 15 at concentrations ranging from 100 nM to 10000 nM. Peak fold-activations relative to DMSO ranged from 3051-fold for TNF-α, to 518-fold for IL-12p70, to 18-fold for IL-18 (Table 5). Example 15 did not induce the following cytokines or chemokines at levels >2-fold compared to DMSO control: IFN-ω, IL-2, IL-4, IL-5, IL-7, IL-15, IL-17A, IL-21, IL-27, or IL-29.

TABLE 5
Activation of Cytokines and Chemokines by TLR8 Example 15 in
PBMC Cultures from HIV-1 Infected Subjects on cART

| Ex 15 | TNF-α | | IL-1β | | MIP-1α | | MIP-1β | |
|---|---|---|---|---|---|---|---|---|
| (nM) | pg/ml | Fold^{a} | pg/ml | Fold | pg/ml | Fold | pg/ml | Fold |
| 10000 | 23017 | 1096 | 6611 | 2204 | 24365 | 487 | 239694 | 196 |
| 3000 | 64062 | 3051 | 5302 | 1767 | 21047 | 421 | 293183 | 240 |
| 1000 | 34880 | 1661 | 4735 | 1578 | 18689 | 374 | 415739 | 340 |
| 100 | 132 | 6 | 135 | 45 | 172 | 3 | 1302 | 1 |
| 10 | 35 | 2 | 2 | 1 | 70 | 1 | 1302 | 1 |
| 1 | n.d. | n.d.^{b} | 5 | 2 | 251 | 5 | 2189 | 2 |
| DMSO | 21 | n/a^{c} | 3 | n/a | 50 | n/a | 1222 | n/a |
| Ex. 15 | IL-10 | | IL-6 | | IFN-γ | | IL-12p70 | |
| (nM) | pg/ml | Fold^{a} | pg/ml | Fold | pg/ml | Fold | pg/ml | Fold |
| 10000 | 5167 | 304 | 18836 | 241 | 1478 | 59 | 113 | 38 |
| 3000 | 4747 | 279 | 15198 | 195 | 3586 | 143 | 381 | 127 |
| 1000 | 3719 | 219 | 22600 | 290 | 5693 | 228 | 524 | 175 |
| 100 | 2459 | 145 | 4424 | 57 | 49 | 2 | 6 | 2 |
| 10 | 21 | 1 | 61 | 1 | 48 | 2 | n.d. | n.d. |
| 1 | 25 | 1 | 61 | 1 | 50 | 2 | n.d. | n.d. |
| DMSO | 17 | n/a | 78 | n/a | 25 | n/a | 3 | n/a |
| Ex. 15 | MIP-3α | | IL-1RA | | MCP-1 | | GRO-α | |
| (nM) | pg/ml | Fold^{a} | pg/ml | Fold | pg/ml | Fold | pg/ml | Fold |
| 10000 | 1680 | 65 | 90289 | 25 | 1796 | 27 | 433 | 19 |
| 3000 | 2141 | 82 | 88340 | 25 | 1646 | 25 | 418 | 18 |
| 1000 | 1085 | 42 | 164731 | 46 | 1464 | 22 | 413 | 18 |
| 100 | 28 | 1 | 41213 | 12 | 2162 | 33 | 469 | 20 |
| 10 | 23 | 1 | 5309 | 1 | 91 | 1 | 29 | 1 |
| 1 | 23 | 1 | 11610 | 3 | 180 | 3 | 31 | 1 |
| DMSO | 26 | n/a | 3546 | n/a | 66 | n/a | 23 | n/a |
| Ex. 15 | IL-23 | | IL-18 | | IL-8 | | MMP-1 | |
| (nM) | pg/ml | Fold^{a} | pg/ml | Fold | pg/ml | Fold | pg/ml | Fold |
| 10000 | 965 | 16 | 218 | 13 | 1118 | 4 | 740 | 8 |
| 3000 | 1163 | 19 | 308 | 18 | 1139 | 4 | 311 | 3 |
| 1000 | 465 | 8 | 191 | 11 | 1565 | 5 | 317 | 3 |
| 100 | 130 | 2 | 20 | 1 | 2561 | 8 | 198 | 2 |
| 10 | 86 | 1 | 26 | 2 | 333 | 1 | 104 | 1 |
| 1 | 131 | 2 | 23 | 1 | 624 | 2 | 119 | 1 |
| DMSO | 60 | n/a | 17 | n/a | 309 | n/a | 98 | n/a |
| Ex. 15 | Granzyme B | | IFN-α | | IP-10 | | I-TAC | |
| (nM) | pg/ml | Fold^{a} | pg/ml | Fold | pg/ml | Fold | pg/ml | Fold |
| 10000 | 644 | 3 | 4 | 2 | 587 | 2 | 69 | 1 |
| 3000 | 857 | 4 | 6 | 3 | 711 | 3 | 98 | 2 |
| 1000 | 878 | 4 | 6 | 3 | 741 | 3 | 105 | 2 |
| 100 | 79 | <1 | 3 | 1 | 226 | 1 | 32 | 1 |
| 10 | 74 | <1 | 4 | 2 | 259 | 1 | 70 | 1 |
| 1 | 80 | <1 | 4 | 2 | 696 | 3 | 82 | 2 |
| DMSO | 236 | n/a | 2 | n/a | 264 | n/a | 51 | n/a |

Data are expressed as geometric mean cytokine values (pg/ml) obtained from 4 subjects' PBMCs. Geometric mean fold-activation from three independent incubation samples for each condition. Fold (fold-activation) is calculated as a ratio of the concentration of cytokine detected in cell culture supernatants from agonist treated samples relative to DMSO treated samples

### Example 123

### Induced Immune Cell Activation in PBMC Cultures from HIV-1 Infected Subjects on cART Treated with the TLR8 Agonist of Example 15.

TLR8 is expressed on multiple immune cell subsets. To determine how treatment with a TLR8 modulating compound affects activation of distinct cell populations, PBMCs were purified from HIV-1 infected subjects on cART, as described in Example 121 (anonymized participant IDs 184, 185, 188, 189, 190, and 191). PBMCs were treated with the TLR8 agonist of Example 15 at 10, 100, or 1000 nM, or with DMSO as a background control, and incubated for 2 days at 37° C. After incubation, PBMCs were harvested and stained with antibodies conjugated to specific fluorophores (BD Biosciences) to delineate immune cell subsets: CD4 T cells (CD3+CD4+CD8−), CD8 T cells (CD3+CD8+CD4−), NK cells (CD3−CD56+CD16+), monocytes (CD3−CD14+), B cells (CD3−CD19+), and peripheral dendritic cells (DCs), defined as positive for HLA-DR and negative for a panel of markers: CD3, CD14, CD16, CD19, CD20, CD56 (Lin-1 lineage panel, BD Biosciences).

Additional markers of activation, maturation, and effector function were included for cell subsets, as follows: CD4 and CD8 T cells (CD25, CD38, CD69, IFN-γ, granzyme B, perforin, TNF-α), NK cells (CD25, CD69, IFN-γ), monocytes (CD11c, CD16, CD80, CD86, CD163, TNF-α), B cells (CD40, CD69, CD80, CD86), and DC (CD11c, CD69, CD80, CD86, CD123, and CD163). Cells were stained for surface markers (CD3, CD4, CD8, CD11c, CD14, CD16, CD25, CD69, CD80, CD86, CD163), washed, treated with fixation and permeabilization buffer (eBioscience), and stained for intracellular markers (IFN-γ, granzyme B, perforin, TNF-α). Cells were evaluated by flow cytometry on a LSR Fortessa (BD Biosciences), and data was analyzed with FlowJo software (TreeStar).

The compound of Example 15 maximally activated CD4+ T cells at concentrations of 1000 or 3000 nM, as indicated by increased expression of CD25, CD69, and perforin (Table 6). Example 15 maximally activated CD8+ T cells at concentrations of 1000 or 3000 nM, as indicated by increased expression of CD25, CD28, CD38, and CD69 (Table 6). Perforin did not increase with Example 15 treatment in CD8+ T cells. The tested compound maximally activated B cells at concentrations of 3000 nM, as indicated by increased expression of CD69 and CD80 (Table 6). The tested compound maximally activated NK cells at concentrations of 1000 nM, as indicated by increased expression of CD25 and CD69 and maximally activated monocytes at concentrations of 100, 3000, and 1000 nM, as indicated by increased expression of CD11c, CD69, and CD80 respectively (Table 6). Finally, the tested compound maximally activated DCs at concentrations of 3000 or 1000 nM, as indicated by increased expression of CD69 and CD80 respectively (Table 6).

TABLE 6
Activation of Immune Cell Subsets by TLR8 Agonist of Example 15 in
PBMC Cultures from HIV-1 Infected Subjects on cART

| Example | CD4+ T Cells (N = 6) | | |
|---|---|---|---|
| 15 | CD25 | CD69 | Perforin |

| (nM) | % | Fold | % | Fold | % | Fold |
|---|---|---|---|---|---|---|
| 3000 | 3.0 | 1.3 | 13.1 | 1.7 | 17.0 | 2.1 |
| 1000 | 4.2 | 1.8 | 11.7 | 1.5 | 14.2 | 1.8 |
| 100 | 3.9 | 1.7 | 7.7 | 1.0 | 10.4 | 1.3 |
| 10 | 1.5 | 0.6 | 5.7 | 0.7 | 11.8 | 1.5 |
| DMSO | 2.3 | n.a. | 7.7 | n.a. | 8.1 | n.a. |

| Example | CD8+ T Cells (N = 6) | | | | | | | |
|---|---|---|---|---|---|---|---|---|
| 15 | CD25 | | CD28 | | CD38 | | CD69 | |
| (nM) | % | Fold^{a} | % | Fold | % | Fold | % | Fold |
| 3000 | 0.9 | 3.0 | 0.5 | 4.7 | 56.8 | 2.0 | 11.6 | 2.5 |
| 1000 | 0.7 | 2.2 | 0.4 | 4.4 | 56.8 | 2.0 | 10.4 | 2.2 |
| 100 | 0.3 | 1.0 | 0.1 | 1.1 | 29.2 | 1.0 | 3.4 | 0.7 |
| 10 | 0.2 | 0.6 | 0.1 | 0.9 | 25.3 | 0.9 | 3.7 | 0.8 |
| DMSO | 0.3 | n.a.^{b} | 0.1 | n.a. | 28.2 | n.a. | 4.7 | n.a. |

| Example | B Cells (N = 4) | |
|---|---|---|
| 15 | CD69 | CD80 |

| (nM) | % | Fold | % | Fold |
|---|---|---|---|---|
| 3000 | 13.6 | 1.7 | 3.0 | 1.9 |
| 1000 | 9.1 | 1.1 | 2.2 | 1.4 |
| 100 | 3.9 | 0.5 | 1.7 | 1.1 |
| 10 | 10.0 | 1.2 | 2.6 | 1.6 |
| DMSO | 8.1 | n.a. | 1.6 | n.a. |

| Example | NK Cells (N = 6) | |
|---|---|---|
| 15 | CD25 | CD69 |

| (nM) | % | Fold | % | Fold |
|---|---|---|---|---|
| 3000 | 4.8 | 9.6 | 20.1 | 11.8 |
| 1000 | 5.2 | 10.4 | 21.1 | 12.4 |
| 100 | 0.8 | 1.6 | 1.1 | 0.6 |
| 10 | 0.2 | 0.4 | 0.8 | 0.4 |
| DMSO | 0.5 | n.a. | 1.7 | n.a. |

| Example | Monocytes (N = 6) | | |
|---|---|---|---|
| 15 | CD11c | CD69 | CD80 |

| (nM) | % | Fold | % | Fold | % | Fold |
|---|---|---|---|---|---|---|
| 3000 | 1.5 | 2.1 | 7.9 | 2.6 | 77.4 | 2.7 |
| 1000 | 1.2 | 1.6 | 6.2 | 2.0 | 86.4 | 3.0 |
| 100 | 3.6 | 5.2 | 1.8 | 0.6 | 47.7 | 1.6 |
| 10 | 0.4 | 0.6 | 0.8 | 0.3 | 33.0 | 1.1 |
| DMSO | 0.7 | n.a. | 3.1 | n.a. | 29.1 | n.a. |

| Example | DCs (N = 4) | |
|---|---|---|
| 15 | CD69 | CD80 |

| (nM) | % | Fold | % | Fold |
|---|---|---|---|---|
| 3000 | 23.2 | 8.9 | 57.1 | 2.0 |
| 1000 | 8.5 | 3.3 | 82.0 | 2.9 |
| 100 | 1.0 | 0.4 | 67.2 | 2.4 |
| 10 | 0.3 | 0.1 | 49.8 | 1.8 |
| DMSO | 2.6 | n.a. | 28.1 | n.a. |

Data are expressed as geometric mean percentage (%) and as geometric mean fold-activation (Fold) for each marker and for each PBMC subset obtained from 4 or 6 subjects, as indicated. Fold activation is calculated as a ratio of the % positive for each marker for each PBMC subset at the indicated concentration of the compound of Example 15 relative to % positive for DMSO treated samples. Fold activations that are 1.5 are in bold.

### Example 124

### Induced HIV-Specific Polyfunctional CD8+ T Cells in PBMC Cultures from HIV-1 Infected Subjects on cART Treated with TLR8 Agonists

Polyfunctional CD8 T cells are capable of killing viral-infected cells via antigen-specific cellular cytotoxicity. To assess the ability to activate polyfunctional HIV-specific CD8 T cells in total PBMC cultures, PBMCs were purified from 5 HIV-1 infected subjects on cART, as previously described. On Day 0, PBMCs were labeled with carboxyfluorescein succinimidyl ester (CFSE, Life technologies), treated with the indicated concentrations of the TLR8 modulating compound of Example 15, and cultured at 37° C. in the presence of antiviral drugs (elvitegravir and efavirenz at 100 nM each). On Day 2, PBMCs were treated with media alone or HIV peptide pools (80 ng/ml of overlapping 15-mer peptides to Gag and Nef, JPT Peptide Technologies). On Day 8, PBMCs were washed and either re-treated with HIV peptide pools, or in parallel cultures, treated with CEFT peptide pools to measure the non-specific CD8 T cell response (15-mer peptides to cytomegalovirus, epstein barr virus, influenza, and tetanus toxoid; PepMix, JPT Peptide Technologies). Peptide recall was done by culturing at 37° C. for 4 hours. After 1 hour, monensin and brefeldin (GolgiStop and GolgiPlug, BD Biosciences) were added. After 3 additional hours, PBMCs were washed and stained with live-dead aqua dye (Life Technologies) and surface stained with antibodies to CD3, CD4, and CD8 (BD Biosciences). Cells were fixed and permeabilized using the Cytofix/Cytoperm kit (BD Bioscience) and stained with antibodies to IFN-γ (BD Biosciences) and TNF-α (e-Biosciences). Stained cells were evaluated by flow cytometry on a LSR Fortessa (BD Biosciences), and data was analyzed with FlowJo software (TreeStar). Polyfunctional CD8 T cells were determined by gating on live, CD8+, CFSE low, IFN-γ+, TNF-α+events. HIV-specific CD8 responses were obtained by subtracting % gated cells responding to CEFT peptide recall group from gated cells numbers responding to HIV peptide recall.

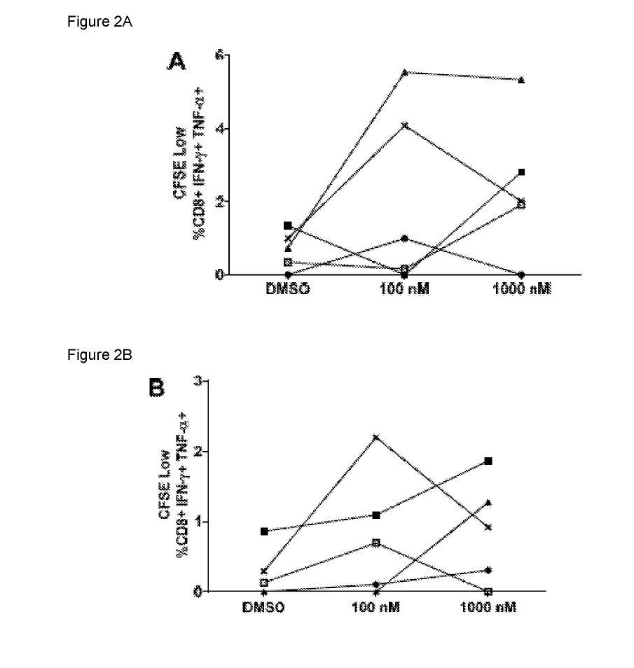

Treatment with the TLR8 agonist of Example 15 (FIG. 2A) and Example 98 (FIG. 2B) activated HIV-specific polyfunctional CD8 T cells, as indicated by an increased % of proliferating CD8+ cells double positive for IFN-γ and TNF-α. For Example 15 (FIG. 2A), the median HIV-specific peptide pool increased from 0.73% with the vehicle control to 0.99% at 100 nM and 2.01% at 1000 nM. With Example 15 treatment, increases were seen in 3 of 5 patients at 100 nM and in 4 of 5 patients at 1000 nM. For Example 98 (FIG. 2B), the median HIV-specific peptide pool increased from 0.13% with the vehicle control to 0.93% at 100 nM and 0.92% at 1000 nM. With Example 98 treatment, increases were seen in 4 of 5 patients at both concentrations. As these compounds were assessed in samples from different patients, the levels of HIV-specific polyfunctional T cells varied between the two groups. However, treatment with 1000 nM of the compounds of Example 15 and Example 98 combined with the HIV specific peptide pool increased median HIV-specific polyfunctional CD8 T cells 2.7-fold and 7.1 fold, respectively (p<0.05, unpaired one-tailed t-test).

### Example 125

### Induced Antibody-Mediated Killing of HIV-Infected CD4 T Cells with a TLR8 Agonist

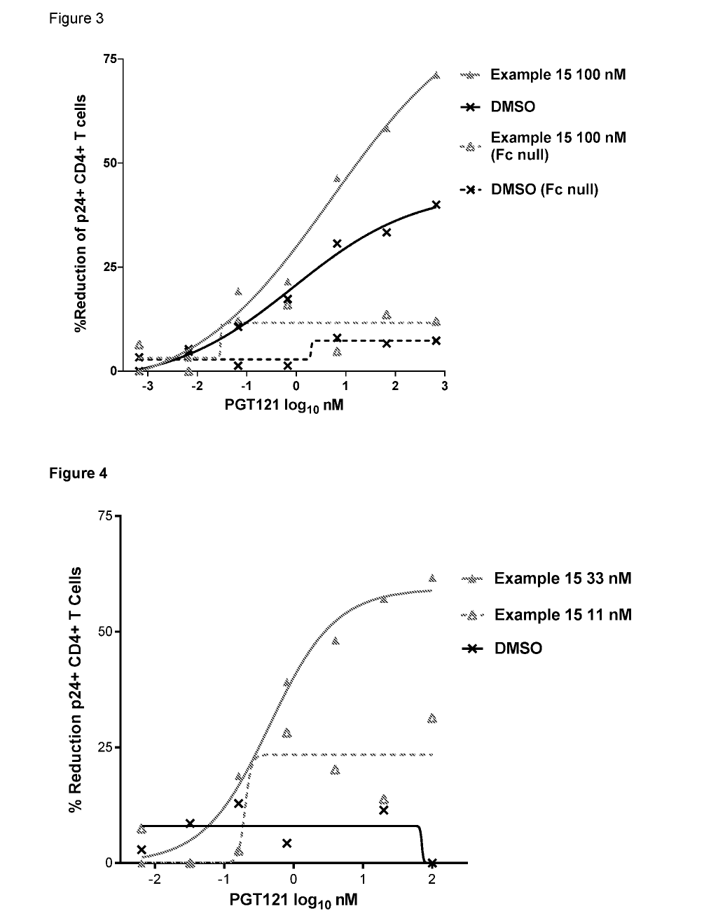

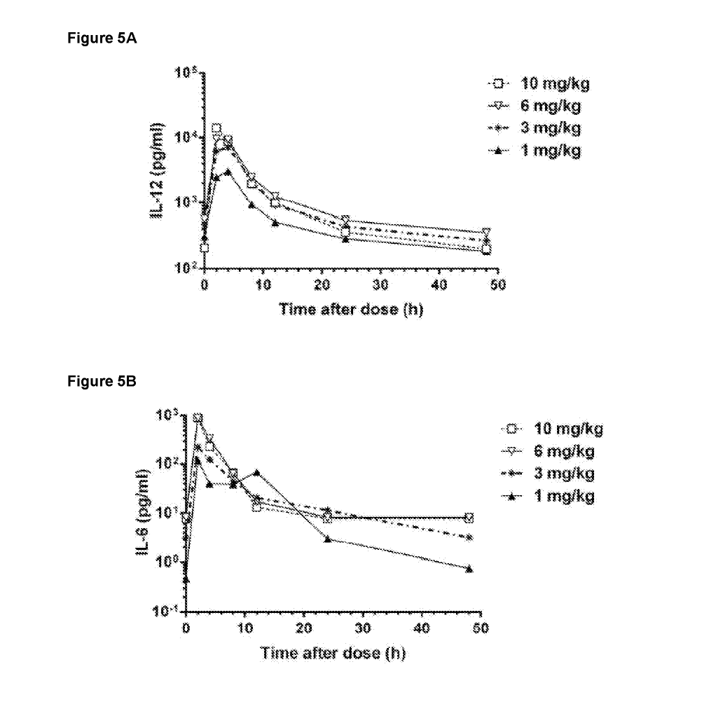

The TLR8 modulating compound of Example 15 activates NK cells and monocytes, as demonstrated above in Example 123. NK cells and monocytes mediate antibody-dependent cellular cytotoxicity (ADCC) and antibody-dependent cellular phagocytosis (ADCP) respectively. To assess the ability of the compound of Example 15 to enhance ADCC and ADCP, two models were developed using monocytes or PBMCs as effector cells and HIV-infected CD4 T cells as target cells. PBMCs were isolated from 3 healthy subjects' leukaphereses (AlICells, participant IDs A4593, A4596, A4606), as described above. CD4 T cells were isolated from PBMCs using negative selection magnetic beads (EasySep, StemCell Technologies). CD4 T cells were spinfected with HIV-1 isolate 92HT593 (NIH AIDS Reagent Program) and cultured for 5 days at 37° C. For the PBMC model, autologous PBMCs were cultured for 5 days with vehicle control (DMSO) or Example 15 at 100 or 1000 nM. For the monocyte model, PBMCs were isolated from 3 healthy subjects' leukaphereses (AllCells, participant IDs A4611, A4668, A4673) and CD4 T cells were purified and infected with HIV-1 isolate 92US657 (NIH AIDS Reagent Program), as described above. Monocytes were isolated using negative selection magnetic beads (EasySep, Stemcell Technologies) from frozen autologous PBMCs that were thawed the day of isolation. Monocytes were treated with DMSO or compounds at 1 to 33 nM and cultured for 1 day at 37° C. All cells were thoroughly washed after the indicated culture times. Pre-treated PBMCs or monocytes were added to HIV-infected CD4 T cells at ratios of 20:1 or 2:1 respectively. PGT121 is a broadly neutralizing anti-HIV Env antibody. Two forms of PGT121 antibody were tested: one with a functional wild type Fc domain and one with a FES mutant Fc that has reduced Fc receptor binding and reduced antibody effector function, referred to as Fc null (Gilead Sciences). PGT121 or PGT121-Fc null were added at 1 ng/ml to 50 pg/ml in combination with purified human IgG fraction at 1 mg/ml to approximate biologically relevant concentrations of competing antibody found in plasma. Cells with or without antibodies were co-cultured at 37° C. for 1 day. After incubation, cells were stained with live/dead Fixable Aqua Dead Cell Stain (Invitrogen) and antibodies to CD3, CD4, and CD16 (BD Biosciences). Cells were fixed and permeabilized (eBioscience) and stained with antibody to HIV Gag p24 (Beckman Coulter). Marker staining was measured by flow cytometry using a LSR Fortessa (BD Biosciences) and analyzed with FlowJo software (TreeStar). The percent killing was determined by calculating the reduction of p24+CD4+ T cells mediated by PGT121 antibody relative to a no antibody control. In PBMCs from 3 subjects, the compound of Example 15 at 100 nM enhanced the potency and maximal PGT121-mediated killing of HIV-infected CD4+ T cells, as indicated by a geometric mean area under the curve (AUC) of 172 compared to 108 for the DMSO control, a 60% increase (FIG. 3). In monocytes from 1 subject, the tested TLR8 modulator at 11 or 33 nM markedly enhanced PGT121-mediated killing of HIV-infected CD4+T cells compared to the DMSO control (FIG. 4).

PBMCs (FIG. 3) or monocytes (FIG. 4) were treated with DMSO or TLR8 agonist for 5 days (FIG. 3) or 1 day (FIG. 4) and then co-cultured for 1 day with autologous HIV-infected CD4 T cells−/+anti-HIV Env antibody PGT121. Reductions in %p24+ CD4+ T cells, relative to no antibody control, were measured by FACS. Representative data from 2 subjects are shown in the respective figures.

### Example 126

### Induction of Cytokines and Chemokines by TLR8 Example 65 in Rhesus Macaques

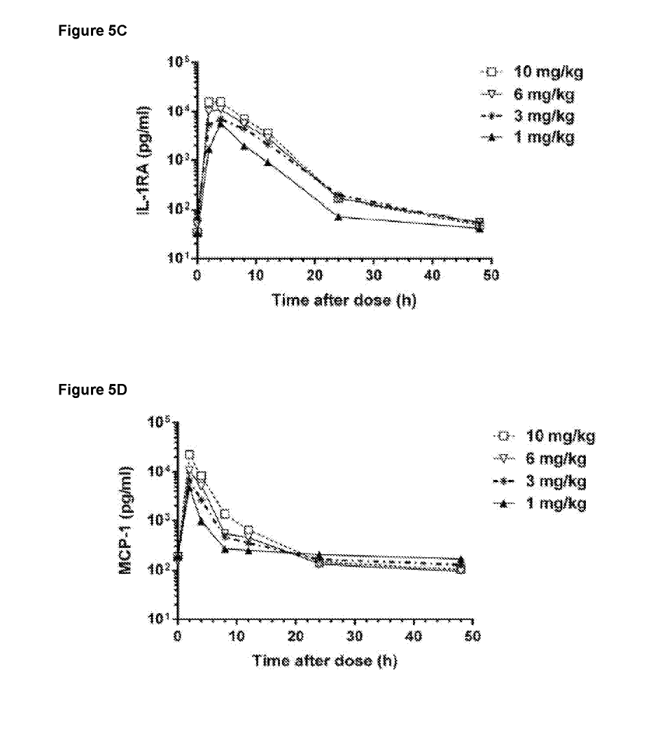

The TLR8 modulator of Example 65 was formulated in 2% ethanol, 40% polyethylene glycol 300, and 58% 0.01 N hydrochloric acid. Oral solutions of formulations were prepared by dissolving a sufficient amount of the compound in the 2% ethanol, 40% polyethylene glycol 300, and 58% 0.01 N hydrochloric acid vehicle to arrive at 0.5, 1.5, or 5 mg/ml of Example 65. Rhesus macaques were dosed orally with 1.2 to 2 mls/kg to achieve dose levels of 1, 3, 6, or 10 mg/kg Example 65. Blood samples were collected at 2, 4, 8, 12, 24 and 48 hours postdose (FIG. 5) and allowed to clot under ambient conditions prior to centrifugation to obtain serum. The indicated cytokines and chemokines were quantified in serum with a multiplexed Luminex® assay following the manufacturer's instructions (Life Technologies). Cytokines associated with TLR8 activation were induced in a dose-dependent manner, including IL-12, IL-6, IL-1RA and MCP-1. Peak concentrations were achieved at 2 hours post-dose. At 10 and 6 mg/kg, IL-12, IL-1RA and MCP-1 peaked near 10 ng/ml and IL-6 peaked at 1 ng/ml.

### Prophetic Example 126 Formulation of TLR8 Modulators

Formulation of TLR8 modulators for oral dosing may be prepared as an oral solution or suspension in a vehicle containing water with additional optional organics such as co-sovents and/or surfactants. The formulation can optionally be pH adjusted using a suitable acid or base to increase the solubility of the TLR8 modulator. The formulation may also be optinally buffered with a suitable buffer system to maintain a specific pH of the final formulation.

Specifically, the TLR8 modulator, or a pharamaceutically acceptable salt thereof, is prepared as an oral solution or suspension in a vehicle containing 0-99% water, with the remaininder consisting of one or more of the following: 0-10% ethanol, 0-60% propylene glycol, 0-60% polyethylene glycol, 0-5% hydroxymethyl propyl methylcellulose, and/or 0-5% surfactants.

A specific example formulation for a TLR8 modulator is prepared by preparing a vehicle of 2% ethanol, 40% polyethylene glycol 300, 58% 0.01N hydrochloric acid. Oral solutions formulations of TLR8 modulator compounds were prepared by dissolving a sufficient amount of the compound in the 2% ethanol, 40% polyethylene glycol 300, 58% 0.01N hydrochloric acid vehicle to arrive at a final concentration of 0.125 mg/mL, 0.25 mg/mL, 0.5 mg/mL, and 1.0 mg/mL of the TLR8 modulator compound.

### Example 126b

Alternatively a suspension formulation suitable for oral dosing of a TLR8 modulator compound was prepared by preparing a vehicle of 0.5% Hydroxypropyl methylcellulose, and 0.5% polysorbate 80, and 99% water. Oral suspension formulations of TLR8 modulator compounds were prepared by suspending a sufficient amount of the compound in the vehicle to arrive at a final concentration of 1.0 mg/mL, 3.0 mg/mL, 10 mg/mL, 30 mg/mL, and 100 mg/mL of the TLR8 modulator compound.

### Prophetic Example 127

### cART Formulation

A cART formulation containing 20 mg/mL tenofovir (TFV, GMP grade), 50 mg/mL emtricitabine (FTC, GMP grade), and 2.5 mg/mL dolutegravir (DTG, 95% purity) is prepared in a solvent containing a final concentration of 25% (v/v) polyethylene glycol 400 (PEG-400, Spectrum Chemical New Brunswick, N.J.), 15% (w/v) Captisol (Ligand Pharmaceuticals, LA Jolla, Calif.) and 0.075N sodium hydroxide (NaOH, Spectrum Chemical, New Brunswick, N.J.) in water. This formulation is prepared by mixing DTG stock solution (10 mg/mL of DTG in PEG-400), TFV and FTC stock solution (80 mg/mL of TFV and 200 mg/mL of FTC in 0.3 N NaOH), and 30% (w/w) captosil solution at a 1:1:2 (v:v:v) ratio. The final solution has a pH ˜6. It is sterile filtered, then aliquoted into sterile glass and frozen at −20° C. until used. Similar formulations of other cART combinations may be prepared in a similar manner.

### Prophetic Example 128

### TLR8 Modulating Compound in SIV+ Viremic Rhesus at Chronic Set Point

A suitable number (e.g. five) of chronically infected, SIV⁺ rhesus macaques are either treated with a TLR8 modulating compound or with dosing vehicle (placebo) in a multiple-dose, dose escalation, placebo controlled study. The macaques are infected with simian immunodeficiency virus for sufficient period of time to establish a chronic set point (i.e., a state of persistent viremia, i.e., post-peak viremia where host immunological control of virus replication is established, although this does not lead to complete viral suppression) prior to dosing with the TLR8 modulating compound. The animals are not administered combination antiretroviral therapy during any part of the study.

The animals are dosed once weekly by oral gavage (via nasogastric tube) with either the TLR8 modulating compo or placebo. Three animals are administered the TLR8 modulating compound prepared as described in Example 126. Dose escalation occurs over a period of three weeks. Each week, the placebo group is administered the dosing vehicle as described in Example 126 at 1 mL/kg.

Each formulation of Example 126 or dosing vehicle only (placebo) is used fresh and thawed to room temperature prior to dosing.

Plasma viral loads (expressed as SIV RNA copies per mL of plasma) are determined by quantitative RT-PCR of the SIV RNA measuring the gag gene transcripts at various times throughout the study for animals administered the TLR8 modulating compound and the placebo animals.

### Prophetic Example 129

### TLR8 Modulating Compound in cART Treated SIV+ Viremic Rhesus with Undetectable Plasma Viremia

A group (e.g. 5) of Indian rhesus macaques (Macaca mulatta) are treated with a TLR8 modulating compound or dosing vehicle (placebo group) in a multiple dose, dose escalation study. The macaques are infected with simian immunodeficiency virus (SlVmac251) by repeat low-dose rectal mucosal challenge.

After establishment of peak viremia and an early viral chronic set point, the animals are treated with cART by sub-cutaneous injection at 1 mL/kg, the administration of which is for approximately one year. During the time of cART administration plasma viral loads decrease to undetectable levels (SIV gag RNA<50 copies/mL) post-infection, and are maintained for approximately 5 months prior to administration of the TLR8 modulating compound by use of daily cART. In addition, animals are maintained on the same cART regimen when dosed with the TLR8 modulating compound.The amount of antiretrovirals administered to each animal, on a daily basis, in the cART formulation may include 20 mg/kg tenofovir, 50 mg/kg emtricitibine (FTC), and 2.5 mg/kg dolutegravir. The animals are administered cART daily from approximately day 65 post-infection, through viral suppression and administration of the TLR8 modulating compound until approximately two weeks post-last dose of the TLR8 modulating compound at which time cART is stopped to monitor plasma viral load rebound kinetics.

At a suitable time post infection, for example after about 300 days, the animals are dosed once every other week with either the TLR8 modulating compound or dosing vehicle (placebo group). Animals were dosed with a TLR8 modulating compound. The first week, a suitable dose, e.g. 1 mL/kg of the 0.1 mg/mL formulation is dosed, corresponding to a dose of 0.1 mg/kg. The third week, a suitable dose, e.g. 1 mL/kg of the 0.2 mg/mL formulation is dosed, corresponding to a dose of 0.2 mg/kg. On weeks five through thirteen, a suitable dose, e.g. 1 mL/kg of the 0.3 mg/mL formulation is dosed, corresponding to a dose of 0.3 mg/kg (seven doses in total). The doses may be modified based on the particular TLR modulating compound used, as understood by one of skill in the art. Control animals, certain of which are given seven doses of saline by oral gavage once every two weeks; the other control animals are left untreated. Approximately two weeks following the last dose of the TLR8 modulating compound, cART is stopped in the saline-dosed placebo animals and the four animals who receive the TLR8 modulating compound. Approximately one month later, cART is stopped in the three untreated control animals. Following cART cessation viral loads are monitored for a period of six months.

Each formulation of the TLR8 modulating compound or dosing vehicle of Example 126 is thawed at room temperature prior to dosing. Animals are dosed by oral gavage via nasogastric tube.

Plasma viral loads (expressed as SIV RNA copies per mL of plasma) are determined by quantitative RT-PCR of the SIV RNA measuring the gag gene transcripts at various times throughout the study.

The data from this study is used to demonstrate that a TLR8 modulating compound reproducibly induces transient plasma viremia in the presence of cART followed by a return to full plasma SIV RNA suppression.

### Prophetic Example 130

### Proviral SIV DNA Measurements in Peripheral Blood Mononuclear Cells (PBMCs), Lymph Node, and Colon

A whole blood sample, an inguinal lymph node sample, and a colon pinch biopsy sample are taken from the animals dosed with a TLR8 modulating compound and from animals dosed with the vehicle of Example 126 (placebo group) in the study described in Example 129. These samples are taken prior to initiation of dosing and two weeks after the last dose; all samples are taken while animals were on cART. Total DNA is isolated from each sample using commercially available blood and tissue DNA extraction kits. Proviral SIV DNA levels are measured by quantitative PCR (qPCR) using probe-primer sets specific to the SIV gag gene and normalization for cellular input by measuring the glyceraldehyde 3-phosphate dehydrogenase (GAPDH) gene levels. A comparisdon of proviral SIV DNA amounts in the pre- and post-treatment PBMC, inguinal lymph node, and colon samples within each animal is made.

### Prophetic Example 131

### Peripheral Lymphocyte Activation

During the course of treatment with a TLR8 modulating compound as described in Example 129, peripheral lymphocyte activation is monitored in the groups dosed with the TLR8 modulating compound or the dosing vehicle of Example 126 (placebo), by measuring the expression of the early activation marker CD69 on various lymphocyte subsets by flow cytometry methodology. This methodology is used to quantify the percentage of immune cell subsets expressing the early activation marker. Standard data acquisition techniques are employed on a flow cytometer and by the use of antibodies conjugated to fluorescent molecules where the antibodies recognize rhesus macaque cell surface proteins on various immune cell subsets. In animals dosed with the TLR8 modulating compound, whole blood samples are collected for each dose at various time points both at pre- and post-administration of compound. Total PBMCs are stained in the whole blood samples using antibodies that are chosen to be reactive to rhesus CD3, CD4, CD8, NKG2A, CD16 and CD56, each conjugated to different fluorescent probes to delineate CD3+CD4+CD8− lymphocytes, CD3+CD4−CD8+ lymphocytes and CD3−CD8+NKG2A+CD16+CD56+ NK cells.

Data from this example will help demonstrate that TLR8 modulating compounds are able to induce transient expression of plasma SIV in the rhesus macaque model of HIV-1 infection, reduce the amount of proviral SIV DNA content in PBMCs, inguinal lymph node, and colon tissue which are markers for reduction of the viral reservoir, and produce an immunological response that is measureable in the absence of cART, as demonstrated by a lower plasma virus chronic set point (post-cART cessation) after the administration of the compound.

We claim:

1. A method of treating an HIV infection in a human, the method comprising administering to a human in need thereof a pharmaceutically effective amount of a TLR8 modulating compound, or a pharmaceutically acceptable salt thereof.

2. A method of treating an HIV infection in a human, the method comprising:
a) administering to a human in need thereof a pharmaceutically effective amount of a combination antiretroviral therapy regimen sufficient to lower the level of HIV detected in the human's blood or plasma from a first level to a second level, the second level comprising a lower concentration of HIV in the human's blood or plasma than the concentration of HIV in the human's blood or plasma in the first level; and
b) administering to the human a pharmaceutically effective amount of a TLR8 modulating compound, or a pharmaceutically acceptable salt thereof.

3. The method of claim 2 wherein the first level of HIV in the human's plasma is below 50 copies of HIV RNA/ml.

4. The method of claim 2 wherein the second level of HIV in the human's plasma is below 30 copies of HIV RNA/ml.

5. The method of claim 2 wherein the first level of HIV in the human's plasma is below 10 copies of HIV RNA/ml.

6. The method of claim 2 wherein the first level of HIV in the human's plasma is below 1 copy of HIV RNA/ml.

7. The method of treating an HIV infection in a human of any of claims 1-6, the method further comprising the step of administering to the human a pharmaceutically effective amount of an HIV antibody.

8. The method of treating an HIV infection in a human of any of claims 1-7, the method further comprising the step of administering to the human in need thereof a pharmaceutically effective amount of an HIV vaccine.

9. The method of any one of claims 1-8, wherein the TLR8 modulating compound is a compound of Formula (IV), 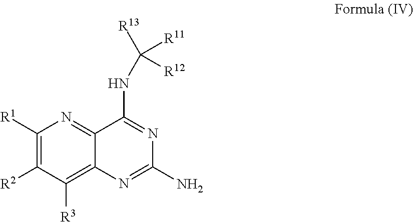
or a pharmaceutically acceptable salt thereof, wherein:
R¹ is selected from the group consisting of hydrogen, halogen, C₁₋₆ alkyl, CN, and OR^{a}, wherein C₁₋₆ alkyl is optionally substituted with 1 to 5 R²⁰ groups;
R² is selected from the group consisting of hydrogen, halogen, C₁₋₆ alkyl, CN, and OR^{a}, wherein C₁₋₆ alkyl optionally substituted with 1 to 5 R²⁰ groups;
R³ is selected from the group consisting of hydrogen, halogen, C₁₋₆ alkyl, CN, and OR^{a}, wherein C₁₋₆ alkyl is optionally substituted with 1 to 5 R²⁰ groups;
R¹¹ is selected from the group consisting of hydrogen, C₁₋₂ alkyl, C₃₋₆ cycloalkyl, and C₁₋₃ haloalkyl;
R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
each R²° is independently selected from the group consisting of halogen, CN, —NR^{a}R^{b}, and OR^{a}; and
each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

10. The method of claim 9, wherein the TLR8 modulating compound is a compound of Formula (IVa)

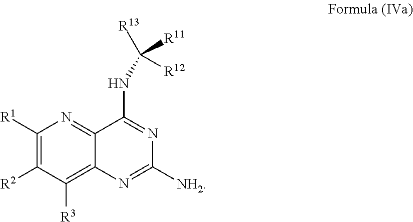

11. The method of claim 9, wherein the TLR8 modulating compound is a compound of Formula (IVb)

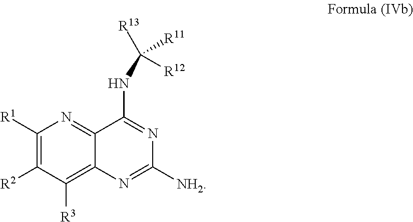

12. The method of claim 10, wherein the moiety

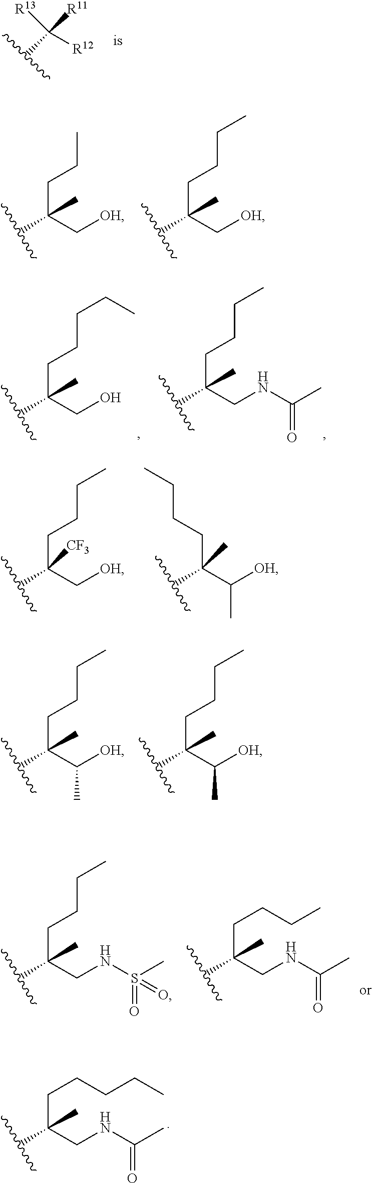

13. The method of claim 11, wherein the moiety

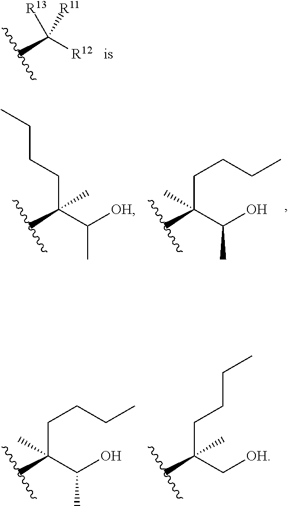

14. The method of any one of claims 1-8, wherein the TLR8 modulating compound is selected from the group consisting of: 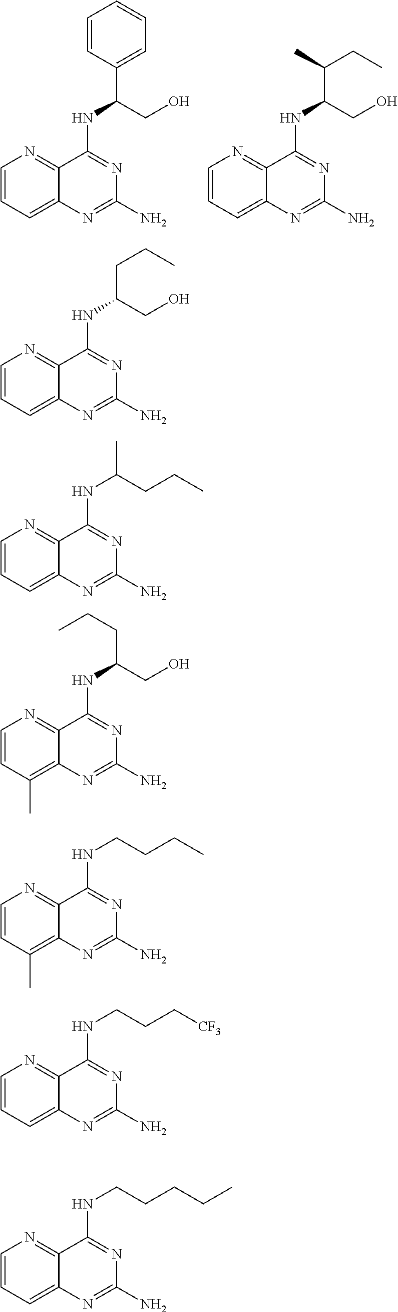 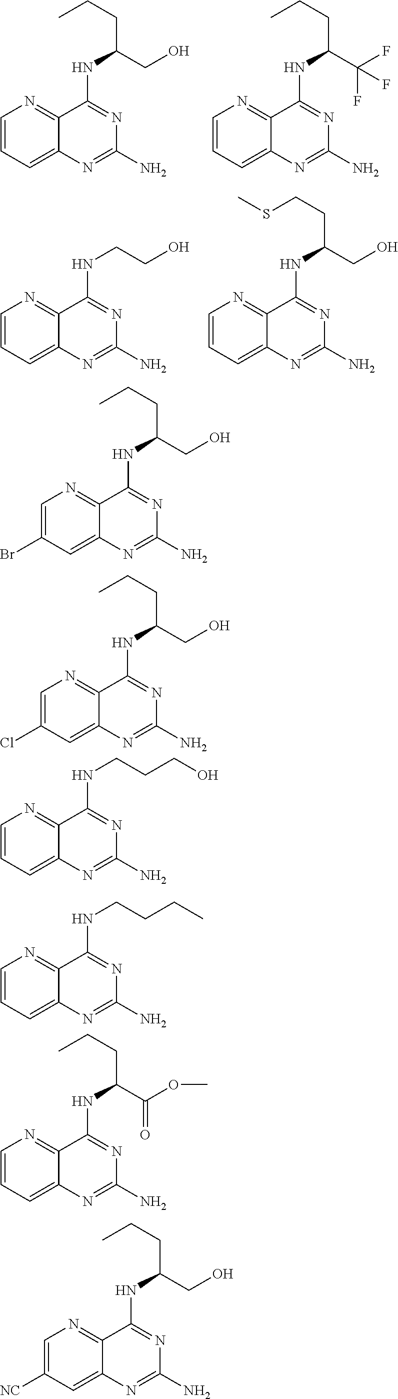 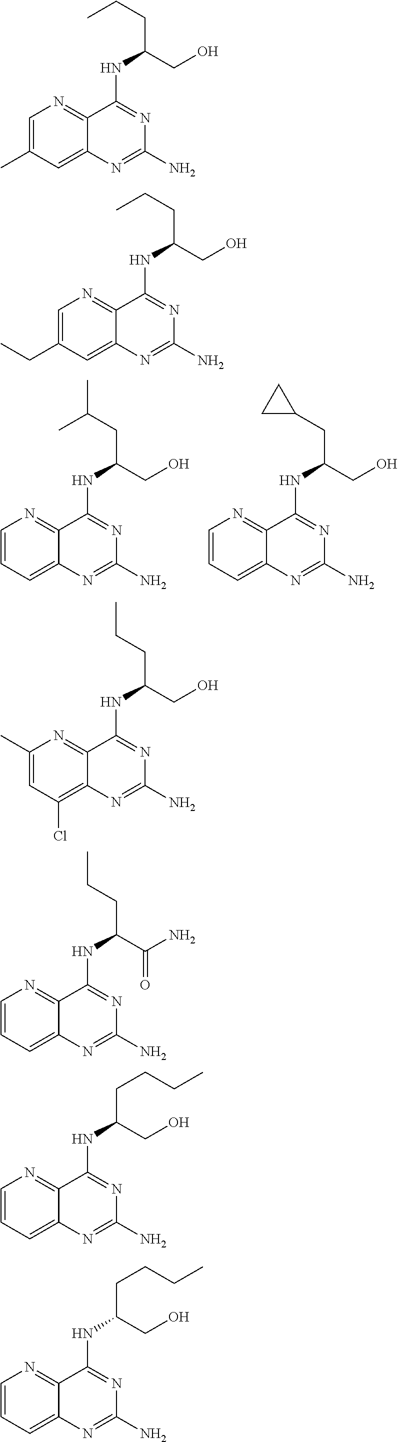 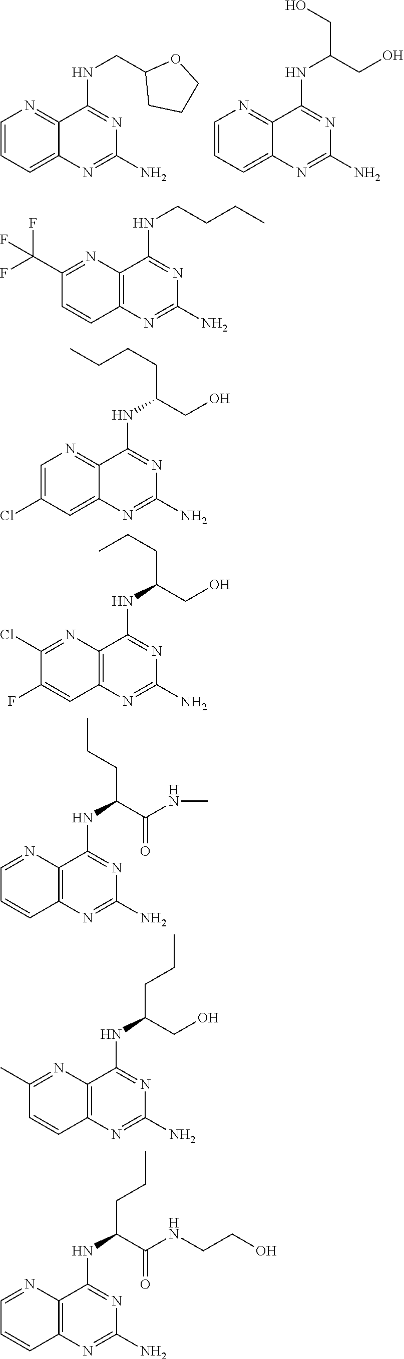 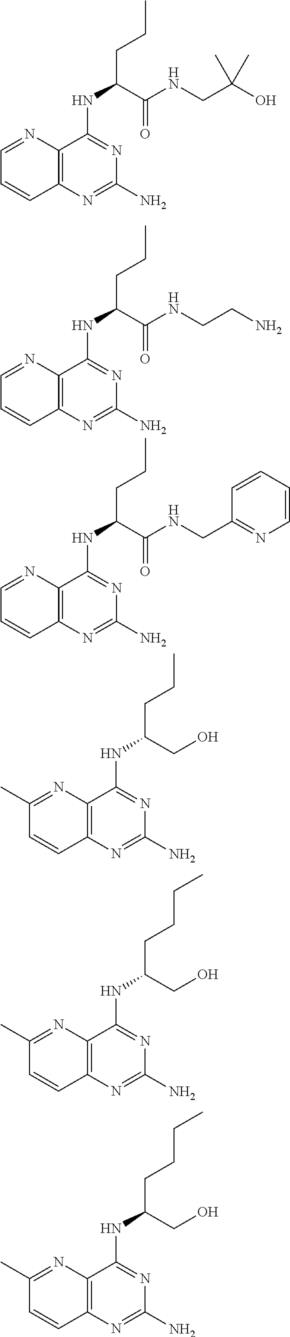 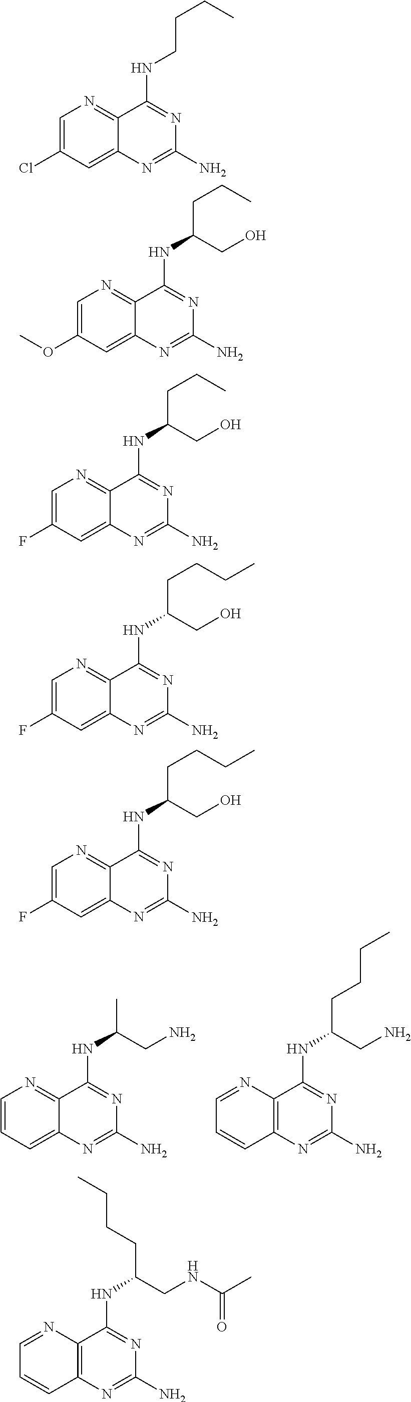 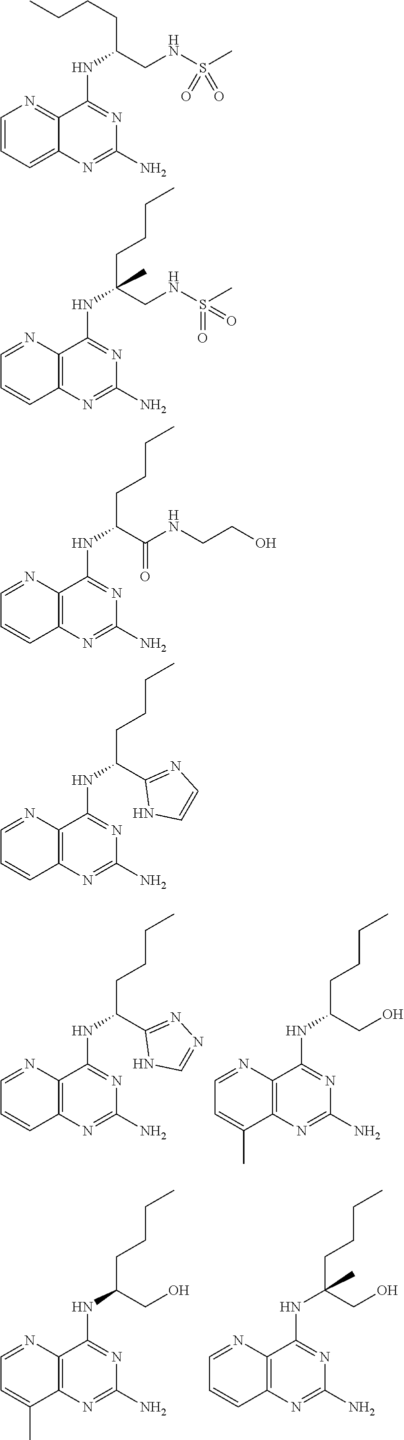 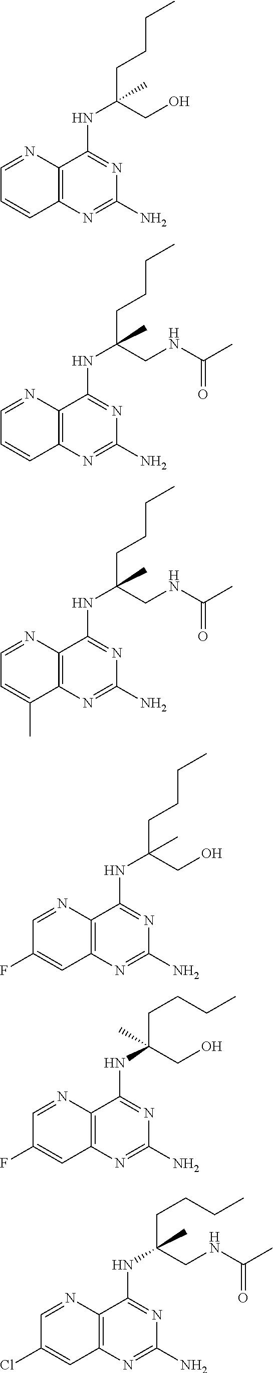 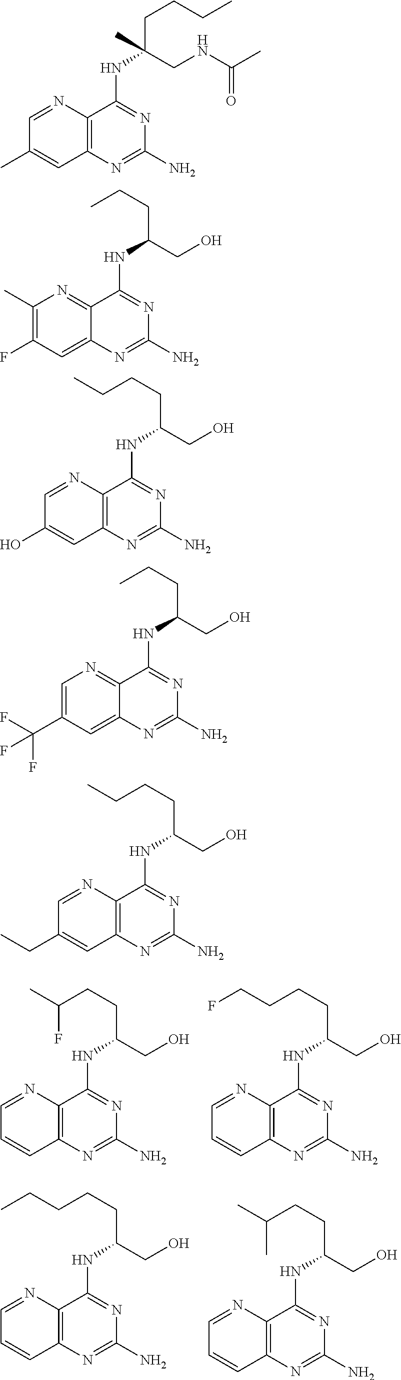 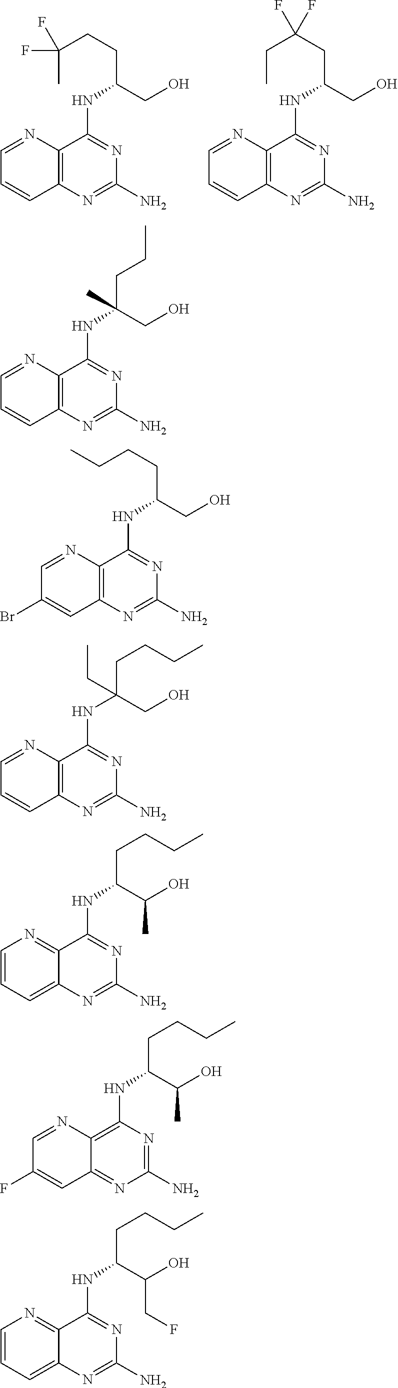 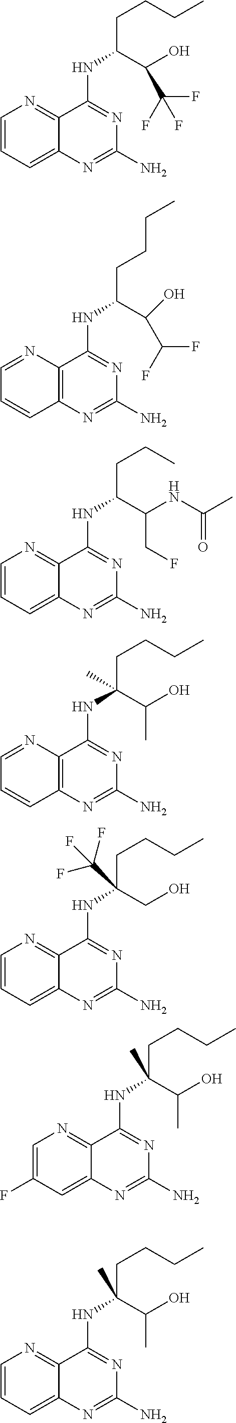 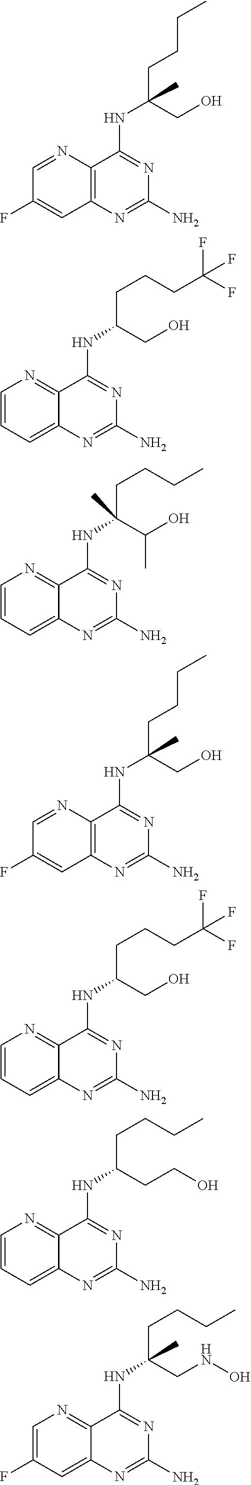 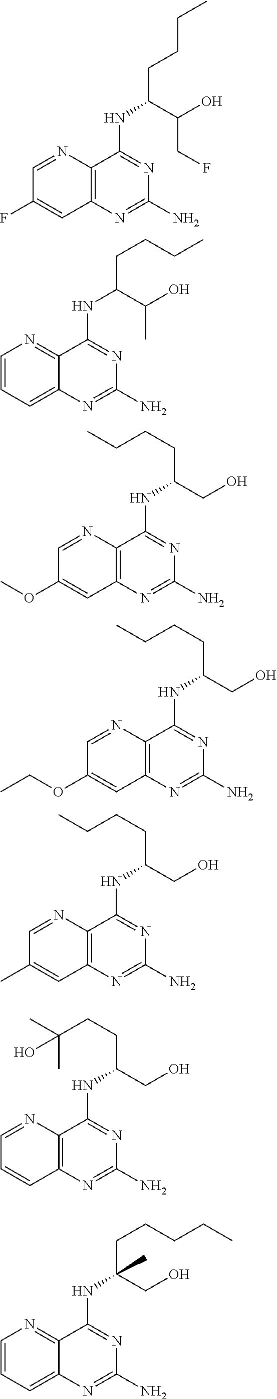 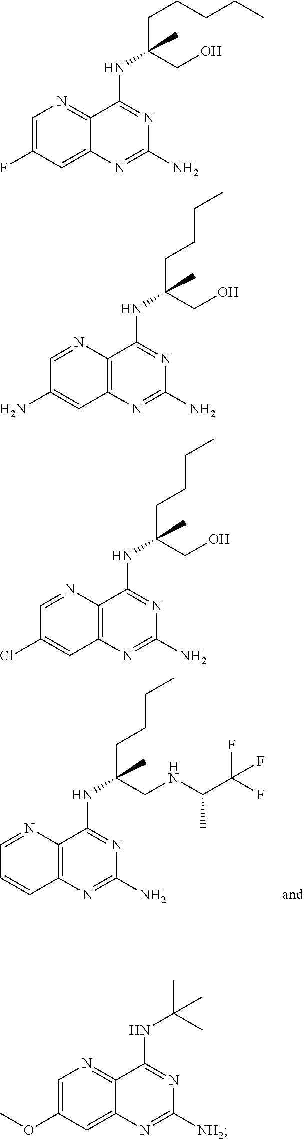
or a pharmaceutically acceptable salt thereof.

15. The method of claim 14, wherein the TLR8 modulating compound is selected from the group consisting of: 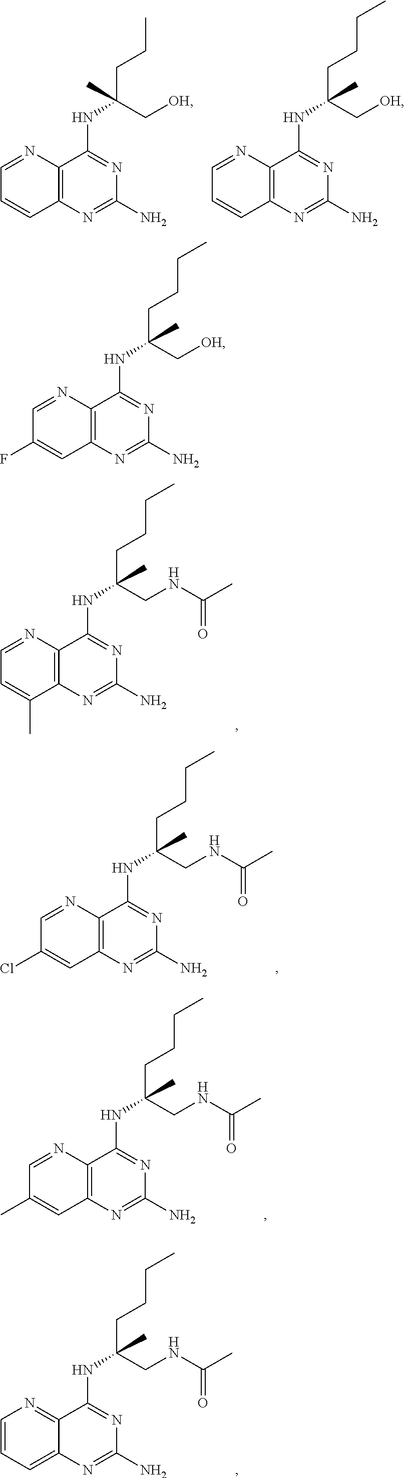 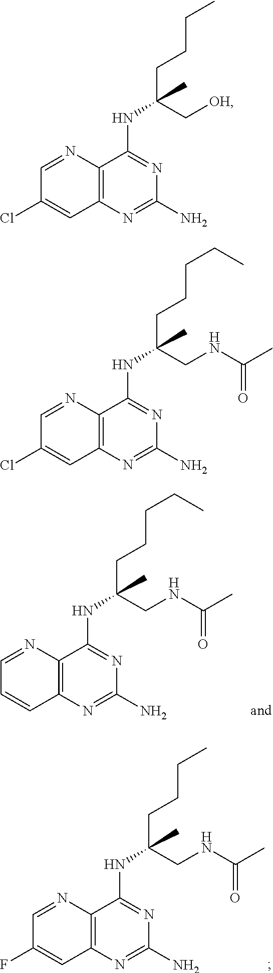
or a pharmaceutically acceptable salt thereof.

16. The method of any of claims 1-8 wherein the TLR8 modulating compound is 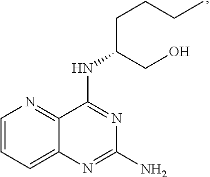 [SMILES: CCCC[C@H](CO)Nc1nc(N)nc2cccnc12 ; ","]
or a pharmaceutically acceptable salt thereof.

17. The method of any of claims 1 through 16 further comprising the step of administering to the human in need thereof a pharmaceutically effective amount of an immunomodulatory cytokine.

18. The method of claim 17 wherein the immunomodulatory cytokine is selected from the group of IL-2, IL-4, IL-6, IL-7, IL-12, IL-15, IL-17, and IL-21.

19. The method of any of claim 17 or 18 wherein the immunomodulatory cytokine is IL-15.

20. The method of any of claims 1 through 16 comprising the further step of administering to the human in need thereof a pharmaceutically effective amount of an agent selected from the group of IFN-α, IFN-β, IFN-γ, GM-CSF, G-CSF, and M-CSF.

21. The method of any of claims 1-16, comprising the further step of administering to the human in need thereof a pharmaceutically effective amount of an immunomodulatory monoclonal antibody or immunomodulatory small molecule agent.

22. The method of claim 21, wherein the immunomodulatory monoclonal antibody is an inhibitory anti-PD-1 monoclonal antibody or inhibitory anti-PD-L1 monoclonal antibody.

23. The method of claim 22, wherein the inhibitory anti-PD-1 monoclonal antibody is Nivolimumab, Pembrolizumab, BMS-936559, MPDL3280A, MED14736, MSB0010718C, or MDX1105-01.

24. The method of claim 21, wherein the immunomodulatory small molecule agent is an IDO inhibitor.

25. The method of claim 24, wherein the immunomodulatory small molecule agent is 1-methyl-D-tryptophan, NLG919, epacadostat, F-001287, resminostat, SN-35837, NLG-919, GDC-0919, or indoximod.

26. A pharmaceutical composition comprising a pharmaceutically acceptable excipient and:
a) a pharmaceutically effective amount of an antiretroviral agent; and
b) a pharmaceutically effective amount of a TLR8 modulating compound.

27. A pharmaceutical composition comprising a pharmaceutically acceptable excipient and:
a) a pharmaceutically effective amount of each of two or more antiretroviral agents; and
b) a pharmaceutically effective amount of a TLR8 modulating compound.

28. The pharmaceutical composition of any of claims 26-27, wherein the TLR8 modulating compound is a compound of Formula (IV), 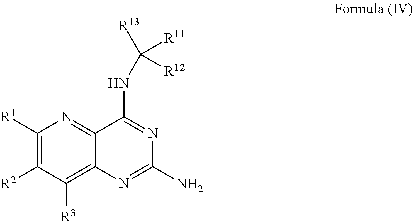
or a pharmaceutically acceptable salt thereof, wherein:
R¹ is selected from the group consisting of hydrogen, halogen, C₁₋₆ alkyl, CN, and OR^{a}, wherein C₁₋₆ alkyl is optionally substituted with 1 to 5 R²⁰ groups;
R² is selected from the group consisting of hydrogen, halogen, C₁₋₆ alkyl, CN, and OR^{a}, wherein C₁₋₆ alkyl optionally substituted with 1 to 5 R²⁰ groups;
R³ is selected from the group consisting of hydrogen, halogen, C₁₋₆ alkyl, CN, and OR^{a}, wherein C₁₋₆ alkyl is optionally substituted with 1 to 5 R²⁰ groups;
R¹¹ is selected from the group consisting of hydrogen, C₁₋₂ alkyl, C₃₋₆ cycloalkyl, and C₁₋₃ haloalkyl;
R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
each R²° is independently selected from the group consisting of halogen, CN, —NR^{a}R^{b}, and OR^{a}; and
each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

29. The pharmaceutical composition of claim 28, wherein the TLR8 modulating compound is a compound of Formula (IVa)

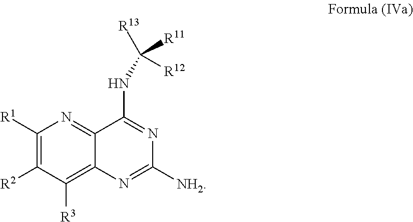

30. The pharmaceutical composition of claim 28, wherein the TLR8 modulating compound is a compound of Formula (IVb)

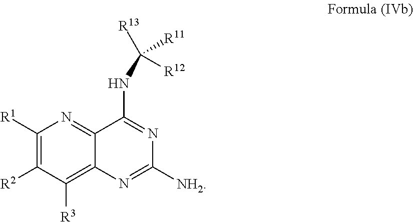

31. The pharmaceutical composition of claim 29, wherein the moiety

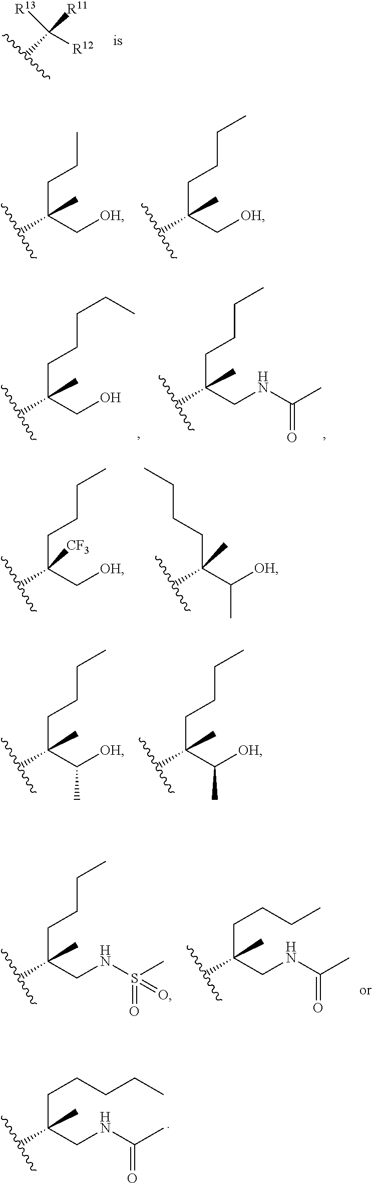

32. The pharmaceutical composition of claim 30, wherein the moiety

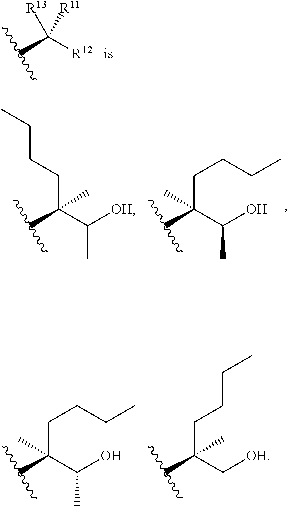

33. The pharmaceutical composition of claims 26-27, wherein the TLR8 modulating compound is selected from the group consisting of: 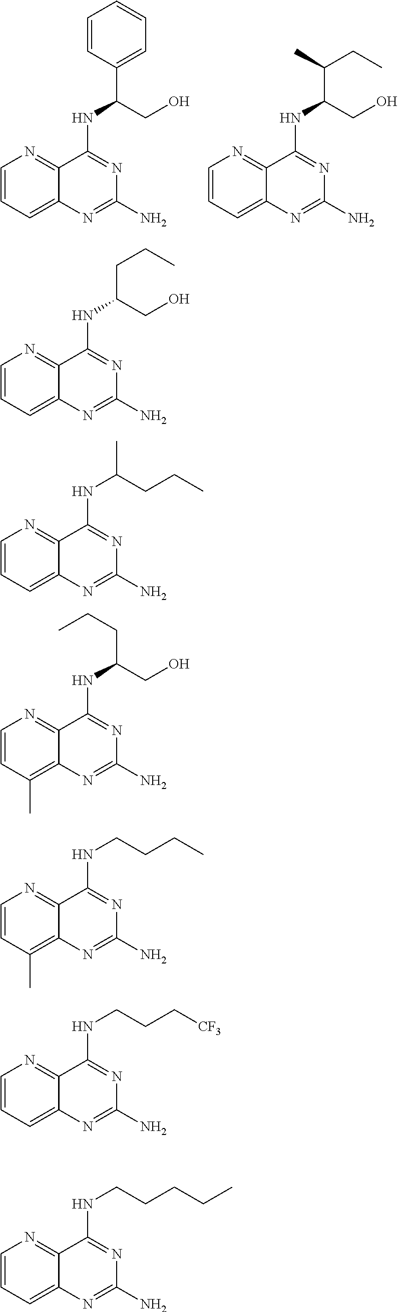 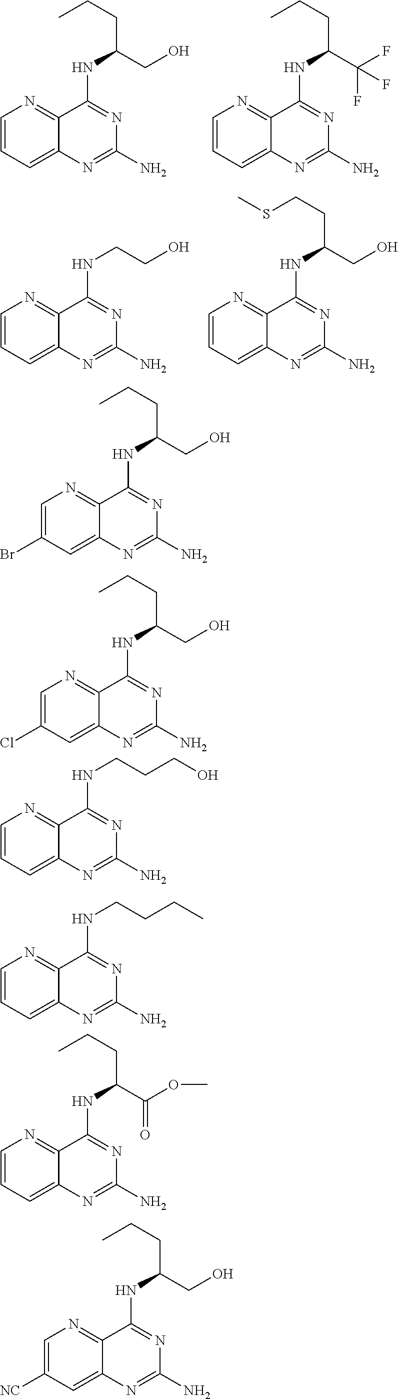 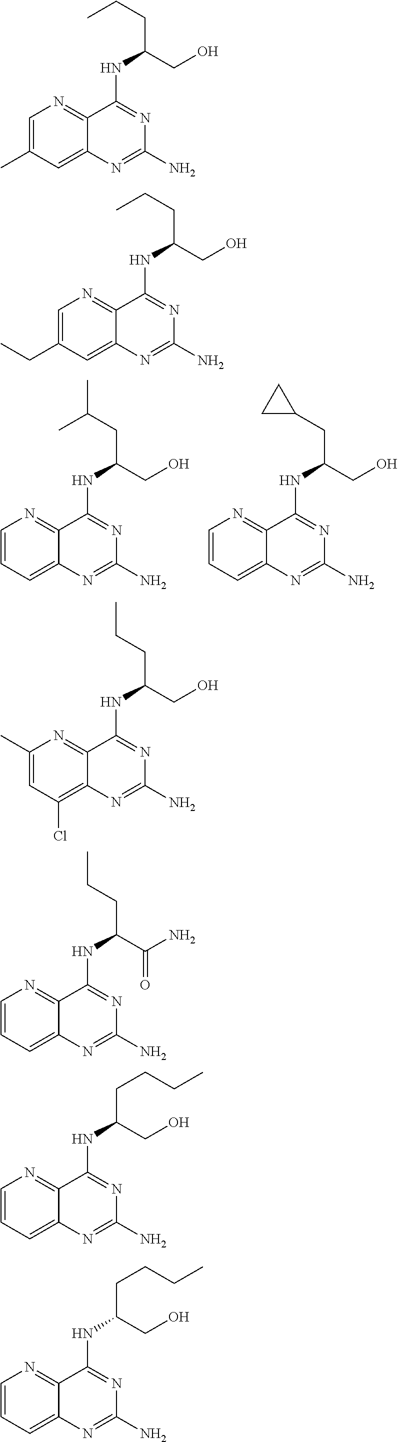 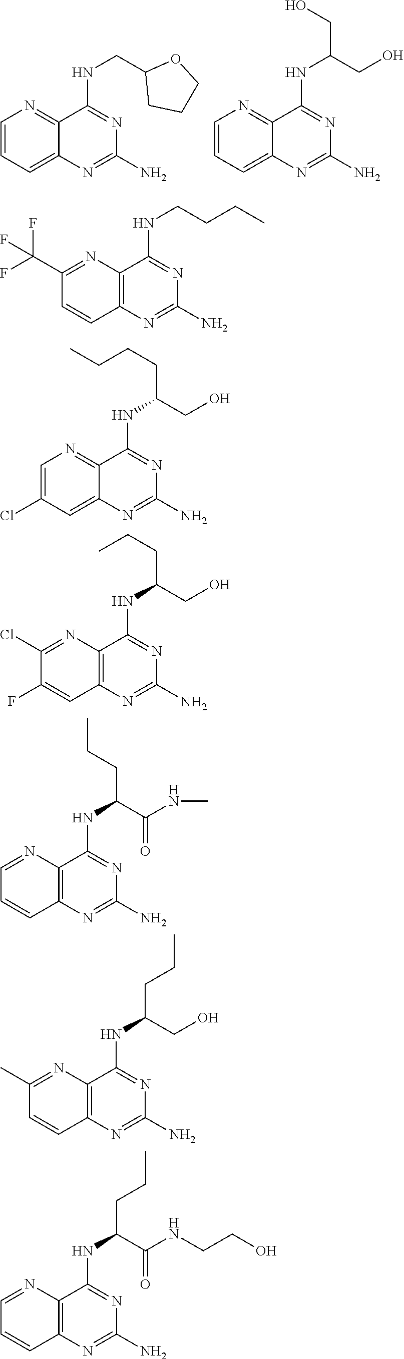 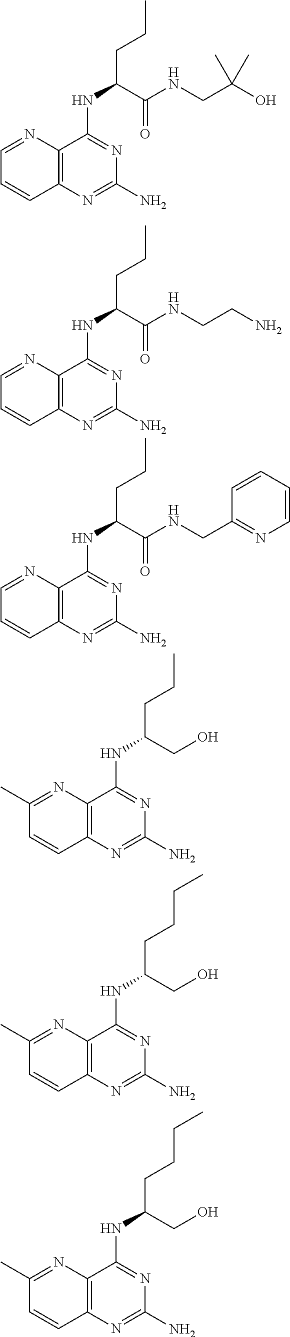 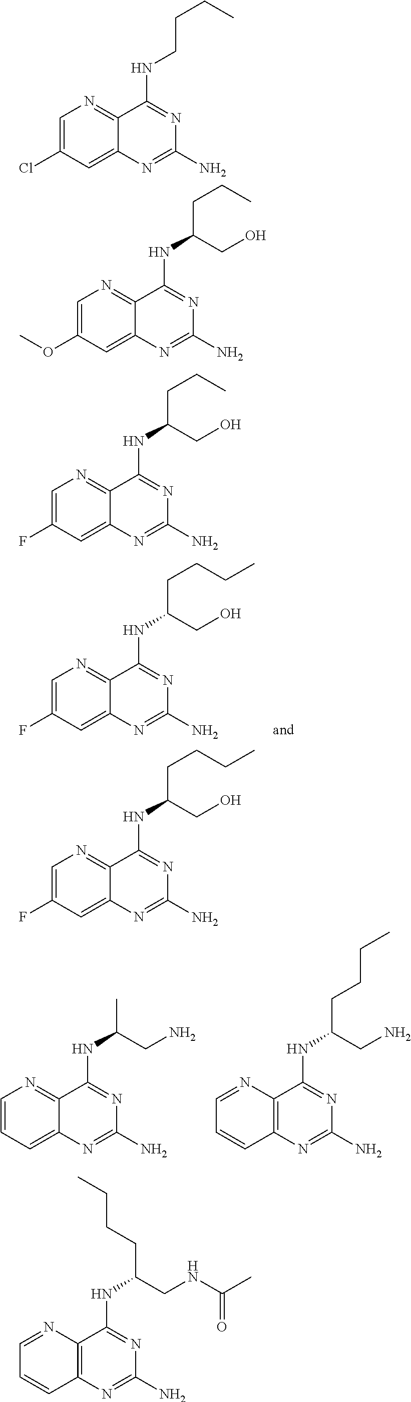 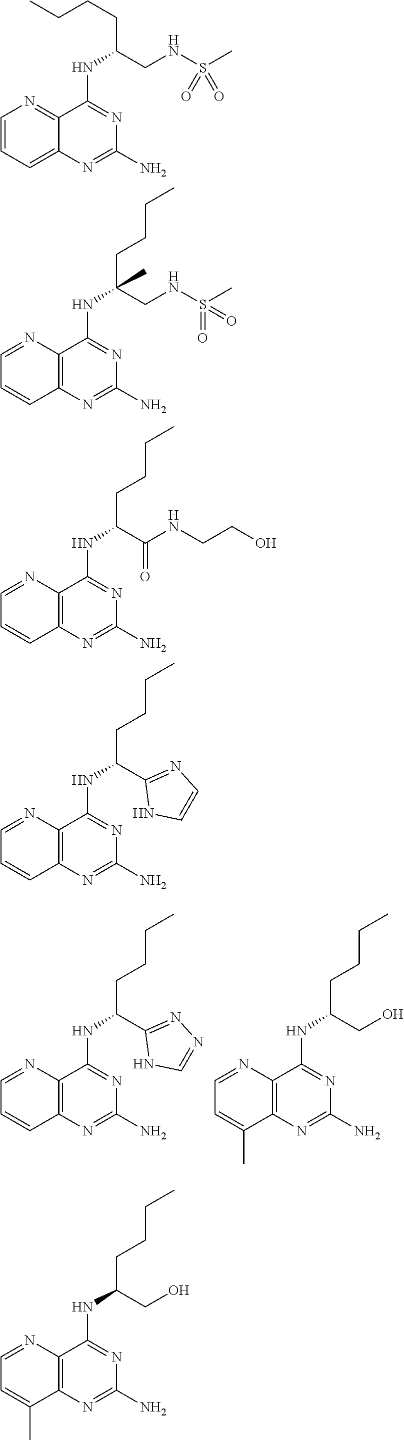 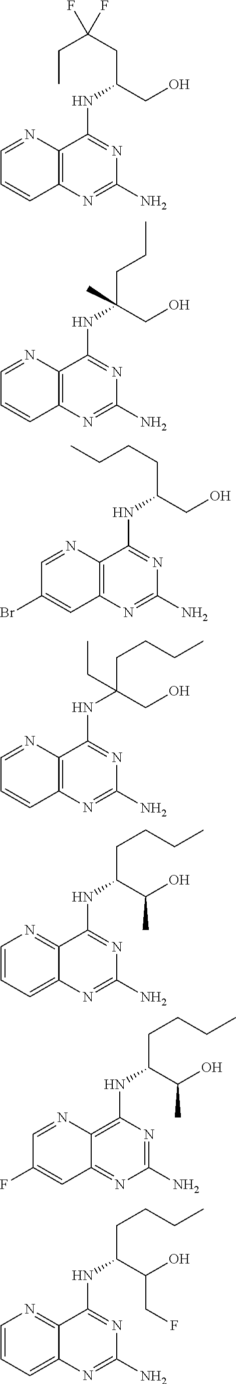 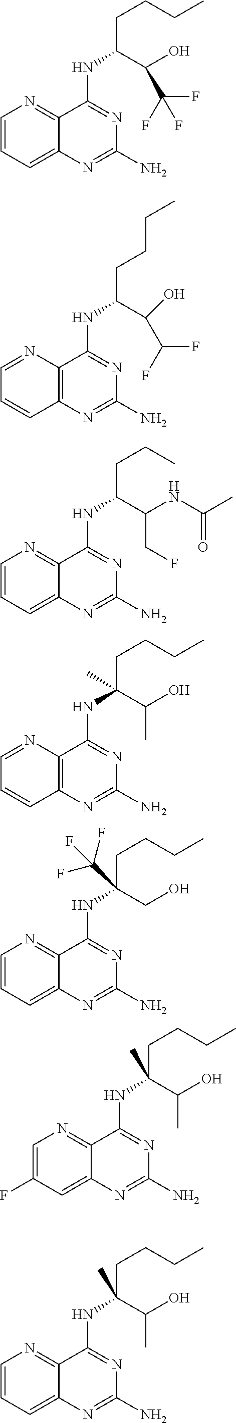 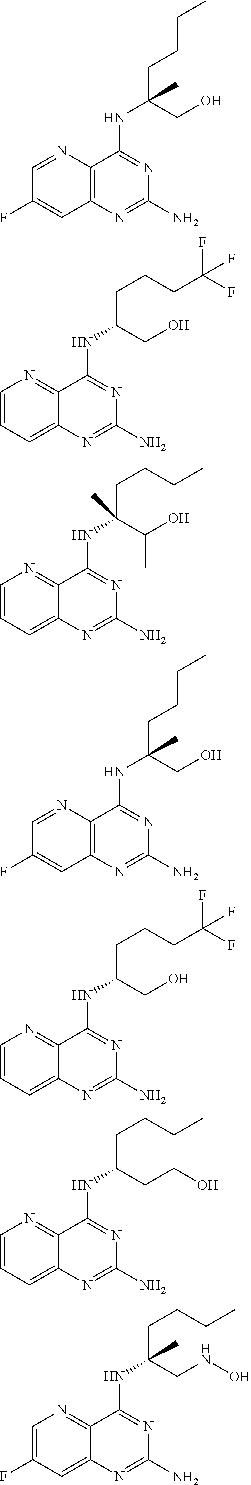 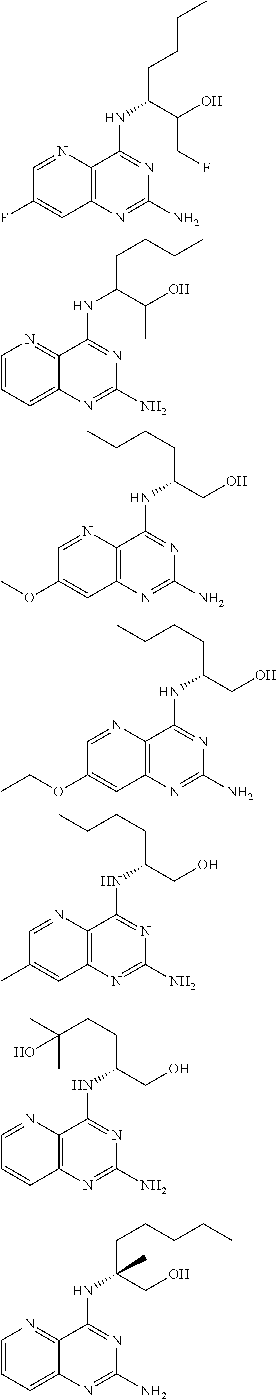 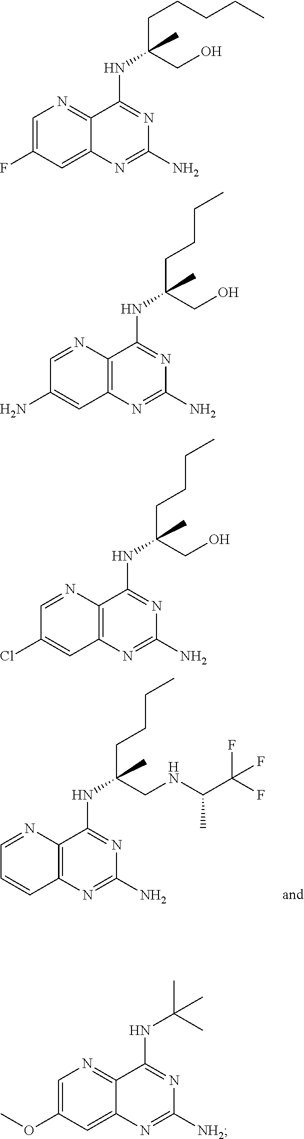
or a pharmaceutically acceptable salt thereof.

34. The pharmaceutical composition of claim 33, wherein the TLR8 modulating compound is selected from the group consisting of: 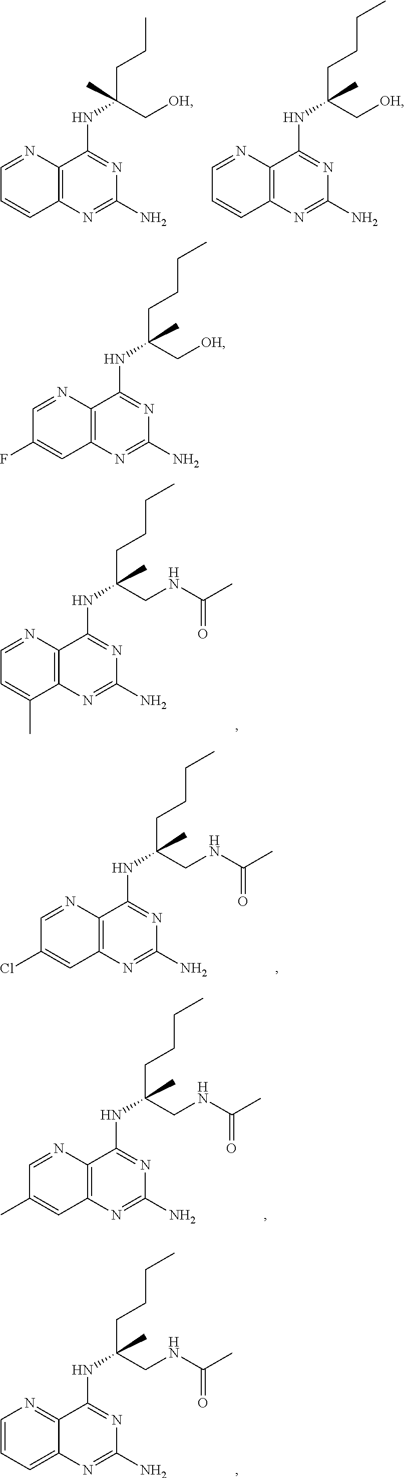 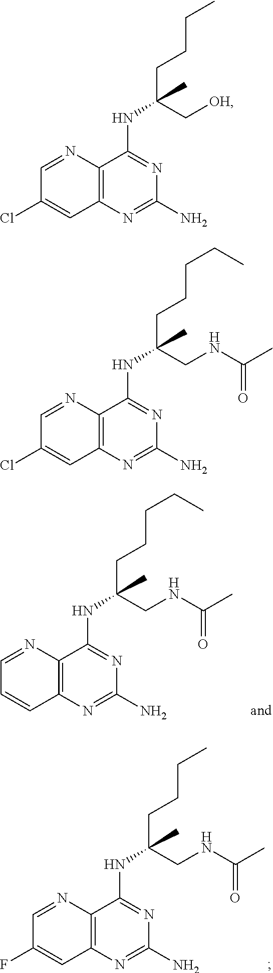
or a pharmaceutically acceptable salt thereof.

35. The pharmaceutical composition of any of claims 26-27, wherein the TLR8 modulating compound is 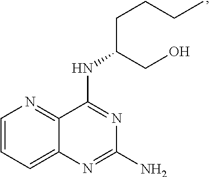 [SMILES: CCCC[C@H](CO)Nc1nc(N)nc2cccnc12 ; ","]
or a pharmaceutically acceptable salt thereof.

36. The pharmaceutical composition of any of claims 27-35 further comprising a pharmaceutically effective amount of a latency-reversing agent.

37. The use of a TLR8 modulating compound and an antiretroviral agent, or a pharmaceutically acceptable salt thereof, in the preparation of a medicament for treating an HIV infection in a human.

38. The use of a TLR8 modulating compound and a latency-reversing agent, or a pharmaceutically acceptable salt thereof, in the preparation of a medicament for treating an HIV infection in a human.

39. The use of a TLR8 modulating compound, or a pharmaceutically acceptable salt thereof, in the preparation of a medicament for enhancing the efficacy of an HIV vaccine.

40. The use of a TLR8 modulating compound, or a pharmaceutically acceptable salt thereof, in the preparation of a medicament for eliminating an HIV infection in a human.

41. The use of a TLR8 modulating compound, or a pharmaceutically acceptable salt thereof, in the preparation of a medicament for enhancing the efficacy of antiviral agent.

42. The use of any of claims 37 through 41 wherein the TLR8 modulating compound is as described in any of claims 9 through 15.

43. A TLR8 modulating compound, or a pharmaceutically acceptable salt thereof, for use in the treatment of an HIV infection in a human.

44. A TLR8 modulating compound, or a pharmaceutically acceptable salt thereof, for use in treating an HIV infection in a virologically suppressed human.

45. A TLR8 modulating compound, or a pharmaceutically acceptable salt thereof, for use in inducing HIV gene expression in a human infected with HIV.

46. A TLR8 modulating compound for use in inducing HIV gene expression in a human infected with HIV wherein active HIV gene expression in the human has been suppressed by administration of antiretroviral therapy.

47. A TLR8 modulating compound for use in inducing HIV gene expression in a latent HIV reservoir in a human infected with HIV.

48. A TLR8 modulating compound for use in enhancing HIV gene expression in HIV infected cells in a human infected with HIV.

49. A TLR8 modulating compound for use in lowering the chronic set point of HIV viral load in a human infected with HIV.

50. A TLR8 modulating compound for use in inducing transient HIV-1 viremia in a virologically suppressed human infected with HIV-1.

51. A TLR8 modulating compound for use in reducing HIV viremia in a human infected with HIV.

52. A TLR8 modulating compound for use in enhancing immune cell activity and increasing HIV gene expression in a human infected with HIV.

53. A TLR8 modulating compound for use in enhancing the efficacy of an antiviral agent in a human infected with HIV.

54. A TLR8 modulating compound for use in enhancing the efficacy of an HIV vaccine.

55. A TLR8 modulating compound for use in eliminating an HIV infection in a human.
